SEQUENCE LISTING (17 sequences)

SEQ ID NO: 1 - DNA, 69 bp, Artificial Sequence
  Description of Artificial Sequence: Synthetic oligonucleotide
cagtctgacacagcagtcgttaatcgtcgctgctacccttgacatccgtgactggctagacagaggtgt

SEQ ID NO: 2 - DNA, 69 bp, Artificial Sequence
  Description of Artificial Sequence: Synthetic oligonucleotide
gacctgacctacagtgaccatagccttgcctgattagccactgtccagtttggctcctggtctcactag

SEQ ID NO: 3 - DNA, 73 bp, Artificial Sequence
  Description of Artificial Sequence: Synthetic oligonucleotide
gagtacacctctgtctagccagtcacggatgtcaagggtagcagcgacgattaacgactgctgtgtcagactg

SEQ ID NO: 4 - DNA, 73 bp, Artificial Sequence
  Description of Artificial Sequence: Synthetic oligonucleotide
actcctagtgagaccaggagccaaactggacagtggctaatcaggcaaggctatggtcactgtaggtcaggtc

SEQ ID NO: 5 - DNA, 20 bp, Artificial Sequence
  Description of Artificial Sequence: Synthetic oligonucleotide
cagtctgacacagcagtcgt

SEQ ID NO: 6 - DNA, 20 bp, Artificial Sequence
  Description of Artificial Sequence: Synthetic oligonucleotide
gacctgacctacagtgacca

SEQ ID NO: 7 - DNA, 21 bp, Artificial Sequence
  Description of Artificial Sequence: Synthetic probe
agaggtgtactcctagtgaga

SEQ ID NO: 8 - DNA, 69 bp, Artificial Sequence
  Description of Artificial Sequence: Synthetic oligonucleotide
cagtctgactcaccactcgttaatcgtcgctgctacccttgacatccgtgactggctagacagaggtgt

SEQ ID NO: 9 - DNA, 69 bp, Artificial Sequence
  Description of Artificial Sequence: Synthetic oligonucleotide
caccagacctacgaagtccatagccttgcctgattagccactgtccagtttggctcctggtctcactag

SEQ ID NO: 10 - DNA, 73 bp, Artificial Sequence
  Description of Artificial Sequence: Synthetic oligonucleotide
gagtacacctctgtctagccagtcacggatgtcaagggtagcagcgacgattaacgagtggtgagtcagactg

SEQ ID NO: 11 - DNA, 73 bp, Artificial Sequence
  Description of Artificial Sequence: Synthetic oligonucleotide
actcctagtgagaccaggagccaaactggacagtggctaatcaggcaaggctatggacttcgtaggtctggtg

SEQ ID NO: 12 - DNA, 20 bp, Artificial Sequence
  Description of Artificial Sequence: Synthetic oligonucleotide
cagtctgactcaccactcgt

SEQ ID NO: 13 - DNA, 20 bp, Artificial Sequence
  Description of Artificial Sequence: Synthetic oligonucleotide
caccagacctacgaagtcca

SEQ ID NO: 14 - DNA, 40 bp, Artificial Sequence
  Description of Artificial Sequence: Synthetic oligonucleotide
cagtctgacacagcagtcgtgactggctagacagaggtgt

SEQ ID NO: 15 - DNA, 40 bp, Artificial Sequence
  Description of Artificial Sequence: Synthetic oligonucleotide
gacctgacctacagtgaccattggctcctggtctcactag

SEQ ID NO: 16 - DNA, 44 bp, Artificial Sequence
  Description of Artificial Sequence: Synthetic oligonucleotide
gagtacacctctgtctagccagtcacgactgctgtgtcagactg

SEQ ID NO: 17 - DNA, 44 bp, Artificial Sequence
  Description of Artificial Sequence: Synthetic oligonucleotide
actcctagtgagaccaggagccaatggtcactgtaggtcaggtc

(19) United States
(12) Patent Application Publication
(10) Pub. No.: US 2023/0243834 A1
(43) Pub. Date: Aug. 3, 2023

(54) COMPOSITIONS AND METHODS FOR DETECTION OF OVARIAN CANCER

(71) Applicant: Mercy BioAnalytics, Inc., Natick, MA (US)

(72) Inventors: Joseph Charles Sedlak, Cambridge, MA (US); Laura Teresa Bortolin, Newtonville, MA (US); Daniel Parker Salem, Somerville, MA (US)

(21) Appl. No.: 17/793,382

(22) PCT Filed: Jan. 16, 2021
(86) PCT No.: PCT/US2021/013776

§ 371 Date: Jul. 15, 2022

Related U.S. Application Data
(60) Provisional application No. 62/962,711, filed on Jan. 17, 2020.
(60) Provisional application No. 63/049,063, filed on Jul. 7, 2020.

(51) Int. Cl. G01N 33/574; C12Q 1/6886; C12Q 1/6804

(52) U.S. Cl. CPC G01N 33/57449; C12Q 1/6804; C12Q 1/6886; C12Q 2600/158

(57) ABSTRACT

The present disclosure in one aspect provides technologies for detection of ovarian cancer, e.g., early detection of ovarian cancer. In another aspect, technologies provided herein are useful for selecting and/or monitoring and/or evaluating efficacy of, a treatment administered to a subject determined to have or susceptible to ovarian cancer. In some embodiments, technologies provided herein are useful for development of companion diagnostics, e.g., by measuring tumor burdens and changes in tumor burdens in conjunction with therapeutics. In some embodiments, technologies provided herein are useful for development of companion diagnostics, e.g., by identifying biomarkers in woman’s blood samples that are associated with therapeutic response.

### CROSS REFERENCE TO RELATED APPLICATIONS

This application claims the benefit of U.S. Provisional Application No. 62/962,711 filed Jan. 17, 2020, and U.S. Provisional Application No. 63/049,063 filed Jul. 07, 2020, the contents of each of which are hereby incorporated herein in their entirety.

### BACKGROUND

Early detection of cancer greatly increases the chance of successful treatment. However, many cancers including ovarian cancer still lack effective screening recommendations. Typical challenges for cancer-screening tests include limited sensitivity and specificity. A high rate of false-positive results can be of particular concern, as it can create difficult management decisions for clinicians and patients who would not want to unnecessarily administer (or receive) anti-cancer therapy that may potentially have undesirable side effects. Conversely, a high rate of false-negative results fails to satisfy the purpose of the screening test, as patients who need therapy are missed, resulting in a treatment delay and consequently a reduced possibility of success.

### SUMMARY

The present disclosure, among other things, provides insights and technologies for achieving effective ovarian cancer screening. In some embodiments, provided technologies are effective for detection of early stage ovarian cancers. In some embodiments, provided technologies are effective even when applied to populations comprising or consisting of asymptomatic individuals (e.g., due to sufficiently high sensitivity and/or low rates of false positive and/or false negative results). In some embodiments, provided technologies are effective when applied to populations comprising or consisting of individuals (e.g., asymptomatic individuals) without hereditary risk in developing ovarian cancer. In some embodiments, provided technologies are effective when applied to populations comprising or consisting of symptomatic individuals (e.g., individuals suffering from one or more symptoms of ovarian cancer). In some embodiments, provided technologies are effective when applied to populations comprising or consisting of individuals at risk for ovarian cancer (e.g., individuals with hereditary and/or life-history associated risk factors for ovarian cancer). In some embodiments, provided technologies may be or include one or more compositions (e.g., molecular entities or complexes, systems, cells, collections, combinations, kits, etc.) and/or methods (e.g., of making, using, assessing, etc.), as will be clear to one skilled in the art reading the disclosure provided herein.

In some embodiments, the present disclosure identifies the source of a problem with certain prior technologies including, for example, certain conventional approaches to detection and diagnosis of ovarian cancer. For example, the present disclosure appreciates that many conventional diagnostic assays, e.g., based on cell-free nucleic acids, serum proteins (e.g., CA-125, which is a portion of a MUC16 polypeptide), and/or bulk analysis of extracellular vesicles, can be time-consuming, costly, and/or lacking sensitivity and/or specificity sufficient to provide a reliable and comprehensive diagnostic assessment. In some embodiments, the present disclosure provides technologies (including systems, compositions, and methods) that solve such problems, among other things, by detecting co-localization of a target biomarker signature of ovarian cancer in individual extracellular vesicles, which comprises at least one extracellular vesicle-associated membrane-bound polypeptide and at least one target biomarker selected from the group consisting of surface protein biomarkers, internal protein biomarkers, and RNA biomarkers. In some embodiments, the present disclosure provides technologies (including systems, compositions, and methods) that solve such problems, among other things, by detecting such target biomarker signature of ovarian cancer using a target entity detection approach that was developed by Applicant and described in U.S. Application No. 16/805,637, and International Application PCT/US2020/020529, both filed Feb. 28, 2020 and entitled “Systems, Compositions, and Methods for Target Entity Detection,” which are based on interaction and/or co-localization of at least two or more target entities (e.g., a target biomarker signature) in individual extracellular vesicles.

In some embodiments, the present disclosure, among other things, provides insights that screening of asymptotic individuals, e.g., regular screening prior to or otherwise in absence of developed symptom(s), can be beneficial, and even important for effective management (e.g., successful treatment) of ovarian cancer. In some embodiments, the present disclosure provides ovarian cancer screening systems that can be implemented to detect ovarian cancer, including early-stage cancer, in some embodiments in asymptomatic individuals (e.g., without hereditary risks in ovarian cancer). In some embodiments, provided technologies are implemented to achieve regular screening of asymptomatic individuals (e.g., without hereditary risks in ovarian cancer). The present disclosure provides, for example, compositions (e.g., reagents, kits, components, etc.), and methods of providing and/or using them, including strategies that involve regular testing of one or more individuals (e.g., asymptomatic individuals). The present disclosure defines usefulness of such systems, and provides compositions and methods for implementing them.

In some embodiments, provided technologies achieve detection (e.g., early detection, e.g., in asymptomatic individual(s) and/or population(s)) of one or more features (e.g., incidence, progression, responsiveness to therapy, recurrence, etc.) of ovarian cancer, with sensitivity and/or specificity (e.g., rate of false positive and/or false negative results) appropriate to permit useful application of provided technologies to single-time and/or regular (e.g., periodic) assessment. In some embodiments, provided technologies are useful in conjunction with women’s periodic physical examination such as mammogram, HPV, and/or Pap smear screening. In some embodiments, provided technologies are useful in conjunction with treatment regimen(s); in some embodiments, provided technologies may improve one or more characteristics (e.g., rate of success according to an accepted parameter) of such treatment regimen(s).

In some aspects, provided are technologies for use in classifying a subject (e.g., an asymptomatic subject) as having or being susceptible to ovarian cancer. In some embodiments, the present disclosure provides methods or assays for classifying a subject (e.g., an asymptomatic subject) as having or being susceptible to ovarian cancer. In some embodiments, a provided method or assay comprises (a) detecting, in a blood-derived sample from a subject in need thereof, extracellular vesicles expressing a target biomarker signature of ovarian cancer, the target biomarker signature comprising: at least one extracellular vesicle-associated membrane-bound polypeptide and at least one target biomarker selected from the group consisting of: surface protein biomarkers, intravesicular protein biomarkers, and intravesicular RNA biomarkers, wherein the surface protein biomarkers are selected from AQP5, CDH6, CHODL, CLDN3, CLDN6, CLDN16, EpCAM, FOLR1, HTR3A, LEMD1, LRRTM1, MUC16, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof; the intravesicular protein biomarkers are selected from CRABP2, KLK7, MIF, PRAME, and S100A1, and combinations thereof; and the intravesicular RNA (e.g., mRNA) biomarkers are selected from CLDN6, CRABP2, KLK7, MIF, PRAME, S100A1, and combinations thereof; (b) comparing sample information indicative of level of the target biomarker signature-expressing extracellular vesicles in the blood-derived sample to reference information including a reference threshold level; and (c) classifying the subject as having or being susceptible to ovarian cancer when the blood-derived sample shows an elevated level of target biomarker signature-expressing extracellular vesicles relative to a classification cutoff referencing the reference threshold level.

In some embodiments, methods or assays described herein may be performed for one more additional target biomarker signature. In some such embodiments, a classification cutoff may reference additional reference threshold level(s) corresponding to the additional target biomarker signature.

In some embodiments, an extracellular vesicle-associated membrane-bound polypeptide for use in a target biomarker signature of ovarian cancer used and/or described herein may be or comprise a tumor-specific biomarker and/or a tissue-specific biomarker (e.g., an ovarian tissue-specific biomarker). In some embodiments, such an extracellular vesicle-associated membrane-bound polypeptide may be or comprise a non-specific marker, e.g., it is present in one or more non-target tumors, and/or in one or more non-target tissues. In some embodiments, such an extracellular vesicle-associated membrane-bound polypeptide biomarker may be or comprise one or more of AQP5, CDH6, CHODL, CLDN3, CLDN6, CLDN16, EpCAM, FOLR1, HTR3A, LEMD1, LRRTM1, MUC16, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and SLC34A2.

In some embodiments, an extracellular vesicle-associated membrane-bound polypeptide biomarker may be or comprise a SLC34A2 polypeptide, an AQP5 polypeptide, a MUC16 polypeptide, a CLDN3 polypeptide, a CLDN6 polypeptide, a FOLR1 polypeptide, an ALPL polypeptide, a BST2 polypeptide, a CD24 polypeptide, a MSLN polypeptide, a MUC1 polypeptide, a PTGS1 polypeptide, a sTn polypeptide glycosylation, a TACSTD2 polypeptide, and/or a LRRTM1 polypeptide. In some embodiments, such an extracellular vesicle-associated membrane-bound polypeptide may be or comprise a SLC34A2 polypeptide. In some embodiments, such an extracellular vesicle-associated membrane-bound polypeptide may be or comprise a MUC16 polypeptide. In some embodiments, such an extracellular vesicle-associated membrane-bound polypeptide may be or comprise a FOLR1 polypeptide. In some embodiments, such an extracellular vesicle-associated membrane-bound polypeptide may be or comprise a LRRTM1 polypeptide. In some embodiments, such an extracellular vesicle-associated membrane-bound polypeptide may be or comprise a TACSTD2 polypeptide. In some embodiments, such an extracellular vesicle-associated membrane-bound polypeptide may be or comprise a CD24 polypeptide. In some embodiments, such an extracellular vesicle-associated membrane-bound polypeptide may be or comprise a PTGS1 polypeptide. In some embodiments, such an extracellular vesicle-associated membrane-bound polypeptide may be or comprise a MUC1 polypeptide. In some embodiments, such an extracellular vesicle-associated membrane-bound polypeptide may be or comprise a sTn polypeptide glycosylation. In some embodiments, such an extracellular vesicle-associated membrane-bound polypeptide may be or comprise a MSLN polypeptide. In some embodiments, such an extracellular vesicle-associated membrane-bound polypeptide may be or comprise an ALPL polypeptide. In some embodiments, such an extracellular vesicle-associated membrane-bound polypeptide may be or comprise a BST2 polypeptide. In some embodiments, such an extracellular vesicle-associated membrane-bound polypeptide may be or comprise a CLDN3 polypeptide.

In some embodiments, a target biomarker signature of ovarian cancer may comprise an extracellular vesicle-associated membrane-bound polypeptide (e.g., ones described herein) and at least one additional target surface protein biomarker, which, in some embodiments, may be or comprise AQP5, CDH6, CHODL, CLDN3, CLDN6, CLDN16, EpCAM, FOLR1, HTR3A, LEMD1, LRRTM1, MUC16, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and/or any combinations thereof.

In some embodiments, a target biomarker signature of ovarian cancer may comprise an extracellular vesicle-associated membrane-bound polypeptide (e.g., ones described herein) and at least one target intravesicular RNA (e.g., mRNA) biomarker, which, in some embodiments, may be or comprise CLDN6, CRABP2, KLK7, MIF, PRAME, S100A1, and combinations thereof.

In some embodiments, a target biomarker signature of ovarian cancer may comprise an extracellular vesicle-associated membrane-bound polypeptide (e.g., ones described herein) and at least one additional target intravesicular protein biomarker, which, in some embodiments, may be or comprise CRABP2, KLK7, MIF, PRAME, and S100A1, and combinations thereof.

In some embodiments, a target biomarker signature comprises at least one extracellular vesicle-associated membrane-bound polypeptide, which is or comprises a SCL34A2 polypeptide and/or a CLDN6 polypeptide; and at least one target biomarker MUC16.

In some embodiments, a target biomarker signature comprises at least one extracellular vesicle-associated membrane-bound polypeptide, which is or comprises a MUC16 polypeptide; and at least one target biomarker MUC16.

In some embodiments, a target biomarker signature comprises at least one extracellular vesicle-associated membrane-bound polypeptide, which is or comprises a SLC34A2 polypeptide; and at least two target biomarkers MUC16 and FOLR1.

In some embodiments, a target biomarker signature comprises at least one extracellular vesicle-associated membrane-bound polypeptide, which is or comprises a SLC34A2 polypeptide; and at least two target biomarkers SLC34A2 and FOLR1.

In some embodiments, a target biomarker signature comprises at least one extracellular vesicle-associated membrane-bound polypeptide, which is or comprises a SLC34A2 polypeptide; and at least one target biomarker MUC16.

In some embodiments, a target biomarker signature comprises at least one extracellular vesicle-associated membrane-bound polypeptide, which is or comprises a SCL34A2 polypeptide; and at least one target biomarker FOLR1.

In some embodiments, a target biomarker signature comprises at least one extracellular vesicle-associated membrane-bound polypeptide, which is or comprises a MUC16 polypeptide; and at least two target biomarkers MUC16 and CLDN6.

In some embodiments, a target biomarker signature comprises at least one extracellular vesicle-associated membrane-bound polypeptide, which is or comprises a MUC16 polypeptide; and at least two target biomarkers MUC16 and CLDN3.

In some embodiments, a target biomarker signature comprises at least one extracellular vesicle-associated membrane-bound polypeptide, which is or comprises a MUC16 polypeptide; and at least two target biomarkers FOLR1 and CLDN3.

In some embodiments, a target biomarker signature comprises at least one extracellular vesicle-associated membrane-bound polypeptide, which is or comprises a MUC16 polypeptide; and at least two target biomarkers MUC16 and FOLR1.

In some embodiments, a target biomarker signature comprises at least one extracellular vesicle-associated membrane-bound polypeptide, which is or comprises a MUC16 polypeptide; and at least one target biomarker FOLR1.

In some embodiments, a target biomarker signature comprises at least one extracellular vesicle-associated membrane-bound polypeptide, which is or comprises a MUC16 polypeptide; and at least two target biomarkers SLC34A2 and MUC16.

In some embodiments, a target biomarker signature comprises at least one extracellular vesicle-associated membrane-bound polypeptide, which is or comprises a MUC16 polypeptide; and at least two target biomarkers SLC34A2 and FOLR1.

In some embodiments, a target biomarker signature comprises at least one extracellular vesicle-associated membrane-bound polypeptide, which is or comprises a MUC16 polypeptide; and at least two target biomarkers MUC16 and AQP5.

In some embodiments, a target biomarker signature comprises at least one extracellular vesicle-associated membrane-bound polypeptide, which is or comprises a MUC16 polypeptide; and at least two target biomarkers FOLR1 and AQP5.

In some embodiments, a target biomarker signature comprises at least one extracellular vesicle-associated membrane-bound polypeptide, which is or comprises a FOLR1 polypeptide; and at least one target biomarker MUC16.

In some embodiments, a target biomarker signature comprises at least one extracellular vesicle-associated membrane-bound polypeptide, which is or comprises a FOLR1 polypeptide; and at least one target biomarker FOLR1.

In some embodiments, a target biomarker signature comprises at least one extracellular vesicle-associated membrane-bound polypeptide, which is or comprises a FOLR1 polypeptide; and at least two target biomarkers FOLR1 and CLDN6.

In some embodiments, a target biomarker signature comprises at least one extracellular vesicle-associated membrane-bound polypeptide, which is or comprises a FOLR1 polypeptide; and at least two target biomarkers SLC34A2 and CLDN3.

In some embodiments, a target biomarker signature comprises at least one extracellular vesicle-associated membrane-bound polypeptide, which is or comprises a FOLR1 polypeptide; and at least two target biomarkers MUC16 and FOLR1.

In some embodiments, a target biomarker signature comprises at least one extracellular vesicle-associated membrane-bound polypeptide, which is or comprises a FOLR1 polypeptide; and at least two target biomarkers MUC16 and CLDN3.

In some embodiments, a target biomarker signature comprises at least one extracellular vesicle-associated membrane-bound polypeptide, which is or comprises a FOLR1 polypeptide; and at least two target biomarkers SLC34A2 and MUC16.

In some embodiments, a target biomarker signature comprises at least one extracellular vesicle-associated membrane-bound polypeptide, which is or comprises a FOLR1 polypeptide; and at least two target biomarkers FOLR1 and CLDN3.

In some embodiments, a target biomarker signature comprises at least one extracellular vesicle-associated membrane-bound polypeptide, which is or comprises a FOLR1 polypeptide; and at least two target biomarkers FOLR1 and AQP5.

In some embodiments, a target biomarker signature comprises at least one extracellular vesicle-associated membrane-bound polypeptide, which is or comprises a LRRTM1 polypeptide; and at least two target biomarkers MUC16 and MUC16.

In some embodiments, a target biomarker signature comprises at least one extracellular vesicle-associated membrane-bound polypeptide, which is or comprises a CLDN3 polypeptide; and at least one target biomarker FOLR1.

In some embodiments, a target biomarker signature comprises at least one extracellular vesicle-associated membrane-bound polypeptide, which is or comprises a CLDN3 polypeptide; and at least one target biomarker MUC16.

In some embodiments, a target biomarker signature comprises at least one extracellular vesicle-associated membrane-bound polypeptide, which is or comprises a CLDN3 polypeptide; and at least two target biomarkers SLC34A2 and MUC16.

In some embodiments, a target biomarker signature comprises at least one extracellular vesicle-associated membrane-bound polypeptide, which is or comprises a CLDN3 polypeptide; and at least two target biomarkers MUC16 and FOLR1.

In some embodiments, a target biomarker signature comprises at least one extracellular vesicle-associated membrane-bound polypeptide, which is or comprises a CLDN3 polypeptide; and at least two target biomarkers MUC16 and CLDN3.

In some embodiments, a target biomarker signature comprises at least one extracellular vesicle-associated membrane-bound polypeptide, which is or comprises a CLDN3 polypeptide; and at least two target biomarkers MUC16 and CLDN6.

In some embodiments, a reference threshold level for use in a provided method or assay described herein is determined by levels of target biomarker signature-expressing extracellular vesicles observed in comparable samples from a population of non-ovarian cancer subjects.

In some embodiments, an extracellular vesicle-associated membrane-bound polypeptide included in a target biomarker signature may be detected using antibody-based agents. In some embodiments, such an extracellular vesicle-associated membrane-bound polypeptide may be detected using a capture assay comprising an antibody-based agent. For example, in some embodiments, a capture assay for detecting the presence of an extracellular vesicle-associated membrane-bound polypeptide in an extracellular vesicle may involve contacting a blood-derived sample comprising extracellular vesicles with a capture agent directed to such an extracellular vesicle-associated membrane-bound polypeptide. In some embodiments, such a capture agent may comprise a binding moiety directed to an extracellular vesicle-associated membrane-bound polypeptide (e.g., ones described herein), which may be optionally conjugated to a solid substrate. Without limitations, an exemplary capture agent for an extracellular vesicle-associated membrane-bound polypeptide may be or comprising a solid substrate (e.g., a magnetic bead) and a binding moiety (e.g., an antibody agent) directed to an extracellular vesicle-associated membrane-bound polypeptide.

In some embodiments, a target biomarker included in a target biomarker signature may be detected using appropriate methods known in the art, which may vary with types of analytes to be detected (e.g., surface proteins, intravesicular proteins, intravesicular RNA (e.g., mRNA)). For example, a person skilled in the art, reading the present disclosure, will appreciate that a surface protein biomarker and/or an intravesicular protein biomarker may be detected using antibody-based agents in some embodiments, while in some embodiments, an intravesicular RNA (e.g., mRNA) biomarker may be detected using nucleic acid-based agents, e.g., using quantitative reverse transcription PCR.

For example, in some embodiments where a target biomarker is or comprises a surface protein biomarker and/or an intravesicular protein marker, such a target biomarker may be detected involving a proximity ligation assay, e.g., following a capture assay (e.g., ones as described herein) to capture extracellular vesicles that express an extracellular vesicle-associated membrane-bound polypeptide (e.g., ones as used and/or described herein). In some embodiments, such a proximity ligation assay may comprise contacting a blood-derived sample comprising extracellular vesicles with a set of detection probes, each directed to a target biomarker, which set comprises at least two distinct detection probes, so that a combination comprising the extracellular vesicles and the set of detection probes is generated, wherein the two detection probes each comprise: (i) a binding moiety directed to a surface protein biomarker and/or an intravesicular protein biomarker; and (ii) an oligonucleotide domain coupled to the binding moiety, the oligonucleotide domain comprising a double-stranded portion and a single-stranded overhang portion extended from one end of the oligonucleotide domain. Such single-stranded overhang portions of the detection probes are characterized in that they can hybridize to each other when the detection probes are bound to the same extracellular vesicle. Such a combination comprising the extracellular vesicles and the set of detection probes is then maintained under conditions that permit binding of the set of detection probes to their respective targets on the extracellular vesicles such that the detection probes can bind to the same extracellular vesicle to form a double-stranded complex. Such a double-stranded complex can be detected by contacting the double-stranded complex with a nucleic acid ligase to generate a ligated template; and detecting the ligated template. The presence of such a ligated template is indicative of presence of extracellular vesicles that are positive for a target biomarker signature of ovarian cancer. While such a proximity ligation assay may perform better, e.g., with higher specificity and/or sensitivity, than other existing proximity ligation assays, a person skilled in the art reading the present disclosure will appreciate that other forms of proximity ligation assays that are known in the art may be used instead.

In some embodiments where a target biomarker is or comprises an intravesicular RNA (e.g., mRNA) marker, such a target biomarker may be detected involving a nucleic acid detection assay. In some embodiments, an exemplary nucleic acid detection assay may be or comprise reverse-transcription PCR.

In some embodiments where a target biomarker is or comprises an intravesicular biomarker (e.g., an intravesicular protein biomarker and/or an intravesicular RNA (e.g., mRNA) biomarker), such a target biomarker may be detected involving, prior to a detection assay (e.g., a proximity ligation assay as described herein), a sample treatment (e.g., fixation and/or permeabilization) to expose intravesicular biomarker(s) for subsequent detection.

The present disclosure, among other things, recognizes that detection of a single ovarian cancer-associated serum protein or a plurality of ovarian cancer-associated biomarkers based on a bulk sample (e.g., a bulk sample of extracellular vesicles), rather than at a resolution of a single extracellular vesicle, typically does not provide sufficient specificity and/or sensitivity in determination of whether a subject from whom the sample is obtained is likely to be suffering from or susceptible to ovarian cancer. The present disclosure, among other things, provides technologies, including systems, compositions, and/or methods, that solve such problems, including for example by specifically requiring that individual extracellular vesicles for detection be characterized by presence of a target biomarker signature comprising a combination of at least one or more extracellular vesicle-associated membrane-bound polypeptides and at least one or more target biomarkers. In particular embodiments, the present disclosure teaches technologies that require such individual extracellular vesicles be characterized by presence (e.g., by expression) of such a target biomarker signature of ovarian cancer, while extracellular vesicles that do not comprise the target biomarker signature do not produce a detectable signal (e.g., a level that is above a reference level, e.g., by at least 10% or more, where in some embodiments, a reference level may be a level observed in a negative control sample, such as a sample in which individual extracellular vesicles comprising such a target biomarker signature are absent).

Accordingly, in some embodiments, technologies provided herein can be useful for detection of incidence or recurrence of ovarian cancer in a subject and/or across a population of subjects. In some embodiments, a target biomarker signature may be selected for detection of ovarian cancer. In some embodiments, a target biomarker signature may be selected for detection of a specific category of ovarian cancer, including, e.g., but not limited to high-grade serous ovarian cancer, endometrioid ovarian cancer, clear-cell ovarian cancer, low-grade serous ovarian cancer, and/or mucinous ovarian cancer. In some embodiments, technologies provided herein can be used periodically (e.g., every year) to screen a human subject or across a population of human subjects for early-stage ovarian cancer or ovarian cancer recurrence.

In some embodiments, a subject that is amenable to technologies provided herein for detection of incidence or recurrence of ovarian cancer may be an asymptomatic human subject and/or across an asymptomatic population. Such an asymptomatic subject may be a subject who has a family history of ovarian cancer, who has a life history which places them at increased risk for ovarian cancer, who is post-menopausal, who has been previously treated for ovarian cancer, who is at risk of ovarian cancer recurrence after cancer treatment, who is in remission after ovarian cancer treatment, and/or who has been previously or periodically screened for the presence of at least one ovarian cancer biomarker, e.g., but not limited to CA-125 serum proteins. In some embodiments, such an asymptomatic subject may be a subject who is determined to have a normal serum CA-125 level (e.g., a serum CA-125 level of less than 35 U/mL). In some embodiments, such an asymptomatic subject may be a subject who is determined to have a serum CA-125 level of equal to or higher than a normal serum CA-125 level. Alternatively, in some embodiments, an asymptomatic subject may be a subject who has not been previously screened for ovarian cancer, who has not been diagnosed for ovarian cancer, and/or who has not previously received ovarian cancer therapy.

In some embodiments, a subject or population of subjects may be selected based on one or more characteristics such as age, race, genetic history, personal and/or medical history (e.g., smoking, alcohol, drugs, carcinogenic agents, diet, obesity, diabetes, physical activity, sun exposure, radiation exposure, perineal talc use, hormone replacement therapy (HRT), exposure to infectious agents such as viruses, and/or occupational hazard).

In some embodiments, technologies provided herein can be useful for selecting therapy for a subject who is suffering from or susceptible to ovarian cancer. In some embodiments, an ovarian cancer therapy and/or an adjunct therapy can be selected in light of findings based on technologies provided herein.

In some embodiments, technologies provided herein can be useful for monitoring and/or evaluating efficacy of therapy administered to a subject (e.g., an ovarian cancer subject).

In some embodiments, the present disclosure provides technologies for managing patient care, e.g., for one or more individual subjects and/or across a population of subjects. To give but a few examples, in some embodiments, the present disclosure provides technologies that may be utilized in screening (e.g., temporally or incidentally motivated screening and/or non-temporally or incidentally motivated screening, e.g., periodic screening such as annual, semi-annual, bi-annual, or with some other frequency). For example, in some embodiments, provided technologies for use in temporally motivated screening can be useful for screening one or more individual subjects or across a population of subjects (e.g., asymptomatic subjects) who are older than a certain age (e.g., over 50, 55, 60, 65, 70, or older). In some embodiments, provided technologies for use in incidentally motivated screening can be useful for screening individual subjects who may have experienced an incident or event that motivates screening for ovarian cancer as described herein. For example, in some embodiments, an incidental motivation relating to determination of one or more indicators of cancer or susceptibility thereto may be or comprise, e.g., an incident based on their family history (e.g., a close relative such as blood-related relative was previously diagnosed for ovarian cancer), identification of one or more risk factors associated with ovarian cancer (e.g., life history risk factors including, e.g., but not limited to smoking, alcohol, diet, obesity, occupational hazard, etc.) and/or prior incidental findings from genetic tests (e.g., genome sequencing), and/or imaging diagnostic tests (e.g., ultrasound, computerized tomography (CT) and/or magnetic resonance imaging (MRI) scans), development of one or more signs or symptoms characteristic of ovarian cancer (e.g., abnormal bleeding in-between a woman’s period potentially indicative of ovarian cancer, etc.).

In some embodiments, provided technologies for managing patient care can inform treatment and/or payment (e.g., reimbursement for treatment) decisions and/or actions. For example, in some embodiments, provided technologies can provide determination of whether individual subjects have one or more indicators of incidence or recurrence of ovarian cancer, thereby informing physicians and/or patients when to initiate therapy in light of such findings. Additionally or alternatively, in some embodiments, provided technologies can inform physicians and/or patients of treatment selection, e.g., based on findings of specific responsiveness biomarkers (e.g., ovarian cancer responsiveness biomarkers). In some embodiments, provided technologies can provide determination of whether individual subjects are responsive to current treatment, e.g., based on findings of changes in one or more levels of molecular targets associated with ovarian cancer, thereby informing physicians and/or patients of efficacy of such therapy and/or decisions to maintain or alter therapy in light of such findings.

In some embodiments, provided technologies can inform decision making relating to whether health insurance providers reimburse (or not), e.g., for (1) screening itself (e.g., reimbursement available only for periodic/regular screening or available only for temporally and/or incidentally motivated screening); and/or for (2) initiating, maintaining, and/or altering therapy in light of findings by provided technologies. For example, in some embodiments, the present disclosure provides methods relating to (a) receiving results of a screening as described herein and also receiving a request for reimbursement of the screening and/or of a particular therapeutic regimen; (b) approving reimbursement of the screening if it was performed on a subject according to an appropriate schedule or response to a relevant incident and/or approving reimbursement of the therapeutic regimen if it represents appropriate treatment in light of the received screening results; and, optionally (c) implementing the reimbursement or providing notification that reimbursement is refused. In some embodiments, a therapeutic regimen is appropriate in light of received screening results if the received screening results detect a biomarker that represents an approved biomarker for the relevant therapeutic regimen (e.g., as may be noted in a prescribing information label and/or via an approved companion diagnostic). Alternatively or additionally, the present disclosure contemplates reporting systems (e.g., implemented via appropriate electronic device(s) and/or communications system(s)) that permit or facilitate reporting and/or processing of screening results, and/or of reimbursement decisions as described herein.

Some aspects provided herein relate to systems and kits for use in provided technologies. In some embodiments, a system or kit may comprise detection agents for a tumor biomarker signature of ovarian cancer (e.g., ones described herein). In some embodiments, such a system or kit may comprise a capture agent for an extracellular vesicle-associated membrane-bound polypeptide present in extracellular vesicles associated with ovarian cancer (e.g., ones used and/or described herein); and (b) at least one or more detection agents directed to one or more target biomarkers of a target biomarker signature of ovarian cancer, which may be or comprise additional surface protein biomarker(s) (e.g., ones as used and/or described herein), intravesicular protein biomarker(s) (e.g., ones as used and/or described herein), and/or intravesicular RNA (e.g., mRNA) biomarker(s)(e.g., ones as used and/or described herein).

In some embodiments, a capture agent included in a system and/or kit may comprise a binding moiety directed to an extracellular vesicle-associated membrane-bound polypeptide (e.g., ones described herein). In some embodiments, such a binding moiety may be conjugated to a solid substrate, which in some embodiments may be or comprise a solid substrate. In some embodiments, such a solid substrate may be or comprise a magnetic bead. In some embodiments, an exemplary capture agent included in a provided system and/or kit may be or comprise a solid substrate (e.g., a magnetic bead) and an antibody agent directed to an extracellular vesicle-associated membrane-bound polypeptide conjugated thereto.

In some embodiments where a target biomarker includes a surface protein biomarker and/or an intravesicular protein biomarker, a system and/or kit may include detection agents for performing a proximity ligation assay (e.g., ones as described herein). In some embodiments, such detection agents for performing a proximity ligation assay may comprise a set of detection probes, each directed to a target biomarker of a target biomarker signature, which set comprises at least two detection probes, wherein the two detection probes each comprise: (i) a polypeptide-binding moiety directed to a target biomarker; and (ii) an oligonucleotide domain coupled to the binding moiety, the oligonucleotide domain comprising a double-stranded portion and a single-stranded overhang portion extended from one end of the oligonucleotide domain, wherein the single-stranded overhang portions of the detection probes are characterized in that they can hybridize to each other when the detection probes are bound to the same extracellular vesicle.

In some embodiments, a provided system and/or kit may comprise a plurality (e.g., 2, 3, 4, 5, or more) of sets of detection probes, each set of which comprises two or more (e.g., 3, 4, 5, 6, 7, 8, 9, 10, 11, 12, 13, 14, 15, 16, 17, 18, 19, 20 or more) detection probes. In some embodiments, at least one set of detection probes may be directed to detection for ovarian cancer. For example, in some embodiments, a provided system and/kit may comprise at least one set for detection probes for detection of ovarian cancer and at least one set of detection probes for detection of a different cancer (e.g., pancreatic cancer). In some embodiments, two or more detection probes may be directed to different categories of ovarian cancer, e.g., high-grade serous ovarian cancer, endometrioid ovarian cancer, clear-cell ovarian cancer, low-grade serous ovarian cancer, or mucinous ovarian cancer. In some embodiments, two or more sets may be directed to detection of ovarian cancer of different stages. In some embodiments, two or more sets may be directed to detection of ovarian cancer of the same stage.

In some embodiments, detection probes in a provided kit may be provided as a single mixture in a container. In some embodiments, multiple sets of detection probes may be provided as individual mixtures in separate containers. In some embodiments, each detection probe is provided individually in a separate container.

In some embodiments where a target biomarker includes an intravesicular RNA (e.g., mRNA) biomarker, such a system and/or kit may include detection agents for performing a nucleic acid detection assay. In some embodiments, such a system and/or kit may include detection agents for performing a quantitative reverse-transcription PCR, for example, which may comprise primers directed to intravesicular RNA (e.g., mRNA) target(s).

In some embodiments, a provided system and/or kit may comprise at least one chemical reagent, e.g., to process a sample and/or extracellular vesicles therein. In some embodiments, a provided system and/or kit may comprise at least one chemical reagent to process extracellular vesicles in a sample, including, e.g., but not limited to a fixation agent, a permeabilization agent, and/or a blocking agent. In some embodiments, a provided system and/or kit may comprise a nucleic acid ligase and/or a nucleic acid polymerase. In some embodiments, a provided system and/or kit may comprise one or more primers and/or probes. In some embodiments, a provided system and/or kit may comprise one or more pairs of primers, for example for PCR, e.g., quantitative PCR (qPCR) reactions. In some embodiments, a provided system and/or kit may comprise one or more probes such as, for example, hydrolysis probes which may in some embodiments be designed to increase the specificity of qPCR (e.g., TaqMan probes). In some embodiments, a provided system and/or kit may comprise one or more multiplexing probes, for example as may be useful when simultaneous or parallel qPCR reactions are employed (e.g., to facilitate or improve readout).

In some embodiments, a provided system and/or kit can be used for screening (e.g., regular screening) and/or other assessment of individuals (e.g., asymptomatic or symptomatic subjects) for detection (e.g., early detection) of ovarian cancer. In some embodiments, a provided system and/or kit can be used for screening and/or other assessment of individuals susceptible to ovarian cancer (e.g., individuals with a known genetic, environmental, or experiential risk, etc.). In some embodiments, provided system and/or kits can be used for monitoring recurrence of ovarian cancer in a subject who has been previously treated. In some embodiments, provided systems and/or kits can be used as a companion diagnostic in combination with a therapy for a subject who is suffering from ovarian cancer. In some embodiments, provided systems and/or kits can be used for monitoring or evaluating efficacy of a therapy administered to a subject who is suffering from ovarian cancer. In some embodiments, provided systems and/or kits can be used for selecting a therapy for a subject who is suffering from ovarian cancer. In some embodiments, provided systems and/or kits can be used for making a therapy decision and/or selecting a therapy for a subject with one or more symptoms (e.g., non-specific symptoms) associated with ovarian cancer.

Complexes formed by performing methods described herein and/or using systems and/or kits described herein are also within the scope of disclosure. For example, in some embodiments, a complex comprising: (a) an extracellular vesicle expressing a target biomarker signature, at least two of which include at least one extracellular vesicle-associated membrane-bound polypeptide and at least one target biomarker selected from the group consisting of: surface protein biomarkers, intravesicular protein biomarkers, and intravesicular RNA biomarkers, wherein the surface protein biomarkers are selected from AQP5, CDH6, CHODL, CLDN3, CLDN6, CLDN16, EpCAM, FOLR1, HTR3A, LEMD1, LRRTM1, MUC16, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof; the intravesicular protein biomarkers are selected from CRABP2, KLK7, MIF, PRAME, and S100A1, and combinations thereof; and the intravesicular RNA (e.g., mRNA) biomarkers are selected from CLDN6, CRABP2, KLK7, MIF, PRAME, S100A1, and combinations thereof, wherein the extracellular vesicle is immobilized onto a solid substrate comprising a binding moiety directed to such a extracellular vesicle-associated membrane-bound polypeptide. Such a complex further comprises at least two detection probes directed to at least one target biomarker of the target biomarker signature present in the extracellular vesicle, wherein each detection probe is bound to such a target biomarker and each comprises: (i) a binding directed to the target biomarker; and (ii) an oligonucleotide domain coupled to the binding moiety, the oligonucleotide domain comprising a double-stranded portion and a single-stranded overhang portion extended from one end of the oligonucleotide domain, wherein the single-stranded overhang portions of the detection probes are hybridized to each other.

In some embodiments, a extracellular vesicle-associated membrane-bound polypeptide biomarker present in an extracellular vesicle that forms a complex may comprise one or more of AQP5, CDH6, CHODL, CLDN3, CLDN6, CLDN16, EpCAM, FOLR1, HTR3A, LEMD1, LRRTM1, MUC16, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof. In some embodiments, such an extracellular vesicle-associated membrane-bound polypeptide may be or comprise a SLC34A2 polypeptide. In some embodiments, such an extracellular vesicle-associated membrane-bound polypeptide may be or comprise a MUC16 polypeptide. In some embodiments, such an extracellular vesicle-associated membrane-bound polypeptide may be or comprise a CLDN6 polypeptide. In some embodiments, such an extracellular vesicle-associated membrane-bound polypeptide may be or comprise a FOLR1 polypeptide. In some embodiments, such an extracellular vesicle-associated membrane-bound polypeptide may be or comprise a LRRTM1 polypeptide. In some embodiments, such an extracellular vesicle-associated membrane-bound polypeptide may be or comprise a TACSTD2 polypeptide. In some embodiments, such an extracellular vesicle-associated membrane-bound polypeptide may be or comprise a MUC1 polypeptide. In some embodiments, such an extracellular vesicle-associated membrane-bound polypeptide may be or comprise a sTn polypeptide glycosylation. In some embodiments, such an extracellular vesicle-associated membrane-bound polypeptide may be or comprise a MSLN polypeptide. In some embodiments, such an extracellular vesicle-associated membrane-bound polypeptide may be or comprise an ALPL polypeptide. In some embodiments, such an extracellular vesicle-associated membrane-bound polypeptide may be or comprise a BST2 polypeptide. In some embodiments, such an extracellular vesicle-associated membrane-bound polypeptide may be or comprise a CLDN3 polypeptide.

These, and other aspects encompassed by the present disclosure, are described in more detail below and in the claims.

### BRIEF DESCRIPTION OF THE DRAWINGS

FIG. 1 is a schematic diagram illustrating an exemplary workflow of profiling individual extracellular vesicles (EVs). The figure shows purification of EVs from plasma using size exclusion chromatography (SEC) and immunoaffinity capture of EVs displaying a specific membrane-bound protein marker (Panel A); detection of co-localized target markers (e.g., intravesicular proteins or surface proteins) on captured EVs using a target entity detection assay according to some embodiments described herein (Panel B).

FIG. 2 is a schematic diagram illustrating a target entity detection assay according to some embodiments described herein. In some embodiments, a target entity detection assay uses a combination of detection probes, which combination is specific for detection of cancer. In some embodiments, a duplex system includes a first detection probe for a target protein 1 (e.g., cancer marker 1) and a second detection probe for a target protein 2 (e.g., cancer marker 2) are added to a sample comprising a biological entity (e.g., extracellular vesicle). In some embodiments, detection probes each comprise a target binding moiety (e.g., an antibody agent against a target protein) coupled to an oligonucleotide domain, which comprises a double-stranded portion and a single-stranded overhang extended from one end of the oligonucleotide domain. A detection signal is generated when distinct target binding moieties (e.g., antibody agents against target protein 1 and target protein 2, respectively) of the first and second detection probes are localized to the same biological entity (e.g., an extracellular vesicle) in close proximity such that the corresponding single-stranded overhangs hybridize to each other, thus allowing ligation of their oligonucleotide domains to occur. For example, a control entity (e.g., a biological entity from a healthy subject sample) does not express one or both of target protein 1 (e.g., cancer marker 1) and target protein 2 (e.g., cancer marker 2), so no detection of signal can be generated. However, when a biological entity from a cancer sample (e.g., ovarian cancer) expresses target protein 1 and target protein 2, and the target proteins are present within a short enough distance of each other in the same biological entity (e.g., extracellular vesicle), a detection signal is generated.

FIG. 3 show experimental data from qPCR detection of a ligated sample, e.g., using the assay illustrated in FIGS. 1 or 2, in different cell line-derived extracellular vesicle samples. In some embodiments, a target entity detection assay includes agents for capturing extracellular vesicles based on a SLC34A2 marker (“SLC34A2 capture”) and an exemplary duplex system, for example, involving at least two detection probes each comprising a MUC16-binding moiety (e.g., anti-MUC16 antibody) coupled to a distinct oligonucleotide domain, which comprises a double-stranded portion and a single-stranded overhang extended from one end of the oligonucleotide domain (“MUC16+MUC16 antibody probes”). Panel A shows a graph of qPCR data comparing detection of ovarian cancer cell-line EVs (positive cell line) over non-ovarian cancer cell line EVs (negative cell line). Panel B shows corresponding average delta Ct values using a negative control cell line (e.g., non-ovarian cancer cell line) as the baseline.

FIG. 4 is a set of graphs showing demographics of patients included in an ovarian cystadenocarcinoma (OC) patient plasma sample study. (Panel A) Overview of age and cohort size for the female patient cohort evaluated by the exemplary assay. (Panel B) Overview of age and cohort size for the female plasma samples from patients with benign masses.

FIG. 5 is a set of graphs showing correlation of patient age with CA-125 levels in samples from ovarian cancer patients (Panel A) and from patient with benign masses (Panel B).

FIG. 6 is a pie chart showing ovarian cancer prevalence by major ovarian carcinoma subtypes. “Others” refers to mixed or transitional carcinomas where it is not possible to categorize to a single subtype. See, e.g., Gilks et al., 2008, Seidman et al., 2003, 2004, which are each incorporated herein in their entirety by reference for the purpose described herein and for additional information.

FIG. 7 is a set of graphs showing performance of an exemplary assay for detection of ovarian cancer involving a duplex system (e.g., as described in FIGS. 1 or 2) based on SLC34A2 capture with MUC16+MUC16 antibody probes. Panel A refers to consolidated data from various ovarian cancer subtypes. Panel B shows excellent detection of the most common ovarian subtype, high-grade serous ovarian cancer, at two different cutoffs. Cutoff 1 pertains to a 99.8% specificity and Cutoff 2 pertains to a 98% specificity. Corresponding sensitivities for the noted ovarian cancer stages are displayed for the designated cutoffs.

FIG. 8 is a graph showing performance of an exemplary assay for detection of endometrioid ovarian cancer involving a duplex system (e.g., as described in FIGS. 1 or 2) based on SLC34A2 capture with MUC16+MUC16 antibody probes. It shows excellent detection of the second-most common ovarian subtype, endometrioid ovarian cancer, at two different cutoffs. Cutoff 1 pertains to a 99.8% specificity and Cutoff 2 pertains to a 98% specificity.

FIG. 9 is a graph showing performance of an exemplary assay for detection of low-grade serous ovarian cancer involving a duplex system (e.g., as described in FIG. 1) based on SLC34A2 capture with MUC16+MUC16 antibody probes, at two different cutoffs. Cutoff 1 pertains to a 99.8% specificity and Cutoff 2 pertains to a 98% specificity.

FIG. 10 is a graph showing performance of an exemplary assay for detection of clear-cell ovarian cancer involving a duplex system (e.g., as described in FIGS. 1 or 2) based on SLC34A2 capture with MUC16+MUC16 antibody probes, at two different cutoffs. Cutoff 1 pertains to a 99.8% specificity and Cutoff 2 pertains to a 98% specificity.

FIG. 11 is a graph showing performance of an exemplary assay for detection of mucinous ovarian cancer involving a duplex system (e.g., as described in FIGS. 1 or 2) based on SLC34A2 capture with MUC16+MUC16 antibody probes, at two different cutoffs. Cutoff 1 pertains to a 99.8% specificity and Cutoff 2 pertains to a 98% specificity.

FIG. 12 shows readouts of an exemplary assay for detection of ovarian cancer subtypes involving a duplex system (e.g., as described in FIGS. 1 or 2) based on SLC34A2 capture with MUC16+MUC16 antibody probes, relative to serum CA-125 levels. Panel A shows that there is no correlation observed between assay signal and serum CA-125 level. Cutoff 1 was drawn by fitting a log-normal distribution to the Ct value (obtained by the exemplary assay) for a population of healthy control patients and taking the mean of that distribution and 2.879 standard deviations, for which 99.8% of all the healthy patient Ct values will be above. Panel B is a Receiver Operating Characteristic (ROC) Curve for distinguishing patients with ovarian cancer from patients with benign gynecological tumors (patient cohort described in FIG. 4A) using the Ct values determined from the exemplary assay shown in Panel A versus serum CA-125 values shown in Panel A. Benign gynecological tumors include, for example, endometrioid cysts, follicular cysts, mucinous cystadenomas, mature teratomas, leiomyomas, and serous cystadenomas. Moreover, several subtypes of ovarian cancer were evaluated including high-grade serous, low-grade serous, mucinous, endometrioid, and clear-cell.

FIG. 13 is a set of data showing performance of an exemplary assay for detection of stage I and II high-grade serous ovarian cancer (HGSOC) compared to the current standard of care: serum CA-125 and transvaginal ultrasound (TVUS). The specificity (Panels A and D), sensitivity (Panels B and E), and positive predictive value (Panels C and F) were compared for screening woman at hereditary risk (Panels A, B, and C), and average risk (Panels D, E, and F) for HGSOC. In some instance, prevalence for hereditary- and average-risk woman is 1% and 0.057%, respectively. Performance parameters of serum CA-125 and TVUS were taken from Buys et al., 2011, which is incorporated herein by reference for the purpose described herein.

FIG. 14 is a set of graphs showing detection of MIF mRNA in EVs from ovarian cancer cell-lines vs. negative control cell lines. (Panel A) Detection of MIF mRNA in bulk EVs using RT-qPCR. (Panel B) Detection of MIF mRNA in EVs that were captured using anti-EpCAM functionalized beads compared to EVs in bulk.

FIG. 15 is a schematic diagram illustrating a target entity detection assay according to some embodiments described herein. The figure shows an exemplary triplex target entity detection system, in which in some embodiments, three or more detection probes, each for a target protein, can be added to a sample comprising a biological entity (e.g., extracellular vesicle). In some embodiments, detection probes each comprise a target binding moiety (e.g., an antibody agent against a target protein) coupled to an oligonucleotide domain, which comprises a double-stranded portion and a single-stranded overhang extended from one end of the oligonucleotide domain. A detection signal is generated when the corresponding single-stranded overhangs of all three or more detection probes hybridize to each other to form a linear double-stranded complex, and ligation of at least one strand of the double-stranded complex occurs, thus allowing a resulting ligated product to be detected.

FIG. 16 is a non-limiting example of a double-stranded complex comprising four detection probes connected to each other in a linear arrangement through hybridization of their respective single-stranded overhangs.

FIG. 17 is a schematic diagram illustrating a target entity detection assay of an exemplary embodiment described herein. In some embodiments, a plurality of detection probes, each for a distinct target, are added to a sample comprising a biological entity (e.g., extracellular vesicle). In some embodiments, detection probes each comprise a target binding moiety (e.g., an antibody agent) coupled to an oligonucleotide domain, which comprises a double-stranded portion and a single-stranded overhang extended from one end of the oligonucleotide domain. A detection signal is generated when all detection probes are localized to the same biological entity (e.g., an extracellular vesicle or analyte) in close proximity such that the corresponding single-stranded overhangs hybridize to form a linear double-stranded complex, and ligation of at least one strand of the resulting linear double-stranded complex occurs, thereby allowing a ligated product to be detected.

FIG. 18 depicts transcript expression in 442 ovarian cancer samples (stage I/II and stage III/IV) compared to all 17,382 healthy tissue samples for four exemplary surface protein biomarkers, which include (Panel A) SLC34A2, (Panel B) MUC16, (Panel C) FOLR1, and (Panel D) CLDN6.

FIG. 19 depicts performance of two exemplary orthogonal biomarker combinations. Transcript expression cutoffs noted in FIG. 18 were applied to (Panel A) SLC34A2 + MUC16 and (Panel B) FOLR1 + CLDN6, which differentiated 71% and 75% of ovarian cancer samples from over 99.9% of healthy tissue samples, respectively. (Panel C) Venn-diagram showing the percent of ovarian cancer patients identified above the cutoffs (91%) when using both biomarker combinations as shown in Panels A and B to characterize cancer patient samples. It shows that using two or more biomarker combinations can increase sensitivity of an ovarian cancer detection assay (e.g., as described herein).

FIG. 20 depicts preliminary biomarker combination results. (Panel A) Depicts results from a preliminary biomarker combination screen (112 combinations screened) in pooled healthy plasma and pooled ovarian cancer plasma samples. Each data point represents a unique biomarker combination, with red data points shown in Panel B. (Panel B) Difference in assay signal between pooled healthy plasma and pooled ovarian cancer plasma for seven exemplary biomarker combinations.

FIG. 21 depicts the use of multiple orthogonal biomarker combinations and the associated improvements in assay sensitivity. Combination 1 (SLC34A2 capture, MUC16 + MUC16 pliq-PCR detection probe readout) and Combination 2 (SLC34A2 capture, FOLR1 + FOLR1 pliq-PCR detection probe readout) are individually able to distinguish ovarian cancer populations from healthy control and benign tumor cohorts. Both cutoffs are set to achieve 99.5% specificity.

FIG. 22 depicts performance of an exemplary assay described herein involving individual exemplary biomarker combinations to distinguish control subjects (e.g., healthy woman subjects and/or subjects with benign gynecological tumors and/or inflammatory conditions including, e.g., Crohn’s disease, ulcerative colitis, endometriosis, etc.) from ovarian cancer patients. Exemplary individual biomarker combinations include:

| | | |
|---|---|---|
| Target of Capture Probe | Target of Detection Probe 1 | Target of Detection Probe 2 |
| SLC34A2 | MUC16 | MUC16 |
| SLC34A2 | FOLR1 | FOLR1 |
| SLC34A2 | MUC16 | FOLR1 |
| MUC16 | MUC16 | MUC16 |
| MUC16 | MUC16 | FOLR1 |
| MUC16 | MUC16 | CLDN3 |
| MUC16 | FOLR1 | CLDN3 |
| MUC16 | MUC16 | CLDN6 |
| MUC16 | FOLR1 | FOLR1 |
| LRRTM1 | MUC16 | MUC16 |

FIG. 23 depicts performance of an exemplary assay described herein involving an exemplary biomarker combination (e.g., a capture agent directed to SLC34A2 and a set of at least two detection probes each directed to MUC16). The cut-off value was determined by selecting the less restrictive of either (i) 2.93 standard deviations away from mean of healthy control subjects and subjects with inflammatory conditions (e.g., to exclude 99.83% of healthy subjects in the distribution) or (ii) a maximum assay signal from healthy control subjects. In some embodiments, benign ovarian tumor samples may be less of a concern for off-target signals than healthy control subjects and/or subjects with inflammatory conditions (e.g., Crohn’s disease, ulcerative colitis, endometriosis, etc.). Accordingly, in some such embodiments, benign ovarian tumor samples may not be included to determine a cutoff value.

FIG. 24 depicts performance of an exemplary assay described herein involving an exemplary biomarker combination (e.g., a capture agent directed to SLC34A2 and a set of at least a first detection probe directed to FOLR1 and a second detection probe directed to FOLR1). The cut-off value was determined as described in FIG. 23.

FIG. 25 depicts performance of an exemplary assay described herein involving an exemplary biomarker combination (e.g., a capture agent directed to SLC34A2 and a set of at least a first detection probe directed to MUC16 and a second detection probe directed to FOLR1). The cut-off value was determined as described in FIG. 23.

FIG. 26 depicts performance of an exemplary assay described herein involving an exemplary biomarker combination (e.g., a capture agent directed to MUC16 and a set of at least a first detection probe directed to MUC16 and a second detection probe directed to MUC16). The cut-off value was determined as described in FIG. 23.

FIG. 27 depicts performance of an exemplary assay described herein involving an exemplary biomarker combination (e.g., a capture agent directed to MUC16 and a set of at least a first detection probe directed to MUC16 and a second detection probe directed to FOLR1). The cut-off value was determined as described in FIG. 23.

FIG. 28 depicts performance of an exemplary assay described herein involving an exemplary biomarker combination (e.g., a capture agent directed to MUC16 and a set of at least a first detection probe directed to FOLR1 and a second detection probe directed to FOLR1). The cut-off value was determined as described in FIG. 23.

FIG. 29 depicts performance of exemplary assays described herein each involving an exemplary biomarker combination. (Panel A) A capture agent directed to SLC34A2 and a set of at least a first detection probe directed to MUC16 and a second detection probe directed to MUC16; (Panel B) A capture agent directed to SLC34A2 and a set of at least a first detection probe directed to FOLR1 and a second detection probe directed to FOLR1; and (Panel C) a capture agent directed to MUC16 and a set of at least a first detection probe directed to MUC16 and a second detection probe directed to FOLR1.

FIG. 30 depicts that an exemplary assay involving an exemplary biomarker combination (e.g., a capture agent directed to MUC16 with a set of at least a first detection probe directed to MUC16 and a second detection probe directed to FOLR1) can detect ovarian cancer patients with normal serum CA-125 (e.g., under 35 U/mL) and can distinguish many non-ovarian cancer patients with elevated serum CA-125 (e.g., ones having benign gynecological tumors) from ovarian cancer patients.

FIG. 31 depicts that an exemplary assay involving an exemplary biomarker combination (e.g., a capture agent directed to SLC34A2 with a set of at least a first detection probe directed to MUC16 and a second detection probe directed to MUC16) can detect ovarian cancer patients with normal serum CA-125 (e.g., under 35 U/mL) and can distinguish many non-ovarian cancer patients with elevated serum CA-125 (e.g., ones having benign gynecological tumors) from ovarian cancer patients.

FIG. 32 depicts that an exemplary assay involving an exemplary biomarker combination (e.g., a capture agent directed to SLC34A2 with a set of at least a first detection probe directed to FOLR1 and a second detection probe directed to FOLR1) can detect ovarian cancer patients with normal serum CA-125 (e.g., under 35 U/mL) and can distinguish many non-ovarian cancer patients with elevated serum CA-125 (e.g., ones having benign gynecological tumors) from ovarian cancer patients.

FIG. 33 depicts performance of an exemplary assay described herein involving an exemplary biomarker combination (e.g., a capture agent directed to MUC16 and a set of at least a first detection probe directed to MUC16 and a second detection probe directed to CLDN3). The cut-off value was determined as described in FIG. 23.

FIG. 34 depicts performance of an exemplary assay described herein involving an exemplary biomarker combination (e.g., a capture agent directed to MUC16 and a set of at least a first detection probe directed to MUC16 and a second detection probe directed to CLDN6). The cut-off value was determined as described in FIG. 23.

FIG. 35 depicts performance of an exemplary assay described herein involving an exemplary biomarker combination (e.g., a capture agent directed to MUC16 and a set of at least a first detection probe directed to FOLR1 and a second detection probe directed to CLDN3). The cut-off value was determined as described in FIG. 23.

FIG. 36 depicts performance of an exemplary assay described herein involving an exemplary biomarker combination (e.g., a capture agent directed to LRRTM1 and a set of at least a first detection probe directed to MUC16 and a second detection probe directed to MUC16). The cut-off value was determined as described in FIG. 23.

FIG. 37 depicts a series of Receiver Operating Characteristic (ROC) Curves for distinguishing patients with stage I-IV ovarian cancer from healthy patients. Curves were generated using the Ct values determined from the exemplary assays shown in FIGS. 23-28 and FIGS. 33-36 respectively. (Panel A) depicts an exemplary ROC curve with an area under the curve (AUC) of 0.92 when utilizing SLC34A2 capture probe, and MUC16 + MUC16 pliq-PCR detection probes, (Panel B) depicts an exemplary ROC curve with an AUC of 0.87 when utilizing SLC34A2 capture probe, and FOLR1 + FOLR1 pliq-PCR detection probes, (Panel C) depicts an exemplary ROC curve with an AUC of 0.90 when utilizing SLC34A2 capture probe, and MUC16 + FOLR1 pliq-PCR detection probes, (Panel D) depicts an exemplary ROC curve with an AUC of 0.91 when utilizing MUC16 capture probe, and MUC16 + MUC16 pliq-PCR detection probes, (Panel E) depicts an exemplary ROC curve with an AUC of 0.91 when utilizing MUC16 capture probe, and MUC16 + FOLR1 pliq-PCR detection probes, (Panel F) depicts an exemplary ROC curve with an AUC of 0.84 when utilizing MUC16 capture probe, and MUC16 + CLDN3 pliq-PCR detection probes, (Panel G) depicts an exemplary ROC curve with an AUC of 0.90 when utilizing MUC16 capture probe, and MUC16 + CLDN6 pliq-PCR detection probes, (Panel H) depicts an exemplary ROC curve with an AUC of 0.90 when utilizing MUC16 capture probe, and FOLR1 + FOLR1 pliq-PCR detection probes, (Panel I) depicts an exemplary ROC curve with an AUC of 0.67 when utilizing MUC16 capture probe, and FOLR1 + CLDN3 pliq-PCR detection probes, (Panel J) depicts an exemplary ROC curve with an AUC of 0.78 when utilizing LRRTM1 capture probe, and MUC16 + MUC16 pliq-PCR detection probes.

FIG. 38 depicts the use of multiple orthogonal biomarker signature combinations and the associated improvements in assay sensitivity. Combination 1 (SLC34A2 capture probe, and MUC16 + MUC16 pliq-PCR detection probes) and Combination 2 (MUC16 capture probe, and MUC16 + FOLR1 pliq-PCR detection probes) are individually able to distinguish ovarian cancer populations from healthy control and benign tumor cohorts. Both cutoffs are set to achieve 99.5% specificity.

FIG. 39 depicts the use of multiple orthogonal biomarker signature combinations and the associated improvements in assay sensitivity. Combination 1 (SLC34A2 capture probe, and FOLR1 + FOLR1 pliq-PCR detection probes) and Combination 2 (MUC16 capture probe, and MUC16 + FOLR1 pliq-PCR detection probes) are individually able to distinguish ovarian cancer populations from healthy control and benign tumor cohorts. Both cutoffs are set to achieve 99.5% specificity.

### CERTAIN DEFINITIONS

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be parenteral. In some embodiments, administration may be oral. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Amplification: The terms “amplification” and “amplify” refers to a template-dependent process that results in an increase in the amount and/or levels of a nucleic acid molecule relative to its initial amount and/or level. A template-dependent process is generally a process that involves template-dependent extension of a primer molecule, wherein the sequence of the newly synthesized strand of nucleic acid is dictated by the well-known rules of complementary base pairing (see, for example, Watson, J. D. et al., In: Molecular Biology of the Gene, 4th Ed., W. A. Benjamin, Inc., Menlo Park, Calif. (1987); which is incorporated herein by reference for the purpose described herein).

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi- specific antibodies (e.g., Zybodies®, etc.); antibody fragments such as Fab fragments, Fab′ fragments, F(ab′)2 fragments, Fd′ fragments, Fd fragments, and isolated complementary determining regions (CDRs) or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTEs®; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®, KALBITOR®s, and Affimer®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 95%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody agents can be made by the skilled person using methods and commercially available services and kits known in the art. For example, methods of preparation of monoclonal antibodies are well known in the art and include hybridoma technology and phage display technology. Further antibodies suitable for use in the present disclosure are described, for example, in the following publications: Antibodies A Laboratory Manual, Second edition. Edward A. Greenfield. Cold Spring Harbor Laboratory Press (Sep. 30, 2013); Making and Using Antibodies: A Practical Handbook, Second Edition. Eds. Gary C. Howard and Matthew R. Kaser. CRC Press (Jul. 29, 2013); Antibody Engineering: Methods and Protocols, Second Edition (Methods in Molecular Biology). Patrick Chames. Humana Press (Aug. 21, 2012); Monoclonal Antibodies: Methods and Protocols (Methods in Molecular Biology). Eds. Vincent Ossipow and Nicolas Fischer. Humana Press (Feb. 12, 2014); and Human Monoclonal Antibodies: Methods and Protocols (Methods in Molecular Biology). Michael Steinitz. Humana Press (Sep. 30, 2013)).

Antibodies may be produced by standard techniques, for example by immunization with the appropriate polypeptide or portion(s) thereof, or by using a phage display library. If polyclonal antibodies are desired, a selected host animal (e.g., mouse, rabbit, goat, horse, chicken, etc.) is immunized with an immunogenic polypeptide bearing a desired epitope(s), optionally haptenized to another polypeptide. Depending on the host species, various adjuvants may be used to increase immunological response. Such adjuvants include, but are not limited to, Freund’s, mineral gels such as aluminum hydroxide, and surface-active substances such as lysolecithin, pluronic polyols, polyanions, peptides, oil emulsions, keyhole limpet hemocyanin, and dinitrophenol. Serum from the immunized animal is collected and treated according to known procedures. If serum containing polyclonal antibodies to the desired epitope contains antibodies to other antigens, the polyclonal antibodies can be purified by immunoaffinity chromatography or any other method known in the art. Techniques for producing and processing polyclonal antisera are well known in the art.

Approximately or about: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Aptamer: As used herein, the term “aptamer” typically refers to a nucleic acid molecule or a peptide molecule that binds to a specific target molecule (e.g., an epitope). In some embodiments, a nucleic acid aptamer may be described by a nucleotide sequence and is typically about 15-60 nucleotides in length. A nucleic acid aptamer may be or comprise a single stranded and/or double-stranded structure. In some embodiments, a nucleic acid aptamer may be or comprise DNA. In some embodiments, a nucleic acid aptamer may be or comprise RNA. Without wishing to be bound by any theory, it is contemplated that the chain of nucleotides in an aptamer form intramolecular interactions that fold the molecule into a complex three-dimensional shape, and this three-dimensional shape allows the aptamer to bind tightly to the surface of its target molecule. In some embodiments, a peptide aptamer may be described to have one or more peptide loops of variable sequence displayed by a protein scaffold. Peptide aptamers can be isolated from combinatorial libraries and often subsequently improved by directed mutation or rounds of variable region mutagenesis and selection. Given the extraordinary diversity of molecular shapes that exist within the universe of all possible nucleotide and/or peptide sequences, aptamers may be obtained for a wide array of molecular targets, including proteins and small molecules. In addition to high specificity, aptamers typically have very high affinities for their targets (e.g., affinities in the picomolar to low nanomolar range for proteins or polypeptides). Because aptamers are typically synthetic molecules, aptamers are amenable to a variety of modifications, which can optimize their function for particular applications.

Associated with: Two events or entities are “associated” with one another, as that term is used herein, if the presence, level and/or form of one is correlated with that of the other. For example, a particular biological phenomenon (e.g., expression of a specific biomarker) is considered to be associated with ovarian cancer (e.g., a specific type of ovarian cancer and/or stage of ovarian cancer), if its presence correlates with incidence of and/or susceptibility of the ovarian cancer (e.g., across a relevant population).

Biological entity: In appropriate circumstances, as will be clear from context to those skilled in the art, the term “biological entity” may be utilized to refer to an entity or component that is present in a biological sample, e.g., in some embodiments derived or obtained from a subject, which, in some embodiments, may be or comprise a cell or an organism, such as an animal or human, or, in some embodiments, may be or comprise a biological tissue or fluid. In some embodiments, a biological entity is or comprises a cell or microorganism, or a fraction, extract, or component thereof (including, e.g., intracellular components and/or molecules secreted by a cell or microorganism). For example, in some embodiments, a biological entity is or comprises a cell. In some embodiments, a biological entity is or comprises an extracellular vesicle. In some embodiments, a biological entity is or comprises a biological analyte (e.g., a metabolite, carbohydrate, protein or polypeptide, enzyme, lipid, organelle, cytokine, receptor, ligand, and any combinations thereof). In some embodiments, a biological entity present in a sample is in a native state (e.g., proteins or polypeptides remain in a naturally occurring conformational structure). In some embodiments, a biological entity is processed, e.g., by isolating from a sample or deriving from a naturally occurring biological entity. For example, a biological entity can be processed with one or more chemical agents such that it is more desirable for detection utilizing technologies provided herein. As an example only, a biological entity may be a cell or extracellular vesicle that is contacted with a fixative agent (e.g., but not limited to methanol and/or formaldehyde) to cause proteins and/or peptides present in the cell or extracellular vesicle to form crosslinks. In some embodiments, a biological entity is in an isolated or pure form (e.g., isolated from a bodily fluid sample such as, e.g., a blood, serum, plasma sample, etc.). In some embodiments, a biological entity may be present in a complex matrix (e.g., a bodily fluid sample such as, e.g., a blood, serum, or plasma sample, etc.).

Biomarker: The term “biomarker” typically refers to an entity, event, or characteristic whose presence, level, degree, type, and/or form, correlates with a particular biological event or state of interest, so that it is considered to be a “marker” of that event or state. To give but a few examples, in some embodiments, a biomarker may be or comprise a marker for a particular disease state, or for likelihood that a particular disease, disorder or condition may develop, occur, or reoccur. In some embodiments, a biomarker may be or comprise a marker for a particular disease or therapeutic outcome, or likelihood thereof. In some embodiments, a biomarker may be or comprise a marker for a particular tissue (e.g., but not limited to brain, breast, colon, ovary and/or other tissues associated with a female reproductive system, pancreas, prostate and/or other tissues associated with a male reproductive system, liver, lung, and skin). Such a marker for a particular tissue, in some embodiments, may be specific for a healthy tissue, specific for a diseased tissue, or in some embodiments may be present in a normal healthy tissue and diseased tissue (e.g., a tumor); those skilled in the art, reading the present disclosure, will appreciate appropriate contexts for each such type of biomarker. In some embodiments, a biomarker may be or comprise a cancer-specific marker (e.g., a marker that is specific to a particular cancer). In some embodiments, a biomarker may be or comprise a non-specific cancer marker (e.g., a marker that is present in at least two or more cancers). A non-specific cancer marker may be or comprise, in some embodiments, a generic marker for cancers (e.g., a marker that is typically present in cancers, regardless of tissue types), or in some embodiments, a marker for cancers of a specific tissue (e.g., but not limited to brain, breast, colon, ovary and/or other tissues associated with a female reproductive system, pancreas, prostate and/or other tissues associated with a male reproductive system, liver, lung, and skin). Thus, in some embodiments, a biomarker is predictive; in some embodiments, a biomarker is prognostic; in some embodiments, a biomarker is diagnostic, of the relevant biological event or state of interest. A biomarker may be or comprise an entity of any chemical class, and may be or comprise a combination of entities. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is or comprises a portion of a particular molecule, complex, or structure; e.g., in some embodiments, a biomarker may be or comprise an epitope. In some embodiments, a biomarker is a surface marker (e.g., a surface protein marker) of an extracellular vesicle associated with ovarian cancer. In some embodiments, a biomarker is intravesicular (e.g., a protein or RNA marker that is present within an extracellular vesicle). In some embodiments, a biomarker may be or comprise a genetic or epigenetic signature. In some embodiments, a biomarker may be or comprise a gene expression signature. In some embodiments, a “biomarker” appropriate for use in accordance with the present disclosure may refer to presence, level, and/or form of a molecular entity (e.g., epitope) present in a target marker. For example, in some embodiments, two or more “biomarkers” as molecular entities (e.g., epitopes) may be present on the same target marker (e.g., a marker protein such as a surface protein present in an extracellular vesicle).

Blood-derived sample: The term “blood-derived sample,” as used herein, refers to a sample derived from a blood sample (i.e., a whole blood sample) of a subject in need thereof. Examples of blood-derived samples include, but are not limited to, blood plasma (including, e.g., fresh frozen plasma), blood serum, blood fractions, plasma fractions, serum fractions, blood fractions comprising red blood cells (RBC), platelets, leukocytes, etc., and cell lysates including fractions thereof (for example, cells, such as red blood cells, white blood cells, etc., may be harvested and lysed to obtain a cell lysate). In some embodiments, a blood-derived sample that is used with methods, systems, and/or kits described herein is a plasma sample.

Cancer: The term “cancer” is used herein to generally refer to a disease or condition in which cells of a tissue of interest exhibit relatively abnormal, uncontrolled, and/or autonomous growth, so that they exhibit an aberrant growth phenotype characterized by a significant loss of control of cell proliferation. In some embodiments, cancer may comprise cells that are precancerous (e.g., benign), malignant, pre-metastatic, metastatic, and/or non-metastatic. The present disclosure provides technologies for detection of ovarian cancer.

Classification cutoff: As used herein, the term “classification cutoff” refers to a level, value, or score, or a set of values, or an indicator that is used to predict a subject’s risk for a disease or condition (e.g., lung cancer), for example, by defining one or more dividing lines among two or more subsets of a population (e.g., normal healthy subjects and subjects with inflammatory conditions vs. lung cancer subjects). In some embodiments, a classification cutoff may be determined referencing at least one reference threshold level (e.g., reference cutoff) for a target biomarker signature described herein, optionally in combination with other appropriate variables, e.g., age, life-history-associated risk factors, hereditary factors, physical and/or medical conditions of a subject. In some embodiments where a classification is based on a single target biomarker signature (e.g., as described herein), a classification cutoff may be the same as a reference threshold (e.g., cutoff) pre-determined for the single target biomarker signature. In some embodiments where a classification is based on two or more target biomarker signatures, a classification cutoff may reference two or more reference thresholds (e.g., cutoffs) each individually pre-determined for the corresponding target biomarker signatures, and optionally incorporate one or more appropriate variables, e.g., age, life-history-associated risk factors, hereditary factors, physical and/or medical conditions of a subject. In some embodiments, a classification cutoff may be determined via a computer algorithm-mediated analysis that references at least one reference threshold level (e.g., reference cutoff) for a target biomarker signature described herein, optionally in combination with other appropriate variables, e.g., age, life-history-associated risk factors, hereditary factors, physical and/or medical conditions of a subj ect.

Close proximity: The term “close proximity” as used herein, refers to a distance between two detection probes (e.g., two detection probes in a pair) that is sufficiently close enough such that an interaction between the detection probes (e.g., through respective oligonucleotide domains) is expected to likely occur. For example, in some embodiments, probability of two detection probes interacting with each other (e.g., through respective oligonucleotide domains) over a period of time when they are in sufficiently close proximity to each other under a specified condition (e.g., when detection probes are bound to respective targets in an extracellular vesicle is at least 50% or more, including, e.g., at least 60%, at least 70%, at least 80%, at least 90% or more. In some embodiments, a distance between two detection probes when they are in sufficiently close proximity to each other may range between approximately 0.1-1000 nm, or 0.5-500 nm, or 1-250 nm. In some embodiments, a distance between two detection probes when they are in sufficiently close proximity to each other may range between approximately 0.1-10 nm or between approximately 0.5-5 nm. In some embodiments, a distance between two detection probes when they are in sufficiently close proximity to each other may be less than 100 nm or shorter, including, e.g., less than 90 nm, less than 80 nm, less than 70 nm, less than 60 nm, less than 50 nm, less than 40 nm, less than 30 nm, less than 20 nm, less than 10 nm, less than 5 nm, less than 1 nm, or shorter.. In some embodiments, a distance between two detection probes when they are in sufficiently close proximity to each other may range between approximately 40-1000 nm or 40 nm-500 nm.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Complementary: As used herein, the term “complementary” is used in reference to oligonucleotide hybridization related by base-pairing rules. For example, the sequence “C—A—G—T” is complementary to the sequence “G—T—C—A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules.

Detecting: The term “detecting” is used broadly herein to include appropriate means of determining the presence or absence of an extracellular vesicle expressing a target biomarker signature of ovarian cancer or any form of measurement indicative of such an extracellular vesicle. Thus, “detecting” may include determining, measuring, assessing, or assaying the presence or absence, level, amount, and/or location of an entity of interest (e.g., a surface protein biomarker, an intravesicular protein biomarker, or an intravesicular RNA biomarker) that corresponds to part of a target biomarker signature in any way. In some embodiments, “detecting” may include determining, measuring, assessing, or quantifying a form of measurement indicative of an entity of interest (e.g., a ligated template indicative of a surface protein biomarker and/or an intravesicular protein biomarker, or a PCR amplification product indicative of an intravesicular mRNA). Quantitative and qualitative determinations, measurements or assessments are included, including semi-quantitative. Such determinations, measurements or assessments may be relative, for example when an entity of interest (e.g., a surface protein biomarker, an intravesicular protein biomarker, or an intravesicular RNA biomarker) or a form of measurement indicative thereof is being detected relative to a control reference, or absolute. As such, the term “quantifying” when used in the context of quantifying an entity of interest (e.g., a surface protein biomarker, an intravesicular protein biomarker, or an intravesicular RNA biomarker) or a form of measurement indicative thereof can refer to absolute or to relative quantification. Absolute quantification may be accomplished by correlating a detected level of an entity of interest (e.g., a surface protein biomarker, an intravesicular protein biomarker, or an intravesicular RNA biomarker) or a form of measurement indicative thereof to known control standards (e.g., through generation of a standard curve). Alternatively, relative quantification can be accomplished by comparison of detected levels or amounts between two or more different entities of interest (e.g., different surface protein biomarkers, intravesicular protein biomarkers, or intravesicular RNA biomarkers) to provide a relative quantification of each of the two or more different entities of interest, i.e., relative to each other.

Detection label: The term “detection label” as used herein refers to any element, molecule, functional group, compound, fragment or moiety that is detectable. In some embodiments, a detection label is provided or utilized alone. In some embodiments, a detection label is provided and/or utilized in association with (e.g., joined to) another agent. Examples of detection labels include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr, etc.), fluorescent dyes, chemiluminescent agents (such as, for example, acridinium esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes, colorimetric labels (such as, for example, dyes, colloidal gold, and the like), biotin, digoxigenin, haptens, and proteins for which antisera or monoclonal antibodies are available.

Detection probe: The term “detection probe” typically refers to a probe directed to detection and/or quantification of a specific target. In some embodiments, a detection probe is a quantification probe, which provides an indicator representing level of a specific target. In accordance with the present disclosure, a detection probe refers to a composition comprising a target binding entity, directly or indirectly, coupled to an oligonucleotide domain, wherein the target binding entity specifically binds to a respective target (e.g., molecular target), and wherein at least a portion of the oligonucleotide domain is designed to permit hybridization with a portion of an oligonucleotide domain of another detection probe for a distinct target. In many embodiments, an oligonucleotide domain appropriate for use in the accordance with the present disclosure comprises a double-stranded portion and at least one single-stranded overhang. In some embodiments, an oligonucleotide domain may comprise a double-stranded portion and a single-stranded overhang at each end of the double-stranded portion.

Double-stranded: As used herein, the term “double-stranded” in the context of oligonucleotide domain is understood by those of skill in the art that a pair of oligonucleotides exist in a hydrogen-bonded, helical arrangement typically associated with, for example, nucleic acid such as DNA. In addition to the 100% complementary form of double-stranded oligonucleotides, the term “double-stranded” as used herein is also meant to refer to those forms which include mismatches (e.g., partial complementarity) and/or structural features as bulges, loops, or hairpins.

Double-stranded complex: As used herein, the term “double-stranded complex” typically refers to a complex comprising at least two or more (including, e.g., 2, 3, 4, 5, 6, 7, 8, 9, 10, 11, 12, 13, 14, 15, 16, 17, 18, 19, 20, or more) detection probes (e.g., as provided and/or utilized herein), each directed to a target (which can be the same target or a distinct target), connected or coupled to one another in a linear arrangement through hybridization of complementary single-stranded overhangs of the detection probes. In some embodiments, such a double-stranded complex may comprise an extracellular vesicle, wherein respective target binding moieties of the detection probes are simultaneously bound to the extracellular vesicle.

Epitope: As used herein, the term “epitope” includes any moiety that is specifically recognized by an immunoglobulin (e.g., antibody or receptor) binding component or an aptamer. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Extracellular vesicle: As used herein, the term “extracellular vesicle” typically refers to a vesicle outside of a cell, e.g., secreted by a cell. Examples of secreted vesicles include, but are not limited to exosomes, microvesicles, microparticles, ectosomes, oncosomes, and apoptotic bodies. Without wishing to be bound by theory, exosomes are nanometer-sized vesicles (e.g., between 40 nm and 120 nm) of endocytic origin that may form by inward budding of the limiting membrane of multivesicular endosomes (MVEs), while microvesicles typically bud from the cell surface and their size may vary between 50 nm and 1000 nm. In some embodiments, an extracellular vesicle is or comprises an exosome and/or a microvesicle. In some embodiments, a sample comprising an extracellular vesicle is substantially free of apoptotic bodies. In some embodiments, a sample comprising extracellular vesicles may comprise extracellular vesicles shed or derived from one or more tissues (e.g., cancerous tissues and/or non-cancerous or healthy tissues). In some embodiments, an extracellular vesicle in a sample may be shed or derived from an ovarian cancer tumor; in some embodiments, an extracellular vesicle is shed or derived from a tumor of a non-ovarian cancer. In some embodiments, an extracellular vesicle is shed or derived from a healthy tissue. In some embodiments, an extracellular vesicle is shed or derived from a benign gynecological tumor. In some embodiments, an extracellular vesicle is shed or derived from a tissue of a subject with symptoms (e.g., non-specific symptoms) associated with ovarian cancer.

Extracellular vesicle-associated membrane-bound polypeptide: As used herein, such a term refers to a polypeptide that is present in the membrane of an extracellular vesicle. In some embodiments, such a polypeptide may be tumor-specific. In some embodiments, such a polypeptide may be tissue-specific (e.g., ovarian tissue-specific). In some embodiments, such a polypeptide may be non-specific, e.g., it is present in one or more non-target tumors, and/or in one or more non-target tissues.

Hybridization: As used herein, the term “hybridizing”, “hybridize”, “hybridization”, “annealing”, or “anneal” are used interchangeably in reference to pairing of complementary nucleic acids using any process by which a strand of nucleic acid joins with a complementary strand through base pairing to form a hybridization complex. Hybridization and the strength of hybridization (e.g., strength of the association between the nucleic acids) is impacted by various factors including, e.g., the degree of complementarity between the nucleic acids, stringency of the conditions involved, the melting temperature (T) of the formed hybridization complex, and the G:C ratio within the nucleic acids.

Intravesicular protein biomarker: As used herein, the term “intravesicular protein biomarker” refers to a marker indicative of the state (e.g., presence, level, and/or activity) of a polypeptide that is present within a biological entity (e.g., a cell or an extracellular vesicle). In many embodiments, an intravesicular protein biomarker is associated with or present within an extracellular vesicle.

Intravesicular RNA biomarker: As used herein, the term “intravesicular RNA biomarker” refers to a marker indicative of the state (e.g., presence and/or level) of a RNA (e.g., mRNA) that is present within a biological entity (e.g., a cell or an extracellular vesicle). In many embodiments, an intravesicular RNA biomarker is associated with or present within an extracellular vesicle.

Ligase: As used herein, the term “ligase” or “nucleic acid ligase” refers to an enzyme for use in ligating nucleic acids. In some embodiments, a ligase is enzyme for use in ligating a 3′-end of a polynucleotide to a 5′-end of a polynucleotide. In some embodiments, a ligase is an enzyme for use to perform a sticky-end ligation. In some embodiments, a ligase is an enzyme for use to perform a blunt-end ligation. In some embodiments, a ligase is or comprises a DNA ligase.

Life-history-associated risk factors: As used herein, the term “life-history risk factors” refers to individuals’ actions, experiences, medical history, and/or exposures in their lives which may directly or indirectly increase such individuals’ risk for a condition, e.g., ovarian cancer, relative to individuals who do not have such actions, experiences, medical history, and/or exposures in their lives. In some embodiments, non-limiting examples of life-history-associated risk factors include smoking, alcohol, drugs, carcinogenic agents, diet, obesity, diabetes, polycystic ovarian syndrome (PCOS), endometriosis, pelvic inflammatory disease (PID), nulliparousness/infertility, no history/short history of oral contraceptive use, physical activity, sun exposure, radiation exposure, perineal talc use, hormone replacement therapy (HRT), exposure to infectious agents such as viruses, and/or occupational hazard (Reid et al., 2017; which is incorporated herein by reference for the purpose described herein). One skilled in the art recognizes that the above list of life-history-associated risk factors contributing to cancer (e.g., ovarian cancer) susceptibility is not exhaustive but constantly evolving.

Ligation: As used herein, the term “ligate”, “ligating or “ligation” refers to a method or composition known in the art for joining two oligonucleotides or polynucleotides. A ligation may be or comprise a sticky-end ligation or a blunt-end ligation. In some embodiments, ligation involved in provided technologies is or comprises a sticky-end ligation. In some embodiments, ligation refers to joining a 3′ end of a polynucleotide to a 5′ end of a polynucleotide. In some embodiments, ligation is facilitated by use of a nucleic acid ligase.

Non-cancer subjects: As used herein, the term “non-cancer subjects” generally refers to female subjects who do not have non-benign ovarian cancer. For example, in some embodiments, a non-cancer subject is a healthy female subject (e.g., a healthy woman subject). In some embodiments, a non-cancer subject is a healthy female subject (e.g., a healthy woman subject) below age 55. In some embodiments, a non-cancer subject is a healthy female subject (e.g., a healthy woman subject) with age 55 or above. In some embodiments, a non-cancer subject is a female subject (e.g., woman subject) with non-ovarian related health diseases, disorders, or conditions. In some embodiments, a non-cancer subject is a female subject (e.g., a woman subject) having a benign ovarian tumor (e.g., a benign mass observed in a fallopian tube and/or on an ovary).

Nucleic acid/ Oligonucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3 -methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5 -propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of size, e.g., of an oligonucleotide, a certain number of nucleotides refers to the number of nucleotides on a single strand, e.g., of an oligonucleotide.

Patient: As used herein, the term “patient” refers to any organism who is suffering or at risk of a disease or disorder or condition. Typical patients include animals (e.g., mammals such as mice, rats, rabbits, non-human primates, and/or humans). In some embodiments, a patient is a human. In some embodiments, a patient is suffering from or susceptible to one or more diseases or disorders or conditions. In some embodiments, a patient displays one or more symptoms of a disease or disorder or condition. In some embodiments, a patient has been diagnosed with one or more diseases or disorders or conditions. In some embodiments, a disease or disorder or condition that is amenable to provided technologies is or includes cancer, or presence of one or more tumors. In some embodiments, a patient is receiving or has received certain therapy to diagnose and/or to treat a disease, disorder, or condition.

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics).

Prevent or prevention: As used herein, “prevent” or “prevention,” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Primer: As used herein, the term “primer” refers to an oligonucleotide capable of acting as a point of initiation of synthesis when placed under conditions in which synthesis of a primer extension product which is complementary to a nucleic acid strand is induced (e.g., in the presence of nucleotides and an inducing agent such as DNA polymerase and at a suitable temperature and pH). A primer is preferably single stranded for maximum efficiency in amplification. A primer must be sufficiently long to prime the synthesis of extension products in the presence of the inducing agent. The exact lengths of a primer can depend on many factors, e.g., temperature.

Reference: As used herein, “reference” describes a standard or control relative to which a comparison is performed. For example, in some embodiments, an agent, animal, individual, population, sample, sequence or value of interest is compared with a reference or control agent, animal, individual, population, sample, sequence, or value. In some embodiments, a reference or control is tested and/or determined substantially simultaneously with the testing or determination of interest. In some embodiments, a reference or control is a historical reference or control, optionally embodied in a tangible medium. In some embodiments, a reference or control in the context of a reference level of a target refers to a level of a target in a normal healthy subject or a population of normal healthy subjects. In some embodiments, a reference or control in the context of a reference level of a target refers to a level of a target in a subject prior to a treatment. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more.

Sample: As used herein, the term “sample” typically refers to an aliquot of material obtained or derived from a source of interest. In some embodiments, a sample is obtained or derived from a biological source (e.g., a tissue or organism or cell culture) of interest. In some embodiments, a source of interest may be or comprise a cell or an organism, such as an animal or human. In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravesicular fluid (blood plasma), an interstitial fluid, a lymphatic fluid, and/or a transcellular fluid. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchoalveolar, ductal, nasal, ocular, oral, uterine, vaginal, or other washing or lavage). In some embodiments, a biological sample is or comprises a liquid biopsy. In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, a sample is a preparation that is processed by using a semi-permeable membrane or an affinity-based method such antibody-based method to separate a biological entity of interest from other non-target entities. Such a “processed sample” may comprise, for example, in some embodiments extracellular vesicles, while, in some embodiments, nucleic acids and/or proteins, etc., extracted from a sample. In some embodiments, a processed sample can be obtained by subjecting a primary sample to one or more techniques such as amplification or reverse transcription of nucleic acid, isolation and/or purification of certain components, etc.

Selective or specific: The term “selective” or “specific”, when used herein with reference to an agent having an activity, is understood by those skilled in the art to mean that the agent discriminates between potential target entities, states, or cells. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a target-binding moiety for one or more other potential target entities (e.g., competitors). In some embodiments, specificity is evaluated relative to that of a reference specific binding moiety. In some embodiments, specificity is evaluated relative to that of a reference non-specific binding moiety. In some embodiments, a target-binding moiety does not detectably bind to the competing alternative target under conditions of binding to its target entity. In some embodiments, a target-binding moiety binds with higher on-rate, lower off-rate, increased affinity, decreased dissociation, and/or increased stability to its target entity as compared with the competing alternative target(s).

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is biologically active. In some embodiments, suitable small molecules may be identified by methods such as screening large libraries of compounds (Beck- Sickinger & Weber (2001) Combinational Strategies in Biology and Chemistry (John Wiley & Sons, Chichester, Sussex); by structure-activity relationship by nuclear magnetic resonance (Shuker et al. (1996) “Discovering high-affinity ligands for proteins: SAR by NMR.” Science 274: 1531-1534); encoded self-assembling chemical libraries (Melkko et al. (2004) “Encoded self-assembling chemical libraries.” Nature Biotechnol. 22: 568-574); DNA-templated chemistry (Gartner et al. (2004) “DNA-templated organic synthesis and selection of a library of macrocycles.” Science 305: 1601-1605); dynamic combinatorial chemistry (Ramstrom & Lehn (2002) “Drug discovery by dynamic combinatorial libraries.” Nature Rev. Drug Discov. 1: 26-36); tethering (Arkin & Wells (2004) “Small-molecule inhibitors of protein-protein interactions: progressing towards the dream.” Nature Rev. Drug Discov. 3: 301-317); and speed screen (Muckenschnabel et al. (2004) “SpeedScreen: label-free liquid chromatography-mass spectrometry-based high- throughput screening for the discovery of orphan protein ligands.” Anal. Biochem. 324: 241-249). In some embodiments, a small molecule may have a dissociation constant for a target in the nanomolar range.

Specific binding: As used herein, the term “specific binding” refers to an ability to discriminate between possible binding partners in the environment in which binding is to occur. A target-binding moiety that interacts with one particular target when other potential targets are present is said to “bind specifically” to the target with which it interacts. In some embodiments, specific binding is assessed by detecting or determining degree of association between a target-binding moiety and its partner; in some embodiments, specific binding is assessed by detecting or determining degree of dissociation of a target-binding moiety-partner complex; in some embodiments, specific binding is assessed by detecting or determining ability of a target-binding moiety to compete an alternative interaction between its partner and another entity. In some embodiments, specific binding is assessed by performing such detections or determinations across a range of concentrations.

Stage of cancer: As used herein, the term “stage of cancer” refers to a qualitative or quantitative assessment of the level of advancement of a cancer (e.g., ovarian cancer). In some embodiments, criteria used to determine the stage of a cancer may include, but are not limited to, one or more of where the cancer is located in a body, tumor size, whether the cancer has spread to lymph nodes, whether the cancer has spread to one or more different parts of the body, etc. In some embodiments, cancer may be staged using the AJCC staging system. The AJCC staging system is a classification system, developed by the American Joint Committee on Cancer for describing the extent of disease progress in cancer patients, which utilizes in part the TNM scoring system: Tumor size, Lymph Nodes affected, Metastases. In some embodiments, cancer may be staged using a classification system that in part involves the TNM scoring system, according to which T refers to the size and extent of the main tumor, usually called the primary tumor; N refers to the number of nearby lymph nodes that have cancer; and M refers to whether the cancer has metastasized. In some embodiments, a cancer may be referred to as Stage 0 (abnormal cells are present but have not spread to nearby tissue, also called carcinoma in situ, or CIS; CIS is not cancer, but it may become cancer), Stage I-III (cancer is present; the higher the number, the larger the tumor and the more it has spread into nearby tissues), or Stage IV (the cancer has spread to distant parts of the body). In some embodiments, a cancer may be assigned to a stage selected from the group consisting of: in situ (abnormal cells are present but have not spread to nearby tissue); localized (cancer is limited to the place where it started, with no sign that it has spread); regional (cancer has spread to nearby lymph nodes, tissues, or organs): distant (cancer has spread to distant parts of the body); and unknown (there is not enough information to figure out the stage).

Subject: As used herein, the term “subject” refers to an organism from which a sample is obtained, e.g., for experimental, diagnostic, prophylactic, and/or therapeutic purposes. Typical subjects include animals (e.g., mammals such as mice, rats, rabbits, non-human primates, domestic pets, etc.) and humans. In some embodiments, a subject is a human female subject, e.g., a human woman subject. In some embodiments, a subject is suffering from ovarian cancer. In some embodiments, a subject is susceptible to ovarian cancer. In some embodiments, a subject displays one or more symptoms or characteristics of ovarian cancer. In some embodiments, a subject displays one or more non-specific symptoms of ovarian cancer. In some embodiments, a subject does not display any symptom or characteristic of ovarian cancer. In some embodiments, a subject is someone with one or more features characteristic of susceptibility to or risk of ovarian cancer. In some embodiments, a subject is a patient. In some embodiments, a subject is an individual to whom diagnosis and/or therapy is and/or has been administered. In some embodiments, a subject is a female subject (e.g., woman subject) determined to have an adnexal masses. In some embodiments, a subject is an asymptotic subject. Such an symptomatic subject may be a female subject (e.g., woman subject) at average population risk or with hereditary risk. For example, such an asymptomatic subject may be a subject who has a family history of cancer, who has been previously treated for cancer, who is at risk of cancer recurrence after cancer treatment, who is in remission after cancer treatment, and/or who has been previously or periodically screened for the presence of at least one cancer biomarker. Alternatively, in some embodiments, an asymptomatic subject may be a subject who has not been previously screened for cancer, who has not been diagnosed for cancer, and/or who has not previously received cancer therapy. In some embodiments, a subject amenable to provided technologies is an individual selected based on one or more characteristics such as age, race, genetic history, medical history, personal history (e.g., smoking, alcohol, drugs, carcinogenic agents, diet, obesity, physical activity, sun exposure, radiation exposure, exposure to infectious agents such as viruses, and/or occupational hazard).

Suffering from: An individual who is “suffering from” a disease, disorder, and/or condition has been diagnosed with and/or displays one or more symptoms of a disease, disorder, and/or condition.

Surface polypeptide or surface protein: As used interchangeably herein, the terms “surface polypeptide,” “surface protein,” and “membrane-bound polypeptide” refer to a polypeptide or protein with one or more domains or regions present in and/or on the surface of the membrane of a biological entity (e.g., a cell, an extracellular vesicle, etc.). In some embodiments, a surface protein may comprise one or more domains or regions spanning and/or associated with the plasma membrane of a biological entity (e.g., a cell, an extracellular vesicle, etc.). In some embodiments, a surface protein may comprise one or more domains or regions spanning and/or associated with the plasma membrane of a biological entity (e.g., a cell, an extracellular vesicle, etc.) and also protruding into the intracellular and/or intravesicular space. In some embodiments, a surface protein may comprise one or more domains or regions associated with the plasma membrane of a biological entity (e.g., a cell, an extracellular vesicle, etc.), for example, via one or more non-peptidic linkages. In some embodiments, a surface protein may comprise one or more domains or regions that is/are anchored into either side of plasma membrane of a biological entity (e.g., a cell, an extracellular vesicle, etc.). In some embodiments, a surface protein is associated with or present within an extracellular vesicle. In some embodiments, a surface polypeptide or membrane-bound polypeptide may be associated with or present within an ovarian cancer-associated extracellular vesicle (e.g., an extracellular vesicle obtained or derived from a blood or blood-derived sample of a subject suffering from or susceptible to ovarian cancer). As will be understood by a skilled artisan, detection of the presence of at least a portion of a surface polypeptide or surface protein on/within extracellular vesicles can facilitate separation and/or isolation of ovarian cancer-associated extracellular vesicles from a biological sample (e.g., a blood or blood-derived sample) from a subject. In some embodiments, detection of the presence of a surface polypeptide or surface protein may be or comprise detection of an intravesicular portion (e.g., an intravesicular epitope) of such a surface polypeptide or surface protein. In some embodiments, detection of the presence of a surface polypeptide or surface protein may be or comprise detection of a membrane-spanning portion of such a surface polypeptide or surface protein. In some embodiments, detection of the presence of a surface polypeptide or surface protein may be or comprise detection of an extravesicular portion of such a surface polypeptide or surface protein.

Surface protein biomarker: As used herein, the term “surface protein biomarker” refers to a marker indicative of the state (e.g., presence, level, and/or activity) of a surface protein (e.g., as described herein) of a biological entity (e.g., a cell or an extracellular vesicle). In some embodiments, a surface protein refers to a polypeptide or protein with one or more domains or regions located in or on the surface of the membrane of a biological entity (e.g., a cell or an extracellular vesicle). In some embodiments, a surface protein biomarker may be or comprise an epitope that is present on the interior side (intravesicular) or the exterior side (extravesicular) of the membrane. In some embodiments, a surface protein biomarker is associated with or present in an extracellular vesicle.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder, and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Target-binding moiety: In general, the terms “target-binding moiety” and “binding moiety” are used interchangeably herein to refer to any entity or moiety that binds to a target of interest (e.g., molecular target of interest such as a biomarker or an epitope). In many embodiments, a target-binding moiety of interest is one that binds specifically with its target (e.g., a target biomarker) in that it discriminates its target from other potential binding partners in a particular interaction context. In general, a target-binding moiety may be or comprise an entity or moiety of any chemical class (e.g., polymer, non-polymer, small molecule, polypeptide, carbohydrate, lipid, nucleic acid, etc.). In some embodiments, a target-binding moiety is a single chemical entity. In some embodiments, a target-binding moiety is a complex of two or more discrete chemical entities associated with one another under relevant conditions by non-covalent interactions. For example, those skilled in the art will appreciate that in some embodiments, a target-binding moiety may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple target binding moieties through linkage of different specific binding moieties with a generic binding moiety partner.

Target biomarker signature: The term “target biomarker signature”, as used herein, refers to a combination of (e.g., at least 2 or more, including, e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, or more) biomarkers, which combination correlates with a particular biological event or state of interest, so that one skilled in the art will appreciate that it may appropriately be considered to be a “signature” of that event or state. To give but a few examples, in some embodiments, a target biomarker signature may correlate with a particular disease or disease state, and/or with likelihood that a particular disease, disorder or condition may develop, occur, or reoccur. In some embodiments, a target biomarker signature may correlate with a particular disease or therapeutic outcome, or likelihood thereof. In some embodiments, a target biomarker signature may correlate with a specific cancer and/or stage thereof. In some embodiments, a target biomarker signature may correlate with ovarian cancer and/or a stage and/or a subtype thereof. In some embodiments, a target biomarker signature comprises a combination of (e.g., at least 2 or more, including, e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, or more) biomarkers that together are specific for an ovarian cancer or a subtype and/or a disease stage thereof), though one or more biomarkers in such a combination may be directed to a target (e.g., a surface protein biomarker, an intravesicular protein biomarker, and/or an intravesicular RNA) that is not specific to the ovarian cancer. For example, in some embodiments, a target biomarker signature may comprise at least one biomarker specific to an ovarian cancer or a stage and/or subtype thereof (i.e., an ovarian cancer-specific target), and may further comprise a biomarker that is not necessarily or completely specific for the ovarian cancer (e.g., that may also be found on some or all biological entities such as, e.g., cells, extracellular vesicles, etc., that are not cancerous, are not of the relevant cancer, and/or are not of the particular stage and/or subtype of interest). That is, as will be appreciated by those skilled in the art reading the present specification, so long as a combination of biomarkers utilized in a target biomarker signature is or comprises a plurality of biomarkers that together are specific for the relevant target biological entities of interest (e.g., ovarian cancer cells of interest or extracellular vesicles secreted by ovarian cancer cells) (i.e., sufficiently distinguish the relevant target biological entities (e.g., ovarian cancer cells of interest or extracellular vesicles secreted by ovarian cancer cells) for detection from other biological entities not of interest for detection), such a combination of biomarkers is a useful target biomarker signature in accordance with certain embodiments of the present disclosure.

Therapeutic agent: As used interchangeably herein, the phrase “therapeutic agent” or “therapy” refers to an agent or intervention that, when administered to a subject or a patient, has a therapeutic effect and/or elicits a desired biological and/or pharmacological effect. In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent or therapy is a medical intervention (e.g., surgery, radiation, phototherapy) that can be performed to alleviate, relieve, inhibit, present, delay onset of, reduce severity of, and/or reduce incidence of one or more symptoms or features of a disease, disorder, and/or condition.

Threshold level (e.g., cutoff): As used herein, the term “threshold level” refers to a level that are used as a reference to attain information on and/or classify the results of a measurement, for example, the results of a measurement attained in an assay. For example, in some embodiments, a threshold level (e.g., a cutoff) means a value measured in an assay that defines the dividing line between two subsets of a population (e.g., normal and/or non-ovarian cancer vs. ovarian cancer). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition. In some embodiments, treatment may be administered to a subject at a later-stage of disease, disorder, and/or condition.

Standard techniques may be used for recombinant DNA, oligonucleotide synthesis, and tissue culture and transformation (e.g., electroporation, lipofection). Enzymatic reactions and purification techniques may be performed according to manufacturer’s specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for the purpose described herein.

### DETAILED DESCRIPTION OF CERTAIN EMBODIMENTS

Ovarian cancer was responsible for an estimated 14,070 deaths in 2018 in the United States (Torre et al., 2018; which is incorporated herein by reference for the purpose described herein). The majority of these deaths are attributable to late diagnosis; ovarian cancer has an estimated five-year survival rate of 93% if caught at its earliest stage versus 26% if caught at its latest stage (Torre et al., 2018; which is incorporated herein by reference for the purpose described herein). The detection of high-grade serous ovarian cancer (HGSOC) is particularly important given that HGSOC accounts for 70% to 80% of all ovarian cancer deaths, while other subtypes are slower growing and susceptible to over diagnosis when using current technologies (Temkin et al., 2017; which is incorporated herein by reference for the purpose described herein). Unfortunately, despite being the fifth largest killer of women among all cancers (Howlader et al., 2019; which is incorporated herein by reference for the purpose described herein), there are no recommended ovarian cancer screening tests for average-risk women. While many women at hereditary risk and/or who may be experiencing one or more symptoms of ovarian cancer (e.g., fluid in the peritoneal cavity (ascites), general gastrointestinal dysfunction, constipation, bowel obstruction, nausea, vomiting, diarrhea, gastrointestinal reflux, increased abdominal size, urinary symptoms, abdominal bloating, abdominal and/or pelvic pain, fatigue, and/or shortness of breath) are currently screened by serum CA-125 and/or transvaginal ultrasound (TVUS), these tests are suboptimal for screening, because they have low sensitivity (~20%) for stage I and II disease and poor specificity. For example, the Prostate, Lung, Colorectal and Ovarian Cancer Screening Randomized Trial found serum CA-125 and TVUS increases the number of unnecessary surgeries and provides no mortality benefit for average-risk women (Buys et al., 2011; which is incorporated herein by reference for the purpose described herein). Despite this poor performance, serum CA-125 and TVUS are currently common screening tools for triaging post-menopausal women with nonspecific pelvic pain, which may be potentially indicative of ovarian cancer.

The present disclosure, among other things, identifies the source of a problem with certain prior technologies including, for example, certain conventional approaches to detection and diagnosis of ovarian cancer. For example, the present disclosure appreciates that many conventional diagnostic assays, e.g., based on cell-free nucleic acids, serum proteins (e.g., CA-125), and/or bulk analysis of extracellular vesicles, can be time-consuming, costly, and/or lacking sensitivity and/or specificity sufficient to provide a reliable and comprehensive diagnostic assessment. In some embodiments, the present disclosure provides technologies (including systems, compositions, and methods) that solve such problems, among other things, by identification of biomarker combinations that are predicted to exhibit high sensitivity and specificity for ovarian cancer based on bioinformatics analysis. In some embodiments, the present disclosure provides technologies (including systems, compositions, and methods) that solve such problems, by detecting co-localization of a target biomarker signature of ovarian cancer (e.g., identified by bioinformatics analysis) in individual extracellular vesicles, which comprises at least one extracellular vesicle-associated membrane-bound polypeptide and at least one target biomarker selected from the group consisting of surface protein biomarkers, internal protein biomarkers, and RNA biomarkers present in extracellular vesicles associated with ovarian cancer. In some embodiments, the present disclosure provides technologies (including systems, compositions, and methods) that solve such problems, among other things, by detecting such target biomarker signature of ovarian cancer using a target entity detection approach that was developed by Applicant and described in U.S. Application No. 16/805,637, and International Application PCT/US2020/020529, both filed Feb. 28, 2020 and entitled “Systems, Compositions, and Methods for Target Entity Detection,” which are based on interaction and/or co-localization of a target biomarker signature in individual extracellular vesicles. The contents of each of the aforementioned disclosures is incorporated herein by reference in their entirety.

The present disclosure, among other things, provides insights and technologies for achieving effective ovarian cancer screening, e.g., for early detection of ovarian cancer. In some embodiments, the present disclosure provides technologies for early detection of ovarian cancer in women who may be experiencing one more symptoms associated with ovarian cancer. In some embodiments, the present disclosure provides technologies for early detection of ovarian cancer in women who are at hereditary risks for ovarian cancer. In some embodiments, the present disclosure provides technologies for early detection of ovarian cancer in post-menopausal women who may be at hereditary risk and/or experiencing one or more symptoms associated with ovarian cancer. In some embodiments, the present disclosure provides technologies for screening women at hereditary or average risk for early stage high-grade serous ovarian cancer (HGSOC). HGSOC is the most common and lethal subtype of ovarian cancer, in which 84% of cases are detected at an advanced stage (Torre et al., 2018, which is incorporated herein by reference for the purpose described herein). In some embodiments, provided technologies are effective for detection of early stage ovarian cancers. In some embodiments, provided technologies are effective even when applied to populations comprising or consisting of asymptomatic or symptomatic individuals (e.g., due to sufficiently high sensitivity and/or low rates of false positive and/or false negative results). In some embodiments, provided technologies are effective when applied to populations comprising or consisting of individuals (e.g., asymptomatic or symptomatic individuals) without hereditary risk in developing ovarian cancer. In some embodiments, provided technologies are effective when applied to populations comprising or consisting of individuals (e.g., asymptomatic or symptomatic individuals) with hereditary risk in developing ovarian cancer. In some embodiments, provided technologies are effective when applied to populations comprising or consisting of individuals susceptible to ovarian cancer (e.g., individuals with a known genetic, environmental, or experiential risk, etc.). In some embodiments, provided technologies may be or include one or more compositions (e.g., molecular complexes, systems, collections, combinations, kits, etc.) and/or methods (e.g., of making, using, assessing, etc.), as will be clear to one skilled in the art reading the disclosure provided herein.

In some embodiments, provided technologies achieve detection (e.g., early detection, e.g., in asymptomatic individual(s) and/or population(s)) of one or more features (e.g., incidence, progression, responsiveness to therapy, recurrence, etc.) of ovarian cancer, with sensitivity and/or specificity (e.g., rate of false positive and/or false negative results) appropriate to permit useful application of provided technologies to single-time and/or regular (e.g., periodic) assessment. In some embodiments, provided technologies are useful in conjunction with women’s periodic physical examination such as mammogram, HPV, and/or Pap smear screening. In some embodiments, provided technologies are useful in conjunction with one or more screening methodologies (e.g., for ovarian cancer) such as CA-125 measurements (e.g., CA-125 serum level measurements) and/or TVUS. In some embodiments, provided technologies are useful in conjunction with treatment regimen(s); in some embodiments, provided technologies may improve one or more characteristics (e.g., rate of success according to an accepted parameter) of such treatment regimen(s).

In some embodiments, the present disclosure, among other things, provides insights that screening of asymptotic individuals, e.g., regular screening prior to or otherwise in absence of developed symptom(s), can be beneficial, and even important for effective management (e.g., successful treatment) of ovarian cancer. In some embodiments, the present disclosure provides ovarian cancer screening systems that can be implemented to detect ovarian cancer, including early-stage cancer, in some embodiments in asymptomatic individuals (e.g., without hereditary risks in ovarian cancer). In some embodiments, provided technologies are implemented to achieve regular screening of asymptomatic individuals (e.g., with or without hereditary risk(s) in ovarian cancer). In some embodiments, provided technologies are implemented to achieve regular screening of symptomatic individuals (e.g., with or without hereditary risk(s) in ovarian cancer). The present disclosure provides, for example, compositions (e.g., reagents, kits, components, etc.), and methods of providing and/or using them, including strategies that involve regular testing of one or more individuals (e.g., asymptomatic individuals). The present disclosure defines usefulness of such systems, and provides compositions and methods for implementing them.

### I. Ovarian Cancer Detection

Today there is no ovarian cancer screening test of any kind that is FDA approved for asymptomatic women of average risk, while in the US the average lifetime risk of developing ovarian cancer is 1.3%, the equivalent of 1 in 78 women. The overall ovarian cancer prevalence in the US was 5.7 per 10,000 women aged 55 to 74 years (Buys et al., 2011; which is incorporated herein by reference for the purpose described herein). In 2018, there were approximately 22,240 new cases of ovarian cancer diagnosed and 14,070 ovarian cancer deaths in the US (Torre et al., 2018; which is incorporated herein by reference for the purpose described herein). Among others, age and Menopausal state have been identified as a risk factor for ovarian cancer, where the mean age of initial presentation is approximately 68 years.

Epithelial ovarian cancer subtypes account for 90% of all ovarian cancers. Epithelial cancers are classified as serous (52%), endometrioid (10%), mucinous (6%), or clear-cell (6%), (Torre et al., 2018; which is incorporated herein by reference for the purpose described herein). Most serous carcinomas are diagnosed at stage III (51%) or stage IV (29%), when the 5-year survival rate is 42% and 26%, respectively, indicating the need for an early stage screening test. Germ cell and sex cord-stromal tumors make up the majority of non-epithelial cancers, but account for only 3% and 2%, respectively, of all ovarian cancers. Ovarian cancer affects women of all ethnicities.

The strongest risk factor for ovarian cancer is a family history of breast or ovarian cancer. Risk of developing invasive epithelial ovarian cancer is increased by approximately 50% among women with a first-degree relative with a history of ovarian cancer, and by 10% with a first-degree relative with breast cancer. Approximately 18% of epithelial ovarian cancer cases, particularly high-grade serous carcinomas, are estimated to be due to inherited mutations that confer elevated risk. Mutations in BRCA1 and BRCA2 account for almost 40% of ovarian cancer cases in women with a family history of the disease. Among women with BRCA1 or BRCA2 mutations, the risk of developing ovarian cancer by age 80 is 44% and 17%, respectively. Rare moderate-penetrance gene mutations for epithelial ovarian cancer include genes that are involved in the Fanconi anemia/BRCA pathway such as PALB2, BARD1, BRIP1, RAD51C, and RAD51D, for example, as described in Matulonis et al., 2016, which is incorporated herein by reference for the purpose described herein. Families with Lynch syndrome are characterized by a germline mutation in a DNA mismatch repair gene (e.g., MLH1, MSH2, MSH6 or PMS2). Women with Lynch syndrome have approximately an 8% risk of developing ovarian cancer (usually non-serous epithelial tumors) by age 70 compared to 0.7% in the general population (Torre, et al., 2018; which is incorporated herein by reference for the purpose described herein). Inherited mutations in other genes involved in DNA repair, such as CHEK2, MRE11A, RAD50, ATM, and TP53 may also increase the risk of developing ovarian cancer. Additional common, low penetrance alleles may also be associated with epithelial ovarian cancer susceptibility as suggested by genome wide association studies. Such genes and loci include: WNT4, RSPO1, BCL2L11, HOXD3, HAGLR, TIPARP, SYNPO2, TERT, GPX6, CHMP4C, LINC00824, COL15A1, SMC2-AS1, MLLT10, INCENP, RCCD1, ATAD5, HNF1B, PLEKHM1, SKAP1, ANKLE1, GATAD2A, Cytobands and SNPs 2q13 rs752590, 4q32.3 rs4691139, 9p22 rs3814113, 9q34.2 rs635634, 10p11.21 rs1192691, and/or 19q13.2 rs688187 (Reid et al., 2017; which is incorporated herein by reference for the purpose described herein).

The number of younger women identified with hereditary risk is expected to increase in the coming years. The NCCN guidelines for pancreatic cancer were updated in December 2019 to include a recommendation to test all patients for germline mutations in ATM, BRCA1, BRCA2, CDKN2A, MSH2, MLH1, MSH2, EPCAM, PALB2, STK11 and TP53. Given the overlap of this gene list with the genes conferring hereditary risk for ovarian cancer, it is likely that more daughters of pancreatic patients will become aware of their own genetic risk for both pancreatic and ovarian cancer, moving them from the general risk category into the hereditary risk category. In addition, a recent cost effectiveness study in breast cancer patients concluded that it is cost effective to screen all breast cancer patients in the US and the UK for germline mutations in BRCA1 and/or BRCA2 and PALB2 (Sun, et al., 2019; which is incorporated herein by reference for the purpose described herein). Implementation of germline genetic testing for all women with breast cancer into practice guidelines will identify additional risk-mutation carriers whose daughters are also at hereditary risk for breast and ovarian cancer. Currently, there is no recommended screening test for ovarian cancer in women (e.g., without hereditary risk). Among other things, in certain embodiments the present disclosure provides an insight that there is a need for development of an ovarian cancer liquid biopsy assay (e.g., as described herein) that can be utilized to provide an ovarian cancer risk assessment. In certain embodiments, assays and/or technologies described herein can provide a score relative to a reference threshold (e.g., as described herein). In certain embodiments, such a score can be or comprise an ovarian cancer risk score. In some embodiments, such a score can be used in conjunction with other ovarian cancer screening assessment(s) such as, e.g., but not limited to CA-125 measurements (e.g., CA-125 serum level measurements and/or TVUS) and/or ovarian cancer-associated risk factor(s) to provide an overall assessment.

The Prostate, Lung, Colorectal, and Ovarian Cancer Screening Trial (PLCO), which assessed the use of transvaginal ultrasound (TVUS) and a fixed cut-point (≥35 U/mL) in the tumor marker CA-125 for early detection, did not observe a reduction in ovarian cancer mortality after up to 19 years of follow-up. The UK Collaborative Trial of Ovarian Cancer Screening evaluated TVUS combined with a risk algorithm incorporating changes in CA-125 levels and found reduced mortality in average-risk women after 15 years. Despite the contradiction, the U.S. Preventive Services Task Force (USPSTF) continues to recommend against screening for ovarian cancer in the general population, concluding that there is adequate evidence that annual screening does not reduce ovarian cancer mortality and can lead to important harms, mainly surgical interventions in women without ovarian cancer.

Among other things, in certain embodiments the present disclosure provides an insight that there is a need for development of an ovarian cancer liquid biopsy assay for screening women with a hereditary risk for ovarian cancer and/or women who may be experiencing one or more symptoms associated with ovarian cancer. In certain embodiments, the present disclosure provides an insight that there is a need for development of an ovarian cancer liquid biopsy assay for screening symptomatic or asymptomatic women e.g., prior to other screening methods, e.g., TVUS. In certain embodiments, the present disclosure provides an insight that there is a need for development of an ovarian cancer liquid biopsy assay for screening asymptomatic women e.g., prior to other screening methods, e.g., TVUS. In certain embodiments, the present disclosure provides an insight that there is a need for development of an ovarian cancer liquid biopsy assay for screening women with an average risk for ovarian cancer. In certain embodiments, the present disclosure provides an insight that there is a need for development of an ovarian cancer liquid biopsy assay for screening women with life-history associated risk of ovarian cancer. In certain embodiments, the present disclosure provides an insight that there is a need for development of an ovarian cancer liquid biopsy assay for screening women who are post-menopausal, e.g., post-menopausal women who may be experiencing one or more symptoms associated with ovarian cancer. Despite being the fifth largest killer of women among all cancers (Howlader et al., 2019; which is incorporated herein by reference for the purpose described herein), there is currently no recommended ovarian cancer screening tool for average-risk women, while the current standard of care screening assays (e.g., TVUS and serum marker CA-125 levels) for stage 1 and II disease in women at hereditary risk and/or women who may be experiencing symptoms of ovarian cancer exhibit low sensitivity (~20%) and low specificity (NCCN, 2019; Buys et al., 2011; which are each incorporated herein by reference for the purpose described herein). These low rates of sensitivity and specificity pose a barrier to efficient and timely diagnosis. Given the incidence of ovarian cancer in average-risk women, inadequate test specificities (e.g., z<99.5%) result in false positive results that outnumber true positives by more than an order of magnitude. This places a significant burden on the healthcare system and on the women being screened as false positive results lead to additional tests, unnecessary surgeries, and emotional/physical distress (Buys et al., 2011; which is incorporated herein by reference for the purpose described herein).

In some embodiments, the present disclosure provides an insight that a particularly useful ovarian cancer screening test may be characterized by: (1) ultrahigh specificity (>98%) to minimize the number of false positives, and (2) high sensitivity (>40%) for stage I and II ovarian cancer (i.e., when prognosis is most favorable). For example, in some embodiments, a particularly useful ovarian cancer screening test may be characterized by a specificity of >98% and a sensitivity of >50%, for example, for stage I and II ovarian cancer. In some embodiments, a particularly useful ovarian cancer screening test may be characterized by a specificity of >98% and a sensitivity of >60%, for example, for stage I and II ovarian cancer. In some embodiments, a particularly useful ovarian cancer screening test may be characterized by a specificity of >98% and a sensitivity of >70%, for example, for stage I and II ovarian cancer. In some embodiments, a particularly useful ovarian cancer screening test may be characterized by a specificity of >99.5% and a sensitivity of >65%, for example, for stage I and II ovarian cancer. In some embodiments, a particularly useful ovarian cancer screening test may be characterized by a specificity of >99.5% and a sensitivity of >60%, for example, for stage I and II ovarian cancer.

In some embodiments, the present disclosure provides an insight that an ovarian cancer screening test involving more than one set of biomarker combinations (e.g., at least two orthogonal biomarker combinations as described herein) can increase sensitivity of such an assay, as compared to that is achieved by one set of biomarker combination. For example, in some embodiments, an ovarian cancer screening test involving at least two orthogonal biomarker combinations can achieve a specificity of at least 98% and a sensitivity of at least 50%. In some embodiments, an ovarian cancer screening test involving at least two orthogonal biomarker combinations can achieve a specificity of at least 98% and a sensitivity of at least 60%.

In some embodiments, the present disclosure provides an insight that a particularly useful ovarian cancer screening test may be characterized by an acceptable positive predictive value (PPV) at an economically justifiable cost. PPV is the likelihood a patient has the disease following a positive test, and is influenced by sensitivity, specificity, and/or disease prevalence. One clinician consensus for the minimum PPV needed to screen for ovarian cancer is 10% (Nossov et al., 2008; which is incorporated herein by reference for the purpose described herein). With a 10% PPV, there would be nine false positives for every one true positive. These false positives place a significant burden on both the healthcare system and the women being screened as they lead to additional tests, unnecessary surgeries, and emotional and physical distress (Buys et al., 2011; which is incorporated herein by reference for the purpose described herein). In some embodiments, assays described herein are particularly useful for early ovarian cancer detection that achieves a PPV of greater than 10% or higher, including, e.g., greater than 15%, greater than 20%, or greater than 25% or higher, with a specificity cutoff of at least 98% for women at hereditary risk for ovarian cancer, or with a specificity cutoff of at least 99.5% for women experiencing one or more symptoms associated with ovarian cancer.

In some embodiments, assays described herein can be useful for early ovarian cancer detection that achieves a PPV of greater than 2% or higher, including, e.g., greater than 3%, greater than 4%, greater than 5%, greater than 6% greater than 7%, greater than 8%, greater than 9%, greater than 10%, greater than 15%, greater than 20%, or greater than 25% or higher. In some such embodiments, assays described herein can achieve a specificity cutoff of at least 95% or higher (e.g., a specificity cutoff of at least 98% for women at hereditary risk for ovarian cancer, or with a specificity cutoff of at least 99.5% for women experiencing one or more symptoms associated with ovarian cancer).

Several different biomarker classes have been studied for an ovarian cancer liquid biopsy assay including circulating tumor DNA (ctDNA), circulating tumor cells (CTCs), bulk proteins, and extracellular vesicles (EVs). EVs are particularly promising due to their abundance and stability in the bloodstream relative to ctDNA and CTCs, suggesting improved sensitivity for early stage cancers. Moreover, EVs contain cargo (i.e., proteins, RNA, metabolites) that originated from the same cell, providing superior specificity over bulk protein measurements. While the diagnostic utility EVs has been studied, much of this work has pertained to bulk EV measurements or low-throughput single-EV analyses.

### II. Provided Biomarkers and/or Target Biomarker Signatures for Detection of Ovarian Cancer

The present disclosure, among other things, provides various target biomarkers or combinations thereof (e.g., target biomarker signatures) for ovarian cancer. Such target biomarker signatures that are predicted to exhibit high sensitivity and specificity for ovarian cancer were discovered by a multi-pronged bioinformatics analysis and biological approach, which for example, in some embodiments involve computational analysis of a diverse set of data, e.g., in some embodiments comprising one or more of sequencing data, expression data, mass spectrometry, histology, post-translational modification data, and/or in vitro and/or in vivo experimental data through machine learning and/or computational modeling.

In some embodiments, a target biomarker signature of ovarian cancer comprises at least one or more (e.g., 1, 2, 3, 4, 5, 6, 7, 8, or more) extracellular vesicle-associated membrane-bound polypeptide (e.g., surface polypeptide present in extracellular vesicles associated with ovarian cancer) and at least one or more (e.g., 1, 2, 3, 4, 5, 6, 7, 8, or more) target biomarkers selected from the group consisting of surface protein biomarker(s), intravesicular protein biomarker(s), and intravesicular RNA biomarker(s), such that the combination of such extracellular vesicle-associated membrane-bound polypeptide(s) and such target biomarker(s) present a target biomarker signature of ovarian cancer that provides (a) high specificity (e.g., greater than 98% or higher such as greater than 99%, or greater than 99.5%) to minimize the number of false positives, and (b) high sensitivity (e.g., greater than 40%, greater than 50%, greater than 60%, greater than 70%, greater than 80%) for stage I and II ovarian cancer when prognosis is most favorable. In some embodiments, a target biomarker signature of ovarian cancer comprises at least one extracellular vesicle-associated membrane-bound polypeptide (e.g., surface polypeptide present in extracellular vesicles associated with ovarian cancer) and at least one target biomarker selected from the group consisting of surface protein biomarker(s), intravesicular protein biomarker(s), and intravesicular RNA biomarker(s), such that the combination of such extracellular vesicle-associated membrane-bound polypeptide(s) and such target biomarker(s) present a target biomarker signature of ovarian cancer that provides a positive predictive value (PPV) at least 15% or higher, at least 20% or higher, at least 25% or higher, and/or at least 30% or higher. In some embodiments, a target biomarker signature of ovarian cancer comprises at least one extracellular vesicle-associated membrane-bound polypeptide (e.g., surface polypeptide present in extracellular vesicles associated with ovarian cancer) and at least one target biomarker selected from the group consisting of surface protein biomarker(s), intravesicular protein biomarker(s), and intravesicular RNA biomarker(s), such that the combination of such extracellular vesicle-associated membrane-bound polypeptide(s) and such target biomarker(s) present a target biomarker signature of ovarian cancer that provides a positive predictive value (PPV) of greater than 2% or higher, including, e.g., greater than 3%, greater than 5%, greater than 7%, greater than 10%, greater than 15% or higher, greater than 20% or higher, greater than 25% or higher, and/or greater than 30% or higher.

In some embodiments, the present disclosure recognizes that in certain embodiments, sensitivity and specificity rates for women with different ovarian risk levels may vary depending upon the risk tolerance of the attending physician and/or the guidelines set forth by interested medical consortia. In certain embodiments, women with hereditary risk of ovarian cancer may be best served with a 99.5% specificity rate with 70% sensitivity or a 98% specificity rate with 80% sensitivity. In certain embodiments, post-menopausal non-symptomatic women may be best served with a 99.5% specificity rate with 70% sensitivity or a 98% specificity rate with 80% sensitivity. In certain embodiments, post-menopausal symptomatic women may be best served with a 99.5% specificity rate with 70% sensitivity or a 98% specificity rate with 80% sensitivity. In certain embodiments, women with life-history risk may be best served with a 99.5% specificity rate with 70% sensitivity or a 98% specificity rate with 80% sensitivity. In some embodiments, technologies and/or assays described herein for detection of ovarian cancer in a symptomatic woman may have a lower sensitivity and/or specificity requirement than those for detection of ovarian cancer in an asymptomatic woman. In some embodiments, an assay described herein for detection of ovarian cancer in a symptomatic woman may have a set specificity rate that is lower than 99.5% specificity, including e.g., less than 99% sensitivity, less than 95%, less than 90%, or less than 85% specificity rate. In some embodiments, an assay described herein for detection of ovarian cancer in a symptomatic woman may have a set sensitivity rate that is lower than 80% sensitivity, including e.g., less than 70%, or less than 60% sensitivity rate.

In some embodiments, extracellular vesicle-associated membrane-bound polypeptide(s) included in a target biomarker signature of ovarian cancer is or comprises aquaporin-5 (AQP5) polypeptide, cadherin-6 (CDH6) polypeptide, chondrolectin (CHODL) polypeptide, claudin-3 (CLDN3) polypeptide, claudin-6 (CLDN6) polypeptide, claudin-16 (CLDN16) polypeptide, epithelial cell adhesion molecule (EpCAM), folate receptor alpha (FOLR1) polypeptide, 5-hydroxytryptamine receptor 3A (HTR3A) polypeptide, LEM domain-containing 1 (LEMD1) polypeptide, leucine-rich repeat transmembrane neuronal protein 1 (LRRTM1) polypeptide, mucin-16 (MUC16) polypeptide, sodium-dependent phosphate transport protein 2B (SLC34A2) polypeptide, alkaline phosphatase (ALPL) polypeptide, bone marrow stromal cell antigen 2 (BST2) polypeptide, small cell lung carcinoma cluster 4 antigen (CD24) polypeptide, mesothelin (MSLN) polypeptide, mucin-1 (MUC1) polypeptide, prostaglandin-endoperoxide synthase 1 (PTGS1) polypeptide, ST14 transmembrane serine protease matriptase (ST14) polypeptide, cancer-associated sialyl-Thompsen-nouvelle (Tn) (sTn) polypeptide glycosylation, tumor associated calcium signal transducer 2 (TACSTD2) polypeptide, basal cell adhesion molecule (BCAM) polypeptide, CD74 antigen (CD74) polypeptide, lymphocyte antigen 6 family member E (LY6E) polypeptide, solute carrier family 2 member 1 (SLC2A1) polypeptide, C-X-C motif chemokine receptor 4 (CXCR4) polypeptide, discoidin domain receptor tyrosine kinase 1 (DDR1) polypeptide, ephrin B 1 (EFNB 1) polypeptide, notch receptor 3 (NOTCH3) polypeptide, plexin B1 (PLXNB1) polypeptide, serine peptidase inhibitor kunitz type 2 (SPINT2) polypeptide, TNF receptor superfamily member 12A (TNFRSF12A) polypeptide, or combinations thereof.

In some embodiments, a target biomarker included in a target biomarker signature of ovarian cancer is or comprises a surface protein biomarker selected from the group consisting of: aquaporin-5 (AQP5) polypeptide, cadherin-6 (CDH6) polypeptide, chondrolectin (CHODL) polypeptide, claudin-3 (CLDN3) polypeptide, claudin-6 (CLDN6) polypeptide, claudin-16 (CLDN16) polypeptide, epithelial cell adhesion molecule (EpCAM), folate receptor 1 (FOLR1) polypeptide, 5-hydroxytryptamine receptor 3A (HTR3A) polypeptide, LEM domain-containing 1 (LEMD1) polypeptide, leucine-rich repeat transmembrane neuronal protein 1 (LRRTM1) polypeptide, mucin-16 (MUC16) polypeptide, sodium-dependent phosphate transport protein 2B (SLC34A2) polypeptide, alkaline phosphatase (ALPL) polypeptide, bone marrow stromal cell antigen 2 (BST2) polypeptide, small cell lung carcinoma cluster 4 antigen (CD24) polypeptide, mesothelin (MSLN) polypeptide, mucin-1 (MUC1) polypeptide, prostaglandin-endoperoxide synthase 1 (PTGS1) polypeptide, ST14 transmembrane serine protease matriptase (ST14) polypeptide, cancer-associated sialyl-Tn (sTn) polypeptide glycosylation, tumor associated calcium signal transducer 2 (TACSTD2) polypeptide, basal cell adhesion molecule (BCAM) polypeptide, CD74 antigen (CD74) polypeptide, lymphocyte antigen 6 family member E (LY6E) polypeptide, solute carrier family 2 member 1 (SLC2A1) polypeptide, C-X-C motif chemokine receptor 4 (CXCR4) polypeptide, discoidin domain receptor tyrosine kinase 1 (DDR1) polypeptide, ephrin B1 (EFNB 1) polypeptide, notch receptor 3 (NOTCH3) polypeptide, plexin B1 (PLXNB 1) polypeptide, serine peptidase inhibitor kunitz type 2 (SPINT2) polypeptide, TNF receptor superfamily member 12A (TNFRSF12A) polypeptide, or combinations thereof.

In some embodiments, extracellular vesicle-associated membrane-bound polypeptide(s) included in a target biomarker signature of ovarian cancer is or comprises aquaporin-5 (AQP5) polypeptide, cadherin-6 (CDH6) polypeptide, chondrolectin (CHODL) polypeptide, claudin-3 (CLDN3) polypeptide, claudin-6 (CLDN6) polypeptide, claudin-16 (CLDN16) polypeptide, epithelial cell adhesion molecule (EpCAM), folate receptor alpha (FOLR1) polypeptide, 5-hydroxytryptamine receptor 3A (HTR3A) polypeptide, LEM domain-containing 1 (LEMD1) polypeptide, leucine-rich repeat transmembrane neuronal protein 1 (LRRTM1) polypeptide, mucin-16 (MUC16) polypeptide, sodium-dependent phosphate transport protein 2B (SLC34A2) polypeptide, or combinations thereof.

In some embodiments, a target biomarker signature may comprise targets of a combination as depicted in Table 1, wherein a target may be used in a capture probe and/or detection probe. In some embodiments, a target biomarker signature may comprise a target of capture probe as depicted in Table 1 and at least one or more (including, e.g., at least two or more) targets of detection probes (e.g., detection probe 1 and/or detection probe 2). By way of example only, in some embodiments, a target biomarker signature may comprise ALPL (a target of capture probe depicted in Table 1), sTn (a target of detection probe 1 or 2 depicted in Table 1) and FOLR1 (a target of detection probe 1 or 2 depicted in Table 1). In some embodiments, a target biomarker signature may comprise targets of a combination of capture and detection probes as depicted in Table 1.

TABLE 1

exemplary target biomarker signature probe combinations

| Combination | Target of Capture Probe | Target of Detection Probe 1 | Target of Detection Probe 2 |
|---|---|---|---|
| 1 | ALPL | sTn | sTn |
| 2 | ALPL | FOLR1 | FOLR1 |
| 3 | BST2 | FOLR1 | FOLR1 |
| 4 | BST2 | MSLN | MSLN |
| 5 | BST2 | sTn | sTn |
| 6 | BST2 | MUC16 | MUC16 |
| 7 | FOLR1 | MUC1 | MUC1 |
| 8 | FOLR1 | TACSTD2 | TACSTD2 |
| 9 | FOLR1 | FOLR1 | MUC16 |
| 10 | FOLR1 | FOLR1 | MSLN |
| 11 | FOLR1 | SLC2A1 | SLC2A1 |
| 12 | FOLR1 | FOLR1 | BST2 |
| 13 | FOLR1 | SLC34A2 | FOLR1 |
| 14 | FOLR1 | FOLR1 | TACSTD2 |
| 15 | FOLR1 | FOLR1 | CD24 |
| 16 | FOLR1 | FOLR1 | SLC2A1 |
| 17 | FOLR1 | FOLR1 | sTn |
| 18 | FOLR1 | FOLR1 | ALPL |
| 19 | FOLR1 | MUC16 | FOLR1 |
| 20 | FOLR1 | sTn | sTn |
| 21 | FOLR1 | MSLN | MSLN |
| 22 | FOLR1 | MUC16 | MUC16 |
| 23 | FOLR1 | FOLR1 | MUC1 |
| 24 | MSLN | SLC34A2 | MSLN |
| 25 | MSLN | MSLN | sTn |
| 26 | MSLN | MSLN | BST2 |
| 27 | MSLN | MSLN | TACSTD2 |
| 28 | MSLN | sTn | sTn |
| 29 | MSLN | TACSTD2 | TACSTD2 |
| 30 | MSLN | FOLR1 | MSLN |
| 31 | MSLN | MSLN | SLC2A1 |
| 32 | MSLN | MUC1 | MSLN |
| 33 | MSLN | MSLN | CD24 |
| 34 | MSLN | MUC16 | MSLN |
| 35 | MSLN | MSLN | MUC16 |
| 36 | MSLN | FOLR1 | FOLR1 |
| 37 | MSLN | MUC16 | MUC16 |
| 38 | MSLN | MUC1 | MUC1 |
| 39 | MSLN | SLC2A1 | SLC2A1 |
| 40 | MUC1 | MUC1 | sTn |
| 41 | MUC1 | MUC16 | MUC16 |
| 42 | MUC1 | FOLR1 | FOLR1 |
| 43 | MUC1 | MUC16 | MUC1 |
| 44 | MUC1 | sTn | sTn |
| 45 | MUC1 | FOLR1 | MUC1 |
| 46 | MUC1 | MUC1 | MSLN |
| 47 | MUC1 | MSLN | MSLN |
| 48 | MUC1 | MUC1 | MUC16 |
| 49 | MUC16 | SLC34A2 | MUC16 |
| 50 | MUC16 | MUC16 | TACSTD2 |
| 51 | MUC16 | MUC16 | SLC2A1 |
| 52 | MUC16 | MUC1 | MUC1 |
| 53 | MUC16 | MUC16 | MSLN |
| 54 | MUC16 | TACSTD2 | TACSTD2 |
| 55 | MUC16 | MSLN | MSLN |
| 56 | MUC16 | MUC16 | sTn |
| 57 | MUC16 | sTn | sTn |
| 58 | MUC16 | FOLR1 | FOLR1 |
| 59 | MUC16 | MUC16 | MUC1 |
| 60 | MUC16 | MUC16 | FOLR1 |
| 61 | MUC16 | MUC16 | MUC16 |
| 62 | MUC16 | MUC16 | BST2 |
| 63 | MUC16 | MUC16 | CD24 |
| 64 | MUC16 | SLC2A1 | SLC2A1 |
| 65 | PTGS 1 | FOLR1 | FOLR1 |
| 66 | PTGS 1 | sTn | sTn |
| 67 | PTGS 1 | MSLN | MSLN |
| 68 | PTGS 1 | MUC16 | MUC16 |
| 69 | SLC34A2 | SLC2A1 | SLC2A1 |
| 70 | SLC34A2 | MSLN | MSLN |
| 71 | SLC34A2 | sTn | sTn |
| 72 | SLC34A2 | FOLR1 | FOLR1 |
| 73 | SLC34A2 | MUC16 | MUC16 |
| 74 | sTn | TACSTD2 | TACSTD2 |
| 75 | sTn | TACSTD2 | sTn |
| 76 | sTn | MUC16 | sTn |
| 77 | sTn | CD24 | sTn |
| 78 | sTn | ALPL | sTn |
| 79 | sTn | SLC34A2 | sTn |
| 80 | sTn | BST2 | sTn |
| 81 | sTn | SLC2A1 | sTn |
| 82 | sTn | SLC2A1 | SLC2A1 |
| 83 | sTn | MUC16 | MUC16 |
| 84 | sTn | MUC16 | sTn |
| 85 | sTn | MUC1 | sTn |
| 86 | sTn | FOLR1 | FOLR1 |
| 87 | sTn | MUC1 | MUC1 |
| 88 | sTn | FOLR1 | sTn |
| 89 | sTn | MSLN | sTn |
| 90 | sTn | MSLN | MSLN |
| 91 | TACSTD2 | FOLR1 | TACSTD2 |
| 92 | TACSTD2 | TACSTD2 | MUC16 |
| 93 | TACSTD2 | FOLR1 | FOLR1 |
| 94 | TACSTD2 | MUC16 | TACSTD2 |
| 95 | TACSTD2 | MSLN | TACSTD2 |
| 96 | TACSTD2 | TACSTD2 | sTn |
| 97 | TACSTD2 | MSLN | MSLN |
| 98 | TACSTD2 | MUC16 | MUC16 |
| 99 | TACSTD2 | sTn | sTn |
| 100 | SLC34A2 | SLC34A2 | SLC34A2 |
| 101 | SLC34A2 | SLC34A2 | MUC16 |
| 102 | SLC34A2 | SLC34A2 | FOLR1 |
| 103 | SLC34A2 | SLC34A2 | CLDN3 |
| 104 | SLC34A2 | SLC34A2 | CLDN6 |
| 105 | SLC34A2 | SLC34A2 | AQP5 |
| 106 | SLC34A2 | MUC16 | MUC16 |
| 107 | SLC34A2 | MUC16 | FOLR1 |
| 108 | SLC34A2 | MUC16 | CLDN3 |
| 109 | SLC34A2 | MUC16 | CLDN6 |
| 110 | SLC34A2 | MUC16 | AQP5 |
| 111 | SLC34A2 | FOLR1 | FOLR1 |
| 112 | SLC34A2 | FOLR1 | CLDN3 |
| 113 | SLC34A2 | FOLR1 | CLDN6 |
| 114 | SLC34A2 | FOLR1 | AQP5 |
| 115 | SLC34A2 | CLDN3 | CLDN3 |
| 116 | SLC34A2 | CLDN3 | CLDN6 |
| 117 | SLC34A2 | CLDN3 | AQP5 |
| 118 | SLC34A2 | CLDN6 | CLDN6 |
| 119 | SLC34A2 | CLDN6 | AQP5 |
| 120 | SLC34A2 | AQP5 | AQP5 |
| 121 | MUC16 | SLC34A2 | SLC34A2 |
| 122 | MUC16 | SLC34A2 | MUC16 |
| 123 | MUC16 | SLC34A2 | FOLR1 |
| 124 | MUC16 | SLC34A2 | CLDN3 |
| 125 | MUC16 | SLC34A2 | CLDN6 |
| 126 | MUC16 | SLC34A2 | AQP5 |
| 127 | MUC16 | MUC16 | MUC16 |
| 128 | MUC16 | MUC16 | FOLR1 |
| 129 | MUC16 | MUC16 | CLDN3 |
| 130 | MUC16 | MUC16 | CLDN6 |
| 131 | MUC16 | MUC16 | AQP5 |
| 132 | MUC16 | FOLR1 | FOLR1 |
| 133 | MUC16 | FOLR1 | CLDN3 |
| 134 | MUC16 | FOLR1 | CLDN6 |
| 135 | MUC16 | FOLR1 | AQP5 |
| 136 | MUC16 | CLDN3 | CLDN3 |
| 137 | MUC16 | CLDN3 | CLDN6 |
| 138 | MUC16 | CLDN3 | AQP5 |
| 139 | MUC16 | CLDN6 | CLDN6 |
| 140 | MUC16 | CLDN6 | AQP5 |
| 141 | MUC16 | AQP5 | AQP5 |
| 142 | FOLR1 | SLC34A2 | SLC34A2 |
| 143 | FOLR1 | SLC34A2 | MUC16 |
| 144 | FOLR1 | SLC34A2 | FOLR1 |
| 145 | FOLR1 | SLC34A2 | CLDN3 |
| 146 | FOLR1 | SLC34A2 | CLDN6 |
| 147 | FOLR1 | SLC34A2 | AQP5 |
| 148 | FOLR1 | MUC16 | MUC16 |
| 149 | FOLR1 | MUC16 | FOLR1 |
| 150 | FOLR1 | MUC16 | CLDN3 |
| 151 | FOLR1 | MUC16 | CLDN6 |
| 152 | FOLR1 | MUC16 | AQP5 |
| 153 | FOLR1 | FOLR1 | FOLR1 |
| 154 | FOLR1 | FOLR1 | CLDN3 |
| 155 | FOLR1 | FOLR1 | CLDN6 |
| 156 | FOLR1 | FOLR1 | AQP5 |
| 157 | FOLR1 | CLDN3 | CLDN3 |
| 158 | FOLR1 | CLDN3 | CLDN6 |
| 159 | FOLR1 | CLDN3 | AQP5 |
| 160 | FOLR1 | CLDN6 | CLDN6 |
| 161 | FOLR1 | CLDN6 | AQP5 |
| 162 | FOLR1 | AQP5 | AQP5 |
| 163 | CLDN3 | SLC34A2 | SLC34A2 |
| 164 | CLDN3 | SLC34A2 | MUC16 |
| 165 | CLDN3 | SLC34A2 | FOLR1 |
| 166 | CLDN3 | SLC34A2 | CLDN3 |
| 167 | CLDN3 | SLC34A2 | CLDN6 |
| 168 | CLDN3 | SLC34A2 | AQP5 |
| 169 | CLDN3 | MUC16 | MUC16 |
| 170 | CLDN3 | MUC16 | FOLR1 |
| 171 | CLDN3 | MUC16 | CLDN3 |
| 172 | CLDN3 | MUC16 | CLDN6 |
| 173 | CLDN3 | MUC16 | AQP5 |
| 174 | CLDN3 | FOLR1 | FOLR1 |
| 175 | CLDN3 | FOLR1 | CLDN3 |
| 176 | CLDN3 | FOLR1 | CLDN6 |
| 177 | CLDN3 | FOLR1 | AQP5 |
| 178 | CLDN3 | CLDN3 | CLDN3 |
| 179 | CLDN3 | CLDN3 | CLDN6 |
| 180 | CLDN3 | CLDN3 | AQP5 |
| 181 | CLDN3 | CLDN6 | CLDN6 |
| 182 | CLDN3 | CLDN6 | AQP5 |
| 183 | CLDN3 | AQP5 | AQP5 |
| 184 | LRRTM1 | SLC34A2 | SLC34A2 |
| 185 | LRRTM1 | SLC34A2 | MUC16 |
| 186 | LRRTM1 | SLC34A2 | FOLR1 |
| 187 | LRRTM1 | SLC34A2 | CLDN3 |
| 188 | LRRTM1 | SLC34A2 | CLDN6 |
| 189 | LRRTM1 | SLC34A2 | AQP5 |
| 190 | LRRTM1 | MUC16 | MUC16 |
| 191 | LRRTM1 | MUC16 | FOLR1 |
| 192 | LRRTM1 | MUC16 | CLDN3 |
| 193 | LRRTM1 | MUC16 | CLDN6 |
| 194 | LRRTM1 | MUC16 | AQP5 |
| 195 | LRRTM1 | FOLR1 | FOLR1 |
| 196 | LRRTM1 | FOLR1 | CLDN3 |
| 197 | LRRTM1 | FOLR1 | CLDN6 |
| 198 | LRRTM1 | FOLR1 | AQP5 |
| 199 | LRRTM1 | CLDN3 | CLDN3 |
| 200 | LRRTM1 | CLDN3 | CLDN6 |
| 201 | LRRTM1 | CLDN3 | AQP5 |
| 202 | LRRTM1 | CLDN6 | CLDN6 |
| 203 | LRRTM1 | CLDN6 | AQP5 |
| 204 | LRRTM1 | AQP5 | AQP5 |

In some embodiments, certain biomarker combinations as depicted in Table 1 that may be particularly useful (e.g., with higher sensitivity, specificity and/or PPV) for ovarian cancer detection can undergo an initial round of screening using an advanced stage (e.g., late stage, e.g., stage III and/or IV) ovarian cancer sample pool and the healthy control sample pool as a reference. In some embodiments, select combinations can be further tested using early-stage ovarian cancer sample pools (e.g., stage I and/or II, optionally differentiated by low or high CA-125 content), benign gynecological tumor plasma sample pools (e.g., as described herein), non-ovarian cancer sample pools (e.g., as described herein), and/or any combination thereof. In some embodiments, biomarker combination performance can be determined by calculating the difference in assay signal (e.g., on a Ct basis) between the healthy sample pools and ovarian cancer sample pools.

In some embodiments, certain biomarker combinations for ovarian cancer detection can be selected with a delta Ct greater than inter-assay variability. For example, in some embodiments, biomarker combinations with a delta Ct greater than 2.0 (corresponding to a fourfold difference) or 1.0 (corresponding to a twofold difference) are considered to provide particularly effective diagnostic utility (e.g., providing a signal greater than inter-assay variability). See, e.g., Example 7, which provides an exemplary analysis of certain combinations described herein.

In some embodiments, a target biomarker included in a target biomarker signature of ovarian cancer is or comprises an intravesicular protein biomarker selected from the group consisting of: cellular retinoic acid-binding protein 2 (CRABP2) polypeptide, kallikrein-7 (KLK7) polypeptide, macrophage migration inhibitory factor (MIF) polypeptide, preferentially expressed antigen in melanoma (PRAME) polypeptide, S100 calcium-binding protein A1 (S100A1) polypeptide, and combinations thereof.

In some embodiments, a target biomarker included in a target biomarker signature of ovarian cancer is or comprises an intravesicular RNA (e.g., mRNA) biomarker selected from the group consisting of: CLDN6 RNA, CRABP2 RNA, KLK7 RNA, MIF RNA, PRAME RNA, S100A1 RNA, and combinations thereof.

In some embodiments, a target biomarker signature for ovarian cancer comprises at least one or more (e.g., 1, 2, 3, 4, 5, 6, 7, 8, or more) extracellular vesicle-associated membrane-bound polypeptides (e.g., ones described herein) and at least one or more (e.g., 1, 2, 3, 4, 5, 6, 7, 8, or more) surface protein biomarkers (e.g., ones described herein). In some such embodiments, an extracellular vesicle-associated membrane-bound polypeptide and at least one surface protein biomarker are the same. For example, in some embodiments, a target biomarker signature for ovarian cancer comprises at least one extracellular vesicle-associated membrane-bound polypeptide, which is a MUC16 polypeptide, and at least one surface protein biomarker, which is a MUC16 polypeptide. In some embodiments, a target biomarker signature for ovarian cancer comprises at least one extracellular vesicle-associated membrane-bound polypeptide, which is a FOLR1 polypeptide, and at least one surface protein biomarker, which is a FOLR1 polypeptide.

In some embodiments, at least one extracellular vesicle-associated membrane-bound polypeptide and at least one surface protein biomarker(s) of a target biomarker signature for ovarian cancer are distinct. For example, in some embodiments, a target biomarker signature for ovarian cancer comprises at least one extracellular vesicle-associated membrane-bound polypeptide, which is or comprises a FOLR1 polypeptide, and at least one surface protein biomarker, which is or comprises a CLDN3 polypeptide, a CLDN6 polypeptide, and/or a SLC34A2 polypeptide. In some embodiments, a target biomarker signature for ovarian cancer comprises at least one extracellular vesicle-associated membrane-bound polypeptide, which is or comprises a MUC16 polypeptide, and at least one surface protein biomarker, which is or comprises a CLDN3 polypeptide, a CLDN6 polypeptide, and/or a FOLR1 polypeptide. In some embodiments, a target biomarker signature for ovarian cancer comprises at least one extracellular vesicle-associated membrane-bound polypeptide, which is or comprises a SLC34A2 polypeptide, and at least one surface protein biomarker, which is or comprises a MUC16 polypeptide, and/or a FOLR1 polypeptide. In some embodiments, a target biomarker signature for ovarian cancer comprises at least one extracellular vesicle-associated membrane-bound polypeptide, which is or comprises a LRRTM1 polypeptide, and at least one surface protein biomarker, which is or comprises a MUC16 polypeptide.

In some embodiments, a target biomarker signature for ovarian cancer comprises at least one or more (e.g., 1, 2, 3, 4, 5, 6, 7, 8, or more) extracellular vesicle-associated membrane-bound polypeptides (e.g., ones described herein) and at least one or more (e.g., 1, 2, 3, 4, 5, 6, 7, 8, or more) intravesicular protein biomarkers (e.g., ones described herein). In some such embodiments, the extracellular vesicle-associated membrane-bound polypeptide(s) and the intravesicular protein biomarker(s) can be encoded by the same gene, while the former is expressed in the membrane of the extracellular vesicle and the latter is expressed within the extracellular vesicle. In some such embodiments, the extracellular vesicle-associated membrane-bound polypeptide(s) and the intravesicular protein biomarker(s) can be encoded by different genes.

In some embodiments, a target biomarker signature for ovarian cancer comprises at least one or more (e.g., 1, 2, 3, 4, 5, 6, 7, 8, or more) extracellular vesicle-associated membrane-bound polypeptides (e.g., ones described herein) and at least one or more (e.g., 1, 2, 3, 4, 5, 6, 7, 8, or more) intravesicular RNA (e.g., mRNA) biomarkers (e.g., ones described herein). In some such embodiments, the extracellular vesicle-associated membrane-bound polypeptide(s) and the intravesicular RNA (e.g., mRNA) biomarker(s) can be encoded by the same gene. In some such embodiments, the extracellular vesicle-associated membrane-bound polypeptide(s) and the intravesicular RNA (e.g., mRNA) biomarker(s) can be encoded by different genes.

In some embodiments, a target biomarker signature for ovarian cancer can be or comprise targets of a combination as described in Tables 3-5. In certain embodiments, a target biomarker signature for ovarian cancer is one that differentiates late stage ovarian cancer samples from a control sample (e.g., compared to healthy samples, compared to benign gynecological tumor samples, and/or compared to other cancer samples; see e.g., Tables 3-5). In certain embodiments, a target biomarker signature for ovarian cancer is one that differentiates early stage ovarian cancer samples (e.g., with low and/or high serum CA-125) from a control sample (e.g., compared to healthy samples, compared to benign gynecological tumor samples, and/or compared to other cancer samples; see e.g., Tables 3-5). In some embodiments, an assay directed to detection of a target biomarker signature for ovarian cancer can comprise a combination of capture and detection probes as described in Tables 3-5.

In some embodiments, a target biomarker signature for ovarian cancer comprises at least a SLC34A2 polypeptide (as an extracellular vesicle-associated membrane-bound polypeptide) and a MUC16 polypeptide (as a target surface protein biomarker).

In some embodiments, a target biomarker signature for ovarian cancer comprises at least a SLC34A2 polypeptide (as an extracellular vesicle-associated membrane-bound polypeptide) and a FOLR1 polypeptide (as a target surface protein biomarker).

In some embodiments, a target biomarker signature for ovarian cancer comprises at least a SLC34A2 polypeptide (as an extracellular vesicle-associated membrane-bound polypeptide) and at least two target surface protein biomarkers, which may be or comprise a MUC16 polypeptide and a FOLR1 polypeptide.

In some embodiments, a target biomarker signature for ovarian cancer comprises at least a MUC16 polypeptide (as an extracellular vesicle-associated membrane-bound polypeptide) and a MUC16 polypeptide (as a target surface protein biomarker).

In some embodiments, a target biomarker signature for ovarian cancer comprises at least a MUC16 polypeptide (as an extracellular vesicle-associated membrane-bound polypeptide) and at least two target surface protein biomarkers, which may be or comprise a MUC16 polypeptide and a CLDN6 polypeptide.

In some embodiments, a target biomarker signature for ovarian cancer comprises at least a MUC16 polypeptide (as an extracellular vesicle-associated membrane-bound polypeptide) and at least two target surface protein biomarkers, which may be or comprise a SLC34A2 polypeptide and a FOLR1 polypeptide.

In some embodiments, a target biomarker signature for ovarian cancer comprises at least a MUC16 polypeptide (as an extracellular vesicle-associated membrane-bound polypeptide) and at least two target surface protein biomarkers, which may be or comprise a MUC16 polypeptide and a CLDN3 polypeptide.

In some embodiments, a target biomarker signature for ovarian cancer comprises at least a MUC16 polypeptide (as an extracellular vesicle-associated membrane-bound polypeptide) and at least two target surface protein biomarkers, which may be or comprise a FOLR1 polypeptide and a CLDN3 polypeptide.

In some embodiments, a target biomarker signature for ovarian cancer comprises at least a MUC16 polypeptide (as an extracellular vesicle-associated membrane-bound polypeptide) and at least one target surface protein biomarker, which may be or comprise a FOLR1 polypeptide.

In some embodiments, a target biomarker signature for ovarian cancer comprises at least a MUC16 polypeptide (as an extracellular vesicle-associated membrane-bound polypeptide) and at least one target surface protein biomarker, which may be or comprise an AQP5 polypeptide.

In some embodiments, a target biomarker signature for ovarian cancer comprises at least a MUC16 polypeptide (as an extracellular vesicle-associated membrane-bound polypeptide) and at least two target surface protein biomarkers, which may be or comprise a FOLR1 polypeptide and a AQP5 polypeptide

In some embodiments, a target biomarker signature for ovarian cancer comprises at least a FOLR1 polypeptide (as an extracellular vesicle-associated membrane-bound polypeptide) and at least two target surface protein biomarkers, which may be or comprise a FOLR1 polypeptide and a CLDN6 polypeptide.

In some embodiments, a target biomarker signature for ovarian cancer comprises at least a FOLR1 polypeptide (as an extracellular vesicle-associated membrane-bound polypeptide) and at least two target surface protein biomarkers, which may be or comprise a SLC34A2 polypeptide and a CLDN3 polypeptide.

In some embodiments, a target biomarker signature for ovarian cancer comprises at least a FOLR1 polypeptide (as an extracellular vesicle-associated membrane-bound polypeptide) and at least two target surface protein biomarkers, which may be or comprise a SLC34A2 polypeptide and a MUC16 polypeptide.

In some embodiments, a target biomarker signature for ovarian cancer comprises at least a FOLR1 polypeptide (as an extracellular vesicle-associated membrane-bound polypeptide) and at least one target surface protein biomarker, which may be or comprise an MUC16 polypeptide.

In some embodiments, a target biomarker signature for ovarian cancer comprises at least a FOLR1 polypeptide (as an extracellular vesicle-associated membrane-bound polypeptide) and at least one target surface protein biomarker, which may be or comprise an FOLR1 polypeptide.

In some embodiments, a target biomarker signature for ovarian cancer comprises at least a FOLR1 polypeptide (as an extracellular vesicle-associated membrane-bound polypeptide) and at least two target surface protein biomarkers, which may be or comprise a FOLR1 polypeptide and a CLDN3 polypeptide.

In some embodiments, a target biomarker signature for ovarian cancer comprises at least a FOLR1 polypeptide (as an extracellular vesicle-associated membrane-bound polypeptide) and at least two target surface protein biomarkers, which may be or comprise a FOLR1 polypeptide and a AQP5 polypeptide.

In some embodiments, a target biomarker signature for ovarian cancer comprises at least a FOLR1 polypeptide (as an extracellular vesicle-associated membrane-bound polypeptide) and at least one target surface protein biomarker, which may be or comprise an CLDN6 polypeptide.

In some embodiments, a target biomarker signature for ovarian cancer comprises at least a FOLR1 polypeptide (as an extracellular vesicle-associated membrane-bound polypeptide) and at least two target surface protein biomarkers, which may be or comprise a MUC16 polypeptide and a FOLR1 polypeptide.

In some embodiments, a target biomarker signature for ovarian cancer comprises at least a FOLR1 polypeptide (as an extracellular vesicle-associated membrane-bound polypeptide) and at least two target surface protein biomarkers, which may be or comprise a MUC16 polypeptide and a CLDN3 polypeptide.

In some embodiments, a target biomarker signature for ovarian cancer comprises at least a LRRTM1 polypeptide (as an extracellular vesicle-associated membrane-bound polypeptide) and at least one target surface protein biomarker, which may be or comprise a MUC16 polypeptide.

In some embodiments, a target biomarker signature for ovarian cancer comprises at least a CLDN3 polypeptide (as an extracellular vesicle-associated membrane-bound polypeptide) and at least one target surface protein biomarker, which may be or comprise a FOLR1 polypeptide.

In some embodiments, a target biomarker signature for ovarian cancer comprises at least a CLDN3 polypeptide (as an extracellular vesicle-associated membrane-bound polypeptide) and at least two target surface protein biomarkers, which may be or comprise a SLC34A2 polypeptide and a MUC16 polypeptide.

In some embodiments, a target biomarker signature for ovarian cancer comprises at least a CLDN3 polypeptide (as an extracellular vesicle-associated membrane-bound polypeptide) and at least two target surface protein biomarkers, which may be or comprise a MUC16 polypeptide and a CLDN3 polypeptide.

In some embodiments, a target biomarker signature for ovarian cancer comprises at least a CLDN3 polypeptide (as an extracellular vesicle-associated membrane-bound polypeptide) and at least one target surface protein biomarker, which may be or comprise a MUC16 polypeptide.

In some embodiments, a target biomarker signature for ovarian cancer comprises at least a CLDN3 polypeptide (as an extracellular vesicle-associated membrane-bound polypeptide) and at least two target surface protein biomarkers, which may be or comprise a MUC16 polypeptide and a FOLR1 polypeptide.

In some embodiments, a target biomarker signature for ovarian cancer comprises at least a CLDN3 polypeptide (as an extracellular vesicle-associated membrane-bound polypeptide) and at least two target surface protein biomarkers, which may be or comprise a MUC16 polypeptide and a CLDN3 polypeptide.

In some embodiments, a target biomarker signature for ovarian cancer comprises at least a CLDN3 polypeptide (as an extracellular vesicle-associated membrane-bound polypeptide) and at least two target surface protein biomarkers, which may be or comprise a MUC16 polypeptide and a CLDN6 polypeptide.

In some embodiments, a target biomarker signature for ovarian cancer comprises at least a CLDN6 polypeptide (as an extracellular vesicle-associated membrane-bound polypeptide) and at least one target surface protein biomarker, which may be or comprise a MUC16 polypeptide.

In some embodiments, a target biomarker signature for ovarian cancer comprises at least an ALPL polypeptide (as an extracellular vesicle-associated membrane-bound polypeptide) and at least one target surface protein biomarker, which may be or comprise a FOLR1 polypeptide.

In some embodiments, a target biomarker signature for ovarian cancer comprises at least a BST2 polypeptide (as an extracellular vesicle-associated membrane-bound polypeptide) and at least one target surface protein biomarker, which may be or comprise a MUC16 polypeptide.

In some embodiments, a target biomarker signature for ovarian cancer comprises at least a FOLR1 polypeptide (as an extracellular vesicle-associated membrane-bound polypeptide) and at least one target surface protein biomarker, which may be or comprise a MSLN polypeptide.

In some embodiments, a target biomarker signature for ovarian cancer comprises at least a FOLR1 polypeptide (as an extracellular vesicle-associated membrane-bound polypeptide) and at least one target surface protein biomarker, which may be or comprise a MUC16 polypeptide.

In some embodiments, a target biomarker signature for ovarian cancer comprises at least a MSLN polypeptide (as an extracellular vesicle-associated membrane-bound polypeptide) and at least one target surface protein biomarker, which may be or comprise a MUC16 polypeptide.

In some embodiments, a target biomarker signature for ovarian cancer comprises at least a MSLN polypeptide (as an extracellular vesicle-associated membrane-bound polypeptide) and at least one target surface protein biomarker, which may be or comprise a MUC1 polypeptide.

In some embodiments, a target biomarker signature for ovarian cancer comprises at least a MSLN polypeptide (as an extracellular vesicle-associated membrane-bound polypeptide) and at least one target surface protein biomarker, which may be or comprise a SLC2A1 polypeptide.

In some embodiments, a target biomarker signature for ovarian cancer comprises at least a MUC1 polypeptide (as an extracellular vesicle-associated membrane-bound polypeptide) and at least one target surface protein biomarker, which may be or comprise a sTn glycosylated polypeptide.

In some embodiments, a target biomarker signature for ovarian cancer comprises at least a MUC1 polypeptide (as an extracellular vesicle-associated membrane-bound polypeptide) and at least one target surface protein biomarker, which may be or comprise a MUC16 polypeptide.

In some embodiments, a target biomarker signature for ovarian cancer comprises at least a MUC1 polypeptide (as an extracellular vesicle-associated membrane-bound polypeptide) and at least one target surface protein biomarker, which may be or comprise a FOLR1 polypeptide.

In some embodiments, a target biomarker signature for ovarian cancer comprises at least a MUC16 polypeptide (as an extracellular vesicle-associated membrane-bound polypeptide) and at least one target surface protein biomarker, which may be or comprise a sTn glycosylated polypeptide.

In some embodiments, a target biomarker signature for ovarian cancer comprises at least a PTGS1 polypeptide (as an extracellular vesicle-associated membrane-bound polypeptide) and at least one target surface protein biomarker, which may be or comprise a MUC16 polypeptide.

In some embodiments, a target biomarker signature for ovarian cancer comprises at least a sTn glycosylated polypeptide (as an extracellular vesicle-associated membrane-bound polypeptide) and at least one target surface protein biomarker, which may be or comprise a FOLR1 polypeptide.

In some embodiments, a target biomarker signature for ovarian cancer comprises at least a sTn glycosylated polypeptide (as an extracellular vesicle-associated membrane-bound polypeptide) and at least one target surface protein biomarker, which may be or comprise a MSLN polypeptide.

In some embodiments, a target biomarker signature for ovarian cancer comprises at least a TACSTD2 polypeptide (as an extracellular vesicle-associated membrane-bound polypeptide) and at least one target surface protein biomarker, which may be or comprise a MUC16 polypeptide.

In some embodiments, a target biomarker signature for ovarian cancer comprises at least a TACSTD2 polypeptide (as an extracellular vesicle-associated membrane-bound polypeptide) and at least one target surface protein biomarker, which may be or comprise a sTn glycosylated polypeptide.

In some embodiments, a target biomarker signature for ovarian cancer comprises at least a TACSTD polypeptide (as an extracellular vesicle-associated membrane-bound polypeptide) and at least two target surface protein biomarkers, which may be or comprise a MUC16 polypeptide and a sTn glycosylated polypeptide.

In some embodiments, a target biomarker signature for ovarian cancer comprises at least a TACSTD polypeptide (as an extracellular vesicle-associated membrane-bound polypeptide) and at least two target surface protein biomarkers, which may be or comprise a MUC1 polypeptide and a sTn glycosylated polypeptide.

In some embodiments, a target biomarker signature for ovarian cancer comprises at least a TACSTD polypeptide (as an extracellular vesicle-associated membrane-bound polypeptide) and at least two target surface protein biomarkers, which may be or comprise a MSLN polypeptide and a sTn glycosylated polypeptide.

In some embodiments, a target biomarker signature for ovarian cancer comprises at least a TACSTD polypeptide (as an extracellular vesicle-associated membrane-bound polypeptide) and at least two target surface protein biomarkers, which may be or comprise a FOLR1 polypeptide and a sTn glycosylated polypeptide.

In some embodiments, a target biomarker signature for ovarian cancer comprises at least a TACSTD polypeptide (as an extracellular vesicle-associated membrane-bound polypeptide) and at least two target surface protein biomarkers, which may be or comprise a MUC16 polypeptide and a MUC1 polypeptide.

In some embodiments, a target biomarker signature for ovarian cancer comprises at least a TACSTD polypeptide (as an extracellular vesicle-associated membrane-bound polypeptide) and at least two target surface protein biomarkers, which may be or comprise a MUC16 polypeptide and a MSLN polypeptide.

In some embodiments, a target biomarker signature for ovarian cancer comprises at least a TACSTD polypeptide (as an extracellular vesicle-associated membrane-bound polypeptide) and at least two target surface protein biomarkers, which may be or comprise a MUC16 polypeptide and a FOLR1 polypeptide.

In some embodiments, a target biomarker signature for ovarian cancer comprises at least a TACSTD polypeptide (as an extracellular vesicle-associated membrane-bound polypeptide) and at least two target surface protein biomarkers, which may be or comprise a MUC1 polypeptide and a MSLN polypeptide.

In some embodiments, a target biomarker signature for ovarian cancer comprises at least a TACSTD polypeptide (as an extracellular vesicle-associated membrane-bound polypeptide) and at least two target surface protein biomarkers, which may be or comprise a MUC1 polypeptide and a FOLR1 polypeptide.

In some embodiments, a target biomarker signature for ovarian cancer comprises at least a TACSTD polypeptide (as an extracellular vesicle-associated membrane-bound polypeptide) and at least two target surface protein biomarkers, which may be or comprise a MSLN polypeptide and a FOLR1 polypeptide.

In some embodiments, a target biomarker signature for ovarian cancer comprises at least a MUC1 polypeptide (as an extracellular vesicle-associated membrane-bound polypeptide) and at least two target surface protein biomarkers, which may be or comprise a MUC16 polypeptide and a sTn glycosylated polypeptide.

In some embodiments, a target biomarker signature for ovarian cancer comprises at least a MUC1 polypeptide (as an extracellular vesicle-associated membrane-bound polypeptide) and at least two target surface protein biomarkers, which may be or comprise a MUC16 polypeptide and a MSLN polypeptide.

In some embodiments, a target biomarker signature for ovarian cancer comprises at least a MUC1 polypeptide (as an extracellular vesicle-associated membrane-bound polypeptide) and at least two target surface protein biomarkers, which may be or comprise a MUC16 polypeptide and a FOLR1 polypeptide.

In some embodiments, a target biomarker signature for ovarian cancer comprises at least a MUC1 polypeptide (as an extracellular vesicle-associated membrane-bound polypeptide) and at least two target surface protein biomarkers, which may be or comprise a MSLN polypeptide and a sTn glycosylated polypeptide.

In some embodiments, a target biomarker signature for ovarian cancer comprises at least a MUC1 polypeptide (as an extracellular vesicle-associated membrane-bound polypeptide) and at least two target surface protein biomarkers, which may be or comprise a FOLR1 polypeptide and a sTn glycosylated polypeptide.

In some embodiments, a target biomarker signature for ovarian cancer comprises at least a MUC1 polypeptide (as an extracellular vesicle-associated membrane-bound polypeptide) and at least two target surface protein biomarkers, which may be or comprise a MSLN polypeptide and a FOLR1 polypeptide.

In some embodiments, a target biomarker signature for ovarian cancer comprises at least a MUC16 polypeptide (as an extracellular vesicle-associated membrane-bound polypeptide) and at least two target surface protein biomarkers, which may be or comprise a MUC1 polypeptide and a sTn glycosylated polypeptide.

In some embodiments, a target biomarker signature for ovarian cancer comprises at least a MUC16 polypeptide (as an extracellular vesicle-associated membrane-bound polypeptide) and at least two target surface protein biomarkers, which may be or comprise a MSLN polypeptide and a sTn glycosylated polypeptide.

In some embodiments, a target biomarker signature for ovarian cancer comprises at least a MUC16 polypeptide (as an extracellular vesicle-associated membrane-bound polypeptide) and at least two target surface protein biomarkers, which may be or comprise a FOLR1 polypeptide and a sTn glycosylated polypeptide.

In some embodiments, a target biomarker signature for ovarian cancer comprises at least a MUC16 polypeptide (as an extracellular vesicle-associated membrane-bound polypeptide) and at least two target surface protein biomarkers, which may be or comprise a MUC1 polypeptide and a MSLN polypeptide.

In some embodiments, a target biomarker signature for ovarian cancer comprises at least a MUC16 polypeptide (as an extracellular vesicle-associated membrane-bound polypeptide) and at least two target surface protein biomarkers, which may be or comprise a MUC1 polypeptide and a FOLR1 polypeptide.

In some embodiments, a target biomarker signature for ovarian cancer comprises at least a MUC16 polypeptide (as an extracellular vesicle-associated membrane-bound polypeptide) and at least two target surface protein biomarkers, which may be or comprise a MSLN polypeptide and a FOLR1 polypeptide.

In some embodiments, a target biomarker signature for ovarian cancer comprises at least a sTn glycosylated polypeptide (as an extracellular vesicle-associated membrane-bound polypeptide) and at least two target surface protein biomarkers, which may be or comprise a MUC16 polypeptide and a MUC1 polypeptide.

In some embodiments, a target biomarker signature for ovarian cancer comprises at least a sTn glycosylated polypeptide (as an extracellular vesicle-associated membrane-bound polypeptide) and at least two target surface protein biomarkers, which may be or comprise a MUC16 polypeptide and a MSLN polypeptide.

In some embodiments, a target biomarker signature for ovarian cancer comprises at least a sTn glycosylated polypeptide (as an extracellular vesicle-associated membrane-bound polypeptide) and at least two target surface protein biomarkers, which may be or comprise a MUC16 polypeptide and a FOLR1 polypeptide.

In some embodiments, a target biomarker signature for ovarian cancer comprises at least a sTn glycosylated polypeptide (as an extracellular vesicle-associated membrane-bound polypeptide) and at least two target surface protein biomarkers, which may be or comprise a MUC1 polypeptide and a MSLN polypeptide.

In some embodiments, a target biomarker signature for ovarian cancer comprises at least a sTn glycosylated polypeptide (as an extracellular vesicle-associated membrane-bound polypeptide) and at least two target surface protein biomarkers, which may be or comprise a MUC1 polypeptide and a FOLR1 polypeptide.

In some embodiments, a target biomarker signature for ovarian cancer comprises at least a sTn glycosylated polypeptide (as an extracellular vesicle-associated membrane-bound polypeptide) and at least two target surface protein biomarkers, which may be or comprise a MSLN polypeptide and a FOLR1 polypeptide.

In some embodiments, a target biomarker signature for ovarian cancer comprises at least a MSLN polypeptide (as an extracellular vesicle-associated membrane-bound polypeptide) and at least two target surface protein biomarkers, which may be or comprise a MUC16 polypeptide and a sTn glycosylated polypeptide.

In some embodiments, a target biomarker signature for ovarian cancer comprises at least a MSLN polypeptide (as an extracellular vesicle-associated membrane-bound polypeptide) and at least two target surface protein biomarkers, which may be or comprise a MUC16 polypeptide and a MUC1 polypeptide.

In some embodiments, a target biomarker signature for ovarian cancer comprises at least a MSLN polypeptide (as an extracellular vesicle-associated membrane-bound polypeptide) and at least two target surface protein biomarkers, which may be or comprise a MUC16 polypeptide and a FOLR1 polypeptide.

In some embodiments, a target biomarker signature for ovarian cancer comprises at least a MSLN polypeptide (as an extracellular vesicle-associated membrane-bound polypeptide) and at least two target surface protein biomarkers, which may be or comprise a MUC1 polypeptide and a sTn glycosylated polypeptide.

In some embodiments, a target biomarker signature for ovarian cancer comprises at least a MSLN polypeptide (as an extracellular vesicle-associated membrane-bound polypeptide) and at least two target surface protein biomarkers, which may be or comprise a FOLR1 polypeptide and a sTn glycosylated polypeptide.

In some embodiments, a target biomarker signature for ovarian cancer comprises at least a MSLN polypeptide (as an extracellular vesicle-associated membrane-bound polypeptide) and at least two target surface protein biomarkers, which may be or comprise a MUC1 polypeptide and a FOLR1 polypeptide.

In some embodiments, a target biomarker signature for ovarian cancer comprises at least a SLC34A2 polypeptide (as an extracellular vesicle-associated membrane-bound polypeptide) and at least two target surface protein biomarkers, which may be or comprise a MUC16 polypeptide and a MSLN polypeptide.

In some embodiments, a target biomarker signature for ovarian cancer comprises at least a SLC34A2 polypeptide (as an extracellular vesicle-associated membrane-bound polypeptide) and at least two target surface protein biomarkers, which may be or comprise a MUC16 polypeptide and a sTn glycosylated polypeptide.

In some embodiments, a target biomarker signature for ovarian cancer comprises at least a SLC34A2 polypeptide (as an extracellular vesicle-associated membrane-bound polypeptide) and at least two target surface protein biomarkers, which may be or comprise a FOLR1 polypeptide and a sTn glycosylated polypeptide.

In some embodiments, any one of provided biomarkers can be detected and/or measured by protein and/or RNA (e.g., mRNA) expression levels in wild-type form.

In some embodiments, any one of provided biomarkers can be detected and/or measured by protein and/or RNA (e.g., mRNA) expression levels in mutant form. Thus, in some embodiments, mutant-specific detection of provided biomarkers (e.g., proteins and/or RNA such as, e.g., mRNAs) can be included.

As noted herein, in some embodiments, a biomarker is or comprises a particular form of one or more polypeptides or proteins (e.g., a pro- form, a truncated form, a modified form such as a glycosylated, phosphorylated, lipidated form, etc). In some embodiments, detection of such form detects a plurality (and, in some embodiments, substantially all) polypeptides present in that form (e.g., containing a particular modification such as, for example, a particular glycosylation, e.g., sialyl-Tn (sTn) glycosylation, e.g., a truncated O-glycan containing a sialic acid α-2,6 linked to GalNAc α-O-Ser/Thr.

Accordingly, in some embodiments, a surface protein biomarker can be or comprise a glycosylation moiety (e.g., an sTn moiety). Thompsen-nouvelle (Tn) antigen is an O-linked glycan that is thought to be associated with a broad array of tumors. Tn is a single alpha-linked GalNAc added to Ser or Thr as the first step of a major O-linked glycosylation pathway. In some embodiments, a surface protein biomarker can be or comprise a tumor-associated post-translational modification. In some embodiments, such a post-translational modification can be or comprise tumor-specific glycosylation patterns such as mucins with glycans aberrantly truncated at the initial GalNAc (e.g., Tn), or combinations thereof.

In some embodiments, a biomarker is or comprises a truncated form of a polypeptide. For example, in some embodiments, a MUC16 biomarker is a truncated form of a MUC16 protein.

### III. Exemplary Methods of Detecting Provided Markers and/or Target Biomarker Signatures for Ovarian Cancer

In general, the present disclosure provides technologies according to which a target biomarker signature is analyzed and/or assessed in a blood-derived sample comprising extracellular vesicles from a subject in need thereof; in some embodiments, a diagnosis or therapeutic decision is made based on such analysis and/or assessment.

In some embodiments, methods of detecting a target biomarker signature include methods for detecting one or more provided markers of a target biomarker signature as proteins. Exemplary protein-based methods of detecting one or more provided markers include, but are not limited to, proximity ligation assay, mass spectrometry (MS) and immunoassays, such as immunoprecipitation; Western blot; ELISA; immunohistochemistry; immunocytochemistry; flow cytometry; and immuno-PCR. In some embodiments, an immunoassay can be a chemiluminescent immunoassay. In some embodiments, an immunoassay can be a high-throughput and/or automated immunoassay platform.

In some embodiments, methods of detecting one or more provided markers as proteins in a sample comprise contacting a sample with one or more antibody agents directed to the provided markers of interest. In some embodiments, such methods also comprise contacting the sample with one or more detection labels. In some embodiments, antibody agents are labeled with one or more detection labels.

In some embodiments, detecting binding between a biomarker of interest and an antibody agent for the biomarker of interest includes determining absorbance values or emission values for one or more detection agents. For example, the absorbance values or emission values are indicative of amount and/or concentration of biomarker of interest expressed by extracellular vesicles (e.g., higher absorbance is indicative of higher level of biomarker of interest expressed by extracellular vesicles). In some embodiments, absorbance values or emission values for detection agents are above a threshold value. In some embodiments, absorbance values or emission values for detection agents is at least 1.3, at least 1.4, at least 1.5, at least 1.6, at least 1.7, at least 1.8, at least 1.9, at least 2.0, at least 2.5, at least 3.0, at least 3.5 fold or greater than a threshold value. In some embodiments, the threshold value is determined across a population of a control or reference group (e.g., non-cancer subjects).

In some embodiments, methods of detecting one or more provided markers include methods for detecting one or more provided markers as nucleic acids. Exemplary nucleic acid-based methods of detecting one or more provided markers include, but are not limited to, performing nucleic acid amplification methods, such as polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). In some embodiments, a nucleic acid-based method of detecting one or more provided markers includes detecting hybridization between one or more nucleic acid probes and one or more nucleotides that encode a biomarker of interest. In some embodiments, the nucleic acid probes are each complementary to at least a portion of one of the one or more nucleotides that encode the biomarker of interest. In some embodiments, the nucleotides that encode the biomarker of interest include DNA (e.g., cDNA). In some embodiments, the nucleotides that encode the biomarker of interest include RNA (e.g., mRNA).

In some embodiments, methods of detecting one or more provided markers involve proximity-ligation-immuno quantitative polymerase chain reaction (pliq-PCR). Pliq-PCR can have certain advantages over other technologies to profile EVs. For example, pliq-PCR can have a sensitivity three orders of magnitude greater than other standard immunoassays, such as ELISAs (Darmanis et al., 2010; which is incorporated herein by reference for the purpose described herein). In some embodiments, a pliq-PCR reaction can be designed to have an ultra-low LOD, which enables to detect trace levels of tumor-derived EVs, for example, down to a thousand EVs per mL.

In some embodiments, methods for detecting one or more provided markers may involve other technologies for detecting EVs, including, e.g., Nanoplasmic Exosome (nPLEX) Sensor (Im et al., 2014; which is incorporated herein by reference for the purpose described herein) and the Integrated Magnetic-Electrochemical Exosome (iMEX) Sensor (Jeong et al., 2016; which is incorporated herein by reference for the purpose described herein), which have reported LODs of ~10³ and ~10⁴ EVs, respectively (Shao et al., 2018; which is incorporated herein by reference for the purpose described herein).

In some embodiments, methods for detecting one or more provided biomarkers in extracellular vesicles can be based on bulk EV sample analysis.

In some embodiments, methods for detecting one or more provided biomarkers in extracellular vesicles can be based on profiling individual EVs (e.g., single-EV profiling assays), which is further discussed in the section entitled “Exemplary Methods for Profiling Individual Extracellular Vesicles (EVs)” below.

In some embodiments, extracellular vesicles in a sample may be captured or immobilized on a solid substrate prior to detecting one or more provided biomarkers in accordance with the present disclosure. In some embodiments, extracellular vesicles may be captured on a solid substrate surface by non-specific interaction, including, e.g., adsorption. In some embodiments, extracellular vesicles may be selectively captured on a solid substrate surface. For example, in some embodiments, a solid substrate surface may be coated with an agent that specifically binds to extracellular vesicles (e.g., an antibody agent specifically targeting extracellular vesicles, e.g., associated with ovarian cancer). In some embodiments, a solid substrate surface may be coated with a member of an affinity binding pair and an entity of interest (e.g., extracellular vesicles) to be captured may be conjugated to a complementary member of the affinity binding pair. In some embodiments, an exemplary affinity binding pair includes, e.g., but is not limited to biotin and avidin-like molecules such as streptavidin. As will be understood by those of skilled in the art, other appropriate affinity binding pairs can also be used to facilitate capture of an entity of interest to a solid substrate surface. In some embodiments, an entity of interest may be captured on a solid substrate surface by application of a current, e.g., as described in Ibsen et al. ACS Nano., 11: 6641-6651 (2017) and Lewis et al. ACS Nano., 12: 3311-3320 (2018), both of which are incorporated herein by reference for the purpose described herein, and both of which describe use of an alternating current electrokinetic microarray chip device to isolate extracellular vesicles from an undiluted human blood or plasma sample.

A solid substrate may be provided in a form that is suitable for capturing extracellular vesicles and does not interfere with downstream handling, processing, and/or detection. For example, in some embodiments, a solid substrate may be or comprise a bead (e.g., a magnetic bead). In some embodiments, a solid substrate may be or comprise a surface. For example, in some embodiments, such a surface may be a capture surface of an assay chamber (including, e.g., a tube, a well, a microwell, a plate, a filter, a membrane, a matrix, etc.). Accordingly, in some embodiments, a method described herein comprises, prior to detecting provided biomarkers in a sample, capturing or immobilizing extracellular vesicles on a solid substrate.

In some embodiments, a sample may be processed, e.g., to remove undesirable entities such as cell debris or cells, prior to capturing extracellular vesicles on a solid substrate surface. For example, in some embodiments, such a sample may be subjected to centrifugation, e.g., to remove cell debris, cells, and/or other particulates. Additionally or alternatively, in some embodiments, such a sample may be subjected to size-exclusion-based purification or filtration. Various size-exclusion-based purification or filtration are known in the art and those skilled in the art will appreciate that in some cases, a sample may be subjected to a spin column purification based on specific molecular weight or particle size cutoff. Those skilled in the art will also appreciate that appropriate molecular weight or particle size cutoff for purification purposes can be selected, e.g., based on the size of extracellular vesicles. For example, in some embodiments, size-exclusion separation methods may be applied to samples comprising extracellular vesicles to isolate a fraction of extracellular vesicles that are of a certain size (e.g., greater than 30 nm and no more than 1000 nm, or greater than 70 nm and no more than 200 nm). Typically, extracellular vesicles may range from 30 nm to several micrometers in diameter. See, e.g., Chuo et al., “Imaging extracellular vesicles: current and emerging methods” Journal of Biomedical Sciences 25: 91 (2018) which is incorporated herein by reference for the purpose described herein, which provides information of sizes for different extracellular vesicle (EV) subtypes: migrasomes (0.5-3 µm), microvesicles (0.1-1 µm), oncosomes (1-10 µm), exomeres (<50 nm), small exosomes (60-80 nm), and large exosomes (90-120 nm). In some embodiments, nanoparticles having a size range of about 30 nm to 1000 nm may be isolated, for example, in some embodiments by one or more size-exclusion separation methods, for detection assay. In some embodiments, specific EV subtype(s) may be isolated, for example, in some embodiments by one or more size-exclusion separation methods, for detection assay.

In some embodiments, extracellular vesicles in a sample may be processed prior to detecting one or more provided biomarkers of a target biomarker signature for ovarian cancer. Different sample processing and/or preparation can be performed, e.g., to stabilize targets (e.g., target biomarkers) in extracellular vesicles to be detected, and/or to facilitate exposure of targets (e.g., intravesicular proteins and/or RNA such as mRNA) to a detection assay (e.g., as described herein), and/or to reduce non-specific binding. Examples of such sample processing and/or preparation are known in the art and include, but are not limited to, crosslinking molecular targets (e.g., fixation), permeabilization of biological entities (e.g., cells or extracellular vesicles), and/or blocking non-specific binding sites.

In one aspect, the present disclosure provides a method for detecting whether a target biomarker signature of ovarian cancer is present or absent in a biological sample from a subject in need thereof, which may be in some embodiments a blood-derived sample comprising extracellular vesicles. In some embodiments, such a method comprises (a) detecting, in a biological sample such as a blood-derived sample (e.g., a plasma sample) from a subject, biological entities of interest (including, e.g., extracellular vesicles) expressing a target biomarker signature of ovarian cancer; and (b) comparing sample information indicative of the level of the target biomarker signature-expressing biological entities of interest (e.g., extracellular vesicles) in the biological sample (e.g., blood-derived sample) to reference information including a reference threshold level. In some embodiments, a reference threshold level corresponds to a level of biological entities of interest (e.g., extracellular vesicles) that express such a target biomarker signature in comparable samples from a population of reference subjects, e.g., non-cancer subjects. In some embodiments, exemplary non-cancer subjects include healthy female subjects (e.g., healthy female subjects of specified age ranges, such as e.g., below age 55 or above age 55), female subjects with non-ovarian related health diseases, disorders, or conditions (including, e.g., female subjects having non-ovarian cancer such as lung cancer, colorectal cancer, etc., or female subjects having symptoms of inflammatory bowel diseases or disorders), female subjects having benign ovarian tumors (e.g., a benign mass observed in a fallopian tube and/or on an ovary), and combinations thereof.

In some embodiments, a sample is pre-screened for certain characteristics prior to utilization in an assay as described herein. In some embodiments, a sample meeting certain pre-screening criteria is more suitable for diagnostic applications than a sample failing pre-screening criteria. For example, in some embodiments samples are visually inspected for appearance using known standards, e.g., is the sample normal, hemolyzed (red), icteric (yellow), and/or lipemic (turbid). In some embodiments, samples can then be rated on a known standard scale (e.g., 1, 2, 3, 4, 5) and the results are recorded. In some embodiments, samples are visually inspected for hemolysis (e.g., heme) and rated on a scale from 1-5, where the visual inspection correlates with a known concentration, e.g., where 1 denotes approximately 0 mg/dL, 2 denotes approximately 50 mg/dL, 3 denotes approximately 150 mg/dL, 4 denotes approximately 250 mg/dL, and 5 denotes approximately 525 mg/dL. In some embodiments, samples are visually inspected icteric levels (e.g., bilirubin) and rated on a scale from 1-5, where the visual inspection correlates with a known concentration, e.g., where 1 denotes approximately 0 mg/dL, 2 denotes approximately 1.7 mg/dL, 3 denotes approximately 6.6 mg/dL, 4 denotes approximately 16 mg/dL, and 5 denotes approximately 30 mg/dL. In some embodiments, samples are visually inspected for turbidity (e.g. lipids) and rated on a scale from 1-5, where the visual inspection correlates with a known concentration, e.g., where 1 denotes approximately 0 mg/dL, 2 denotes approximately 125 mg/dL, 3 denotes approximately 250 mg/dL, 4 denotes approximately 500 mg/dL, and 5 denotes approximately 1000 mg/dL.

In some embodiments, samples scoring lower than a certain level on one or more metrics, e.g., equal to or lower than a score of 4, may be utilized in an assay as described herein. In some embodiments, samples scoring lower than a certain level on one or more metrics, e.g., equal to or lower than a score of 3, may be utilized in an assay as described herein. In some embodiments, samples scoring lower than a certain level on one or more metrics, e.g., equal to or lower than a score of 2, may be utilized in an assay as described herein. In some embodiments, samples scoring lower than a certain level on all three metrics (e.g., hemolyzed, icteric, and lipemic) e.g., equal to or lower than a score of 2, may be utilized in an assay as described herein. In some embodiments, low visual inspection scores on pre-screening criteria such as hemolysis, bilirubin, and/or lipemia (e.g., equal to or lower than a score of 2) may have no appreciable effect (e.g., not be correlated with) on diagnostic properties (e.g., Ct values) produced in an assay as described herein.

In some embodiments, a sample is determined to have extracellular vesicles expressing a target biomarker signature (e.g., ones described herein) when it shows an elevated level of target biomarker signature-expressing extracellular vesicles relative to a reference threshold level (e.g., ones described herein). In some embodiments, a sample is determined to be positive for target biomarker signature-expressing extracellular vesicles if its level is at least 30% or higher, including, e.g., at least 40%, at least 50%, at least 60%, at least 70%, at least 80%, at least 90%, at least 95% or higher, as compared to a reference threshold level. In some embodiments, a sample is determined to be positive for target biomarker signature-expressing extracellular vesicles if its level is at least 2-fold or higher, including, e.g., at least 3-fold, at least 4-fold, at least 5-fold, at least 6-fold, at least 7-fold, at least 8-fold, at least 9-fold, at least 10-fold, at least 50-fold, at least 100-fold, at least 250-fold, at least 500-fold, at least 750-fold, at least 1000-fold, at least 2500-fold, at least 5000-fold, or higher, as compared to a reference threshold level.

In some embodiments, a binary classification system may be used to determine whether a sample is positive for target biomarker signature-expressing extracellular vesicles. For example, in some embodiments, a sample is determined to be positive for target biomarker signature-expressing extracellular vesicles if its level is at or above a reference threshold level, e.g., a cutoff value. In some embodiments, such a reference threshold level (e.g., a cutoff value) may be determined by selecting a certain number of standard deviations away from an average value obtained from control subjects such that a desired sensitivity and/or specificity of an ovarian cancer detection assay (e.g., ones described herein) can be achieved. In some embodiments, such a reference threshold level (e.g., a cutoff value) may be determined by selecting a certain number of standard deviations away from a maximum assay signal obtained from control subjects such that a desired sensitivity and/or specificity of an ovarian cancer detection assay (e.g., ones described herein) can be achieved. In some embodiments, such a reference threshold level (e.g., a cutoff value) may be determined by selecting the less restrictive of either (i) a certain number of standard deviations away from an average value obtained from control subjects, or (ii) a certain number of standard deviations away from a maximum assay signal obtained from control subjects, such that a desired sensitivity and/or specificity of an ovarian cancer detection assay (e.g., ones described herein) can be achieved. In some embodiments, control subjects for determination of a reference threshold level (e.g., a cutoff value) may include, but are not limited to healthy subjects, subjects with inflammatory conditions (e.g., Crohn’s disease, ulcerative colitis, endometriosis, etc.), subjects with benign gynecological tumors, and combinations thereof. In some embodiments, healthy subjects and subjects with inflammatory conditions (e.g., Crohn’s disease, ulcerative colitis, endometriosis, etc.) are included in determination of a reference threshold level (e.g., a cutoff value). In some embodiments, subjects with benign gynecological tumors are not included in determination of a reference threshold level (e.g., a cutoff value). In some embodiments, a reference threshold level (e.g., a cutoff value) may be determined by selecting at least 1.5 standard deviations (SDs) or higher (including, e.g., at least 1.6, at least 1.7, at least 1.8, at least 1.9, at least 2, at least 2.1, at least 2.2, at least 2.3, at least 2.4, at least 2.5, at least 2.6, at least 2.7, at least 2.8, at least 2.9, at least 3, at least 3.1, at least 3.2, at least 3.3, at least 3.4, at least 3.5, at least 3.6 or higher SDs) away from (i) an average value obtained from control subjects, or (ii) a maximum assay signal obtained from control subjects, such that a desired specificity (e.g., at least 95% or higher specificity [including, e.g., at least 96%, at least 97%, at least 98%, at least 99%, or higher specificity] such as in some embodiments at least 99.8% specificity) of an ovarian cancer detection assay (e.g., ones described herein) can be achieved. In some embodiments, a reference threshold level (e.g., a cutoff value) may be determined by selecting at least 2.9 SDs (e.g., at least 2.93 SDs) away from (i) an average value obtained from control subjects, or (ii) a maximum assay signal obtained from control subjects, such that a desired specificity (e.g., at least 99%, or higher specificity) of an ovarian cancer detection assay (e.g., ones described herein) can be achieved. In some embodiments, a reference threshold level (e.g., a cutoff value) may be determined by selecting at least 2.9 SDs (e.g., at least 2.93 SDs) away from the less restrictive of (i) an average value obtained from control subjects, or (ii) a maximum assay signal obtained from control subjects, such that a desired specificity (e.g., at least 99%, or higher specificity) of an ovarian cancer detection assay (e.g., ones described herein) can be achieved. In some embodiments, such a reference threshold level (e.g., a cutoff value) may be determined based on expression level (e.g., transcript level) of a target biomarker in normal healthy tissues vs. in ovarian cancer samples such that the specificity and/or sensitivity of interest (e.g., as described herein) can be achieved. In some embodiments, a reference threshold level (e.g., a cutoff value) may vary dependent on, for example, ovarian cancer stages and/or subtypes and/or patient characteristics, for example, patient age, menopausal status, risks factors for ovarian cancer (e.g., hereditary risk vs. average risk, life-history-associated risk factors), symptomatic/asymptomatic status, and combinations thereof.

In some embodiments, a reference threshold level (e.g., a cutoff value) may be determined based on a log-normal distribution around healthy female subjects (e.g., of specified age ranges), and optionally female subjects with inflammatory conditions (e.g., Crohn’s disease, ulcerative colitis, endometriosis, etc.) and selection of the number of standard deviations (SDs) (e.g., at least 1.5, at least 1.6, at least 1.7, at least 1.8, at least 1.9, at least 2, at least 2.1, at least 2.2, at least 2.3, at least 2.4, at least 2.5, at least 2.6, at least 2.7, at least 2.8, at least 2.9, at least 3, at least 3.1, at least 3.2, at least 3.3, at least 3.4, at least 3.5, at least 3.6 or higher SDs) necessary to achieve the specificity of interest (e.g., at least 95% or higher specificity [including, e.g., at least 96%, at least 97%, at least 98%, at least 99%, or higher specificity] such as in some embodiments at least 99.8% specificity), e.g., based on prevalence of ovarian cancer or a subtype thereof. The present disclosure, among other things, also provides technologies for determining whether a subject as having or being susceptible to ovarian cancer. For example, in some embodiments, when a blood-derived sample from a subject in need thereof shows a level of target biomarker signature-expressing extracellular vesicles that is at or above a reference threshold level, e.g., cutoff value (e.g., as determined in accordance with the present disclosure), then the subject is classified as having or being susceptible to ovarian cancer. In some such embodiments, a reference threshold level (e.g., cutoff value) may be determined based on a log-normal distribution around healthy female subjects (e.g., of specified age ranges), and optionally female subjects with inflammatory conditions (e.g., Crohn’s disease, ulcerative colitis, endometriosis, etc.) and selection of the number of standard deviations (SDs) (e.g., at least 1.5, at least 1.6, at least 1.7, at least 1.8, at least 1.9, at least 2, at least 2.1, at least 2.2, at least 2.3, at least 2.4, at least 2.5, at least 2.6, at least 2.7, at least 2.8, at least 2.9, at least 3, at least 3.1, at least 3.2, at least 3.3, at least 3.4, at least 3.5, at least 3.6 or higher SDs) necessary to achieve the specificity of interest (e.g., at least 95% or higher specificity [including, e.g., at least 96%, at least 97%, at least 98%, at least 99%, or higher specificity] such as in some embodiments at least 99.8% specificity), e.g., based on prevalence of ovarian cancer or a subtype thereof. In some such embodiments, a reference threshold level (e.g., a cutoff value) may be determined based on expression level (e.g., transcript level) of individual target biomarker(s) of a target biomarker signature in normal healthy tissues vs. in ovarian cancer samples such that the specificity and/or sensitivity of interest (e.g., as described herein) can be achieved. In some embodiments, a reference threshold level (e.g., a cutoff value) may vary dependent on, for example, ovarian cancer stages and/or subtypes and/or patient characteristics, for example, patient age, menopausal status, risks factors for ovarian cancer (e.g., hereditary risk vs. average risk, life-history-associated risk factors), symptomatic/asymptomatic status, and combinations thereof.

In some embodiments, when a blood-derived sample from a subject in need thereof shows an elevated level of target biomarker signature-expressing extracellular vesicles relative to a reference threshold level, then the subject is classified as having or being susceptible to ovarian cancer. In some embodiments, a subject in need thereof is classified as having or being susceptible to ovarian cancer when her blood-derived sample shows a level of target biomarker signature-expressing extracellular vesicles that is at least 30% or higher, including, e.g., at least 40%, at least 50%, at least 60%, at least 70%, at least 80%, at least 90%, at least 95% or higher, as compared to a reference threshold level. In some embodiments, a subject in need thereof is classified as having or being susceptible to ovarian cancer when her blood-derived sample shows a level of target biomarker signature-expressing extracellular vesicles that is at least 2-fold or higher, including, e.g., at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, at least 250-fold, at least 500-fold, at least 750-fold, at least 1000-fold, or higher, as compared to a reference threshold level. When a blood-derived sample from a subject in need thereof shows a comparable level (e.g., within 10-20%) to a reference threshold level, then the subject is classified as not likely to have or as not likely to be susceptible to ovarian cancer. In some such embodiments, a reference threshold level corresponds to a level of extracellular vesicles that express a target biomarker signature in comparable samples from a population of reference subjects, e.g., non-cancer subjects. In some embodiments, exemplary non-cancer subjects include healthy female subjects (e.g., healthy female subjects of specified age ranges, such as e.g., below age 55 or above age 55), female subjects with non-ovarian related health diseases, disorders, or conditions (including, e.g., female subjects having non-ovarian cancer such as lung cancer, colorectal cancer, etc., or female subjects having symptoms of inflammatory bowel diseases or disorders), female subjects having benign ovarian tumors (e.g., a benign mass observed in a fallopian tube and/or on an ovary), and combinations thereof.

### IV. Exemplary Methods for Profiling Individual Extracellular Vesicles (EVs)

In some embodiments, assays for profiling individual extracellular vesicles (e.g., single EV profiling assays) can be used to detect one or more provided biomarkers of one or more target biomarker signatures for ovarian cancer. For example, in some embodiments, such an assay may involve (i) a capture assay through targeting one or more provided markers of a target biomarker signature for ovarian cancer and (ii) a detection assay for at least one or more additional provided markers of such a target biomarker signature for ovarian cancer, wherein such a capture assay is performed prior to such a detection assay.

In some embodiments, a capture assay is performed to selectively capture tumor-associated extracellular vesicles (e.g., ovarian tumor-associated extracellular vesicles) from a blood or blood-derived sample (e.g., plasma sample) of a subject in need thereof. In some embodiments, a capture assay is performed to selectively capture extracellular vesicles of a certain size range, and/or certain characteristic(s), for example, extracellular vesicles associated with ovarian cancer. In some such embodiments, prior to a capture assay, a blood or blood-derived sample may be pre-processed to remove non-extracellular vesicles, including, e.g., but not limited to soluble proteins and interfering entities such as, e.g., cell debris. For example, in some embodiments, extracellular vesicles are purified from a blood or blood-derived sample of a subject using size exclusion chromatography. In some such embodiments, extracellular vesicles can be directly purified from a blood or blood-derived sample using size exclusion chromatography, which in some embodiments may remove at least 90% or higher (including, e.g., at least 93%, 95%, 97%, 99% or higher) of soluble proteins and other interfering agents such as, e.g., cell debris.

In some embodiments, a capture assay comprises a step of contacting a blood or blood-derived sample with at least one capture agent comprising a target-capture moiety that binds to at least one or more provided biomarkers of a target biomarker signature for ovarian cancer. In some embodiments, a capture assay may be multiplexed, which comprises a step of contacting a blood or blood-derived sample with a set of capture agents, each capture agent comprising a target-capture moiety that binds to a distinct provided biomarker of a target biomarker signature for ovarian cancer. In some embodiments, a target-capture moiety is directed to an extracellular vesicle-associated membrane-bound polypeptide (e.g., ones as described and/or utilized herein).

In some embodiments, such a target-capture moiety may be immobilized on a solid substrate. Accordingly, in some embodiments, a capture agent employed in a capture assay is or comprises a solid substrate comprising at least one or more (e.g., 1, 2, 3, 4, 5, or more) target-capture moiety conjugated thereto, each target-capture moiety directed to an extracellular vesicle-associated membrane-bound polypeptide (e.g., ones as described and/or utilized herein). A solid substrate may be provided in a form that is suitable for capturing extracellular vesicles and does not interfere with downstream handling, processing, and/or detection. For example, in some embodiments, a solid substrate may be or comprise a bead (e.g., a magnetic bead). In some embodiments, a solid substrate may be or comprise a surface. For example, in some embodiments, such a surface may be a capture surface of an assay chamber (including, e.g., a tube, a well, a microwell, a plate, a filter, a membrane, a matrix, etc.). In some embodiments, a capture agent is or comprises a magnetic bead comprising a target-capture moiety conjugated thereto.

In some embodiments, a detection assay is performed to detect one or more provided biomarkers of a target biomarker signature for ovarian cancer (e.g., ones that are different from ones targeted in a capture assay) in extracellular vesicles that are captured by a capture assay (e.g., as described above). In some embodiments, a detection assay may comprise immuno-PCR. In some embodiments, an immuno-PCR may involve at least one probe targeting a single provided biomarker (e.g., ones described herein) of a target biomarker signature for ovarian cancer. In some embodiments, an immuno-PCR may involve a plurality of (e.g., at least two, at least three, at least four, or more) probes directed to different epitopes of the same biomarker (e.g., ones described herein) of a target biomarker signature. In some embodiments, an immuno-PCR may involve a plurality of (e.g., at least two, at least three, at least four, or more) probes, each directed to a different provided biomarker described herein.

In some embodiments, a detection assay may comprise reverse transcription polymerase chain reaction (RT-PCR). In some embodiments, an RT-PCR may involve at least one primer/probe set targeting a single provided biomarker described herein. In some embodiments, an RT-PCR may involve a plurality of (e.g., at least two, at least three, at least four, or more) primer/probe sets, each set directed to a different provided biomarker described herein.

In some embodiments, a detection assay may comprise a proximity-ligation-immuno quantitative polymerase chain reaction (pliq-PCR), for example, to determine co-localization of one or more provided biomarkers of a target biomarker signature for ovarian cancer within extracellular vesicles (e.g., captured extracellular vesicles that express at least one extracellular vesicle-associated membrane-bound polypeptide).

In some embodiments, a detection assay employs a target entity detection system that was developed by Applicant and described in U.S. Application No. 16/805,637, and International Application PCT/US2020/020529, both filed Feb. 28, 2020 and entitled “Systems, Compositions, and Methods for Target Entity Detection” (the “‘637 application” and the “‘529 application”; both of which are incorporated herein by reference in their entirety) which are, in part, based on interaction and/or co-localization of a target biomarker signature in individual extracellular vesicles. For example, such a target entity detection system (as described in the ‘637 application and ‘529 application and also further described below in the section entitled “Provided Target Entity Detection Systems and Methods Involving the Same”) can detect in a sample (e.g., in a biological, environmental, or other sample), in some embodiments at a single entity level, entities of interest (e.g., biological or chemical entities of interest, such as extracellular vesicles or analytes) comprising at least one or more (e.g., at least two or more) targets (e.g., molecular targets). Those skilled in the art, reading the present disclosure, will recognize that provided target entity detection systems are useful for a wide variety of applications and/or purposes, including, e.g., for detection of ovarian cancer. For example, in some embodiments, provided target entity detection systems may be useful for medical applications and/or purposes. In some embodiments, provided target entity detection systems may be useful to screen (e.g., regularly screen) individuals (e.g., in some embodiments which may be asymptomatic individuals, or in some embodiments which may be individuals experiencing one or more symptoms associated with ovarian cancer, or in some embodiments which may be individuals at risk for ovarian cancer such as, e.g., individuals with a hereditary risk for ovarian cancer and/or life-history-associated risk factor, or post-menopausal individuals) for a disease or condition (e.g., ovarian cancer). In some embodiments, provided target entity detection systems may be useful to screen (e.g., regularly screen) individuals (e.g., in some embodiments which may be asymptomatic individuals, or in some embodiments which may be individuals experiencing one or more symptoms associated with ovarian cancer, or in some embodiments which may be individuals at risk for ovarian cancer such as, e.g., individuals with a hereditary risk for ovarian cancer and/or life-history-associated risk factor, or post-menopausal individuals) for different types of cancer (e.g., for a plurality of different cancers, one of which may be ovarian cancer). In some embodiments, provided target entity detection systems are effective even when applied to populations comprising or consisting of asymptomatic individuals (e.g., due to sufficiently high sensitivity and/or low rates of false positive and/or false negative results). In some embodiments, provided target entity detection systems may be useful as a companion diagnostic in conjunction with a disease treatment (e.g., treatment of ovarian cancer).

In some embodiments, a plurality of (e.g., at least two or more) detection assays may be performed to detect a plurality of biomarkers (e.g., at least two or more) of one or more target biomarker signatures for ovarian cancer (e.g., ones that are different from ones targeted in a capture assay) in extracellular vesicles, e.g., ones that are captured by a capture assay (e.g., as described above). For example, in some embodiments, a plurality of detection assays may comprise (i) a provided target entity detection system or a system described in the ‘637 application and ‘529 application; and (ii) immuno-PCR. In some embodiments, a plurality of detection assays may comprise (i) a provided target entity detection system or a system described in the ‘637 application and ‘529 application; and (ii) RT-PCR.

### V. Provided Target Entity Detection Systems and Methods Involving the Same

In some embodiments, a target entity detection system that can be useful in a detection assay for one or more provided biomarkers of one or more target biomarker signatures for ovarian cancer includes a plurality of detection probes each for a specific target (e.g., a provided biomarker of a target biomarker signature). In some embodiments, such a system may comprise at least 2, at least 3, at least 4, at least 5, at least 6, at least 7, at least 8, at least 9, at least 10, at least 15, at least 20, at least 25, at least 30, at least 40, at least 50, or more detection probes each for a specific target (e.g., a provided biomarker of a target biomarker signature). In some embodiments, such a system may comprise 2-50 detection probes each for a specific target (e.g., a provided biomarker of a target biomarker signature). In some embodiments, such a system may comprise 2-30 detection probes each for a specific target (e.g., a provided biomarker of a target biomarker signature). In some embodiments, such a system may comprise 2-25 detection probes each for a specific target (e.g., a provided biomarker of a target biomarker signature). In some embodiments, such a system may comprise 5-30 detection probes each for a specific target (e.g., a provided biomarker of a target biomarker signature). In some embodiments, such a system may comprise 5-25 detection probes each for a specific target (e.g., a provided biomarker of a target biomarker signature). In some embodiments, at least two of such detection probes in a set may be directed to the same biomarker of a target biomarker signature. In some embodiments, at least two of such detection probes in a set may be directed to the same epitope of the same biomarker of a target biomarker signature. In some embodiments, at least two of such detection probes in a set may be directed to different epitopes of the same biomarker of a target biomarker signature.

In some embodiments, detection probes appropriate for use in a target entity detection system provided herein may be used for detection of a single disease or condition, e.g., ovarian cancer. In some embodiments, detection probes appropriate for use in a target entity detection system provided herein may permit detection of at least two or more diseases or conditions, e.g., one of which is ovarian cancer. In some embodiments, detection probes appropriate for use in a target entity detection system provided herein may permit detection of ovarian cancer of certain subtypes including, e.g., but not limited to high-grade serous ovarian cancer, endometrioid ovarian cancer, clear-cell ovarian cancer, low-grade serous ovarian cancer, and/or mucinous ovarian cancer. In some embodiments, detection probes appropriate for use in atarget entity detection system provided herein may permit detection of ovarian cancer of certain stages, including, e.g., stage I, stage II, stage III, and/or stage IV. Accordingly, in some embodiments, detection probes appropriate for use in a target entity detection system provided herein may comprise a plurality (e.g., at least 2, at least 3, at least 4, at least 5, at least 6, at least 7, at least 8, at least 9, at least 10, or more) of sets of detection probes, wherein each set is directed to detection of a different disease or a different type of disease or condition. For example, in some embodiments, detection probes appropriate for use in a target entity detection system provided herein may comprise a plurality (e.g., at least 2, at least 3, at least 4, at least 5, at least 6, at least 7, at least 8, at least 9, at least 10, or more) of sets of detection probes, wherein in some embodiments, each set is directed to detection of a different type of cancer, one of which is ovarian cancer, or in some embodiments, each set is directed to detection of ovarian cancer of various subtypes and/or stages.

### Detection Probes

In some embodiments, a detection probe as provided and/or utilized herein comprises a target-binding moiety and an oligonucleotide domain coupled to the target-binding moiety. In some embodiments, an oligonucleotide domain coupled to a target-binding moiety may comprise a double-stranded portion and a single-stranded overhang extended from at least one end of the oligonucleotide domain. In some embodiments, an oligonucleotide domain coupled to a target-binding moiety may comprise a double-stranded portion and a single-stranded overhang extended from each end of the oligonucleotide domain. In some embodiments, detection probes may be suitable for proximity-ligation-immuno quantitative polymerase chain reaction (pliq-PCR) and be referred to as pliq-PCR detection probes.

### A. Target-Binding Moieties

A target-binding moiety that is coupled to an oligonucleotide domain is an entity or an agent that specifically binds to a target (e.g., a provided biomarker of a target biomarker signature; those skilled in the art will appreciate that, where the target biomarker is a particular form or moiety/component, the target-binding moiety specifically binds to that form or moiety/component). In some embodiments, a target-binding moiety may have a binding affinity (e.g., as measured by a dissociation constant) for a target (e.g., molecular target) of at least about 10⁻⁴ M, at least about 10⁻⁵ M, at least about 10⁻⁶ M, at least about 10⁻⁷ M, at least about 10⁻⁸ M, at least about 10⁻⁹ M, or lower. Those skilled in the art will appreciate that, in some cases, binding affinity (e.g., as measured by a dissociation constant) may be influenced by non-covalent intermolecular interactions such as hydrogen bonding, electrostatic interactions, hydrophobic and Van der Waals forces between the two molecules. Alternatively or additionally, binding affinity between a ligand and its target molecule may be affected by the presence of other molecules. Those skilled in the art will be familiar with a variety of technologies for measuring binding affinity and/or dissociation constants in accordance with the present disclosure, including, e.g., but not limited to ELISAs, gel-shift assays, pull-down assays, equilibrium dialysis, analytical ultracentrifugation, surface plasmon resonance (SPR), bio-layer interferometry, grating-coupled interferometry, and spectroscopic assays.

In some embodiments, a target-binding moiety may be or comprise an agent of any chemical class such as, for example, a carbohydrate, a nucleic acid, a lipid, a metal, a polypeptide, a small molecule, etc., and/or a combination thereof. In some embodiments, a target-binding moiety may be or comprise an antibody agent and/or an aptamer. In some embodiments, a target-binding moiety is or comprises an antibody agent, e.g., an antibody agent that specifically binds to a target or an epitope thereof, e.g., a provided biomarker of a target biomarker signature for ovarian cancer or an epitope thereof. In some embodiments, a target-binding moiety for a provided biomarker may be a commercially available. In some embodiments, a target-binding moiety for a provided biomarker may be designed and created for the purpose of use in assays as described herein. In some embodiments, a target-binding moiety is or comprises an aptamer, e.g., an aptamer that specifically binds to a target or an epitope thereof, e.g., a provided biomarker of a target biomarker signature for ovarian cancer or an epitope thereof. In some embodiments, a target-binding moiety is or comprises an affimer molecule that specifically binds to a target or an epitope thereof, e.g., a provided biomarker of a target biomarker signature for ovarian cancer or an epitope thereof. In some embodiments, such an affimer molecule can be or comprise a peptide or polypeptide that binds to a target or an epitope thereof (e.g., as described herein) with similar specificity and affinity to that of a corresponding antibody. In some embodiments, a target may be or comprise a target that is associated with ovarian cancer. For example, in some such embodiments, a cancer-associated target can be or comprise a target is associated with more than one cancer (i.e., at least two or more cancers). In some embodiments, a cancer-associated target can be or comprise a target that is typically associated with cancers. In some embodiments, a cancer-associated target can be or comprise a target that is associated with cancers of a specific tissue, e.g., ovarian cancer. In some embodiments, a cancer-associated target can be or comprise a target that is specific to a particular cancer, e.g., a particular ovarian cancer.

In some embodiments, a target-binding moiety recognizes and specifically binds to a target present in a biological entity (including, e.g., but not limited to cells and/or extracellular vesicles). For example, in some embodiments, a target-binding moiety may recognize and specifically bind to a tumor-associated antigen or epitope thereof. In some embodiments, a tumor-associated antigen may be or comprise an antigen that is associated with a cancer such as, for example, skin cancer, brain cancer (including, e.g., glioblastoma), breast cancer, colorectal cancer, liver cancer, lung cancer, ovarian cancer, pancreatic cancer, prostate cancer, and skin cancer. In some embodiments, a target-binding moiety may recognize a tumor antigen associated with ovarian cancer (e.g., high-grade serous ovarian cancer, endometrioid ovarian cancer, clear-cell ovarian cancer, low-grade serous ovarian cancer, or mucinous ovarian cancer). In some embodiments, a target-binding moiety may recognize a tumor antigen associated with high-grade serous ovarian cancer.

In some embodiments, a target-binding moiety may specifically bind to an intravesicular target, e.g., a provided intravesicular protein or RNA (e.g., mRNA). In some embodiments, a target-binding moiety may specifically bind to a surface target that is present on/within extracellular vesicles, e.g., a membrane-bound polypeptide present on ovarian cancer-associated extracellular vesicles.

In some embodiments, a target-binding moiety is directed to a biomarker for a specific condition or disease (e.g., cancer), which biomarker is or has been determined, for example, by analyzing a population or library (e.g., tens, hundreds, thousands, tens of thousands, hundreds of thousands, or more) of patient biopsies and/or patient data to identify such a biomarker (e.g., a predictive biomarker).

In some embodiments, a relevant biomarker may be one identified and/or characterized, for example, via data analysis. In some embodiments, for example, a diverse set of data (e.g., in some embodiments comprising one or more of bulk RNA sequencing, single-cell RNA (scRNA) sequencing, mass spectrometry, histology, post-translational modification data, in vitro and/or in vivo experimental data) can be analyzed through machine learning and/or computational modeling to identify biomarkers (e.g., predictive markers) that are highly specific to a disease or condition (e.g., cancer).

In some embodiments, a target-binding moiety is directed to a tissue-specific target, for example, a target that is associated with a specific tissue such as, for example, brain, breast, colon, ovary and/or other tissues associated with a female reproductive system, pancreas, prostate and/or other tissues associated with a male reproductive system, liver, lung, and skin. In some embodiments, such a tissue-specific target may be associated with a normal healthy tissue and/or a diseased tissue, such as a tumor. In some embodiments, a target-binding moiety is directed to a target that is specifically associated with a normal healthy condition of a subject.

In some embodiments, individual target binding entities utilized in a plurality of detection probes (e.g., as described and/or utilized herein) are directed to different targets. In some embodiments, such different targets may represent different marker proteins or polypeptides. In some embodiments, such different targets may represent different epitopes of the same marker proteins or polypeptides. In some embodiments, two or more individual target binding entities utilized in a plurality of detection probes (e.g., as described and/or utilized herein) may be directed to the same target.

In some embodiments, individual target binding entities utilized in a plurality of detection probes for detection of ovarian cancer may be directed to different target biomarkers of a target biomarker signature for ovarian cancer (e.g., ones as described in the section entitled “Provided Biomarkers and/or Target Biomarker Signatures for Detection of Ovarian Cancer” above). For example, in some embodiments, at least two detection probes in a plurality may have their target binding entities directed to MUC16 and FOLR1, respectively. In some embodiments, at least two detection probes in a plurality may have their target binding entities directed to MUC16 and CLDN6, respectively. In some embodiments, at least two detection probes in a plurality may have their target binding entities directed to MUC16 and CLDN3, respectively. In some embodiments, at least two detection probes in a plurality may have their target binding entities directed to FOLR1 and CLDN6, respectively. In some embodiments, at least two detection probes in a plurality may have their target binding entities directed to FOLR1 and CLDN3, respectively. In some embodiments, at least two detection probes in a plurality may have their target binding entities directed to SLC34A2 and CLDN3, respectively. In some embodiments, at least two detection probes in a plurality may have their target binding entities directed to SLC34A2 and CLDN3, respectively.

In some embodiments, individual target binding entities utilized in a plurality of detection probes for detection of ovarian cancer may be directed to the same target biomarker of a target biomarker signature for ovarian cancer (e.g., ones as described in the section entitled “Provided Biomarkers and/or Target Biomarker Signatures for Detection of Ovarian Cancer” above). In some embodiments, such target binding entities may be directed to the same or different epitopes of the same target biomarker of such a target biomarker signature for ovarian cancer. For example, in some embodiments, at least two detection probes in a plurality may have their target binding entities each directed to MUC16 (e.g., in its intact trans-membrane protein form, and/or at the same epitope or at different epitopes). In some embodiments, at least two detection probes in a plurality may have their target binding entities each directed to a FOLR1 peptide (e.g., at the same epitope or at different epitopes).

### B. Oligonucleotide Domains

In some embodiments, an oligonucleotide domain for use in accordance with the present disclosure (e.g., that may be coupled to a target-binding moiety) may comprise a double-stranded portion and a single-stranded overhang extended from one or both ends of the oligonucleotide domain. In some embodiments where an oligonucleotide domain comprises a single-stranded overhang extended from each end, a single-stranded overhang is extended from a different strand of a double-stranded portion. In some embodiments where an oligonucleotide domain comprises a single-stranded overhang extended from one end of the oligonucleotide domain, the other end of the oligonucleotide domain may be a blunt end.

In some embodiments, an oligonucleotide domain may comprise ribonucleotides, deoxyribonucleotides, synthetic nucleotide residues that are capable of participating in Watson-Crick type or analogous base pair interactions, and any combinations thereof. In some embodiments, an oligonucleotide domain is or comprises DNA. In some embodiments, an oligonucleotide domain is or comprises peptide nucleic acid (PNA).

In some embodiments, an oligonucleotide may have a length that is determined, at least in part, for example, by, e.g., the physical characteristics of an entity of interest (e.g., biological entity such as extracellular vesicles) to be detected, and/or selection and localization of molecular targets in an entity of interest (e.g., biological entity such as extracellular vesicles) to be detected. In some embodiments, an oligonucleotide domain of a detection probe is configured to have a length such that when a first detection probe and a second detection probe bind to an entity of interest (e.g., biological entity such as extracellular vesicles), the first single-stranded overhang and the second single-stranded overhang are in sufficiently close proximity to permit interaction (e.g., hybridization) between the single-stranded overhangs. For example, when an entity of interest (e.g., biological entity) is an extracellular vesicle (e.g., an exosome), oligonucleotide domains of detection probes can each independently have a length such that their respective single-stranded overhangs are in sufficiently close proximity to anneal or interact with each other when the corresponding detection probes are bound to the same extracellular vesicle. For example, in some embodiments, oligonucleotide domains of detection probes for use in detecting extracellular vesicles (e.g., an exosome) may each independently have a length of about 20 nm to about 200 nm, about 40 nm to about 500 nm, about 40 nm to about 300 nm, or about 50 nm to about 150 nm. In some embodiments, oligonucleotide domains of detection probes for use in detecting extracellular vesicles (e.g., an exosome) may each independently have a length of about 20 nm to about 200 nm. In some embodiments, lengths of oligonucleotide domains of detection probes in a set can each independently vary to increase and/or maximize the probability of them finding each other when they simultaneously bind to the same entity of interest.

Accordingly, in some embodiments, an oligonucleotide domain for use in technologies provided herein may have a length in the range of about 20 up to about 1000 nucleotides. In some embodiments, an oligonucleotide domain may have a length in the range of about 30 up to about 1000 nucleotides, In some embodiments, an oligonucleotide domain may have a length in the range of about 30 to about 500 nucleotides, from about 30 to about 250 nucleotides, from about 30 to about 200 nucleotides, from about 30 to about 150 nucleotides, from about 40 to about 150 nucleotides, from about 40 to about 125 nucleotides, from about 40 to about 100 nucleotides, from about 40 to about 60 nucleotides, from about 50 to about 90 nucleotides, from about 50 to about 80 nucleotides. In some embodiments, an oligonucleotide domain may have a length of at least 20 or more nucleotides, including, e.g., at least 30, at least 40, at least 50, at least 60, at least 70, at least 80, at least 90, at least 100, at least 250, at least 500, at least 750, at least 1000 nucleotides or more. In some embodiments, an oligonucleotide domain may have a length of no more than 1000 nucleotides or lower, including, e.g., no more than 900, no more than 800, no more than 700, no more than 600, no more than 500, no more than 400, no more than 300, no more than 200, no more than 100, no more than 90, no more than 80, no more than 70, no more than 60, no more than 50, no more than 40, no more than 30 nucleotides, no more than 20 nucleotides or lower.

In some embodiments, an oligonucleotide domain may have a length of about 20 nm to about 500 nm. In some embodiments, an oligonucleotide domain may have a length of about 20 nm to about 400 nm, about 30 nm to about 200 nm, about 50 nm to about 100 nm, about 30 nm to about 70 nm, or about 40 nm to about 60 nm. In some embodiments, an oligonucleotide domain may have a length of at least about 20 nm or more, including, e.g., at least about 30 nm, at least about 40 nm, at least about 50 nm, at least about 60 nm, at least about 70 nm, at least about 80 nm, at least about 90 nm, at least about 100 nm, at least about 200 nm, at least about 300 nm, at least about 400 nm or more. In some embodiments, an oligonucleotide domain may have a length of no more than 1000 nm or lower, including, e.g., no more than 900 nm, no more than 800 nm, no more than 700 nm, no more than 600 nm, no more than 500 nm, no more than 400 nm, no more than 300 nm, no more than 200 nm, no more than 100 nm or lower.

In some embodiments, a double-stranded portion of an oligonucleotide domain for use in technologies provided herein may have a length in the range of about 30 up to about 1000 nucleotides. In some embodiments, a double-stranded portion of an oligonucleotide domain may have a length in the range of about 30 to about 500 nucleotides, from about 30 to about 250 nucleotides, from about 30 to about 200 nucleotides, from about 30 to about 150 nucleotides, from about 40 to about 150 nucleotides, from about 40 to about 125 nucleotides, from about 40 to about 100 nucleotides, from about 50 to about 90 nucleotides, from about 50 to about 80 nucleotides. In some embodiments, a double-stranded portion of an oligonucleotide domain may have a length of at least 30 or more nucleotides, including, e.g., at least 40, at least 50, at least 60, at least 70, at least 80, at least 90, at least 100, at least 250, at least 500, at least 750, at least 1000 nucleotides or more. In some embodiments, a double-stranded portion of an oligonucleotide domain may have a length of no more than 1000 nucleotides or lower, including, e.g., no more than 900, no more than 800, no more than 700, no more than 600, no more than 500, no more than 400, no more than 300, no more than 200, no more than 100, no more than 90, no more than 80, no more than 70, no more than 60, no more than 50, no more than 40 nucleotides or lower.

In some embodiments, a double-stranded portion of an oligonucleotide domain may have a length of about 20 nm to about 500 nm. In some embodiments, a double-stranded portion of an oligonucleotide domain may have a length of about 20 nm to about 400 nm, about 30 nm to about 200 nm, about 50 nm to about 100 nm, about 30 nm to about 70 nm, or about 40 nm to about 60 nm. In some embodiments, a double-stranded portion of an oligonucleotide domain may have a length of at least about 20 nm or more, including, e.g., at least about 30 nm, at least about 40 nm, at least about 50 nm, at least about 60 nm, at least about 70 nm, at least about 80 nm, at least about 90 nm, at least about 100 nm, at least about 200 nm, at least about 300 nm, at least about 400 nm or more. In some embodiments, a double-stranded portion of an oligonucleotide domain may have a length of no more than 1000 nm or lower, including, e.g., no more than 900 nm, no more than 800 nm, no more than 700 nm, no more than 600 nm, no more than 500 nm, no more than 400 nm, no more than 300 nm, no more than 200 nm, no more than 100 nm or lower.

In some embodiments, a double-stranded portion of an oligonucleotide domain is characterized in that when detection probes are connected to each other through hybridization of respective complementary single-stranded overhangs (e.g., as described and/or utilized herein), the combined length of the respective oligonucleotide domains (including, if any, a linker that links a target-binding moiety to an oligonucleotide domain) is long enough to allow respective target binding entities to substantially span the full characteristic length (e.g., diameter) of an entity of interest (e.g., an extracellular vesicle). For example, in some embodiments where extracellular vesicles are entities of interest, a combined length of oligonucleotide domains (including, if any, a linker that links a target-binding moiety to an oligonucleotide domain) of detection probes may be approximately 50 to 200 nm, when the detection probes are fully connected to each other.

In some embodiments, a double-stranded portion of an oligonucleotide domain may comprise a binding site for a primer. In some embodiments, such a binding site for a primer may comprise a nucleotide sequence that is designed to reduce or minimize the likelihood for miss-priming or primer dimers. Such a feature, in some embodiments, can decrease the lower limit of detection and thus increase the sensitivity of systems provided herein. In some embodiments, a binding site for a primer may comprise a nucleotide sequence that is designed to have a similar annealing temperature as another primer binding site.

In some embodiments, a double-stranded portion of an oligonucleotide domain may comprise a nucleotide sequence designed to reduce or minimize overlap with nucleic acid sequences (e.g., DNA and/or RNA sequences) typically associated with genome and/or gene transcripts (e.g., genomic DNA and/or RNA, such as mRNA of genes) of a subject (e.g., a human subject). Such a feature, in some embodiments, may reduce or minimize interference of any genomic DNA and/or mRNA transcripts of a subject that may be present (e.g., as contaminants) in a sample during detection.

In some embodiments, a double-stranded portion of an oligonucleotide domain may have a nucleotide sequence designed to reduce or minimize formation of self-dimers, homo-dimers, or hetero-dimers.

In some embodiments, a single-stranded overhang of an oligonucleotide domain for use in technologies provided herein may have a length of about 2 to about 20 nucleotides. In some embodiments, a single-stranded overhang of an oligonucleotide domain may have a length of about 2 to about 15 nucleotides, from about 2 to about 10 nucleotides, from about 3 to about 20 nucleotides, from about 3 to about 15 nucleotides, from about 3 to about 10 nucleotides. In some embodiments, a single-stranded overhang can have at least 1 to 5 nucleotides in length. In some embodiments, a single-stranded overhang of an oligonucleotide domain may have a length of at least 2 or more nucleotides, including, e.g., at least 3, at least 4, at least 5, at least 6, at least 7, at least 8, at least 9, at least 10, at least 11, at least 12, at least 13, at least 14, at least 15, at least 20 nucleotides, or more. In some embodiments, a single-stranded overhang of an oligonucleotide domain may have a length of no more than 20 nucleotides or lower, including, e.g., no more than 15, no more than 14, no more than 13, no more than 12, no more than 11, no more than 10, no more than 9, no more than 8, no more than 7, no more than 6, no more than 5, no more than 4 nucleotides or lower.

In some embodiments, a single-stranded overhang of an oligonucleotide domain may have a length of about 1 nm to about 10 nm. In some embodiments, a single-stranded overhang of an oligonucleotide domain may have a length of about 1 nm to about 5 nm. In some embodiments, a single-stranded overhang of an oligonucleotide domain may have a length of at least about 0.5 nm or more, including, e.g., at least about 1 nm, at least about 1.5 nm, at least about 2 nm, at least about 3 nm, at least about 4 nm, at least about 5 nm, at least about 6 nm, at least about 7 nm, at least about 8 nm, at least about 9 nm, at least about 10 nm or more. In some embodiments, a single-stranded overhang of an oligonucleotide domain may have a length of no more than 10 nm or lower, including, e.g., no more than 9 nm, no more than 8 nm, no more than 7 nm, no more than 6 nm, no more than 5 nm, no more than 4 nm, no more than 3 nm, no more than 2 nm, no more than 1 nm or lower.

A single-stranded overhang of an oligonucleotide domain is designed to comprise a nucleotide sequence that is complementary to at least a portion of a single-stranded overhang of a second detection probe such that a double-stranded complex comprising a first detection probe and a second detection probe can be formed through hybridization of the complementary single-stranded overhangs. In some embodiments, nucleotide sequences of complementary single-stranded overhangs are selected for optimal ligation efficiency in the presence of an appropriate nucleic acid ligase. In some embodiments, a single-stranded overhang has a nucleotide sequence preferentially selected for efficient ligation by a specific nucleic acid ligase of interest (e.g., a DNA ligase such as a T4 or T7 ligase). For example, such a single-stranded overhang may have a nucleotide sequence of GAGT, e.g., as described in Song et al., “Enzyme-guided DNA sewing architecture” Scientific Reports 5: 17722 (2015), which is incorporated herein by reference for the purpose described herein.

When two detection probes couple together through hybridization of respective complementary single-stranded overhangs, their respective oligonucleotide domains comprising the hybridized single-stranded overhangs can, in some embodiments, have a combined length of about 90%-110% or about 95%-105% of a characteristic length (e.g., diameter) of an entity of interest (e.g., a biological entity). For example, in some embodiments when a biological entity is an exosome, the combined length can be about 50 nm to about 200 nm, or about 75 nm to about 150 nm, or about 80 nm to about 120 nm.

### C. Coupling Between a Target-Binding Moiety and an Oligonucleotide Domain

An oligonucleotide domain and a target-binding moiety can be coupled together in a detection probe by a covalent linkage, and/or by a non-covalent association (such as, e.g., a protein-protein interaction such as streptavidin-biotin interaction and/or an ionic interaction). In some embodiments, a detection probe appropriate for use in accordance with the present disclosure is a conjugate molecule comprising a target-binding moiety and an oligonucleotide domain, where the two components are typically covalently coupled to each other, e.g., directly through a bond, or indirectly through one or more linkers. In some embodiments, a target-binding moiety is coupled to one of two strands of an oligonucleotide domain by a covalent linkage (e.g., directly through a bond or indirectly through one or more linkers) and/or by a non-covalent association (such as, e.g., a protein-protein interaction such as streptavidin-biotin interaction and/or ionic interaction).

Where linkers are employed, in some embodiments, linkers are chosen to provide for covalent attachment of a target-binding moiety to one or both strands of an oligonucleotide domain through selected linkers. In some embodiments, linkers are chosen such that the resulting covalent attachment of a target-binding moiety to one or both strands of an oligonucleotide domain maintains the desired binding affinity of the target-binding moiety for its target. In some embodiments, linkers are chosen to enhance binding specificity of a target-binding moiety for its target. Linkers and/or conjugation methods of interest may vary widely depending on a target-binding moiety, e.g., its size and/or charges. In some embodiments, linkers are biologically inert.

A variety of linkers and/or methods for coupling a target-binding moiety to an oligonucleotide is known to one of ordinary skill in the art and can be used in accordance with the present disclosure. In some embodiments, a linker can comprise a spacer group at either end with a reactive functional group capable of covalent attachment to a target-binding moiety. Examples of spacer groups that can be used in linkers include, but are not limited to, aliphatic and unsaturated hydrocarbon chains (including, e.g., C4, C5, C6, C7, C8, C9, C10, C11, C12, C13, C14, C15, C16, C17, C18, C19, C20, or longer), spacers containing heteroatoms such as oxygen (e.g., ethers such as polyethylene glycol) or nitrogen (polyamines), peptides, carbohydrates, cyclic or acyclic systems that may contain heteroatoms. Non-limiting examples of a reactive functional group to facilitate covalent attachment include nucleophilic functional groups (e.g., amines, alcohols, thiols, and/or hydrazides), electrophilic functional groups (e.g., aldehydes, esters, vinyl ketones, epoxides, isocyanates, and/or maleimides), functional groups capable of cycloaddition reactions, forming disulfide bonds, or binding to metals. In some embodiments, exemplary reactive functional groups, but are not limited to, primary and secondary amines, hydroxamic acids, N- hydroxysuccinimidyl (NHS) esters, dibenzocyclooctyne (DBCO)-NHS esters, azido- NHS esters, azidoacetic acid NHS ester, propargyl-NHS ester, trans-cyclooctene-NHS esters, N-hydroxysuccinimidyl carbonates, oxycarbonylimidazoles, nitrophenylesters, trifluoroethyl esters, glycidyl ethers, vinylsulfones, maleimides, azidobenzoyl hydrazide, N-[4-(p-azidosalicylamino)butyl]-3′-[2′-pyridyldithio]propionamid), bis-sulfosuccinimidyl suberate, dimethyladipimidate, disuccinimidyltartrate, N- maleimidobutyryloxysuccinimide ester, N-hydroxy sulfosuccinimidyl-4- azidobenzoate, N-succinimidyl [4-azidophenyl]-1,3′-dithiopropionate, N- succinimidyl [4-iodoacetyl]aminobenzoate, glutaraldehyde, and succinimidyl 4-[N-maleimidomethyl]cyclohexane-1-carboxylate, 3-(2-pyridyldithio)propionic acid N-hydroxysuccinimide ester (SPDP), 4-(N-maleimidomethyl)- cyclohexane-1-carboxylic acid N-hydroxysuccinimide ester (SMCC), and any combinations thereof.

In some embodiments, a target-binding moiety (e.g., a target binding antibody agent) is coupled or conjugated to one or both strands of an oligonucleotide domain using N-hydrosysuccinimide (NHS) ester chemistry. NHS esters react with free primary amines and result in stable covalent attachment. In some embodiments, a primary amino group can be positioned at a terminal end with a spacer group, e.g., but not limited to an aliphatic and unsaturated hydrocarbon chain (e.g., a C6 or C12 spacer group).

In some embodiments, a target-binding moiety (e.g., a target binding antibody agent) can be coupled or conjugated to one or both strands of an oligonucleotide domain using a site-specific conjugation method known in the art, e.g., to enhance the binding specificity of conjugated target-binding moiety (e.g., conjugated target binding antibody agent). Examples of a site-specific conjugation method include, but are not limited to coupling or conjugation through a disulfide bond, C-terminus, carbohydrate residue or glycan, and/or unnatural amino acid labeling. In some embodiments where a target-binding moiety is or comprises an antibody agent or a peptide aptamer, an oligonucleotide can be coupled or conjugated to the target-binding moiety via at least one or more free amine groups present in the target-binding moiety. In some embodiments, an oligonucleotide can be coupled or conjugated to a target-binding moiety that is or comprises an antibody agent or a peptide aptamer via at least one or more reactive thiol groups present in the target-binding moiety. In some embodiments, an oligonucleotide can be coupled or conjugated to a target-binding moiety that is or comprises an antibody agent or a peptide aptamer via at least one or more carbohydrate residues present in the target-binding moiety.

In some embodiments, a plurality of oligonucleotides (e.g., at least 2, at least 3, at least 4, at least 5, at least 6, at least 7, at least 8, at least 9, at least ten, or more) can be coupled or conjugated to a target-binding moiety (e.g., a target binding antibody agent).

### Exemplary Duplex Target Entity Detection System

In some embodiments, a target entity detection system as provided by the present disclosure (and useful, for example, for detecting, e.g., at a single entity level, extracellular vesicles associated with ovarian cancer) may comprise a first population of first detection probes (e.g., as described and/or utilized herein) for a provided target biomarker (e.g., ones described herein) and a second population of second detection probes (e.g., as described and/or utilized herein) for a provided target biomarker (e.g., ones described herein). In some embodiments, the first detection probes and the second detection probes are directed to the same provided target biomarker. In some embodiments, the first detection probes and the second detection probes are directed to different provided target biomarkers.

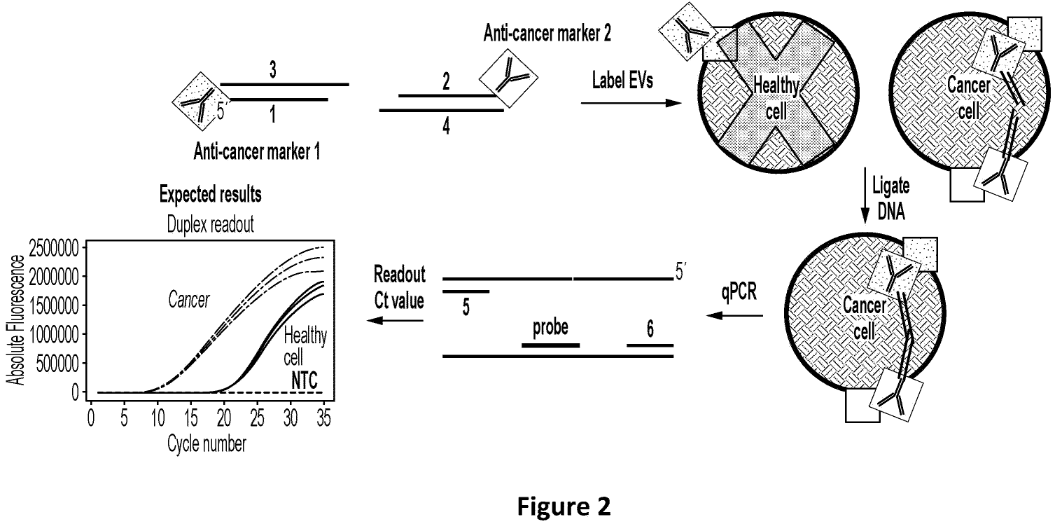

FIG. 2 illustrates an exemplary duplex target entity detection system for detecting, at a single entity level, an entity of interest (e.g., biological entity such as an extracellular vesicle) comprising (i) at least one target (e.g., a provided biomarker of a target biomarker signature for ovarian cancer) which expression level is high enough such that two molecules of the same target (e.g., a provided biomarker of a target biomarker signature for ovarian cancer) are found in close proximity, or (ii) at least two or more distinct targets (e.g.,. provided biomarkers of a target biomarker signature for ovarian cancer). A first detection probe comprises a first target-binding moiety (e.g., directed to a target cancer marker 1) and a first oligonucleotide domain coupled to the first target-binding moiety, the first oligonucleotide domain comprising a first double-stranded portion and a first single-stranded overhang extended from one end of the first oligonucleotide domain. As shown in FIG. 2, a first oligonucleotide domain may be resulted from hybridization of a longer strand (strand 3) and a shorter strand (strand 1), thereby forming a double-stranded portion and a single-stranded overhang at one end. In some embodiments, a first target-binding moiety (e.g., directed to target cancer marker 1) is coupled (e.g., covalently coupled) to a 5′ end or 3′ end of a strand of a first oligonucleotide domain (e.g., strand 1). In some embodiments, a 5′ end or 3′ end of a strand that is coupled to a first target-binding moiety may be modified with a linker (e.g., as described and/or utilized herein with or without a spacer group). In some embodiments, a 5′ end of another strand of a first oligonucleotide domain (e.g., strand 3) has a free phosphate group.

In the embodiment depicted in FIG. 2, a second detection probe comprises a second target-binding moiety (e.g., directed to a target cancer marker 2) and a second oligonucleotide domain coupled to the second target-binding moiety, the second oligonucleotide domain comprising a second double-stranded portion and a second single-stranded overhang extended from one end of the second oligonucleotide domain. As shown in FIG. 2, a second oligonucleotide domain may be resulted from hybridization of a longer strand (strand 4) and a shorter strand (strand 2), thereby forming a double-stranded portion and a single-stranded overhang at one end. In some embodiments, a second target-binding moiety (e.g., directed to a target cancer marker 2) is coupled (e.g., covalently coupled) to a 5′ end of a strand of a second oligonucleotide domain (e.g., strand 2). In some embodiments, a 5′ end of a strand that is coupled to a second target-binding moiety may be modified with a linker (e.g., as described and/or utilized herein with or without a spacer group). In some embodiments, a 5′ end of another strand of a second oligonucleotide domain (e.g., strand 4) has a free phosphate group.

At least portions of a first single-stranded overhang and a second single-stranded overhang are complementary to each other such that they can hybridize to form a double-stranded complex when they are in sufficiently close proximity, e.g., when a first detection probe and a second detection probe simultaneously bind to the same entity of interest (e.g., biological entity such as extracellular vesicle). In some embodiments, a first single-stranded overhang and a second single-stranded overhang have equal lengths such that when they hybridize to form a double-stranded complex, there is no gap (other than a nick to be ligated) between their respective oligonucleotide domains and each respective target-binding moiety is located at an opposing end of the double-stranded complex. For example, in some embodiments, a double-stranded complex forms before ligation occurs, wherein the double-stranded complex comprises a first detection probe and a second detection probe coupled to each other through direct hybridization of their respective single-stranded overhangs (e.g., having 4 nucleotides in length), wherein each respective target-binding moiety (e.g., directed to a target cancer marker 1 and a target cancer marker 2, respectively) is present at opposing ends of the double-stranded complex. In such embodiments, both strands of the double-stranded complex (comprising a nick between respective oligonucleotide domains) are ligatable, e.g., for amplification and detection. In some embodiments, a double-stranded complex (e.g., before ligation occurs) can comprise an entity of interest (e.g., a biological entity such as an extracellular vesicle), wherein a first target-binding moiety (e.g., directed to a target cancer marker 1) and a second target-binding moiety (e.g., directed to a target cancer marker 2) are simultaneously bound to the entity of interest.

In some embodiments of a duplex target entity detection system for detection of ovarian cancer, a first target-binding moiety of a first detection probe may be directed to a first target surface protein biomarker (e.g., ones provided in the section entitled “Provided Biomarkers and/or Target Biomarker Signatures for Detection of Ovarian Cancer”), while a second target-binding moiety of a second detection probe may be directed to a second target surface protein biomarker (e.g., ones provided in the section entitled “Provided Biomarkers and/or Target Biomarker Signatures for Detection of Ovarian Cancer”). In some embodiments, a first target-binding moiety of a first detection probe may be directed to a first target intravesicular protein biomarker (e.g., ones provided in the section entitled “Provided Biomarkers and/or Target Biomarker Signatures for Detection of Ovarian Cancer”), while a second target-binding moiety of a second detection probe may be directed to a second target intravesicular protein biomarker (e.g., ones provided in the section entitled “Provided Biomarkers and/or Target Biomarker Signatures for Detection of Ovarian Cancer”). In some embodiments, the first target-binding moiety and the second target-binding moiety may be directed to the same or different epitopes of the same target surface protein biomarker or of the same target intravesicular protein biomarker. In some embodiments, the first target-binding moiety and the second target-binding moiety may be directed to the different target surface protein biomarkers or different target intravesicular protein biomarkers.

In some embodiments of a duplex target entity detection system for detection of ovarian cancer, a first detection probe comprises a first target-binding moiety directed to MUC16 (e.g., in intact transmembrane protein form) conjugated to a first oligonucleotide domain; whereas a second detection probe comprises a second target-binding moiety detected to MUC16 (e.g., in intact transmembrane protein form) conjugated to a second oligonucleotide domain. In some such embodiments, the first target-binding moiety and the second target-binding moiety can be directed to the same or different epitope(s) of MUC16 (e.g., in intact transmembrane protein form). In some embodiments, the double stranded portion of a first oligonucleotide domain and a second oligonucleotide domain may be the same. In some embodiments, the double stranded portion of a first oligonucleotide domain and a second oligonucleotide domain may be different.

In some embodiments of a duplex target entity detection system for detection of ovarian cancer, a first detection probe comprises a first target-binding moiety directed to a FOLR1 polypeptide conjugated to a first oligonucleotide domain; whereas a second detection probe comprises a second target-binding moiety directed to a FOLR1 polypeptide conjugated to a second oligonucleotide domain. In some such embodiments, the first target-binding moiety and the second target-binding moiety can be directed to the same or different epitope(s) of FOLR1 polypeptide. In some embodiments, the double stranded portion of a first oligonucleotide domain and a second oligonucleotide domain may be the same. In some embodiments, the double stranded portion of a first oligonucleotide domain and a second oligonucleotide domain may be different.

In some embodiments of a duplex target entity detection system for detection of ovarian cancer, a first detection probe comprises a first target-binding moiety directed to MUC16 polypeptide conjugated to a first oligonucleotide domain; whereas a second detection probe comprises a second target-binding moiety directed to FOLR1 polypeptide conjugated to a second oligonucleotide domain. In some embodiments, the double stranded portion of a first oligonucleotide domain and a second oligonucleotide domain may be the same. In some embodiments, the double stranded portion of a first oligonucleotide domain and a second oligonucleotide domain may be different.

In some embodiments of a duplex target entity detection system for detection of ovarian cancer, a first detection probe comprises a first target-binding moiety directed to CLDN6 polypeptide conjugated to a first oligonucleotide domain; whereas a second detection probe comprises a second target-binding moiety directed to MUC16 polypeptide or FOLR1 polypeptide conjugated to a second oligonucleotide domain. In some embodiments, the double stranded portion of a first oligonucleotide domain and a second oligonucleotide domain may be the same. In some embodiments, the double stranded portion of a first oligonucleotide domain and a second oligonucleotide domain may be different.

In some embodiments of a duplex target entity detection system for detection of ovarian cancer, a first detection probe comprises a first target-binding moiety directed to CLDN3 polypeptide conjugated to a first oligonucleotide domain; whereas a second detection probe comprises a second target-binding moiety directed to SLC34A2 polypeptide conjugated to a second oligonucleotide domain. In some embodiments, the double stranded portion of a first oligonucleotide domain and a second oligonucleotide domain may be the same. In some embodiments, the double stranded portion of a first oligonucleotide domain and a second oligonucleotide domain may be different.

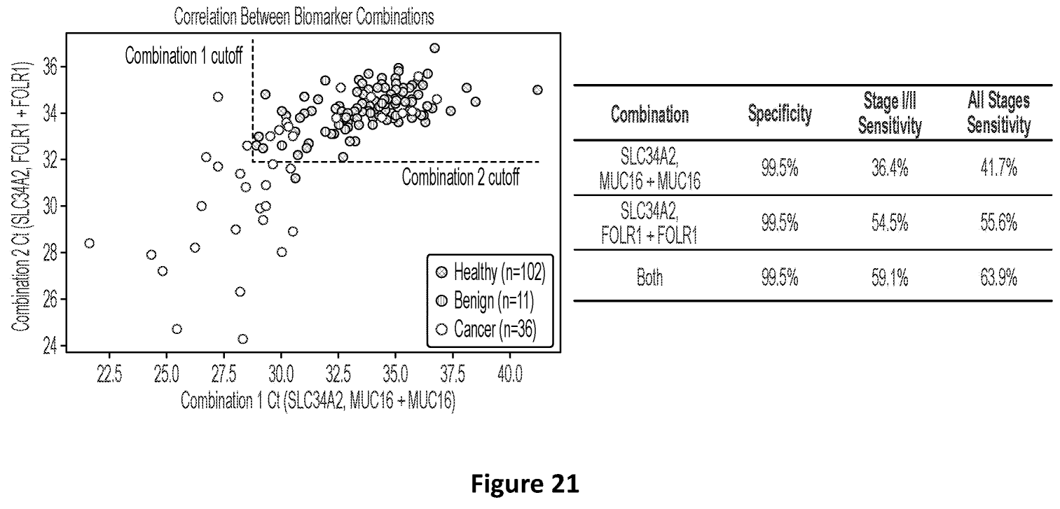

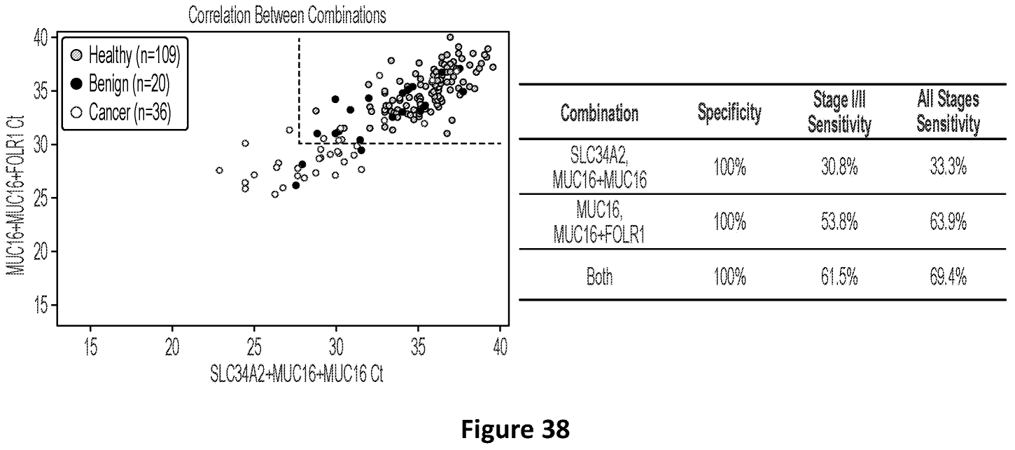

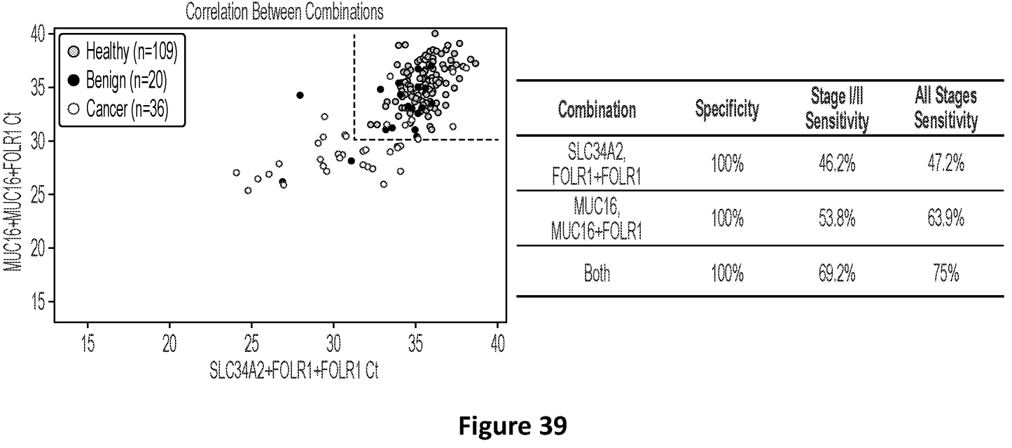

In some embodiments, a duplex target entity detection system for detection of ovarian cancer may comprise at least two distinct sets of detection probes. For example, in some embodiments, each set may be directed to a distinct target biomarker signature comprising one or more target biomarkers (e.g., ones described herein). For example, in some embodiments, as shown in FIG. 21, a duplex target entity detection system may comprise at least two sets of detection probes, wherein a first set comprises at least two detection probes each directed to MUC16 polypeptide, and a second set comprises at least two detection probes each directed to FOLR1 polypeptide. In some embodiments, for example, as shown in FIG. 38, a duplex target entity detection system may comprise at least two sets of detection probes, wherein a first set comprises at least two detection probes directed to FOLR1 polypeptide (e.g., with a capture probe directed to a SLC34A2 polypeptide), and a second set comprises at least two detection probes directed to MUC16 polypeptide and FOLR1 polypeptide respectively (e.g., with a capture probe directed to at least a MUC16 polypeptide). In some embodiments, as shown in FIG. 39, a duplex target entity detection system may comprise at least two sets of detection probes, wherein a first set comprises at least two detection probes each directed to FOLR1 polypeptide (e.g., with a capture probe directed to SLC34A2 polypeptide), and a second set comprises at least two detection probes directed to MUC16 polypeptide and FOLR1 polypeptide respectively (e.g., with a capture probe directed to a MUC16 polypeptide). In some embodiments, each set may be directed to a distinct combination of target biomarkers for ovarian cancer. For example, in some embodiments, a duplex target entity detection system may comprise at least two sets of detection probes, wherein a first set comprises at least two detection probes each directed to a distinct biomarker, and a second set comprises at least two detection probes each directed to a distinct biomarker.

In some embodiments, a duplex target entity detection system for detection of ovarian cancer may comprise at three distinct sets of detection probes. For example, in some embodiments, each set may be directed to a distinct target biomarker signature comprising one or more target biomarkers (e.g., ones described herein). In some such embodiments, at least one set may be directed to a single target biomarker (e.g., ones described herein). In some such embodiments, at least one set may be directed to a combination of at least two distinct target biomarkers (e.g., combinations of at least two target biomarkers described herein). For example, as described in Example 10, a duplex target entity detection system may comprise at least three sets of detection probes, wherein a first set comprises at least two detection probes each directed to MUC16 polypeptide; a second set comprises at least two detection probes each directed to FOLR1 polypeptide; and a third set comprises at least a first detection probe directed to MUC16 polypeptide and a second detection probe directed to FOLR1 polypeptide.

In some embodiments, a duplex target entity detection system comprising at least two distinct sets of detection probes may also comprise a capture assay comprising a capture agent directed to an extracellular vesicle-associated membrane-bound polypeptide.

In some embodiments, any combination of biomarker probes (e.g., a biomarker signature) including capture probes or detection probes as described herein may be utilized in combination with any other set of biomarker probes (e.g., a biomarker signature) including capture probes or detection probes as described herein.

### Exemplary Triplex or Multiplex (n≥3) Target Entity Detection System

In some embodiments, a target entity detection system as provided by the present disclosure (and useful, for example, for detecting, e.g., at a single entity level, extracellular vesicles associated with ovarian cancer) may comprise n populations of distinct detection probes (e.g., as described and/or utilized herein), wherein n ≥3. For example, in some embodiments when n =3, a target entity detection system may comprise a first detection probe (e.g., as described and/or utilized herein) for a first target, a population of a second detection probe (e.g., as described and/or utilized herein) for a second target, and a population of a third detection probe (e.g., as described and/or utilized herein) for a third target.

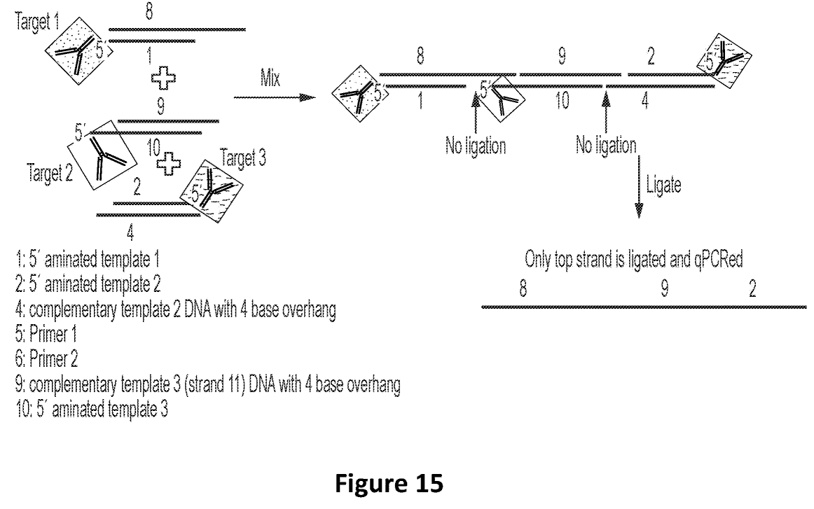

FIG. 15 illustrates an exemplary triplex target entity detection system for detecting, at a single entity level, an entity of interest (e.g., a biological entity such as an extracellular vesicle) comprising three distinct molecular targets. A first detection probe comprises a first target-binding moiety (e.g., anti-cancer marker 1 antibody agent) and a first oligonucleotide domain coupled to the first target-binding moiety, the first oligonucleotide domain comprising a first double-stranded portion and a first single-stranded overhang extended from one end of the first oligonucleotide domain. As shown in FIG. 15, a first oligonucleotide domain may be resulted from hybridization of a longer strand (strand 8) and a shorter strand (strand 1), thereby forming a double-stranded portion and a single-stranded overhang at one end. In some embodiments, a first target-binding moiety (e.g., anti-cancer marker 1 antibody agent) is coupled (e.g., covalently coupled) to a 5′ end of a strand of a first oligonucleotide domain (e.g., strand 1). In some embodiments, a 5′ end of a strand that is coupled to a first target-binding moiety may be modified with a linker (e.g., as described and/or utilized herein with or without a spacer group). In some embodiments, a 5′ end of another strand of a first oligonucleotide domain (e.g., strand 8) has a free phosphate group.

In the embodiment depicted in FIG. 15, a second detection probe comprises a second target-binding moiety (e.g., anti-cancer marker 3 antibody agent) and a second oligonucleotide domain coupled to the second target-binding moiety, the second oligonucleotide domain comprising a second double-stranded portion and a second single-stranded overhang extended from one end of the second oligonucleotide domain. As shown in FIG. 15, a second oligonucleotide domain may be resulted from hybridization of a longer strand (strand 4) and a shorter strand (strand 2), thereby forming a double-stranded portion and a single-stranded overhang at one end. In some embodiments, a second target-binding moiety (e.g., anti-cancer marker 3 antibody agent) is coupled (e.g., covalently coupled) to a 5′ end of a strand of a second oligonucleotide domain (e.g., strand 2). In some embodiments, a 5′ end of a strand that is coupled to a second target-binding moiety may be modified with a linker (e.g., as described and/or utilized herein with or without a spacer group). In some embodiments, a 5′ end of another strand of a second oligonucleotide domain (e.g., strand 4) has no free phosphate group.

A third detection probe comprises a third target-binding moiety (e.g., anti-cancer marker 2 antibody agent) and a third oligonucleotide domain coupled to the third target-binding moiety, the third oligonucleotide domain comprising a third double-stranded portion and a single-stranded overhang extended from each end of the third oligonucleotide domain. For example, a single-stranded overhang is extended from one end of a strand of a third oligonucleotide domain while another single-stranded overhang is extended from an opposing end of a different strand of the third oligonucleotide domain. As shown in FIG. 15, a third oligonucleotide domain may be resulted from hybridization of portions of two strands (e.g., strands 9 and 10), thereby forming a double-stranded portion and a single-stranded overhang at each end. For example, a single-stranded overhang (3A) is formed at a 5′ end of strand 9 of a third detection probe, wherein the 5′ end of strand 9 has a free phosphate group. Additionally, a single-stranded overhang (3B) is formed at a 5′ end of strand 10 of the same third detection probe and a third target-binding moiety (e.g., anti-target 2 antibody agent) is also coupled (e.g., covalently coupled) to the 5′ end of strand 10. In some embodiments, a 5′ end of a strand (e.g., strand 10) that is coupled to a third target-binding moiety may be modified with a linker (e.g., as described and/or utilized herein with or without a spacer group).

When all three detection probes are in sufficiently close proximity, e.g., when all three detection probes simultaneously bind to the same entity of interest (e.g., biological entity), (i) at least a portion of a single-stranded overhang (e.g., 3A) of a third detection probe is hybridized to a corresponding complementary portion of a single-stranded overhang of a second detection probe, and (ii) at least a portion of another single-stranded overhang (e.g., 3B) of the third detection probe is hybridized to a corresponding complementary portion of a single-stranded overhang of a first detection probe. As a result, a double-stranded complex comprising all three detection probes coupled to each other in a linear arrangement is formed by direct hybridization of corresponding single-stranded overhangs. See, e.g., FIG. 15.

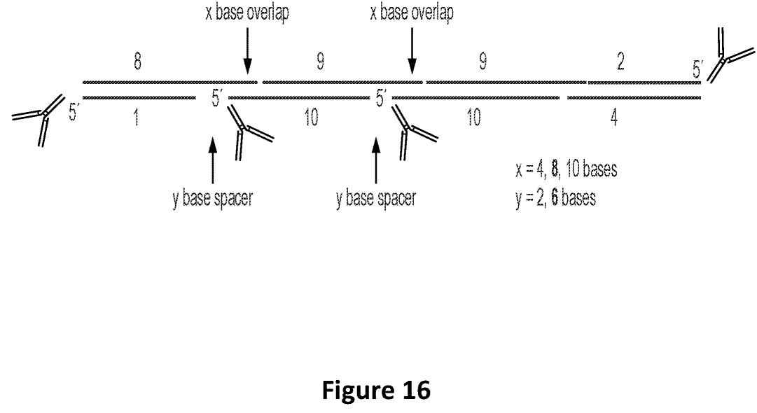

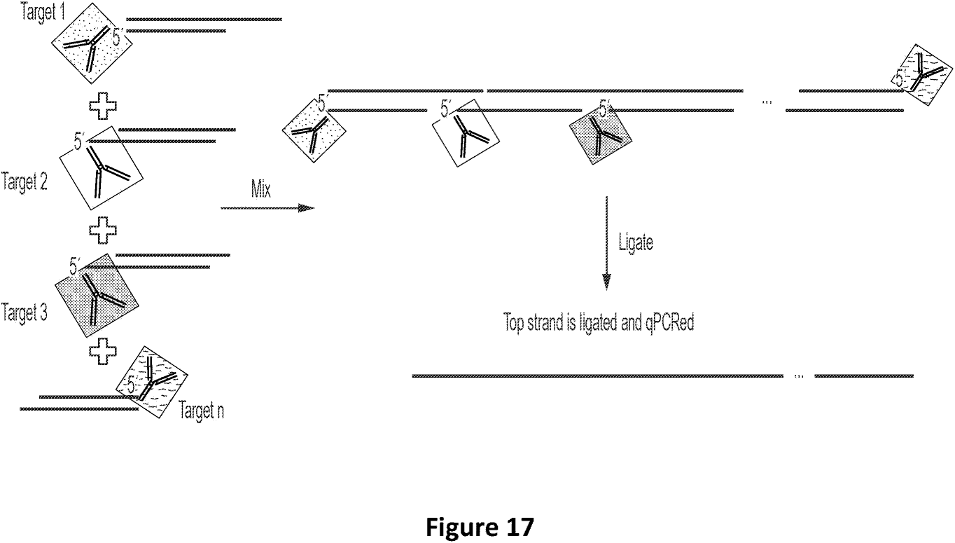

In some embodiments involving use of at least three or more (n ≥3) detection probes in provided technologies, when single-stranded overhangs of detection probes anneal to each respective partner(s) to form a double-stranded complex, at least (n-2) target-binding moiety/moieties is/are present at internal position(s) of the double-stranded complex. In such embodiments, it is desirable to have internal target binding moieties present in a single strand of the double-stranded complex such that another strand of the double-stranded complex is free of any internal target binding moieties and is thus ligatable to form a ligated template. e.g., for amplification and detection. See, e.g., FIG. 15 (using three detection probes), FIG. 16 (using four detection probes), and FIG. 17 (using n detection probes).

In some embodiments where a strand of a double-stranded complex comprises at least one or more internal target binding moieties, the strand comprises a gap between an end of an oligonucleotide strand of a detection probe to which the internal target-binding moiety is coupled and an end of an oligonucleotide strand of another detection probe. The size of the gap is large enough such that the strand becomes non-ligatable in the presence of a nucleic acid ligase. In some embodiments, the gap may be 2 - 8 nucleotides in size or 2-6 nucleotides in size. In some embodiments, the gap is 6 nucleotides in size. In some embodiments, the overlap (hybridization region between single-stranded overhangs) can be 2 - 15 nucleotides in length or 4-10 nucleotides in length. In some embodiments, the overlap (hybridization region between single-stranded overhangs) is 8 nucleotides in length. The size of the gap and/or hybridization region are selected to provide an optimum signal separation from a ligated template (comprising no internal target binding moieties) and non-ligated template (comprising at least one internal target-binding moiety). It should be noted that while FIGS. 15-17 do not show binding of detection probes to an entity of interest (e.g., a biological entity), a double-stranded complex (e.g., before ligation occurs) can comprise an entity of interest (e.g., a biological entity such as extracellular vesicles), wherein at least three or more target binding moieties are simultaneously bound to the entity of interest.

In some embodiments, selection of a combination (e.g., a set) of detection probes (e.g., number of detection probes and/or specific biomarkers) for use in a target entity detection system provided herein (e.g., a duplex, triplex or multiplex target entity detection system described herein) is based on, for example, a desired specificity and/or a desired sensitivity that is deemed to be optimal for a particular application. For example, in some embodiments, a combination of detection probes is selected for detection of ovarian cancer (e.g., for stage I, II, III, or IV) such that it provides a specificity of at least 95% or higher, including, e.g., at least 96%, at least 97%, at least 98%, at least 99%, at least 99.5%, at least 99.7%, at least 99.8% or higher. In some embodiments, a combination of detection probes is selected for detection of ovarian cancer (e.g., for stage I, II, III, or IV) such that it provides a sensitivity of at least 30% or higher, including, e.g., at least 40%, at least 50%, at least 60%, at least 70%, at least 80%, at least 90%, at least 95% or higher. In some embodiments, a combination of detection probes is selected for detection of ovarian cancer (e.g., for stage I, II, III, or IV) such that it provides a positive predictive value of at least 8% or higher, including, e.g., at least 9%, at least 10%, at least 15%, at least 20%, at least 25%, at least 30%, at least 40%, at least 50%, or higher. In some embodiments, a combination of detection probes is selected for detection of ovarian cancer (e.g., for stage I, II, III, or IV) such that it provides a positive predictive value of at least 2% or higher, including, e.g., at least 3%, at least 4%, at least 5%, at least 6%, at least 7%, at least 8%, at least 9%, at least 10%, at least 15%, at least 20%, at least 25%, at least 30%, at least 40%, at least 50%, or higher. In some embodiments, a combination of detection probes is selected for detection of ovarian cancer (e.g., for stage I, II, III, or IV) such that it provides a limit of detection (LOD) below 1x10⁷ EV/mL sample or lower, including, e.g., below 7x10⁶ EV/mL sample, below 6x10⁶ EV/mL sample, below 5x10⁶ EV/mL sample, below 4x10⁶ EV/mL sample, below 3x10⁶ EV/mL sample, below 2x10⁶ EV/mL sample, below 1x10⁶ EV/mL sample, or lower. In some embodiments, such ovarian cancer detection assay may be used to detect different subtypes of ovarian cancer including, e.g., but not limited to high-grade serous ovarian cancer, endometrioid ovarian cancer, clear-cell ovarian cancer, low-grade serous ovarian cancer, or mucinous ovarian cancer. In some embodiments, such ovarian cancer detection assay may be used to detect ovarian cancer of an epithelial origin. In some embodiments, such ovarian cancer detection assay may be used to detect high-grade serous ovarian cancer.

In some embodiments, a combination (e.g., a set) of detection probes, rather than individual detection probes, confers specificity to detection of a disease, disorder, or condition (e.g., a particular ovarian cancer and/or a stage of ovarian cancer as described herein), for example, one or more individual probes may be directed to a target that itself is not specific to ovarian cancer. For example, in some embodiments, a useful combination of detection probes in a target entity detection system provided herein (e.g., a duplex, triplex or multiplex target entity detection system described herein) may comprise at least one detection probe directed to a target specific for the relevant disease, disorder, or condition (i.e., a target that is specific to the relevant disease, disorder, or condition), and may further comprise at least one detection probe directed to a target that is not necessarily or completely specific for the relevant disease, disorder, or condition (e.g., that may also be found on some or all cells that are healthy, are not of the particular disease, disorder, or condition, and/or are not of the particular disease stage of interest). That is, as will be appreciated by those skilled in the art reading the present specification, so long as the set of detection probes utilized in accordance with the present invention is or comprises a plurality of individual detection probes that together are specific for detection of the relevant disease, disorder, or condition (i.e., sufficiently distinguish biological entities for detection that are associated with the relevant disease, disorder, or condition from other biological entities not of interest for detection), the set is useful in accordance with certain embodiments of the present disclosure.

In some embodiments, a target entity detection system provided herein (e.g., a duplex, triplex or multiplex target entity detection system described herein) can comprise at least one or more (e.g., at least 2 or more) control probes (in addition to target-specific detection probes, e.g., as described and/or utilized herein, for example, in some embodiments to recognize disease-specific biomarkers such as cancer-specific biomarkers and/or tissue-specific biomarkers). In some embodiments, a control probe is designed such that its binding to an entity of interest (e.g., a biological entity) inhibits (completely or partially) generation of a detection signal.

In some embodiments, a control probe comprises a control binding moiety and an oligonucleotide domain (e.g., as described and/or utilized herein) coupled to the control binding moiety, the oligonucleotide domain comprising a double-stranded portion and a single-stranded overhang extended from one end of the oligonucleotide domain. A control binding moiety is an entity or moiety that bind to a control reference. In some embodiments, a control reference can be or comprise a biomarker that is preferentially associated with a normal healthy cell. In some embodiments, a control reference can be or comprise a biomarker preferentially associated from a non-target tissue. In some embodiments, inclusion of a control probe can selectively remove or minimize detectable signals generated from false positives (e.g., entities of interest comprising a control reference, optionally in combination with one or more targets to be detected). Other control probes described in U.S. Application No. 16/805,637, and International Application PCT/US2020/020529, both filed Feb. 28, 2020 and entitled “Systems, Compositions, and Methods for Target Entity Detection,” the entire contents of each application are incorporated herein by reference in their entirety, can be useful in provided target entity detections systems.

In some embodiments, the present disclosure provides insights, among other things, that detection probes as described or utilized herein may non-specifically bind to a solid substrate surface and some of them may remain in an assay sample even after multiple washes to remove any excess or unbound detection probes; and that such non-specifically bound detection probes may come off from the solid substrate surface and become free-floating in a ligation reaction, thus allowing them to interact with one another to generate a non-specific ligated template that produces an undesirable background signal. Accordingly, in some embodiments, a target entity detection system provided herein (e.g., a duplex, triplex, or multiplex target entity detection described herein) can comprise at least one or more (e.g., at least 2 or more) inhibitor oligonucleotides that are designed to capture residual detection probes that are not bound to an entity of interest but remain as free agents in a ligation reaction, thereby preventing such free-floating detection probes from interacting with other free-floating complementary detection probes to produce an undesirable background signal. In some embodiments, an inhibitor oligonucleotide may be or comprise a single-stranded or double-stranded oligonucleotide comprising a binding domain for a single-stranded overhang of a detection probe (e.g., as described or utilized herein), wherein the inhibitor oligonucleotide does not comprise a primer binding site. The absence of such a primer binding site in an inhibitor oligonucleotide prevents a primer from binding to a non-specific ligated template resulting from ligation of a detectable probe to an inhibitor oligonucleotide, thereby reducing or inhibiting the non-specific ligated template from amplification and/or detection, e.g., by polymerase chain reaction.

In some embodiments, an inhibitor oligonucleotide comprises a binding domain for a single-stranded overhang of a detection probe (e.g., as described or utilized herein), wherein the binding domain is or comprises a nucleotide sequence that is substantially complementary to the single-stranded overhang of the detection probe such that a free, unbound detection probe having a complementary single-stranded overhang can bind to the binding domain of the inhibitor oligonucleotide. In some embodiments, an inhibitor oligonucleotide may have a hairpin at one end. In some embodiments, an inhibitor oligonucleotide may be a single-stranded oligonucleotide comprising at one end a binding domain for a single-stranded overhang of a detection probe, wherein a portion of the single-stranded oligonucleotide can self-hybridize to form a hairpin at another end.

In some embodiments, a target entity detection system provided herein (e.g., a duplex, triplex or multiplex target entity detection system described herein) does not comprise a connector oligonucleotide that associates an oligonucleotide domain of a detection probe with an oligonucleotide domain of another detection probe. In some embodiments, a connector oligonucleotide is designed to bridge oligonucleotide domains of any two detection probes that would not otherwise interact with each other when they bind to an entity of interest. In some embodiments, a connector oligonucleotide is designed to hybridize with at least a portion of an oligonucleotide domain of a detection probe and at least a portion of an oligonucleotide domain of another detection probe. A connector oligonucleotide can be single-stranded, double-stranded, or a combination thereof. A connector oligonucleotide is free of any target-binding moiety (e.g., as described and/or utilized herein) or control binding moiety. In at least some embodiments, no connector oligonucleotides are necessary to indirectly connect oligonucleotide domains of detection probes; in some embodiments, such connector oligonucleotides are not utilized, in part because detection probes as provided and/or utilized herein are designed such that their respective oligonucleotide domains have a sufficient length to reach and interact with each other when they are in sufficiently close proximity, e.g., when the detection probes simultaneously bind to an entity of interest (e.g., a biological entity such as an extracellular vesicle).

### Methods of Using Provided Target Entity Detection Systems

Provided target entity detection systems are useful in detecting an entity of interest (e.g., a biological entity such as extracellular vesicles) in a sample (e.g., in a biological, environmental, or other sample) for various applications and/or purposes associated with detection of ovarian cancer. Accordingly, some aspects provided herein relate to methods of using a plurality of (e.g., at least 2, at least 3, or more) detection probes appropriate for use in accordance with the present disclosure. In some embodiments, a method comprises contacting an entity of interest (e.g., a biological entity such as extracellular vesicles) in a sample (e.g., a blood or blood-derived sample from a human female subject) with a set of detection probes comprising at least 2 or more (including, e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more) detection probes as described and/or utilized herein. In some embodiments, a method comprises subjecting a sample comprising an entity of interest (e.g., a biological entity such as extracellular vesicles) to a target entity detection system (e.g., as provided herein). A plurality of detection probes (e.g., at least two or more) can be added to a sample comprising an entity of interest (e.g., a biological entity such as extracellular vesicles) at the same time or at different times (e.g., sequentially). In some embodiments, a method may comprise, prior to contacting with a plurality of detection probes, contacting a sample comprising an entity of interest with at least one capture agent directed to an extracellular vesicle-associated membrane-bound polypeptide.

In certain embodiments, a provided target entity detection system for use in a method described herein may comprise a plurality of (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more) distinct sets (e.g., combinations) of detection probes (e.g., as described herein). In some embodiments, a method comprises contacting an entity of interest (e.g., a biological entity such as extracellular vesicles) in a sample (e.g., a blood or blood-derived sample from a human female subject) with a plurality of sets of detection probes, wherein each set may comprise at least 2 or more (including, e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more) detection probes as described and/or utilized herein. In some embodiments, a method comprises subjecting a sample comprising an entity of interest (e.g., a biological entity such as extracellular vesicles) to a target entity detection system (e.g., as provided herein). A plurality of detection probes and/or detection probe combinations (e.g., at least two or more) can be added to a sample comprising an entity of interest (e.g., a biological entity such as extracellular vesicles) at the same time or at different times (e.g., sequentially). In some embodiments, a method may comprise, prior to contacting with a plurality of detection probes, contacting a sample comprising an entity of interest with at least one capture agent directed to an extracellular vesicle-associated membrane-bound polypeptide.

In some embodiments, the relationship between results (e.g., Ct values and/or relative number of ligated nucleic acid templates (e.g., ligated DNA templates)) from profiling one or more biomarker combinations in a sample can be combined with clinical information (including, e.g., but not limited to patient age, past medical history, serum CA-125, etc.) and/or other information to better classify patients with or at risk for ovarian cancer. Various classification algorithms can be used to interpret the relationship between multiple variables to increase an assay’s sensitivity and/or specificity. In some embodiments, such algorithms include, but are not limited to, logistic regression models, support vector machines, gradient boosting machines, random forest algorithms, Naive Bayes algorithms, K-nearest neighborhood algorithms, and combinations thereof. In some embodiments, performance (e.g., accuracy) of assays described herein can be improved, e.g., by selection of biomarker combinations (e.g., as described herein), selection of other factors or variables (e.g., clinical information) to include an algorithm, and/or selection of the type of algorithm itself.

In certain embodiments, technologies described herein utilize a predictive algorithm that is trained and validated using data sets as described herein. In certain embodiments, technologies described herein are utilized to generate a risk score using an algorithm created from training samples which is designed to take into account results from at least two, e.g., at least two, at least 3, at least 4, at least 5, or more than 5 separate assays comprising biomarker signatures (e.g., as described herein). In certain embodiments, an algorithm-generated risk score can be generated at least in part using diagnostic data (e.g., raw and/or normalized Ct values) from at least one individual assay (e.g., individual biomarker signature). In certain embodiments, a reference threshold can be included within a risk score. In certain embodiments, multiple threshold levels denoting multiple different degrees of ovarian cancer risk may be included in a risk score. In some embodiments, separate target biomarker signature assays may be performed as individual assays in a series of assays, and individual assays may be weighted equally or differently in a predictive algorithm. In some embodiments, for example, weighting of individual assays combined in an algorithm (e.g., a cohort of biomarker assays) may be determined by a number of factors including but not limited to the sensitivity of an individual assay, the specificity of an individual assay, the reproducibility of an individual assay, the variability of an individual assay, the positive predictive value of an individual assay, and/or the lowest limit of detection of a specific assay. In some embodiments, a cohort of biomarker assays may be ranked according to a characteristic (e.g., sensitivity, specificity, lowest limit of detection etc.) and the biomarker assays may then be weighted based upon their relative rank.

In some embodiments, a risk score generated by an algorithm (as described herein) can be presented in a suitable manner, e.g., on a nominal scale, e.g., on a scale of 0-100 reflecting a number of likelihoods, e.g., including but not limited to the likelihood a woman has ovarian cancer, the likelihood a woman will develop ovarian cancer, and/or the likely stage of ovarian cancer. In some embodiments, a higher risk score can demonstrate that there is an increasing likelihood of disease pathology, e.g., lower to higher values may reflect healthy controls, benign controls, stage I, stage II, stage III, and stage IV ovarian cancers. In some embodiments, a risk score can be utilized to reduce the potential of cross reactivity of technologies as described herein when compared with other cancer types.

In some embodiments, a risk score may be generated from a combination of data derived from assays as described herein coupled with other applicable diagnostic data such as age, life history, TVUS results, CA-125 levels, or any combination thereof. In some embodiments, a risk score provides predictive value above and beyond that of conventional standard of care diagnostic assay predictive values, e.g., higher than predictive values provided by TVUS and/or CA-125 assays utilized in isolation or in combination. In some embodiments, a risk score may be generated that has high specificity for ovarian cancers (e.g., high-grade serous carcinoma) and has low sensitivity for other cancers.

In some embodiments, a risk score may have an associated clinical cutoff for detection of ovarian cancer. For example, in some embodiments a risk score clinical cutoff for detection may require an assay that yields at least 40%, e.g., at least 50%, at least 60%, or greater sensitivity for detection of both early and late stage ovarian cancer and has a minimum of 95% specificity, e.g., at least 96%, at least 97%, at least 98%, at least 99% or greater specificity in a generally healthy population of women aged 20 to 89 years of age. In some embodiments, sensitivity and specificity targets are the approximate lower bounds of the two-sided 95% confidence interval for the targeted 77% sensitivity and 99.5% specificity.

In some embodiments, a training study is performed to provide the necessary data required to program a risk score algorithm. In some embodiments, such a training study may comprise a cohort of samples from a range of suppliers, including at least commercial suppliers, purpose driven studies, and/or physicians. In some embodiments, a training study may comprise positive samples from high-grade serous ovarian cancer patients (e.g., stage I, stage II, stage III, and/or stage IV), positive control samples from high-grade serous ovarian cancer cell lines, negative samples from benign gynecological tumor patients (e.g., ovarian tumor, uterine tumor, etc.), negative samples from non-ovarian cancer patients (e.g., brain cancer, breast cancer, colorectal cancer, endometrial cancer, lung adenocarcinoma, melanoma, non-Hodgkin’s lymphoma, pancreatic cancer, skin cancer, etc.), negative samples from inflammatory condition patients (e.g., Crohn’s disease, endometriosis, diabetes type II, lupus, pancreatitis, rheumatoid arthritis, ulcerative colitis, etc.), negative samples from healthy patients, or any combination thereof. In some embodiments, a training study may comprise samples from patients of any appropriate age range, e.g., <31 years old, 31-40 years old, 41-50 years old, 51-60 years old, 61-70 years old, 71-80 years old, or >80 years old. In some embodiments, a training study may comprise samples from patients of any race/ethnicity/decent, (e.g., Caucasians, Africans, Asians etc.).

In some embodiments, a validation study is performed to provide the necessary data required to confirm a risk score algorithm’s utility. In some embodiments, such a validation study may comprise a cohort of samples from a range of suppliers, including at least commercial suppliers, purpose driven studies, and/or physicians. In some embodiments, a validation study may comprise positive samples from high-grade serous ovarian cancer patients (e.g., stage I, stage II, stage III, and/or stage IV), positive control samples from high-grade serous ovarian cancer cell lines, negative samples from benign gynecological tumor patients (e.g., ovarian tumor, uterine tumor, etc.), negative samples from non-ovarian cancer patients (e.g., brain cancer, breast cancer, colorectal cancer, endometrial cancer, lung adenocarcinoma, melanoma, non-Hodgkin’s lymphoma, pancreatic cancer, skin cancer, etc.), negative samples from inflammatory condition patients (e.g., Crohn’s disease, endometriosis, diabetes type II, lupus, pancreatitis, rheumatoid arthritis, ulcerative colitis, etc.), negative samples from healthy patients, or any combination thereof. In some embodiments, a validation study may comprise samples from patients of any appropriate age range, e.g., <31 years old, 31-40 years old, 41-50 years old, 51-60 years old, 61-70 years old, 71-80 years old, or >80 years old. In some embodiments, a validation study may comprise samples from patients of any race/ethnicity/decent, (e.g., Caucasians, Africans, Asians etc.).

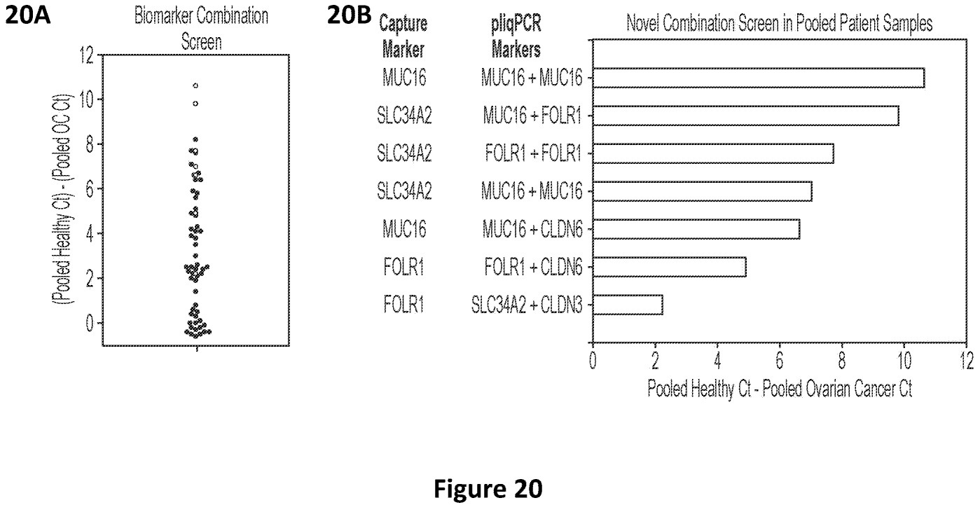

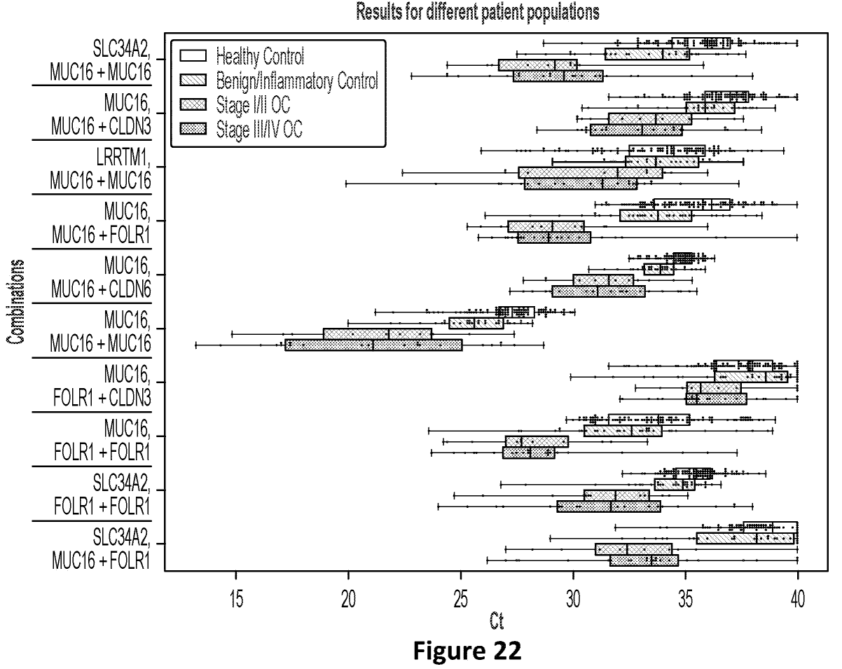

In certain embodiments, at least one target biomarker signature comprising at least one extracellular vesicle-associated membrane-bound polypeptide and at least one (including, e.g., at least two, or more) target biomarker (which may be selected from any of surface protein biomarkers described herein, intravesicular protein biomarkers described herein, and/or intravesicular RNA biomarkers described herein) may be embodied in an ovarian cancer detection assay. In some such embodiments, at least one capture agent is directed to the extracellular vesicle-associated membrane-bound polypeptide, and at least one set of detection probes is directed to one or more of such target biomarkers described herein. For example, FIG. 20 (Panel B) and FIG. 22 disclose certain examples of target biomarker signatures, each of which may be embodied in an ovarian cancer detection assay (e.g., ones described herein).

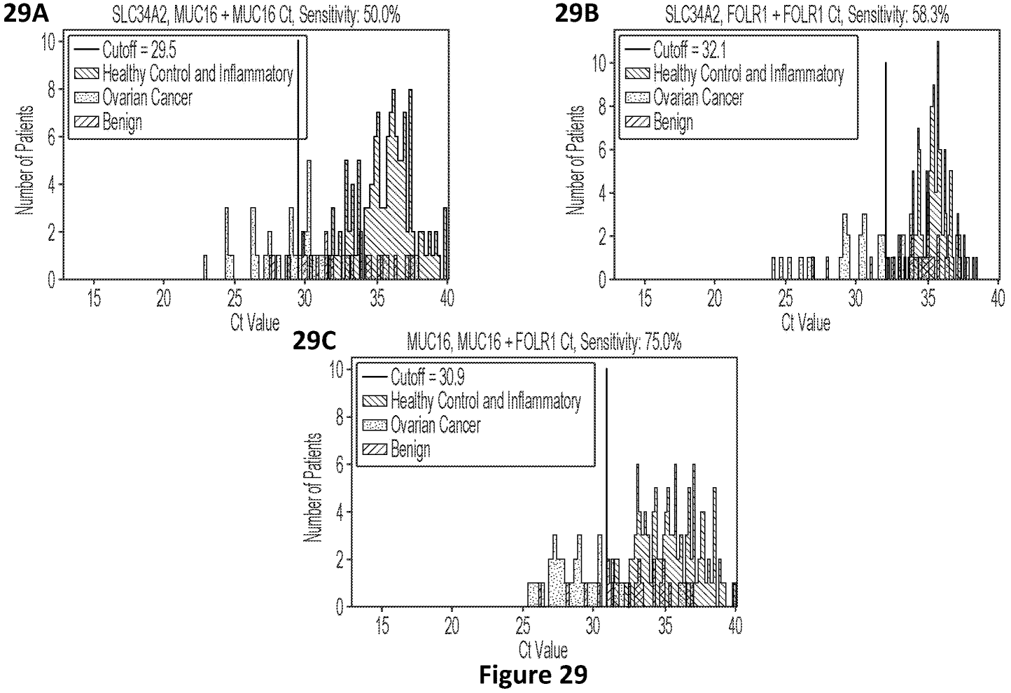

In certain embodiments, at least two (including, e.g., at least three or more) distinct target biomarker signatures each comprising at least one extracellular vesicle-associated membrane-bound polypeptide and at least one (including, e.g., at least two, or more) target biomarker (which may be selected from any of surface protein biomarkers described herein, intravesicular protein biomarkers described herein, and/or intravesicular RNA biomarkers described herein) may be embodied in an ovarian cancer detection assay. For example, FIG. 21 discloses an ovarian cancer detection assay using at least two target biomarker signatures for ovarian cancer, wherein a first target biomarker signature comprises SLC34A1 and MUC16 and a second target biomarker signature comprises SLC34A2 and FOLR1. As illustrated in FIG. 21, in some embodiments, a capture agent directed to SLC34A2, which in some embodiments, may be immobilized on solid substrates, e.g., beads such as magnetic beads), is used to capture extracellular vesicles from a patient’s blood-derived sample. A first aliquot of captured extracellular vesicles is subjected to a first set of detection probes each directed to MUC16, while a second aliquot of captured extracellular vesicles is subjected to a second set of detection probes each directed to FOLR1. FIG. 29 discloses an ovarian cancer detection assay using at least three target biomarker signatures for ovarian cancer, wherein a first target biomarker signature comprises SLC34A1 and MUC16; a second target biomarker signature comprises SLC34A2 and FOLR1; and a third target biomarker signature comprises MUC16 and FOLR1. As illustrated in FIG. 29, in some embodiments, an aliquot of a patient’s blood-derived sample is subjected to a first capture agent directed to SLC34A2, which in some embodiments, may be immobilized on solid substrates, e.g., beads such as magnetic beads) for capture of extracellular vesicles for further analysis. A first aliquot of SLC34A2-captured extracellular vesicles is subjected to a first set of detection probes each directed to MUC16, while a second aliquot of SLC34A2-captured extracellular vesicles is subjected to a second set of detection probes each directed to FOLR1. In some embodiments, another aliquot of such a patient’s blood-derived sample is subjected to a second capture agent directed to MUC16, which in some embodiments, may be immobilized on solid substrates, e.g., beads such as magnetic beads) for capture of extracellular vesicles for further analysis. MUC16-captured extracellular vesicles is then subjected to a set comprising a first detection probe directed to MUC16 and a second detection probe directed to FOLR1.

As shown in FIGS. 21 and 29, in some embodiments, each distinct target biomarker signature may have a different pre-determined cutoff value for individually determining whether a sample is positive for ovarian cancer. In some embodiments, a sample is determined to be positive for ovarian cancer if assay readout is above at least one of cutoff values for a plurality of (e.g., at least 2 or more) target biomarker signatures. In some embodiments, a combination of cutoff values (e.g., at least 2, at least 3, or more) can be utilized to create a diagnostic value with corollarily improved sensitivity and/or specificity.

Accordingly, in some embodiments, a sample can be divided into aliquots such that a different capture agent and/or a different set of detection probes (e.g., each directed to detection of a distinct disease or condition) can be added to a different aliquot. In such embodiments, provided technologies can be implemented with one aliquot at a time or multiple aliquots at a time (e.g., for parallel assays to increase throughput).

In some embodiments, amount of detection probes that is added to a sample provides a sufficiently low concentration of detection probes in a mixture to ensure that the detection probes will not randomly come into close proximity with one another in the absence of binding to an entity of interest (e.g., biological entity), at least not to any great or substantial degree. As such, in many embodiments, when detection probes simultaneously bind to the same entity of interest (e.g., biological entity) through the binding interaction between respective targeting binding moieties of the detection probes and the binding sites of an entity of interest (e.g., a biological entity), the detection probes come into sufficiently close proximity to one another to form double-stranded complex (e.g., as described herein). In some embodiments, the concentration of detection probes in a mixture following combination with a sample may range from about 1 fM to 1 µM, such as from about 1 pM to about 1 nM, including from about 1 pM to about 100 nM.

In some embodiments, the concentration of an entity of interest (e.g., a biological entity) in a sample is sufficiently low such that a detection probe binding to one entity of interest (e.g., a biological entity) will not randomly come into close proximity with another detection probe binding to another entity of interest (e.g., biological entity) in the absence of respective detection probes binding to the same entity of interest (e.g., biological entity), at least not to any great or substantial degree. By way of example only, the concentration of an entity of interest (e.g., biological entity) in a sample is sufficiently low such that a first target detection probe binding to a non-target entity of interest (e.g., a non-cancerous biological entity such as an extracellular vesicle comprising a first target) will not randomly come into close proximity with another different target detection probe that is bound to another non-target entity of interest (e.g., a non-cancerous biological entity such as an extracellular vesicle), at least not to any great or substantial degree, to generate a false positive detectable signal.

Following contacting an entity of interest (e.g., biological entity) in a sample with a set of detection probes, such a mixture may be incubated for a period of time sufficient for the detection probes to bind corresponding targets (e.g., molecular targets), if present, in the entity of interest to form a double-stranded complex (e.g., as described herein). In some embodiments, such a mixture is incubated for a period of time ranging from about 5 min to about 5 hours, including from about 30 min to about 2 hours, at a temperature ranging from about 10 to about 50° C., including from about 20° C. to about 37° C.

A double-stranded complex (resulted from contacting an entity of interest such as a biological entity with detection probes) can then be subsequently contacted with a nucleic acid ligase to perform nucleic acid ligation of a free 3′ end hydroxyl and 5′ end phosphate end of oligonucleotide strands of detection probes, thereby generating a ligated template comprising oligonucleotide strands of at least two or more detection probes. In some embodiments, prior to contacting an assay sample comprising a double-stranded complex with a nucleic acid ligase, at least one or more inhibitor oligonucleotide (e.g., as described herein) can be added to the assay sample such that the inhibitor oligonucleotide can capture any residual free-floating detection probes that may otherwise interact with each other during a ligation reaction.

As is known in the art, ligases catalyze the formation of a phosphodiester bond between juxtaposed 3′-hydroxyl and 5′-phosphate termini of two immediately adjacent nucleic acids when they are annealed or hybridized to a third nucleic acid sequence to which they are complementary. Any known nucleic acid ligase (e.g., DNA ligases) may be employed, including but not limited to temperature sensitive and/or thermostable ligases. Non-limiting examples of temperature sensitive ligases include bacteriophage T4 DNA ligase, bacteriophage T7 ligase, and E. coli ligase. Non-limiting examples of thermostable ligases include Taq ligase, Tth ligase, and Pfu ligase. Thermostable ligase may be obtained from thermophilic or hyperthermophilic organisms, including but not limited to, prokaryotic, eukaryotic, or archael organisms. In some embodiments, a nucleic acid ligase is a DNA ligase. In some embodiments, a nucleic acid ligase can be a RNA ligase.

In some embodiments, in a ligation step, a suitable nucleic acid ligase (e.g., a DNA ligase) and any reagents that are necessary and/or desirable are combined with the reaction mixture and maintained under conditions sufficient for ligation of the hybridized ligation oligonucleotides to occur. Ligation reaction conditions are well known to those of skill in the art. During ligation, a reaction mixture, in some embodiments, may be maintained at a temperature ranging from about 20° C. to about 45° C., such as from about 25° C. to about 37° C. for a period of time ranging from about 5 minutes to about 16 hours, such as from about 1 hour to about 4 hours. In yet other embodiments, a reaction mixture may be maintained at a temperature ranging from about 35° C. to about 45° C., such as from about 37° C. to about 42° C., e.g., at or about 38° C., 39° C., 40° C. or 41° C., for a period of time ranging from about 5 minutes to about 16 hours, such as from about 1 hour to about 10 hours, including from about 2 to about 8 hours.

Detection of such a ligated template can provide information as to whether an entity of interest (e.g., a biological entity) in a sample is positive or negative for targets to which detection probes are directed. For example, a detectable level of such a ligated template is indicative of a tested entity of interest (e.g., a biological entity) comprising targets (e.g., molecular targets) of interest. In some embodiments, a detectable level is a level that is above a reference level, e.g., by at least 10% or more, including, e.g., at least 20%, at least 30%, at least 40%, at least 50%, at least 60%, at least 70%, at least 80%, at least 90% or more. In some embodiments, a reference level may be a level observed in a negative control sample, such as a sample in which an entity of interest comprising such targets is absent. Conversely, a non-detectable level (e.g., a level that is below the threshold of a detectable level) of such a ligated template indicates that at least one of targets (e.g., molecular targets) of interest is absent from a tested entity of interest (e.g., a biological entity). Those of skill in the art will appreciate that a threshold that separates a detectable level from a non-detectable level may be determined based on, for example, a desired sensitivity level, and/or a desired specificity level that is deemed to be optimal for each application and/or purpose. For example, in some embodiments, a specificity of 99.7% may be achieved using a system provided herein, for example by setting a threshold that is three standard deviations above a reference level (e.g., a level observed in a negative control sample, such as, e.g., a sample derived from one or more normal healthy individuals). Additionally or alternatively, those of skill in the art will appreciate that a threshold of a detectable level (e.g., as reflected by a detection signal intensity) may be 1 to 100-fold above a reference level.

In some embodiments, a method provided herein comprises, following ligation, detecting a ligated template, e.g., as a measure of the presence and/or amount of an entity of interest in a sample. In various embodiments, detection of a ligated template may be qualitative or quantitative. As such, in some embodiments where detection is qualitative, a method provides a reading or evaluation, e.g., assessment, of whether or not an entity of interest (e.g., a biological entity) comprising at least two or more targets (e.g., molecular targets) is present in a sample being assayed. In other embodiments, a method provides a quantitative detection of whether an entity of interest (e.g., a biological entity) comprising at least two or more targets (e.g., molecular targets) is present in a sample being assayed, e.g., an evaluation or assessment of the actual amount of an entity of interest (e.g., a biological entity) comprising at least two or more targets (e.g., molecular targets) in a sample being assayed. In some embodiments, such quantitative detection may be absolute or relative.

A ligated template formed by using technologies provided herein may be detected by an appropriate method known in the art. Those of skill in the art will appreciate that appropriate detection methods may be selected based on, for example, a desired sensitivity level and/or an application in which a method is being practiced. In some embodiments, a ligated template can be directly detected without any amplification, while in other embodiments, ligated template may be amplified such that the copy number of the ligated template is increased, e.g., to enhance sensitivity of a particular assay. Where detection without amplification is practicable, a ligated template may be detected in a number of different ways. For example, oligonucleotide domains of detection probes (e.g., as described and/or utilized herein) may have been directly labeled, e.g., fluorescently or radioisotopically labeled, such that a ligated template is directly labeled. For example, in some embodiments, an oligonucleotide domain of a detection probe (e.g., as provided and/or utilized herein) can comprise a detectable label. A detectable label may be a composition detectable by spectroscopic, photochemical, biochemical, immunochemical, electrical, optical or chemical means. Such labels include biotin for staining with labeled Streptavidin conjugate, magnetic beads (e.g., Dynabeads®), fluorescent dyes (e.g., fluorescein, texas red, rhodamine, green fluorescent protein, and the like), radiolabels (e.g., ³H, ¹²⁵I, ³⁴S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. In some embodiments, a directly labeled ligated template may be size separated from the remainder of the reaction mixture, including unligated directly labeled ligation oligonucleotides, in order to detect the ligated template.

In some embodiments, detection of a ligated template can include an amplification step, where the copy number of ligated nucleic acids is increased, e.g., in order to enhance sensitivity of the assay. The amplification may be linear or exponential, as desired, where amplification can include, but are not limited to polymerase chain reaction (PCR); quantitative PCR, isothermal amplification, NASBA, digital droplet PCR, etc.

Various technologies for achieving PCR amplification are known in the art; those skilled in the art will be well familiar with a variety of embodiments of PCR technologies, and will readily be able to select those suitable to amplify a ligated template generated using technologies provided herein. For example, in some embodiments, a reaction mixture that includes a ligated template is combined with one or more primers that are employed in the primer extension reaction, e.g., PCR primers (such as forward and reverse primers employed in geometric (or exponential) amplification or a single primer employed in a linear amplification). Oligonucleotide primers with which one or more ligated templates are contacted should be of sufficient length to provide for hybridization to complementary template DNA under appropriate annealing conditions. Primers are typically at least 10 bp in length, including, e.g., at least 15 bp in length, at least 20 bp in length, at least 25 bp in length, at least 30 bp in length or longer. In some embodiments, the length of primers can typically range from about 15 to 50 bp in length, from about 18 to 30 bp, or about 20 to 35 bp in length. Ligated templates may be contacted with a single primer or a set of two primers (forward and reverse primers), depending on whether primer extension, linear, or exponential amplification of the template DNA is desired.

In addition to the above components, a reaction mixture comprising a ligated template typically includes a polymerase and deoxyribonucleoside triphosphates (dNTPs). The desired polymerase activity may be provided by one or more distinct polymerase enzymes. In preparing a reaction mixture, e.g., for amplification of a ligated template, various constituent components may be combined in any convenient order. For example, an appropriate buffer may be combined with one or more primers, one or more polymerases and a ligated template to be detected, or all of the various constituent components may be combined at the same time to produce the reaction mixture.

### VI. Uses

In some embodiments, one or more provided biomarkers of one or more target biomarker signatures for ovarian cancer can be detected in a sample comprising biological entities (including, e.g., cells, circulating tumor cells, cell-free DNA, extracellular vesicles, etc.), for example, using methods of detecting and/or assays as described herein. In some embodiments, one or more provided biomarkers of one or more target biomarker signatures for ovarian cancer can be detected in a sample comprising extracellular vesicles, for example, using methods of detecting and/or assays as described herein.

In some embodiments, a sample may be or comprise a biological sample. In some embodiments, a biological sample can be derived from a blood or blood-derived sample of a subject (e.g., a human female subject such as a human woman subject) in need of such an assay. In some embodiments, a biological sample can be or comprise a primary sample (e.g., a tissue or tumor sample) from a subject (e.g., a human subject) in need of such an assay. In some embodiments, a biological sample can be processed to separate one or more entities of interest (e.g., biological entity) from non-target entities of interest, and/or to enrich one or more entities of interest (e.g., biological entity). In some embodiments, an entity of interest present in a sample may be or comprise a biological entity, e.g., a cell or an extracellular vesicle (e.g., an exosome). In some embodiments, such a biological entity (e.g., extracellular vesicle) may be processed or contacted with a chemical reagent, e.g., to stabilize and/or crosslink targets (e.g., provided target biomarkers) to be assayed in the biological entity and/or to reduce non-specific binding with detection probes. In some embodiments, a biological entity is or comprises a cell, which may be optionally processed, e.g., with a chemical reagent for stabilizing and/or crosslinking targets (e.g., molecular targets) and/or for reducing non-specific binding. In some embodiments, a biological entity is or comprises an extracellular vesicle (e.g., an exosome), which may be optionally processed, e.g., with a chemical reagent for stabilizing and/or crosslinking targets (e.g., molecular targets) and/or for reducing non-specific binding.

In some embodiments, technologies provided herein can be useful for managing patient care, e.g., for one or more individual subjects and/or across a population of subjects. By way of example only, in some embodiments, provided technologies may be utilized in screening, which for example, may be performed periodically, such as annually, semi-annually, bi-annually, or with some other frequency as deemed to be appropriate by those skilled in the art. In some embodiments, such a screening may be temporally motivated or incidentally motivated. For example, in some embodiments, provided technologies may be utilized in temporally motivated screening for one or more individual subjects or across a population of subjects (e.g., asymptomatic female subjects) who are older than a certain age (e.g., over 40, 45, 50, 55, 60, 65, 70, 75, 80, or older). As will be appreciated by those skilled in the art, in some embodiments, the screening age and/or frequency may be determined based on, for example, but not limited to prevalence of a disease, disorder, or condition (e.g., cancer such as ovarian cancer). In some embodiments, provided technologies may be utilized in incidentally-motivated screening for individual subjects who may have experienced an incident or event that motivates screening for a particular disease, disorder, or condition (e.g., cancer such as ovarian cancer). For example, in some embodiments, an incidental motivation relating to determination of one or more indicators of a disease, disorder, or condition (e.g., cancer such as ovarian cancer) or susceptibility thereto may be or comprise, e.g., an incident based on their family history (e.g., a close relative such as blood-related relative was previously diagnosed for such a disease, disorder, or condition such as ovarian cancer or breast cancer), identification of one or more risk factors for a disease, disorder, or condition (e.g., ovarian cancer) and/or prior incidental findings from genetic tests (e.g., genome sequencing), and/or imaging diagnostic tests (e.g., ultrasound, computerized tomography (CT) and/or magnetic resonance imaging (MRI) scans), development of one or more signs or symptoms characteristic of a particular disease, disorder, or condition (e.g., abnormal bleeding during a woman’s period potentially indicative of ovarian cancer, etc.) and/or other incidents or events as will be appreciated by those skilled in the art.

In some embodiments, provided technologies for managing patient care can inform treatment and/or payment (e.g., reimbursement for treatment) decisions and/or actions. For example, in some embodiments, provided technologies can provide determination of whether individual subjects have one or more indicators of risk, incidence, or recurrence of a disease disorder, or condition (e.g., cancer such as ovarian cancer), thereby informing physicians and/or patients when to provide/receive therapeutic or prophylactic recommendations and/or to initiate such therapy in light of such findings. In some embodiments, such individual subjects may be asymptomatic subjects, who may be temporally-motivated or incidentally-motivated screened at a regular frequency (e.g., annually, semi-annually, bi-annually, or other frequency as deemed to be appropriate by those skilled in the art). In some embodiments, such individual subjects may be experiencing one or more symptoms that may be associated with ovarian cancer, who may be temporally-motivated or incidentally-motivated screened at a regular frequency (e.g., annually, semi-annually, bi-annually, or other frequency as deemed to be appropriate by those skilled in the art). In some embodiments, such individual subjects may be subjects having a benign gynecological tumor and/or a chronic inflammatory condition, who may be temporally-motivated or incidentally-motivated screened at a regular frequency (e.g., annually, semi-annually, bi-annually, or other frequency as deemed to be appropriate by those skilled in the art). In some embodiments, such individual subjects may be subjects at hereditary risk for ovarian cancer, who may be temporally-motivated or incidentally-motivated screened at a regular frequency (e.g., annually, semi-annually, bi-annually, or other frequency as deemed to be appropriate by those skilled in the art). In some embodiments, such individual subjects may be subjects with life-history associated risk, who may be temporally-motivated or incidentally-motivated screened at a regular frequency (e.g., annually, semi-annually, bi-annually, or other frequency as deemed to be appropriate by those skilled in the art). In some embodiments, such individual subjects may be post-menopausal subjects, who may be temporally-motivated or incidentally-motivated screened at a regular frequency (e.g., annually, semi-annually, bi-annually, or other frequency as deemed to be appropriate by those skilled in the art). In some embodiments, such post-menopausal subjects may be experiencing abdominal pain and/or pelvic pain.

Additionally or alternatively, in some embodiments, provided technologies can inform physicians and/or patients of treatment selection, e.g., based on findings of specific responsiveness biomarkers (e.g., cancer responsiveness biomarkers). In some embodiments, provided technologies can provide determination of whether individual subjects are responsive to current treatment, e.g., based on findings of changes in one or more levels of molecular targets associated with a disease, thereby informing physicians and/or patients of efficacy of such therapy and/or decisions to maintain or alter therapy in light of such findings. In some embodiments, provided technologies can provide determination of whether individual subjects are likely to be responsive to a recommended treatment, e.g., based on findings of molecular targets (e.g., provided biomarkers of one or more target biomarker signatures for ovarian cancer) that predict therapeutic effects of a recommended treatment on individual subjects, thereby informing physicians and/or patients of potential efficacy of such therapy and/or decisions to administer or alter therapy in light of such findings.

In some embodiments, provided technologies can inform decision making relating to whether health insurance providers reimburse (or not), e.g., for (1) screening itself (e.g., reimbursement available only for periodic/regular screening or available only for temporally-and/or incidentally- motivated screening); and/or for (2) initiating, maintaining, and/or altering therapy in light of findings by provided technologies. For example, in some embodiments, the present disclosure provides methods relating to (a) receiving results of a screening that employs provided technologies and also receiving a request for reimbursement of the screening and/or of a particular therapeutic regimen; (b) approving reimbursement of the screening if it was performed on a subject according to an appropriate schedule (based on, e.g., screening age such as older than a certain age, e.g., over 40, 45, 50, 55, 60, 65, 70, 75, 80, or older, and/or screening frequency such as, e.g., every 3 months, every 6 months, every year, every 2 years, every 3 years or at some other frequencies) or response to a relevant incident and/or approving reimbursement of the therapeutic regimen if it represents appropriate treatment in light of the received screening results; and, optionally (c) implementing the reimbursement or providing notification that reimbursement is refused. In some embodiments, a therapeutic regimen is appropriate in light of received screening results if the received screening results detect a biomarker that represents an approved biomarker for the relevant therapeutic regimen (e.g., as may be noted in a prescribing information label and/or via an approved companion diagnostic).

Alternatively or additionally, the present disclosure contemplates reporting systems (e.g., implemented via appropriate electronic device(s) and/or communications system(s)) that permit or facilitate reporting and/or processing of screening results (e.g., as generated in accordance with the present disclosure), and/or of reimbursement decisions as described herein. Various reporting systems are known in the art; those skilled in the art will be well familiar with a variety of such embodiments, and will readily be able to select those suitable for implementation.

### Exemplary Uses

### A. Detection of Ovarian Cancer Incidence or Recurrence

The present disclosure, among other things, recognizes that detection of a single cancer-associated biomarker in a biological entity (e.g., extracellular vesicle) or a plurality of cancer-associated biomarkers based on a bulk sample, rather than at a resolution of a single biological entity (e.g., individual extracellular vesicles), typically does not provide sufficient specificity and/or sensitivity in determination of whether a subject from whom the biological entity is obtained is likely to be suffering from or susceptible to cancer (e.g., ovarian cancer). The present disclosure, among other things, provides technologies, including compositions and/or methods, that solve such problems, including for example by specifically requiring that an entity (e.g., an extracellular vesicle) for detection be characterized by presence of a combination of at least two or more targets (e.g., at least two or more provided biomarkers of a target biomarker signature for ovarian cancer). In particular embodiments, the present disclosure teaches technologies that require such an entity (e.g., an extracellular vesicle) be characterized by presence (e.g., by expression) of a combination of molecular targets that is specific to cancer (i.e., “target biomarker signature” of a relevant cancer, e.g., ovarian cancer), while biological entities (e.g., extracellular vesicles) that do not comprise the targeted combination (e.g., target biomarker signature) do not produce a detectable signal. Accordingly, in some embodiments, technologies provided herein can be useful for detection of risk, incidence, and/or recurrence of cancer in a subject. In some such embodiments, technologies provided herein are useful for detection of risk, incidence, and/or recurrence of ovarian cancer in a female subject. For example, in some embodiments, a combination of two or more provided biomarkers are selected for detection of a specific cancer (e.g., ovarian cancer) or various cancers (one of which includes ovarian cancer). In some embodiments, a specific combination of provided biomarkers for detection of ovarian cancer can be determined by analyzing a population or library (e.g., tens, hundreds, thousands, tens of thousands, hundreds of thousands, or more) of ovarian cancer patient biopsies and/or patient data to identify such a predictive combination. In some embodiments, a relevant combination of biomarkers may be one identified and/or characterized, for example, via data analysis. In some embodiments, for example, a diverse set of ovarian cancer-associated data (e.g., in some embodiments comprising one or more of bulk RNA sequencing, single-cell RNA (scRNA) sequencing, mass spectrometry, histology, post-translational modification data, in vitro and/or in vivo experimental data) can be analyzed through machine learning and/or computational modeling to identify a combination of predictive markers that is highly specific to ovarian cancer. In some embodiments, a combination of predictive markers to distinguish stages of cancer (e.g., ovarian cancer) can be determined in silico based on comparing and analyzing diverse data (e.g., in some embodiments comprising bulk RNA sequencing, scRNA sequencing, mass spectrometry, histology, post-translational modification data, in vitro and/or in vivo experimental data) relating to different stages of cancer (e.g., ovarian cancer). For example, in some embodiments, technologies provided herein can be used to distinguish ovarian cancer subjects from non-ovarian cancer subjects, including, e.g., healthy women subjects, women subjects diagnosed with benign tumors or adnexal masses, and women subjects with non-ovarian-related diseases, disorders, and/or conditions (e.g., women subjects with non-ovarian cancer, or women subjects with inflammatory bowel diseases or disorders). In some embodiments, technologies provided herein can be useful for early detection of ovarian cancer, e.g., detection of ovarian cancer of stage I or stage II. In some embodiments, technologies provided herein can be useful for detection of one or more ovarian cancer subtypes, including, e.g., high-grade serous ovarian cancer, endometrioid ovarian cancer, clear-cell ovarian cancer, low-grade serous ovarian cancer, or mucinous ovarian cancer. In some embodiments, technologies provided herein can be useful for screening women at hereditary risk or average risk for early stage high-grade serous ovarian cancer.

In some embodiments, technologies provided herein can be useful for screening a subject for risk, incidence, or recurrence of a specific cancer in a single assay. For example, in some embodiments, technologies provided herein is useful for screening a subject for risk, incidence, or recurrence of ovarian cancer. In some embodiments, technologies provided herein can be used to screen a subject for risk or incidence of a specific cancer or a plurality of (e.g., at least 2, at least 3, or more) cancers in a single assay. For example, in some embodiments, technologies provided herein can be used to screen a subject for a plurality of cancers in a single assay, one of which includes ovarian cancer and other cancers to be screened can be selected from the group consisting of brain cancer (including, e.g., glioblastoma), breast cancer, colorectal cancer, pancreatic cancer, prostate cancer, liver cancer, lung cancer, and skin cancer.

In some embodiments, provided technologies can be used periodically (e.g., every year, every two years, every three years, etc.) to screen a human subject (e.g., a human female subject) for ovarian cancer (e.g., early-stage ovarian cancer) or cancer recurrence. In some embodiments, a human subject amenable to such screening may be an adult or an elderly. In some embodiments, a human subject amenable to such screening may be a post-menopausal woman. In some embodiments, a human subject amenable to such screening may be an elderly woman, e.g., age 65 above, age 70 above, at least 75 above, at least 80, or above. In some embodiments, a human subject amenable to such screening may have an age of about 50 or above. In some embodiments, a human subject amenable to such screening may have an age of 50 or less. In some embodiments, a human subject amenable to such screening may have an age over 35.

In some embodiments, a subject that is amenable to provided technologies for detection of incidence or recurrence of ovarian cancer may be a post-menopausal human female subject, who in some embodiments may be experiencing abdominal pain and/or pelvic pain. In some embodiments, a subject that is amenable to provided technologies for detection of incidence or recurrence of ovarian cancer may be a human female subject who is at least 55 years old and is determined to have a benign gynecological tumor and/or one or more chronic inflammatory conditions. In some embodiments, a subject that is amenable to provided technologies for detection of incidence or recurrence of ovarian cancer may be a post-menopausal human female subject or a human female subject at age of least 55 years old, who has a family history of breast and/or ovarian cancer (e.g., women having one or more first-degree relatives with a history of breast cancer and/or ovarian cancer), who has been previously treated for cancer (e.g., ovarian cancer), who is at risk of ovarian cancer recurrence after cancer treatment, who is in remission after ovarian cancer treatment, and/or who has been previously or periodically screened for ovarian cancer, e.g., by screening for the presence of at least one ovarian cancer biomarker (e.g., by detecting serum protein CA-125 and/or by transvaginal ultrasound (TVUS)). Alternatively, in some embodiments, a post-menopausal human female subject or a human female subject at age of least 55 years old may be a female subject who has not been previously screened for ovarian cancer, who has not been diagnosed for ovarian cancer, and/or who has not previously received ovarian cancer therapy. In some embodiments, a post-menopausal human female subject or a human female subject at age of least 55 years old may be a female subject with a benign gynecological tumor. In some embodiments, a post-menopausal subject may be a subject who is susceptible to ovarian cancer (e.g., at an average population risk, at increased risk due to life-history factors, at increased risk due to menopause, or with hereditary risk for ovarian cancer).

In some embodiments, the present disclosure, among other things, provides insights that technologies described and/or utilized herein may be particularly useful for screening certain populations of female subjects, e.g., female subjects who are at higher susceptibility to developing ovarian cancer. In some embodiments, the present disclosure, among other things, recognizes that the resulting PPVs of technologies described and/or utilized herein for HGSOC detection may be higher in ovarian cancer prone or susceptible populations. In some embodiments, the present disclosure, among other things, provides insights that screening of post-menopausal individuals, e.g., regular screening prior to or otherwise in absence of developed symptom(s), can be beneficial, and even important for effective management (e.g., successful treatment) of ovarian cancer. In some embodiments, the present disclosure provides ovarian cancer screening systems that can be implemented to detect ovarian cancer, including early-stage cancer, in some embodiments in post-menopausal individuals (e.g., with or without hereditary and/or life-history risks in ovarian cancer and/or with or without symptoms such as abdominal and/or pelvic pain). In some embodiments, provided technologies can be implemented to achieve regular screening of post-menopausal individuals (e.g., with or without hereditary and/or life-history risks in ovarian cancer and/or with or without symptoms such as abdominal and/or pelvic pain). In some embodiments, provided technologies achieve detection (e.g., early detection, e.g., in symptomatic or asymptomatic individual(s) and/or population(s)) of one or more features (e.g., incidence, progression, responsiveness to therapy, recurrence, etc.) of ovarian cancer, with sensitivity and/or specificity (e.g., rate of false positive and/or false negative results) appropriate to permit useful application of provided technologies to single-time and/or regular (e.g., periodic) assessment. In some embodiments, provided technologies are useful in conjunction with women’s periodic physical examination such as mammograms, HPV, and/or Pap smear screening (e.g., every year, every other year, or at an interval approved by the attending physician). In some embodiments, provided technologies are useful in conjunction with treatment regimen(s); in some embodiments, provided technologies may improve one or more characteristics (e.g., rate of success according to an accepted parameter) of such treatment regimen(s).

In some embodiments, a subject that is amenable to provided technologies for detection of incidence or recurrence of ovarian cancer may be an asymptomatic human female subject and/or across an asymptomatic population of female subjects. Such an asymptomatic subject and/or across an asymptomatic population of female subjects may be subject(s) who has/have a family history of breast and/or ovarian cancer (e.g., women having one or more first-degree relatives with a history of breast cancer and/or ovarian cancer), who has been previously treated for cancer (e.g., ovarian cancer), who is at risk of ovarian cancer recurrence after cancer treatment, who is in remission after ovarian cancer treatment, and/or who has been previously or periodically screened for ovarian cancer, e.g., by screening for the presence of at least one ovarian cancer biomarker (e.g., by detecting serum protein CA-125 and/or by transvaginal ultrasound (TVUS)). Alternatively, in some embodiments, an asymptomatic subject may be a female subject who has not been previously screened for ovarian cancer, who has not been diagnosed for ovarian cancer, and/or who has not previously received ovarian cancer therapy. In some embodiments, an asymptomatic subject may be a female subject with a benign gynecological tumor. In some embodiments, an asymptomatic subject may be a subject who is susceptible to ovarian cancer (e.g., at an average population risk or with hereditary risk for ovarian cancer).

In some embodiments, a subject or population of subjects that are amenable to provided technologies for detection of ovarian cancer may be selected based on one or more characteristics such as age, race, genetic history, medical history, personal history (e.g., smoking, alcohol, drugs, carcinogenic agents, diet, obesity, physical activity, sun exposure, radiation exposure, exposure to infectious agents such as viruses, and/or occupational hazard). For example, in some embodiments, a subject or population of subjects that are amenable to provided technologies for detection of ovarian cancer may be a female subject (e.g., a woman) or a population of female subjects (e.g., women) determined to have one or more germline mutations in ovarian cancer-associated genes, including but not limited to, e.g., BARD1, BRIP1, RAD51C, RAD51D, CHEK2, MRE11A, RAD50, ATM, BRCA1, BRCA2, CDKN2A, MSH2, MLH1, MSH2, EPCAM, PALB2, STK11, TP53, and combinations thereof. In some embodiments, a subject or population of subjects that are amenable to provided technologies for detection of ovarian cancer may be a female subject (e.g., a woman) or a population of female subjects (e.g., women) determined to have one or more germline single nucleotide polymorphisms at specific loci or within certain genes determined by genome wide association studies to be associated with ovarian-cancer, including but not limited to e.g., WNT4, RSPO1, BCL2L11, HOXD3, HAGLR, TIPARP, SYNPO2, TERT, GPX6, CHMP4C, LINC00824, COL15A1, SMC2-AS1, MLLT10, INCENP, RCCD1, ATAD5, HNF1B, PLEKHM1, SKAP1, ANKLE1, GATAD2A, Cytobands and SNPs 2q13 rs752590, 4q32.3 rs4691139, 9p22 rs3814113, 9q34.2 rs635634, 10p11.21 rs1192691, and/or 19q13.2 rs688187 (Reid et al., 2017; which is incorporated herein by reference for the purpose described herein).

In some embodiments, a subject or population of subjects that are amenable to provided technologies for detection of ovarian cancer may be a female subject (e.g., a woman) or a population of female subjects (e.g., women) with breast cancer determined to have germline mutations in BRCA1, BRCA2 and/or PALB2.

In some embodiments, a subject or population of subjects that are amenable to provided technologies for detection of ovarian cancer may be selected based on one or more characteristics such as age, race, genetic history, medical history, personal history (e.g., smoking, alcohol, drugs, carcinogenic agents, diet, obesity, diabetes, nulliparousness/infertility, no history/short history of oral contraceptive use, physical activity, sun exposure, radiation exposure, perineal talc use, hormone replacement therapy (HRT), exposure to infectious agents such as viruses, and/or occupational hazard). For example, in some embodiments, a subject or population of subjects that are amenable to provided technologies for detection of ovarian cancer may be a female subject (e.g., a woman) or a population of female subjects (e.g., women) determined to have one or more life-history associated risk factors.

In some embodiments, a subject or population of subjects that are amenable to provided technologies for detection of ovarian cancer may be a female subject (e.g., a woman) or a population of female subjects (e.g., women) diagnosed with an imaging-confirmed adnexal mass or pelvic mass.

In some embodiments, a subject or population of subjects that are amenable to provided technologies for detection of ovarian cancer may be a female subject (e.g., a woman) or a population of female subjects (e.g., women) at hereditary risk before undergoing a risk-reducing bilateral salpingo-oophorectomy.

In some embodiments, a subject or population of subjects that are amenable to provided technologies for detection of ovarian cancer may be a female subject (e.g., a woman) or a population of female subjects (e.g., women) with one or more non-specific symptoms of ovarian cancer. In some embodiments, exemplary non-specific symptoms of ovarian cancer may include symptoms similar to one or more symptoms for irritable bowel syndrome.

In some embodiments, a subject that is amenable to provided technologies for detection of incidence or recurrence of ovarian cancer may be a symptomatic human female subject and/or across a symptomatic population of female subjects. Such a symptomatic subject and/or across a symptomatic population of female subjects may be subject(s) who has/have a family history of breast and/or ovarian cancer (e.g., women having one or more first-degree relatives with a history of breast cancer and/or ovarian cancer), who has been previously treated for cancer (e.g., ovarian cancer), who is at risk of ovarian cancer recurrence after cancer treatment, who is in remission after ovarian cancer treatment, and/or who has been previously or periodically screened for ovarian cancer, e.g., by screening for the presence of at least one ovarian cancer biomarker (e.g., by detecting serum protein CA-125 and/or by transvaginal ultrasound (TVUS)). Alternatively, in some embodiments, a symptomatic subject may be a female subject who has not been previously screened for ovarian cancer, who has not been diagnosed for ovarian cancer, and/or who has not previously received ovarian cancer therapy. In some embodiments, a symptomatic subject may be a female subject with a benign gynecological tumor. In some embodiments, a symptomatic subject may be a subject who is susceptible to ovarian cancer (e.g., at an average population risk, with hereditary risk for ovarian cancer, with life-history associated risk for ovarian cancer, and/or with age associated risk for ovarian cancer).

In some embodiments, a subject or population of subjects that are amenable to provided technologies for detection of ovarian cancer may be a female subject (e.g., a woman) or a population of female subjects (e.g., women) of Asians, African Americans, Caucasians, Native Hawaiians or other Pacific Islanders, Hispanics or Latinos, American Indians or Alaska natives, non-Hispanic blacks, or non-Hispanic whites. In some embodiments, a subject or population of subjects that are amenable to provided technologies for detection of ovarian cancer may be a female subject (e.g., a woman) or a population of female subjects (e.g., women) of Asian Pacific Islanders, Hispanics, American Indian/Alaska natives, non-Hispanic black, or non-Hispanic white. In some embodiments, a subject or population of subjects that are amenable to provided technologies for detection of ovarian cancer may be a female subject (e.g., a woman) or a population of female subjects (e.g., women) of any race and/or any ethnicity.

In some embodiments, a subject or population of subjects that are amenable to provided technologies for detection of ovarian cancer may be determined to have a normal serum CA-125 level (e.g., a level of less than 35 U/mL). In some embodiments, a subject or population of subjects that are amenable to provided technologies for detection of ovarian cancer may be determined to have a serum CA-125 level that is equal to or higher than a normal serum CA-125 level (e.g., a level of less than 35 U/mL).

In some embodiments, technologies provided herein can be used in combination with other diagnostics assays including, e.g., but not limited to (i) a woman’s annual physical examination (e.g., including a HPV, and/or Pap smear screening for cervical cancer and a mammogram screening for breast cancer); (ii) serum CA-125 and/or TVUS screening test; (iii) a genetic assay to screen blood plasma for genetic mutations in circulating tumor DNA and/or protein biomarkers linked to cancer; (iv) an assay involving immunofluorescent staining to identify cell phenotype and marker expression, followed by amplification and analysis by next-generation sequencing; and (v) BRCA1 and/or BRCA2 germline and somatic mutation assays, or assays involving cell-free tumor DNA, liquid biopsy, serum protein and cell-free DNA, OVA1® and OVERA® tests, and/or circulating tumor cells.

### B. Selection of Cancer Therapy (e.g., Ovarian Cancer Therapy)

In some embodiments, provided technologies can be used for selecting an appropriate treatment for a cancer patient (e.g., a patient suffering from or susceptible to ovarian cancer). For example, some embodiments provided herein relate to a companion diagnostic assay for classification of patients for cancer therapy (e.g., ovarian cancer and/or adjunct treatment) which comprises assessment in a patient sample (e.g., a blood or blood-derived sample from an ovarian cancer patient) of a selected combination of provided biomarkers using technologies provided herein. Based on such an assay outcome, patients who are determined to be more likely to respond to a cancer therapy (e.g., an ovarian cancer therapy and/or an adjunct therapy, including, e.g., olaparib, cisplatin, rucaparib, niraparib, talazoparib) can be administered such a therapy, or patients who are determined to be non-responsive to a specific such therapy can be administered a different therapy.

### C. Evaluation of Treatment Efficacy (e.g., Cancer Treatment Efficacy)

In some embodiments, technologies provided herein can be used for monitoring and/or evaluating efficacy of an anti-cancer therapy administered to a cancer patient (e.g., ovarian cancer patient). For example, a blood or blood-derived sample can be collected from an ovarian cancer patient prior to or receiving an anti-cancer therapy (e.g., olaparib, cisplatin, rucaparib, niraparib, talazoparib) at a first time point to detect or measure tumor burdens, e.g., by detecting presence or amount of extracellular vesicles comprising a selected combination of biomarkers that is specific to detection of ovarian cancer. After a period of treatment, a second blood or blood-derived sample can be collected from the same ovarian cancer patient to detect changes in tumor burdens, e.g., by detecting absence or reduction in amount of extracellular vesicles comprising a selected combination of biomarkers that is specific to detection of ovarian cancer. By monitoring levels and/or changes in tumor burdens over the course of treatment, appropriate course of action, e.g., increasing or decreasing the dose of a therapeutic agent, and/or administering a different therapeutic agent, can be taken.

### VII Kits

Also provided are kits that find use in practicing technologies as described above. In some embodiments, a kit comprises a plurality of detection probes (e.g., as described and/or utilized herein). In some embodiments, a provided kit may comprise two or more (e.g., 3, 4, 5, 6, 7, 8, 9, 10, 11, 12, 13, 14, 15, 16, 17, 18, 19, 20 or more) detection probes. In some embodiments, individual detection probes may be directed at different targets. In some embodiments, two or more individual detection probes may be directed to the same target. In some embodiments, a provided kit comprises two or more different detection probes directed at different targets, and optionally may include at least one additional detection probe also directed at a target to which another detection probe is directed. In some embodiments, a provided kit comprises a plurality of subsets of detection probes, each of which comprises two or more detection probes directed at the same target. In some embodiments, a plurality of detection probes may be provided as a mixture in a container. In some embodiments, multiple subsets of detection probes may be provided as individual mixtures in separate containers. In some embodiments, each detection probe is provided individually in a separate container.

In some embodiments, a kit for detection of ovarian cancer comprises: (a) a capture agent comprising a target-capture moiety directed to an extracellular vesicle-associated membrane-bound polypeptide; and (b) a set of detection probes, which set comprises at least two detection probes each directed to a target biomarker of a target biomarker signature for ovarian cancer, wherein the detection probes each comprise:(i) a target binding moiety directed the target biomarker of the target biomarker signature for ovarian cancer; and (ii) an oligonucleotide domain coupled to the target binding moiety, the oligonucleotide domain comprising a double-stranded portion and a single-stranded overhang portion extended from one end of the oligonucleotide domain, wherein the single-stranded overhang portions of the at least two detection probes are characterized in that they can hybridize to each other when the at least two detection probes are bound to the same extracellular vesicle. In these embodiments, such a target biomarker signature for ovarian cancer comprises:
at least one extracellular vesicle-associated membrane-bound polypeptide biomarker and at least one target biomarker selected from the group consisting of: surface protein biomarkers, intravesicular protein biomarkers, and intravesicular RNA biomarkers, wherein:
   the surface protein biomarkers are selected from CLDN3, CLDN6, AQP5, CLDN16, EpCAM, FOLR1, LEMD1, LRRTM1, MUC16, CHODL, CDH6, HTR3A, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof;
   the intravesicular protein biomarkers are selected from CRABP2, KLK7, MIF, PRAME, and S100A1, and combinations thereof;
   the intravesicular RNA biomarkers are selected from CRABP2, MIF, CLDN6, PRAME, S100A1, KLK7, and combinations thereof;

In some embodiments, when at least one target biomarker is selected from one or more of the provided surface protein biomarkers, the selected surface protein biomarker(s) and the at least one extracellular vesicle-associated membrane-bound polypeptide are different. In some embodiments, when at least one target biomarker is selected from one or more of the provided surface protein biomarkers, the selected surface protein biomarker(s) and the at least one extracellular vesicle-associated membrane-bound polypeptide are the same (with the same or different epitopes).

In some embodiments, a capture agent provided in a kit comprises a target-capture moiety directed to an extracellular vesicle-associated membrane-bound polypeptide biomarker, which is or comprises CLDN3, CLDN6, AQP5, CLDN16, EpCAM, FOLR1, LEMD1, LRRTM1, MUC16, CHODL, CDH6, HTR3A, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof.

In some embodiments, a capture agent provided in a kit comprises a target-capture moiety directed to an extracellular vesicle-associated membrane-bound polypeptide, which is or comprises a SLC34A2 polypeptide. In some embodiments, a capture agent provided in a kit comprises a target-capture moiety directed to an extracellular vesicle-associated membrane-bound polypeptide, which is or comprises a MUC16 polypeptide. In some embodiments, a capture agent provided in a kit comprises a target-capture moiety directed to an extracellular vesicle-associated membrane-bound polypeptide, which is or comprises a FOLR1 polypeptide. In some embodiments, a capture agent provided in a kit comprises a target-capture moiety directed to an extracellular vesicle-associated membrane-bound polypeptide, which is or comprises a LRRTM1 polypeptide. In some embodiments, a capture agent provided in a kit comprises a target-capture moiety directed to an extracellular vesicle-associated membrane-bound polypeptide, which is or comprises a TACSTD2 polypeptide. In some embodiments, a capture agent provided in a kit comprises a target-capture moiety directed to an extracellular vesicle-associated membrane-bound polypeptide, which is or comprises a CD24 polypeptide. In some embodiments, a capture agent provided in a kit comprises a target-capture moiety directed to an extracellular vesicle-associated membrane-bound polypeptide, which is or comprises a PTGS1 polypeptide. In some embodiments, a capture agent provided in a kit comprises a target-capture moiety directed to an extracellular vesicle-associated membrane-bound polypeptide, which is or comprises a MUC1 polypeptide. In some embodiments, a capture agent provided in a kit comprises a target-capture moiety directed to an extracellular vesicle-associated membrane-bound polypeptide, which is or comprises a sTn polypeptide glycosylation. In some embodiments, a capture agent provided in a kit comprises a target-capture moiety directed to an extracellular vesicle-associated membrane-bound polypeptide, which is or comprises a MSLN polypeptide. In some embodiments, a capture agent provided in a kit comprises a target-capture moiety directed to an extracellular vesicle-associated membrane-bound polypeptide, which is or comprises an ALPL polypeptide. In some embodiments, a capture agent provided in a kit comprises a target-capture moiety directed to an extracellular vesicle-associated membrane-bound polypeptide, which is or comprises a BST2 polypeptide. In some embodiments, a capture agent provided in a kit comprises a target-capture moiety directed to an extracellular vesicle-associated membrane-bound polypeptide, which is or comprises a CLDN3 polypeptide. In some embodiments, a capture agent provided in a kit comprises a target-capture moiety directed to an extracellular vesicle-associated membrane-bound polypeptide, which is or comprises a CLDN6 polypeptide.

In some embodiments, a target binding moiety of at least two detection probes provided in a kit is each directed to the same target biomarker of a target biomarker signature. In some embodiments, such the same target biomarker is or comprises MUC16. In some embodiments, such the same target biomarker is or comprises FOLR1. In some embodiments, such the same target biomarker is or comprises CLDN3. In some embodiments, such the same target biomarker is or comprises CLDN6. In some embodiments, such the same target biomarker is or comprises SLC34A2. In some embodiments, such the same target biomarker is or comprises SLC2A1. In some embodiments, such the same target biomarker is or comprises MUC1. In some embodiments, such the same target biomarker is or comprises MSLN. In some embodiments, such the same target biomarker is or comprises AQP5. In some embodiments, such the same target biomarker is or comprises sTn. In some embodiments, such the same target biomarker is or comprises TACSTD2. In some such embodiments, an oligonucleotide domain of such at least two detection probes are different

In some embodiments, a target binding moiety of at least two detection probes provided in a kit is each directed to a distinct target biomarker of a target biomarker signature. For example, in some embodiments, a kit comprises at least two detection probes directed to MUC16 polypeptide and FOLR1 polypeptide. In some embodiments, a kit comprises at least two detection probes directed to MUC16 polypeptide and CLDN3 polypeptide. In some embodiments, a kit comprises at least two detection probes directed to FOLR1 polypeptide and CLDN3 polypeptide. In some embodiments, a kit comprises at least two detection probes directed to MUC16 polypeptide and CLDN6 polypeptide.

In some embodiments, a target binding moiety of a detection probe may be or comprise an antibody (e.g., a monoclonal antibody).

In some embodiments, a kit may comprise at least one chemical reagent such as a fixation agent, a permeabilization agent, and/or a blocking agent.

In some embodiments, a kit may comprise one or more nucleic acid ligation reagents (e.g., a nucleic acid ligase such as a DNA ligase and/or a buffer solution).

In some embodiments, a kit may comprise at least one or more amplification reagents such as PCR amplification reagents. In some embodiments, a kit may comprise one or more nucleic acid polymerases (e.g., DNA polymerases), one or more pairs of primers, nucleotides, and/or a buffered solution.

In some embodiments, a kit may comprise a solid substrate for capturing an entity (e.g., biological entity) of interest. For example, such a solid substrate may be or comprise a bead (e.g., a magnetic bead). In some embodiments, such a solid substrate may be or comprise a surface. In some embodiments, a surface may be or comprise a capture surface (e.g., an entity capture surface) of an assay chamber, such as, e.g., a filter, a matrix, a membrane, a plate, a tube, a well (e.g., but not limited to a microwell), etc. In some embodiments, a surface (e.g., a capture surface) of a solid substrate can be coated with a capture agent (e.g., polypeptide or antibody agent) for an entity (e.g., biological entity) of interest.

In some embodiments, a set of detection probes provided in a kit may be selected for diagnosis of ovarian cancer.

In some embodiments, a kit may comprise a plurality of sets of detection probes, wherein each set of detection probes is directed for detection of a specific cancer and comprises at least 2 or more detection probes. For example, such a kit can be used to screen a subject for various cancers, one of which is ovarian cancer while other cancers may be selected from skin cancer, lung cancer, breast cancer, colorectal cancer, pancreatic cancer, prostate cancer, brain cancer, and liver cancer) in a single assay.

In some embodiments, kits provided herein may include instructions for practicing methods described herein. These instructions may be present in kits in a variety of forms, one or more of which may be present in the kits. One form in which these instructions may be present is as printed information on a suitable medium or substrate, e.g., a piece or pieces of paper on which the information is printed, in the packaging of kits, in a package insert, etc. Yet another means may be a computer readable medium, e.g., diskette, CD, USB drive, etc., on which instructional information has been recorded. Yet another means that may be present is a website address which may be used via the internet to access instructional information. Any convenient means may be present in the kits.

In some embodiments where kits are for use as companion diagnostics, such kits can include instructions for identifying patients that are likely to respond to a therapeutic agent (e.g., identification of biomarkers that are indicative of patient responsiveness to the therapeutic agent). In some embodiments, such kits can comprise a therapeutic agent for use in tandem with the companion diagnostic test.

Other features of the invention will become apparent in the course of the following description of exemplary embodiments, which are given for illustration of the invention and are not intended to be limiting thereof.

### EXEMPLIFICATION

### Example 1: Detection of an Exemplary Target Biomarker Signature in Individual Extracellular Vesicles Associated with Ovarian Cancer

The present Example describes synthesis of detection probes for targets (e.g., target biomarker(s)) each comprising a target-binding moiety and an oligonucleotide domain (comprising a double-stranded portion and a single stranded overhang) coupled to the target-binding moiety. The present Example further demonstrates that use of such detection probes to detect the presence or absence of biological entities (e.g., extracellular vesicles) comprising two or more distinct targets.

In some embodiments, a detection probe can comprise a double-stranded oligonucleotide with an antibody agent specific to a target cancer biomarker at one end and a single stranded overhang at another end. When two or more detection probes are bound to the same biological entity (e.g., an extracellular vesicle), the single-stranded overhangs of the detection probes are in close proximity such that they can hybridize to each other to form a double-stranded complex, which can be subsequently ligated and amplified for detection.

This study employed at least two detection probes in a set. In some embodiments such at least two detection probes are directed to the same target, which may be directed to different epitopes of the same target or the same epitope of the same target. In some embodiments, such at least two detection probes are directed to distinct targets. A skilled artisan reading the present disclosure will understand that two detection probes can be directed to different target biomarkers, or that three or more detection probes, each directed towards a distinct target protein, may be used. Further, compositions and methods described in this Example can be extended to applications in different biological samples (e.g., comprising extracellular vesicles).

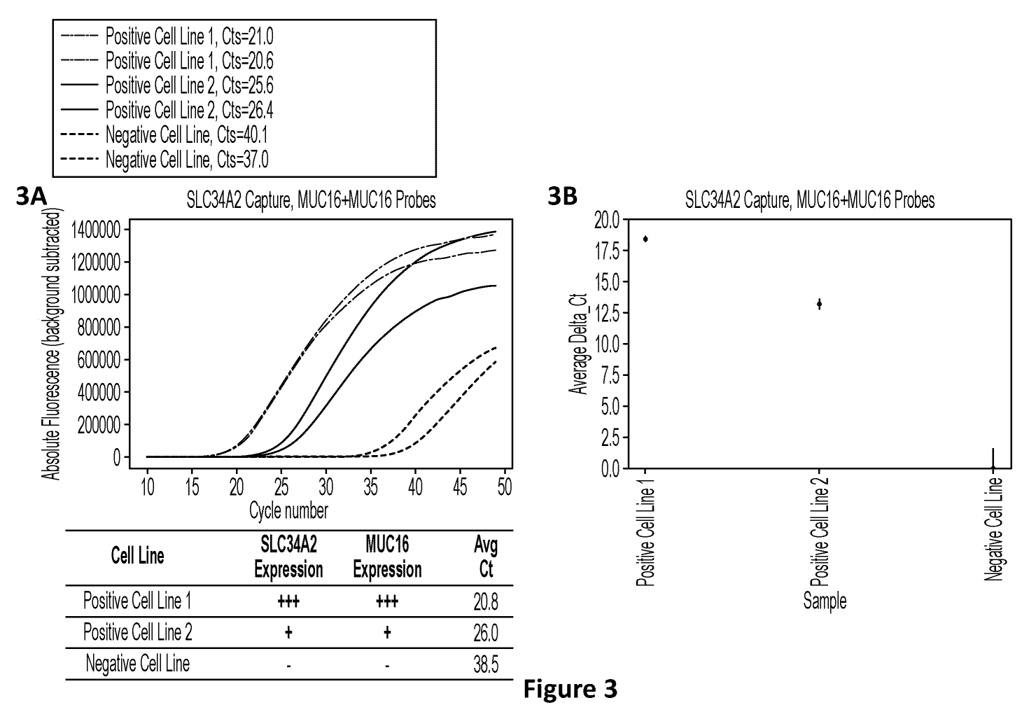

The present Example shows experimental data from certain experiments demonstrating technologies provided herein are capable of detecting ovarian cancer (e.g., ovarian cystadenocarcinoma, and/or high-grade serous ovarian cancer) in patient samples using an exemplary biomarker combinations as described herein (e.g., SLC34A2 and MUC16, e.g., in some embodiments, using SLC34A2 capture with MUC16 + MUC16 antibody based detection probes). The first experiment demonstrated the detection of two different ovarian cancer cell-line-derived-extracellular vesicles (CLD-EVs) in PBS using a duplex system assay described herein. See, for example, FIG. 3.

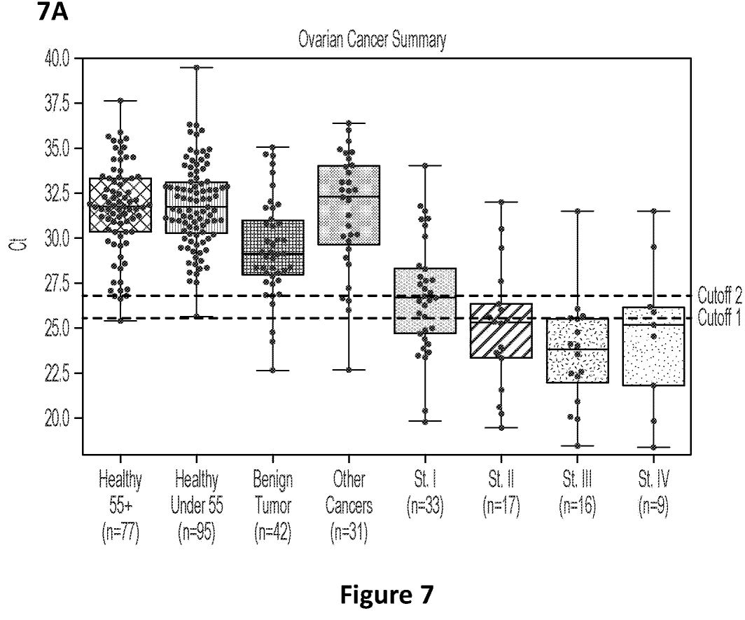

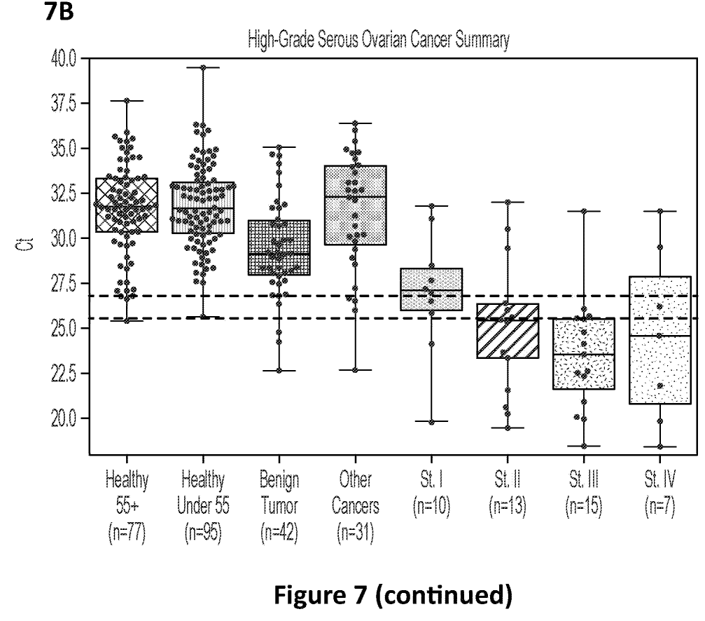

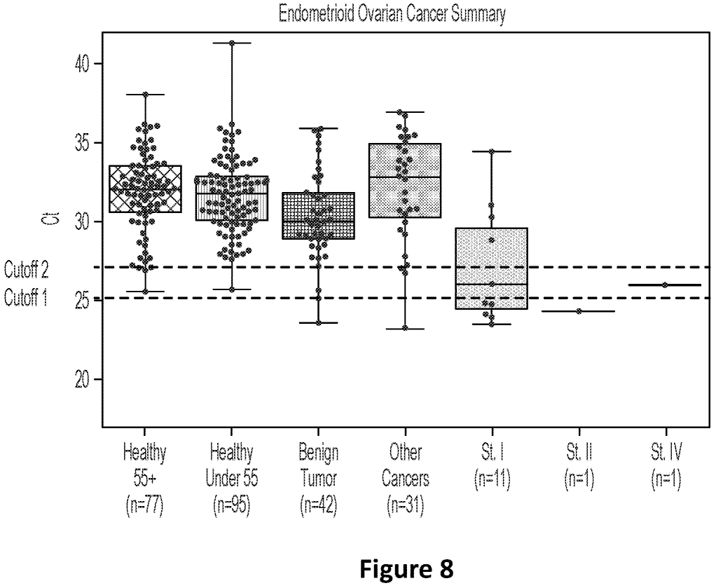

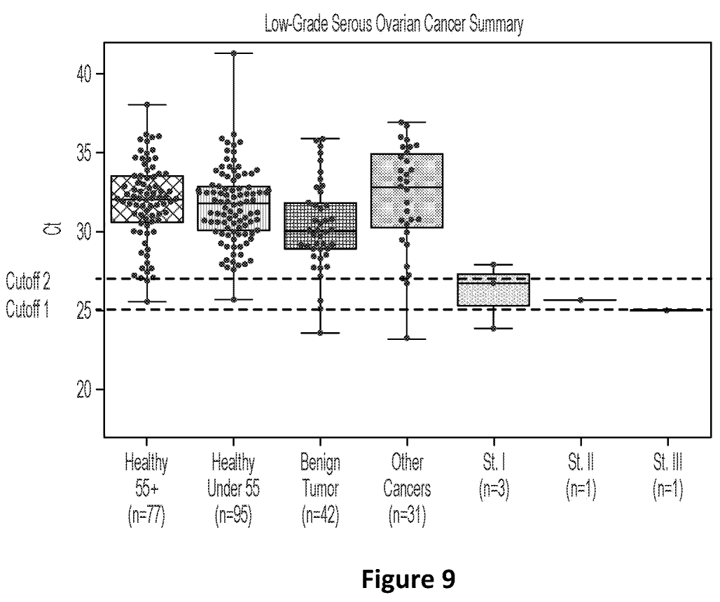

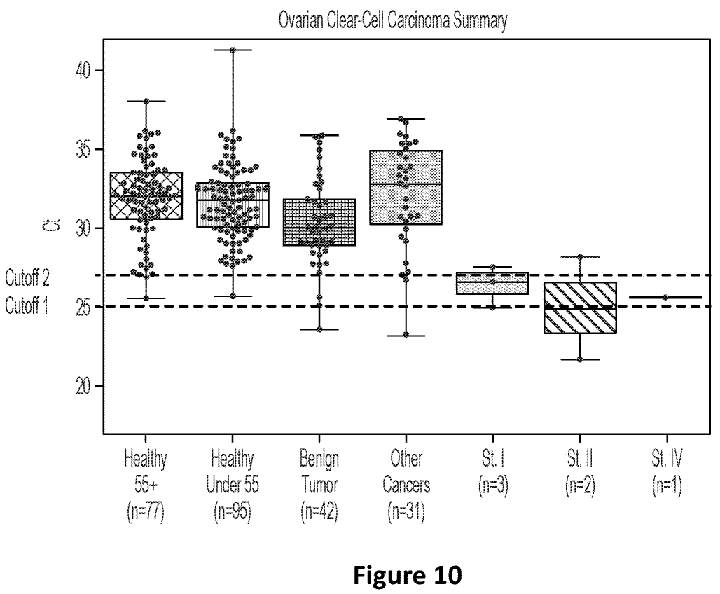

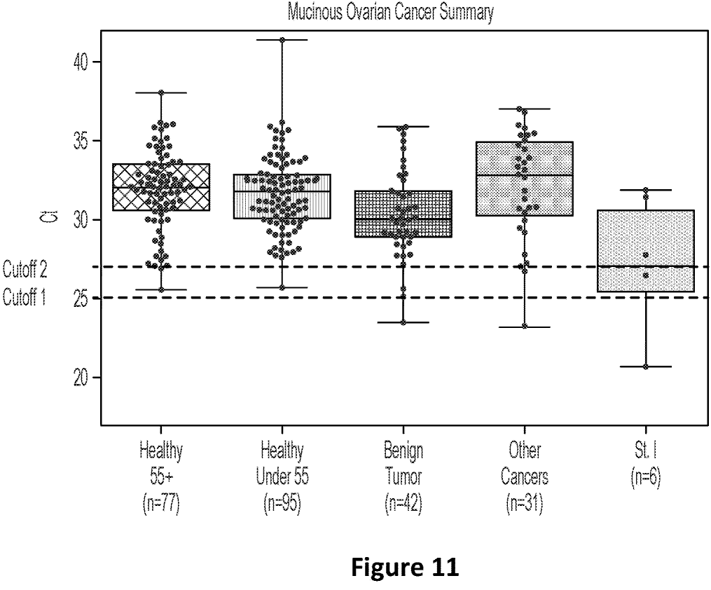

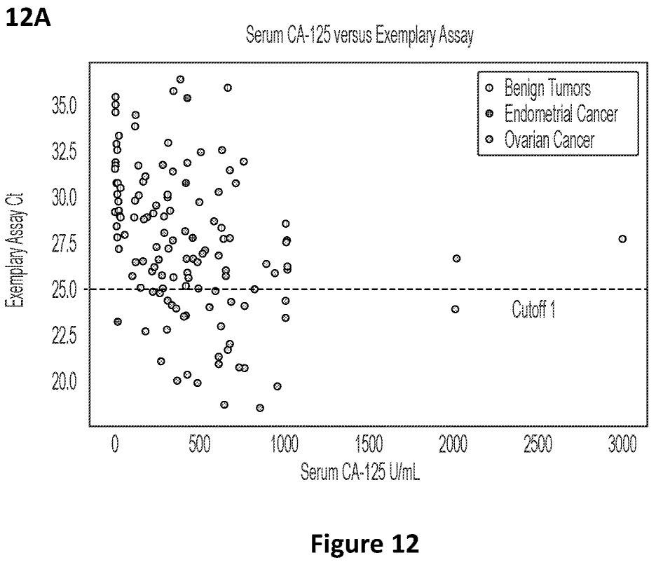

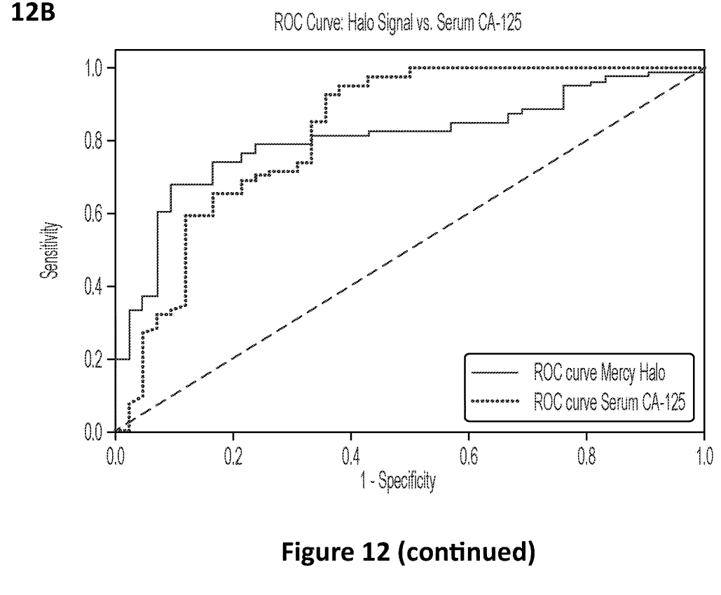

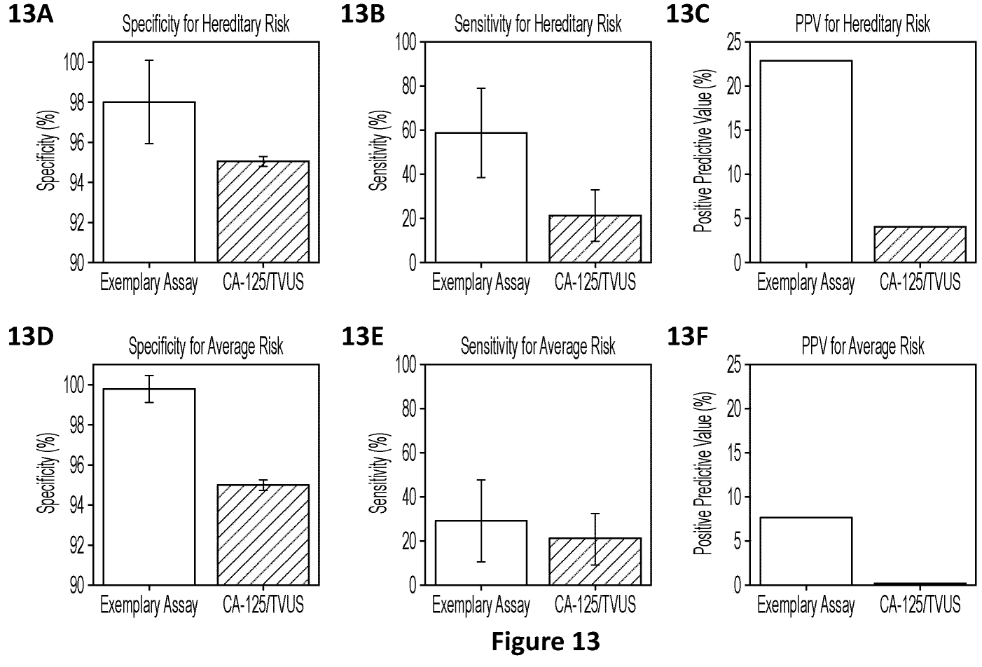

With such a duplex system assay capable of detecting CLD-EVs, a study containing patient samples from ovarian cancer of each stage (Stage I-IV) and/or subtypes and from various control groups (e.g., healthy subjects, subjects with benign gynecological tumors, and non-ovarian cancers) was performed. Please see Example 6, FIGS. 7-13 showing performance of an exemplary duplex system assay involving a target biomarker signature comprising SLC34A2 and MUC16.

### Overview of an Exemplary Assay

In some embodiments, a target entity detection system described herein is a duplex system. In some embodiments, such a duplex system, e.g., as illustrated in FIG. 2, utilizes two antibodies that each recognize a different epitope. Paired double-stranded template DNAs are also utilized in qPCR, each of which has specific four-base 5′ overhangs complementary to the 5′ overhang on its partner. Each antibody is conjugated with one of the two double-stranded DNA templates. When the antibodies bind their target epitopes, the sticky ends of the respective templates can hybridize. These sticky ends are then ligated together by T7 ligase, prior to PCR amplification. For hybridization between the two DNA templates to occur, the two antibodies need to be bound close enough to each other (within 50 to 60 nm, the length of the DNA linker and antibody). Any templates that bind but remain unligated will not produce PCR product, as shown in FIG. 2.

### Healthy Controls Versus Stage I, II, III, and IV Ovarian Cystadenocarcinoma (OC) Plasma

Plasma samples from healthy controls and OC patients were processed to obtain purified extracellular vesicles, which were interrogated using an exemplary assay as described below.

Purified EVs were captured using magnetic beads covalently conjugated with anti-SLC34A2 antibodies. The EVs captured by the beads were profiled using a set of two detection probes, each comprising an anti-MUC16 antibody and a distinct oligonucleotide domain (e.g., ones as described herein).

The biomarker combination of SLC34A2 and MUC16 was carefully selected to minimize cross-reactivity with healthy-tissue-derived extracellular vesicles. The cross-reactivity of such a biomarker combination with healthy tissues was bioinformatically predicted, in part, by using a heatmap of differentially expressed mRNAs in ovarian cystadenocarcinoma. Thus, different combinations of markers can be predicted to be much more abundant on the surface of ovarian cancer extracellular vesicles than on the surface of extracellular vesicles from healthy tissues. In some embodiments, such a biomarker combination includes SLC34A2 and MUC16.

TABLE 2

The transcript expression scores for the following biomarker combination, as expressed in certain ovarian cancer cell lines vs. negative control cell line (e.g., non-ovarian cancer line)

| | | | |
|---|---|---|---|
| Genes | Ovarian Cancer Cell Line 1 | Ovarian Cancer Cell Line 2 | Negative Non-Ovarian Cancer |
| SLC34A2 | +++ | + | - |
| MUC16 | +++ | + | - |

### Exemplary Methods

### Oligonucleotides

In some embodiments, oligonucleotides can have the following sequence structure and modifications. It is noted that the strand numbers below correspond to the numerical values associated with strands shown in FIG. 2.

### Strand 1 V1

/5AzideN/CAGTCTGACACAGCAGTCGTTAATCGTCGCTGCTACCCTTGACATCCGTGACTGG CTAGACAGAGGTGTwhere /5AzideN/ refers to an azide group linkedto the 5′ oligonucleotide terminus via a NHS ester linker, or /5AmMC12/CAGTCTGACACAGCAGTCGTTAATCGTCGCTGCTACCCTTGACATCCGTGACTGG CTAGACAGAGGTGT,where /5AmMC12/ refers to an amine group (e.g., a primary amino group) linked to the 5′ oligonucleotide terminus via a 12–carbon spacer, or /5ThiolMC6/CAGTCTGACACAGCAGTCGTTAATCGTCGCTGCTACCCTTGACATCCGTGACT GGCTAGACAGAGGTGT,where /5ThiolMC6/ refers to a thiol linked to the 5′ oligonucleotide terminus via a 6–carbon spacer.

### Strand 2 V1

/5AzideN/GACCTGACCTACAGTGACCATAGCCTTGCCTGATTAGCCACTGTCCAGTTTGGCT CCTGGTCTCACTAG,where /5AzideN/ refers to an azide group linkedto the 5′ oligonucleotide terminus via a NHS ester linker, or /5AmMC12/GACCTGACCTACAGTGACCATAGCCTTGCCTGATTAGCCACTGTCCAGTTTGGCT CCTGGTCTCACTAG,where /5AmMC12/ refers to an amine group (e.g., a primary amino group) linked to the 5′ oligonucleotide terminus via a 12–carbon spacer, or /5ThiolMC6/GACCTGACCTACAGTGACCATAGCCTTGCCTGATTAGCCACTGTCCAGTTTGG CTCCTGGTCTCACTAG,where /5ThiolMC6/ refers to a thiol linked to the 5′ oligonucleotide terminus via a 6–carbon spacer.

### Strand 3 V1

/5Phos/GAGTACACCTCTGTCTAGCCAGTCACGGATGTCAAGGGTAGCAGCGACGATTAACGA CTGCTGTGTCAGACTG,wherein /5Phos/ refers to a phosphate group linked to the 5′ oligonucleotide terminus

### Strand 4 V1

/5Phos/ACTCCTAGTGAGACCAGGAGCCAAACTGGACAGTGGCTAATCAGGCAAGGCTATGGT CACTGTAGGTCAGGTC,wherein /5Phos/ refers to a phosphate group linked to the 5′ oligonucleotide terminus

### Strand 5 V1

CAGTCTGACACAGCAGTCGT

### Strand 6 V1

GACCTGACCTACAGTGACCA

### Strand 7 (Probe) V1

/▫56-FAM/TGGCTAGAC/▫ZEN/AGAGGTGTACTCCTAGTGAGA/▫3▫IABkFQ/,wherein /56–FAM/ refers to a fluorescein (e.g., 6–FAM) at the 5′ oligonucleotide terminus; and /3IABkFQ/ refers to a fluorescein quencher at the 3′ oligonucleotide terminus

In some embodiments, oligonucleotides can have the following sequence structure and modifications. It is noted that the strand numbers below correspond to the numerical values associated with strands shown in FIG. 2.

### Strand 1 V2

/5AzideN/CAGTCTGACTCACCACTCGTTAATCGTCGCTGCTACCCTTGACATCCGTGACTGG CTAGACAGAGGTGT,where /5AzideN/ refers to an azide group linked to the 5′ oligonucleotide terminus via a NHS ester linker, or /5AmMC12/CAGTCTGACTCACCACTCGTTAATCGTCGCTGCTACCCTTGACATCCGTGACTGG CTAGACAGAGGTGT,where /5AmMC12/ refers to an amine group (e.g., a primary amino group) linked to the 5′ oligonucletoide terminus via a 12–carbon spacer, or /5ThiolMC6/CAGTCTGACTCACCACTCGTTAATCGTCGCTGCTACCCTTGACATCCGTGACT GGCTAGACAGAGGTGT,where /5ThiolMC6/ refers to a thiol linked to the 5′ oligonucletoide terminus via a 6–carbon spacer

### Strand 2 V2

/5AzideN/CACCAGACCTACGAAGTCCATAGCCTTGCCTGATTAGCCACTGTCCAGTTTGGCT CCTGGTCTCACTAG,where /5AzideN/ refers to an azide group linked to the 5′ oligonucleotide terminus via a NHS ester linker, or /5AmMC12/CACCAGACCTACGAAGTCCATAGCCTTGCCTGATTAGCCACTGTCCAGTTTGGCT CCTGGTCTCACTAG,where /5AmMC12/ refers to an amine group (e.g., a primary amino group) linked to the 5′ oligonucletoide terminus via a 12–carbon spacer, or /5ThiolMC6/CACCAGACCTACGAAGTCCATAGCCTTGCCTGATTAGCCACTGTCCAGTTTGG CTCCTGGTCTCACTAG,where /5ThiolMC6/ refers to a thiol linked to the 5′ oligonucletoide terminus via a 6–carbon spacer

### Strand 3 V2

/5Phos/GAGTACACCTCTGTCTAGCCAGTCACGGATGTCAAGGGTAGCAGCGACGATTAACGA GTGGTGAGTCAGACTG,wherein /5Phos/ refers to a phosphate group linked to the 5′ oligonucletoide terminus

### Strand 4 V2

/5Phos/ACTCCTAGTGAGACCAGGAGCCAAACTGGACAGTGGCTAATCAGGCAAGGCTATGGA CTTCGTAGGTCTGGTG,wherein /5Phos/ refers to a phosphate group linked to the 5′ oligonucletoide terminus

### Strand 5 V2

CAGTCTGACTCACCACTCGT

### Strand 6 V2

CACCAGACCTACGAAGTCCA

### Strand 7 (Probe) V2

/56-FAM/TGGCTAGAC/ZEN/AGAGGTGTACTCCTAGTGAGA/3IABkFQ/wherein 56–FAM refers to a fluorescein (e.g., 6–FAM) at the 5′ oligonucletoide terminus and /3IABkFQ/ refers to a fluorescein quencher at the 3′ oligonucletoide terminus.

In some embodiments, oligonucleotides can have the following sequence structure and modifications. It is noted that the strand numbers below correspond to the numerical values associated with strands shown in FIG. 2.

### Strand 1 V1–med

/5AzideN/CAGTCTGACACAGCAGTCGTGACTGGCTAGACAGAGGTGT,where /5AzideN/ refers to an azide group linked to the 5′ oligonucleotide terminus via a NHS ester linker, or /5AmMC12/CAGTCTGACACAGCAGTCGTGACTGGCTAGACAGAGGTGT,where /5AmMC12/ refers to an amine group (e.g., a primary amino group) linked to the 5′ oligonucletoide terminus via a 12–carbon spacer, or /5ThiolMC6/CAGTCTGACACAGCAGTCGTGACTGGCTAGACAGAGGTGT, where /5ThiolMC6/ refers to a thiol linked to the 5′ oligonucletoide terminus via a 6–carbon spacer

### Strand 2 V1–med

/5AzideN/GACCTGACCTACAGTGACCATTGGCTCCTGGTCTCACTAG,▫where /5AzideN/ refers to an azide group linked to the 5′ oligonucleotide terminus via a NHS ester linker, or /5AmMC12/GACCTGAGCTAGAGTGAGCATTGGCTCCTGGTCTGAGTAG,where /5AmMC12/ refers to an amine group (e.g., a primary amino group) linked to the 5′ oligonucletoide terminus via a 12–carbon spacer, or /5ThiolMC6/GACCTGACCTACAGTGACCATTGGCTCCTGGTCTCACTAG,▫ where /5ThiolMC6/ refers to a thiol linked to the 5′ oligonucletoide terminus via a 6–carbon spacer

### Strand 3 V1–med

/5Phos/GAGTAGACCTCTGTCTAGCCAGTCACGACTGCTGTGTCAGACTG,▫wherein /5Phos/ refers to a phosphate group linked to the 5′ oligonucletoide terminus

### Strand 4 V1–med

/5Phos/ACTCCTAGTGAGACCAGGAGCCAATGGTCACTGTAGGTCAGGTC,▫wherein /5Phos/ refers to a phosphate group linked to the 5′ oligonucletoide terminus

### Strand 5 V1

CAGTCTGACACAGCAGTCGT

### Strand 6 V1

GACCTGACCTACAGTGACCA

### Strand 7 (Probe) V1

/56-FAM/TGGCTAGAC/ZEN/AGAGGTGTACTCCTAGTGAGA/3IABkFQ/,▫wherein 56–FAM refers to a fluorescein (e.g., 6–FAM) at the 5′ oligonucletoide terminus and /3IABkFQ/ refers to a fluorescein quencher at the 3′ oligonucletoide terminus.

### Antibody-Oligonucleotide (e.g., Antibody-DNA) Conjugation

Antibody aliquots ranging from 25-100 µg were conjugated with oligonucleotide strands, for example, 60 µg aliquots of anti-MUC16 antibody was conjugated with hybridized strands 1+3 and 2+4, for example, using copper-free click chemistry. The first step was to prepare DBCO-functionalized antibodies to participate in the conjugation reaction with azide-modified oligonucleotide domain (e.g., DNA domain). This began with reacting the antibodies with the DBCO-PEG5-NHS heterobifunctional cross linker. The reaction between the NHS ester and available lysine groups was allowed to take place at room temperature for 2 hours, after which unreacted crosslinker was removed using centrifugal ultrafiltration. To complete the conjugation, azide-modified oligonucleotide domains (e.g., DNA domain) and the DBCO-functionalized antibodies were allowed to react overnight at room temperature. The concentration of conjugated antibody was measured using the Qubit protein assay.

### Cell Culture

Negative control cells (e.g., non-ovarian cancer cells such as melanoma cells or healthy cells) were grown in Eagle’s Minimum Essential Medium (EMEM) with 10% exosome-free FBS and 50 units of penicillin/streptomycin per mL. Ovarian cystadenocarcinoma cells were grown in Roswell Park Memorial Institute (RPMI 1640) with 10% exosome-free FBS and 50 units of penicillin/streptomycin per mL. Exemplary ovarian cancer cell lines that may be useful to develop an assay for detection of ovarian cancer (e.g., ones as described herein) include, but are not limited to, A2780, Caov-3, COV413A, ES2, OVCAR-3, OV90, PA-1, SK-OV-3, SW 626, TOV-112, and cells lines described in Ince et al., “Characterization of twenty-five ovarian tumor cell lines that phenocopy primary tumours” Nature Communications 6: 7419 (2015) which is incorporated herein by reference for the purpose described herein. All cell lines were maintained at 5% CO₂ and 37° C. and the passage number was below 20.

### Purification of Extracellular Vesicles From Cell Culture Medium

In some embodiments, ovarian cancer cells and negative control cells were grown in their respective media until they reached ~80% confluence. The cell culture medium was collected and spun at 300 x rcf for 5 minutes at room temperature (RT) to removes cells and debris. The supernatant was then collected and frozen at -80° C.

Prior to use, the frozen supernatant stored at -80° C. was thawed and then clarified of cells and large (e.g., greater than 1 micron in diameter) cellular fragments. The thawed supernatant was clarified using centrifugation.

In some embodiments, the clarified cell culture medium (e.g., ~500 µL) was run through a size-exclusion purification column. Nanoparticles having a size range of about 65 nm to about 1000 nm were collected for each sample. In some embodiments, a smaller particle range may be desirable.

### Particle Counts

Particle counts were obtained, e.g., using a SpectroDyne particle counting instrument using the TS400 chips, to measure nanoparticle range between 65 and 1000 nm. In some embodiments, a smaller particle range may be desirable.

### Generation of Patient Plasma Pools

In some embodiments, pooled patient plasma pools were utilized. In brief, 1 mL aliquots of patient plasma were thawed at room temperature for at least 30 minutes. The tubes were vortexed briefly and spun down to consolidate plasma to the bottom of each tube. Plasma samples from a given patient cohort were combined in an appropriately sized container and mixed thoroughly by end-over-end mixing. Each plasma pool was split into 1 mL aliquots in Protein Lo-bind 1.5 mL Eppendorf tubes and refrozen at -80° C.

### Whole-Plasma Clarification (Optional)

In some embodiments, prior to EVs purification, samples were blinded by personnel who would not participate in sample-handling. The patient-identification information was only revealed after the experiment was completed to enable data analysis. 1 mL aliquots of whole plasma were removed from storage at -80° C. and subjected to three clarification spins to remove cells, platelets, and debris.

### Size-Exclusion Chromatography Purification of EVs From Clarified Plasma

Each clarified plasma sample (individual samples or pooled samples) was run through a single-use, size-exclusion purification column to isolate the EVs. Nanoparticles having a size range of about 65 nm to about 1000 nm were collected for each sample. In some embodiments, smaller particle range may be desirable.

### Capture-Antibody Conjugation to Magnetic-Capture Beads

Antibodies were conjugated to magnetic beads (e.g., epoxy-functionalized Dynabeads™). Briefly, beads were weighed in a sterile environment and resuspended in buffer. Antibodies were mixed with the functionalized beads and the conjugation reaction took place with end-over-end mixing. The beads were washed several times using the wash buffer provided by the conjugation kit and were stored at 4° C. in the provided storage buffer.

### Direct Capture of Purified Plasma EVs Using Antibody-Conjugated Magnetic Beads

In certain embodiments purified plasma EVs were directly captured from clarified plasma samples. For example, for SLC34A2 capture, a diluted sample of purified plasma EVs were incubated with magnetic beads conjugated with anti-SLC34A2 antibodies for an appropriate time period, e.g., at room temperature.

### Binding of Antibody-Oligonucleotide Conjugates to EVs Bound on Magnetic Capture Beads

Antibody-oligonucleotide conjugates (e.g., anti-MUC16 antibody-oligonucleotide conjugates; “antibody probes”) were diluted in an appropriate buffer at their optimal concentrations. Antibody probes were allowed to interact with a sample comprising EVs bound on magnetic capture beads.

### Post-Binding Washes

In some embodiments, samples were washed, e.g., multiple times, in an appropriate buffer.

### Ligation

After the wash to remove unbound antibody-oligonucleotide conjugates, the beads with bound extracellular vesicles and bound antibody-oligonucleotide conjugates were contacted with a ligation mix. The mixtures were incubated for 20 minutes at RT.

### Pcr

Following ligation, the beads with bound extracellular vesicles and bound antibody-oligonucleotide conjugates were contacted with a PCR mix. PCR was performed in a 96-well plate, e.g., on the Quant Studio 3, with the following exemplary PCR protocol: hold at 95° C. for 1 minute, perform 50 cycles of 95° C. for 5 seconds and 62° C. for 15 seconds. The rate of temperature change was chosen to be standard (2° C. per second). A single qPCR reaction was performed for each experimental replicate and ROX was used as the passive reference to normalize the qPCR signals. Data was then downloaded from the Quant Studio 3 machine and analyzed and plotted in Python 3.7.

### Data Analysis

In some embodiments, a binary classification system can be used for data analysis. In some embodiments, signals from a detection assay may be normalized based on a reference signal. For example, in some embodiments, normalized signals for a single antibody duplex were calculated by choosing a reference sample. In some embodiments, the equations used to calculate the normalized signal for an arbitrary sample i are given below, where Signalₘₐₓ is the signal from the highest concentration cell-line EVs standard.

$$Δ C {t}_{i} = C {t}_{r e f} - C {t}_{i}$$

$$S i g n a {l}_{i} = {2}^{Δ C {t}_{i}}$$

$$N o r m S i g n a {l}_{i} = \frac{S i g n a {l}_{i}}{S i g n a {l}_{m a x}}$$

### Representative Results

### OC Cell Line Experiments

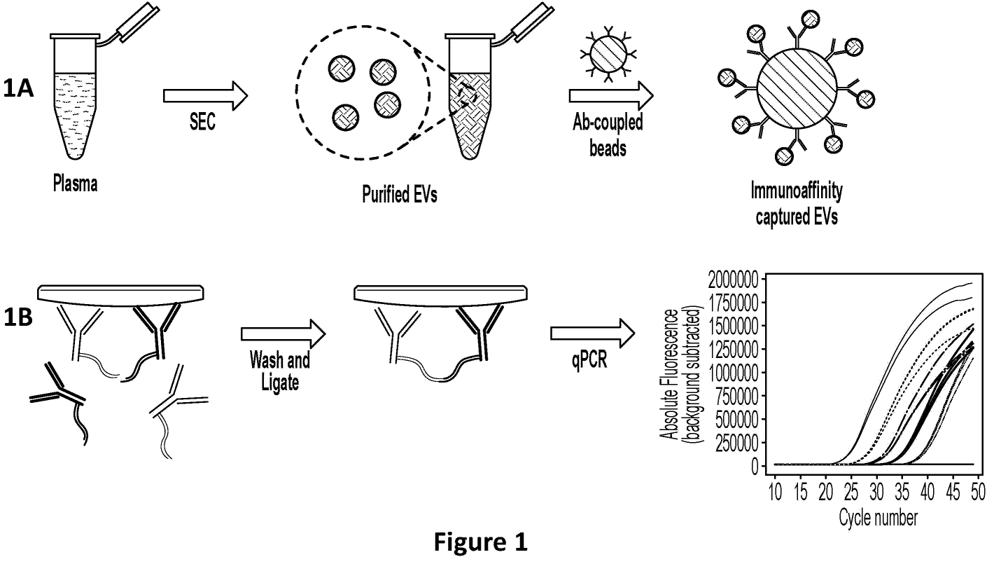

Purified cell-line EVs were diluted to an optimal concentration in an appropriate buffer and captured using SLC34A2-functionalized beads (e.g., replicates of 1 mL or less). Captured EVs were analyzed using MUC16 + MUC16 antibody probes (also known as “MUC16 + MUC16 antibody probes”). Representative qPCR data and ΔCt values are provided in FIG. 3. The data show that the biomarker combination of SLC34A2 and MUC16 (e.g., in combination with an exemplary assay such as, e.g., as described in the present Example and illustrated in FIGS. 1-2) is capable of distinguishing OC-derived EVs from the negative control cell line, with a signal strength that is well-correlated with the expression of the two markers (see Table 2).

### OC Pilot Patient Plasma Study

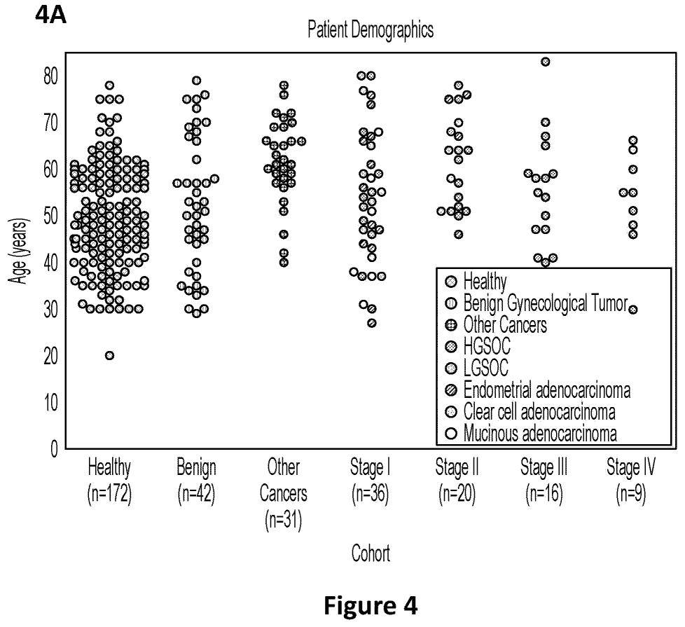

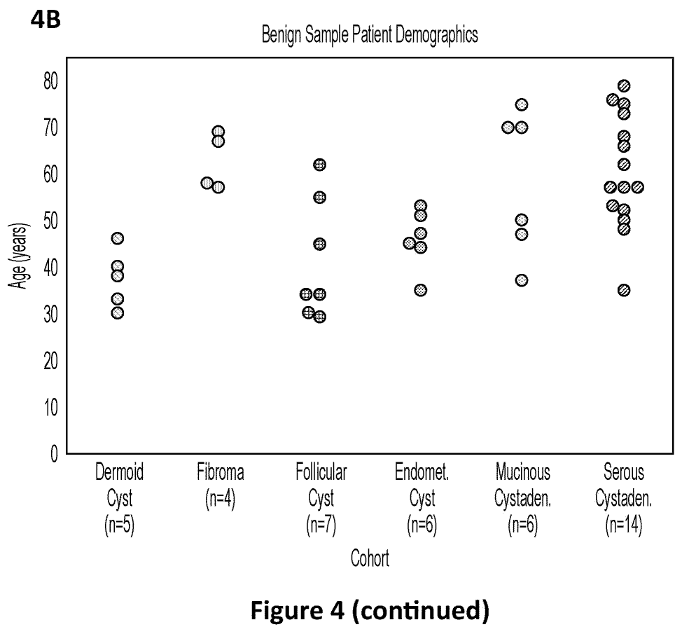

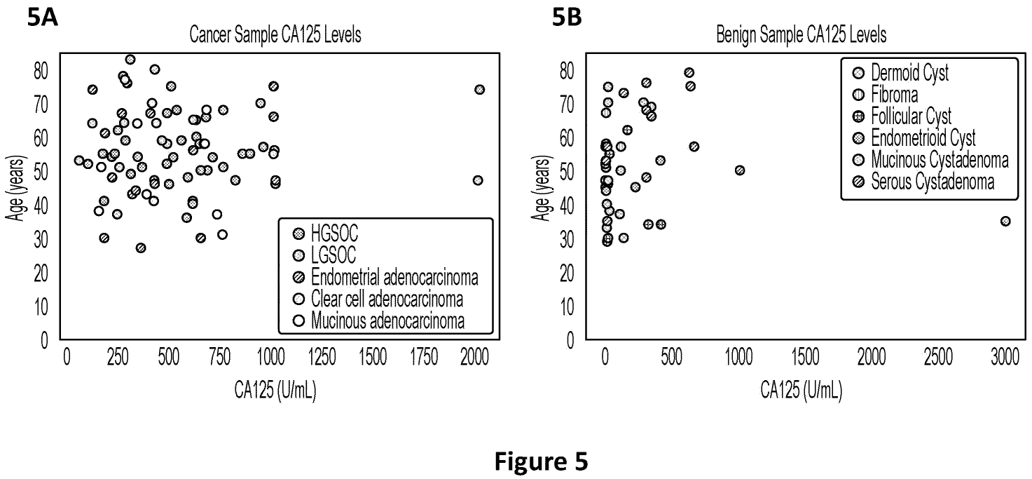

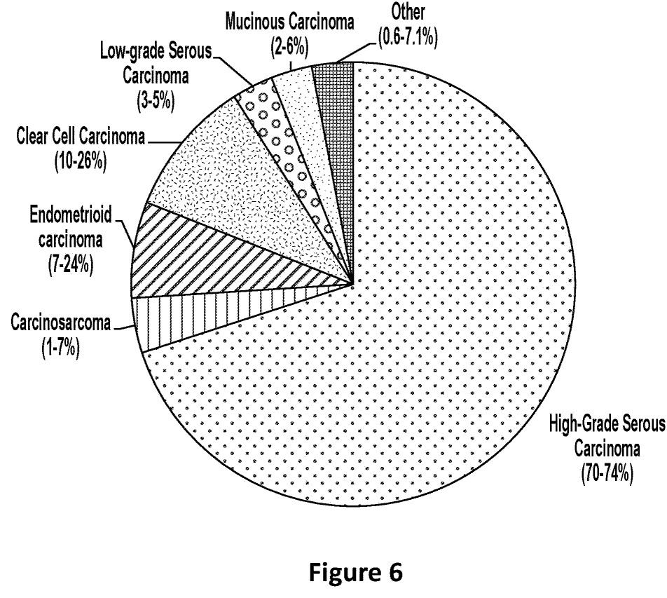

The demographics of the patients included in the OC patient plasma sample pilot study are provided in FIG. 4. Care was taken to match age and gender as closely as possible across the different sample cohorts.

Replicates of one milliliter or less (e.g., 500 µl or less, 400 µl or less, 300 µl or less, 200 µl or less, or 100 µl or less) of patient sample plasma was clarified as described above and EVs were purified using size-exclusion chromatography. EVs were captured using anti-SLC34A2 magnetic beads. EVs captured by the anti-SLC34A2 magnetic beads were profiled using MUC16 + MUC16 antibody probes. Please see Example 6 and FIGS. 7-13 showing performance of an exemplary duplex system assay involving a target biomarker signature comprising SLC34A2 and MUC16.

### Discussion

The present Example demonstrates a biomarker combination of SLC34A2 and MUC16 (e.g., in combination with a duplex assay as described in the present Example and illustrated in FIGS. 1-2) is capable of detecting early stage ovarian cystadenocarcinoma with >99.5% specificity. In some such embodiments, a biomarker combination includes SLC34A2 capture and MUC16 + MUC16 antibody probes. While assay signal was not strongly correlated with serum CA-125 levels, a larger cohort of OC-positive and healthy control patients spanning a wider range of serum CA-125 can be useful to demonstrate the utility of such an assay over the current clinical standard for ovarian cancer diagnosis.

In some embodiments, a biomarker combination of SLC34A2 capture with MUC16 + MUC16 antibody probes can be applied to OC-positive and healthy patient samples with low and intermediate levels of serum CA-125.

In some embodiments, a biomarker combination including CLDN6 (e.g., in combination with SLC34A2 and/or MUC16) can be used in an ovarian cancer detection assay.

In some embodiments, a dendron, which can add up to 16 strands of oligonucleotide domain (e.g., DNA) per antibody, can be used instead of one or two strands of DNA per antibody, for example, to enhance signal-to-noise.

### Example 2: Embodiments of Ovarian Cancer Detection

In some embodiments, a biomarker combination including SLC34A2 and MUC16 (e.g., in some embodiments, SLC34A2 capture (e.g., SLC34A2 immunoaffinity capture with MUC16 + MUC16 antibody probes) can be used for detection of ovarian cancer (e.g., following an assay as described in Example 1) in various subject populations including, e.g., healthy controls, non-smokers (n = 93); benign gynecological tumors; Stage I OC; Stage II OC; Stage III OC; Stage IV OC; and Recurrent OC.

In some embodiments, a biomarker combination including SLC34A2 and FOLR1 (e.g., in some embodiments, SLC34A2 capture (e.g., SLC34A2 immunoaffinity capture with FOLR1 + FOLR1 antibody probes) can be used for detection of ovarian cancer (e.g., following an assay as described in Example 1) in various subject populations (e.g., as described herein).

In some embodiments, a biomarker combination including SLC34A2, MUC16, and FOLR1 (e.g., in some embodiments, SLC34A2 capture (e.g., SLC34A2 immunoaffinity capture) with MUC16 + FOLR1 antibody probes) can be used for detection of ovarian cancer (e.g., following an assay as described in Example 1) in various subject populations (e.g., as described herein).

In some embodiments, a biomarker combination including MUC16 and CLDN6 (e.g., in some embodiments, MUC16 capture (e.g., MUC16 immunoaffinity capture with MUC16 + CLDN6 antibody probes) can be used for detection of ovarian cancer (e.g., following an assay as described in Example 1) in various subject populations (e.g., as described herein).

In some embodiments, a biomarker combination including MUC16 and FOLR1 (e.g., in some embodiments, MUC16 capture (e.g., MUC16 immunoaffinity capture with MUC16 + FOLR1 antibody probes) can be used for detection of ovarian cancer (e.g., following an assay as described in Example 1) in various subject populations (e.g., as described herein). In some embodiments, such a biomarker combination may be embodied in an assay for detection of ovarian cancer in various subject populations (e.g., as described herein) with MUC16 immunoaffinity capture and FOLR1 + FOLR1 antibody probes (e.g., following an assay as described in Example 1).

In some embodiments, a biomarker combination including MUC16 and CLDN3 (e.g., in some embodiments, MUC16 capture (e.g., MUC16 immunoaffinity capture with MUC16 + CLDN3 antibody probes) can be used for detection of ovarian cancer (e.g., following an assay as described in Example 1) in various subject populations (e.g., as described herein).

In some embodiments, a biomarker combination including FOLR1 and CLDN6 (e.g., in some embodiments, FOLR1 capture (e.g., FOLR1 immunoaffinity capture with FOLR1 + CLDN6 antibody probes) can be used for detection of ovarian cancer (e.g., following an assay as described in Example 1) in various subject populations (e.g., as described herein).

In some embodiments, a biomarker combination including LRRTM1 and MUC16 (e.g., in some embodiments, LRRTM1 capture (e.g., LRRTM1 immunoaffinity capture with MUC16 + MUC16 antibody probes) can be used for detection of ovarian cancer (e.g., following an assay as described in Example 1) in various subject populations (e.g., as described herein).

In some embodiments, a biomarker combination including FOLR1, SLC34A2, and CLDN3 (e.g., in some embodiments FOLR1 capture (e.g., FOLR1 immunoaffinity capture with SLC34A2 + CLDN3 antibody probes) can be used for detection of ovarian cancer (e.g., following an assay as described in Example 1) in various subject populations (e.g., as described herein).

In some embodiments, a biomarker combination including MUC16, FOLR1, and CLDN3 (e.g., in some embodiments MUC16 capture (e.g., MUC16 immunoaffinity capture with FOLR1 + CLDN3 antibody probes) can be used for detection of ovarian cancer (e.g., following an assay as described in Example 1) in various subject populations (e.g., as described herein).

In some embodiments, a single biomarker (e.g., any biomarker described herein) may be sufficient for effective detection of ovarian cancer with high specificity and/or sensitivity. (e.g., in some embodiments, MUC16 capture (e.g., MUC16 immunoaffinity capture with MUC16 + MUC16 antibody probes) can be used for detection of ovarian cancer (e.g., following an assay as described in Example 1) in various subject populations (e.g., as described herein).

In some embodiments, two or more biomarker combinations described herein may be used together for detection of ovarian cancer, e.g., to increase sensitivity of an assay. For example, in some embodiments, an assay for ovarian cancer detection may involve at least two biomarker combinations, wherein a first biomarker combination may comprise SLC34A2 and MUC16, and a second biomarker combination may comprise SLC34A2 and FOLR1. For example, in some embodiments, a first biomarker combination comprising SLC34A2 and MUC16 may be embodied in an assay involving SLC34A2 capture (e.g., SLC34A2 immunoaffinity capture with MUC16 + MUC16 antibody probes). In some embodiments, a second biomarker combination comprising SLC34A2 and FOLR1 may be embodied in an assay involving SLC34A2 capture (e.g., SLC34A2 immunoaffinity capture with FOLR1 + FOLR1 antibody probes).

In some embodiments, three or more biomarker combinations described herein may be used together for detection of ovarian cancer, e.g., to increase sensitivity of an assay. For example, in some embodiments, an assay for ovarian cancer detection may involve at least three biomarker combinations, wherein a first biomarker combination may comprise SLC34A2 and MUC16; a second biomarker combination may comprise SLC34A2 and FOLR1; and a third biomarker combination may comprise MUC16 and FOLR1. For example, in some embodiments, a first biomarker combination comprising SLC34A2 and MUC16 may be embodied in an assay involving SLC34A2 capture (e.g., SLC34A2 immunoaffinity capture with MUC16 + MUC16 antibody probes). In some embodiments, a second biomarker combination comprising SLC34A2 and FOLR1 may be embodied in an assay involving SLC34A2 capture (e.g., SLC34A2 immunoaffinity capture with FOLR1 + FOLR1 antibody probes). In some embodiments, a third biomarker combination comprising MUC16 and FOLR1 may be embodied in an assay involving MUC16 capture (e.g., MUC16 immunoaffinity capture with MUC16 + FOLR1 antibody probes).

### Example 3: Assessment of Extracellular Vesicle (EV) Surface Proteins as Ovarian Cancer Biomarkers

In some embodiments, ovarian cancer detection includes detection of at least EV surface protein(s) following immunoaffinity capture of extracellular vesicles.

In some embodiments, one or more surface proteins or extracellular membrane proteins that are present on extracellular vesicles (“capture proteins”) can be used for immunoaffinity capture of ovarian cancer-associated extracellular vesicles. Examples of such capture protein biomarkers may include, but are not limited to CLDN3, CLDN6, AQP5, CLDN16, FOLR1, LEMD1, LRRTM1, MUC16, CHODL, CDH6, HTR3A, SLC34A2, EpCAM, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof.

In some embodiments, EV immunoassay methodology (e.g., ones described herein such as in Example 1) and biomarker-validation process (e.g., ones described herein such as in Example 1) can be used to assess additional surface proteins as biomarkers for ovarian cancer. In some embodiments, an antibody directed to a capture protein (e.g., a surface protein present in ovarian cancer-associated EVs) is conjugated to magnetic beads and evaluated, first on cell-line EVs then on patient samples, for its ability to bind the specific target protein. The antibody-coated bead is assessed for its ability to capture ovarian cancer-associated EVs and the captured EVs by the antibody-coated bead is read out using a target entity detection system (e.g., a duplex system as described herein involving a set of two detection probes, each directed to a target marker that is distinct from the capture protein.

In some embodiments, captured EVs can be read out using at least one or more (e.g., 1, 2, 3, or more) of the following surface protein biomarkers: CLDN3, CLDN6, AQP5, CLDN16, FOLR1, LEMD1, LRRTM1, MUC16, CHODL, CDH6, HTR3A, SLC34A2, EpCAM, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof. In some embodiments, captured EVs can be read out using a set of detection probes (e.g., as utilized and/or described herein), at least two of which are directed to one or more (e.g., 1, 2, 3, or more) of the following surface protein biomarkers: CLDN3, CLDN6, AQP5, CLDN16, FOLR1, LEMD1, LRRTM1, MUC16, CHODL, CDH6, HTR3A, SLC34A2, EpCAM, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof. In some embodiments, a set of detection probes comprises two detection probes each directed to the same surface protein. In some embodiments, a set of detection probes comprises two detection probes each directed to a distinct surface protein.

### Example 4: Assessment of mRNA in Extracellular Vesicles (Intravesicular mRNA) as Ovarian Cancer Biomarkers

In some embodiments, ovarian cancer detection includes detection of at least intravesicular mRNA(s) following immunoaffinity capture of extracellular vesicles.

In some embodiments, one or more surface proteins or extracellular membrane proteins that are present on extracellular vesicles (“capture proteins”) can be used for immunoaffinity capture of ovarian cancer-associated extracellular vesicles. Examples of such capture protein biomarkers may include, but are not limited to AQP5, CLDN3, CLDN6, CLDN16, LEMD1, LRRTM1, MUC16, CHODL, CDH6, HTR3A, SLC34A2, EpCAM, FOLR1, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof.

In some embodiments, EV nucleic acid detection assay (e.g., reverse transcription PCR using primer-probe sets) and biomarker-validation process (e.g., ones described herein such as in Example 1) can be used to assess mRNA biomarker candidates for ovarian cancer. In some embodiments, an antibody directed to a capture protein (e.g., a surface protein present in ovarian cancer-associated EVs) is conjugated to magnetic beads and evaluated, first on cell-line EVs then on patient samples, for its ability to bind the specific target protein. The antibody-coated bead is assessed for its ability to capture ovarian cancer-associated EVs and the captured EVs by the antibody-coated bead is profiled for their mRNA contents, for example, using one-step quantitative reverse transcription PCR (RT-qPCR) master mix.

In some embodiments, captured EVs can be read out by detection of at least one or more (e.g., 1, 2, 3, or more) of the following mRNAs: CRABP2, MIF, CLDN6, PRAME, S100A1, KLK7, and combinations thereof. In some embodiments, captured EVs can be read out by detection of one or more (e.g., 1, 2, 3, or more) of the following mRNAs using RT-qPCR: CRABP2, MIF, CLDN6, PRAME, S100A1, KLK7, and combinations thereof.

In some embodiments, captured EVs can be read out by detection of at least one or more (e.g., 1, 2, 3, or more) of the following mRNAs: CRABP2, MIF, CLDN6, PRAME, S100A1, KLK7, and combinations thereof; and at least one or more (e.g., 1, 2, 3, or more) of EV surface proteins described in Example 3. In some such embodiments, captured EVs can be read out (i) by detection of one or more (e.g., 1, 2, 3, or more) of the following mRNAs using RT-qPCR: CRABP2, MIF, CLDN6, PRAME, S100A1, KLK7, and combinations thereof; and (ii) by using a set of detection probes (e.g., as utilized and/or described herein), at least one of which are directed to one or more (e.g., 1, 2, 3, or more) of EV surface proteins described in Example 3. In some such embodiments, a set of detection probes comprises at least one detection probe directed to an EV surface protein. In some such embodiments, a set of detection probes comprises at least two detection probes directed to the same EV surface protein (with the same or different epitopes). In some such embodiments, a set of detection probes comprises at least two detection probes directed to distinct EV surface proteins. In some embodiments, a sample comprising an EV surface protein and intravesicular mRNA can be contacted with an anti-EV surface protein antibody (e.g., an antibody directed to EV surface protein as described in Example 3) conjugated to a single-stranded oligonucleotide (e.g., DNA) that serves as one of two primers in a pair for an intravesicular mRNA biomarker (e.g., described in Example 4) such that the anti-EV surface protein antibody is bound to the EV surface protein while the conjugated single-stranded oligonucleotide is hybridized with the intravesicular mRNA biomarker present in the same sample. A second primer of the pair and an RT-qPCR probe are then added to perform an RT-qPCR for detection of the presence of an intravesicular mRNA and an EV surface protein in a single sample.

In some embodiments, captured EVs can be read out by detection of at least one or more (e.g., 1, 2, 3, or more) of the following mRNAs: CRABP2, MIF, CLDN6, PRAME, S100A1, KLK7, and combinations thereof; and at least one or more (e.g., 1, 2, 3, or more) of EV intravesicular proteins described in Example 5. In some such embodiments, captured EVs can be read out (i) by detection of one or more (e.g., 1, 2, 3, or more) of the following mRNAs using RT-qPCR: CRABP2, MIF, CLDN6, PRAME, S100A1, KLK7, and combinations thereof; and (ii) by using a set of detection probes (e.g., as utilized and/or described herein), at least one of which are directed to one or more (e.g., 1, 2, 3, or more) of intravesicular proteins described in Example 5. In some embodiments, a set of detection probes comprises at least one detection probe directed to an intravesicular protein (e.g., as described herein). In some embodiments, a set of detection probes comprises at least two detection probes each directed to the same intravesicular protein (e.g., with the same epitope or different epitopes). In some embodiments, a set of detection probes comprises at least two detection probes each directed to a distinct intravesicular protein (e.g., as described herein). In some such embodiments, a sample comprising EV intravesicular protein and intravesicular mRNA) can be contacted with an anti-EV intravesicular protein antibody (e.g., an antibody directed to EV intravesicular protein as described in Example 5) conjugated to a single-stranded oligonucleotide (e.g., DNA) that serves as one of two primers in a pair for an intravesicular mRNA biomarker (e.g., described in Example 4) such that the anti-EV intravesicular protein antibody is bound to the EV intravesicular protein while the conjugated single-stranded oligonucleotide is hybridized with the intravesicular mRNA biomarker present in the same sample. A second primer of the pair and an RT-qPCR probe are then added to perform an RT-qPCR for detection of the presence of an intravesicular mRNA and an intravesicular protein in a single sample.

### Example 5: Assessment of Intravesicular Proteins as Ovarian Cancer Biomarkers

In some embodiments, ovarian cancer detection includes detection of at least intravesicular protein(s) following immunoaffinity capture of extracellular vesicles.

In some embodiments, one or more surface proteins or extracellular membrane proteins that are present on extracellular vesicles (“capture proteins”) can be used for immunoaffinity capture of ovarian cancer-associated extracellular vesicles. Examples of such capture protein biomarkers may include, but are not limited to CLDN6, AQP5, CLDN3, CLDN16, LEMD1, LRRTM1, FOLR1, MUC16, CHODL, CDH6, HTR3A, SLC34A2, EpCAM, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof.

In some embodiments, EV immunoassay methodology (e.g., ones described herein such as in Example 1) and biomarker-validation process (e.g., ones described herein such as in Example 1) can be used to assess intravesicular proteins as biomarkers for ovarian cancer. In some embodiments, an antibody directed to a capture protein (e.g., a surface protein present in ovarian cancer-associated EVs) is conjugated to magnetic beads and evaluated, first on cell-line EVs then on patient samples, for its ability to bind the specific target protein. The antibody-coated bead is assessed for its ability to capture ovarian cancer-associated EVs and the captured EVs by the antibody-coated beads are fixed and/or permeabilized prior to being profiled for their intravesicular proteins using a target entity detection system (e.g., a duplex system as described herein involving a set of two detection probes, each directed to a target marker that is distinct from the capture protein.

In some embodiments, captured EVs after fixation and/or permeabilization can be read out using at least one or more (e.g., 1, 2, 3, or more) of the following intravesicular proteins: CRABP2, KLK7, MIF, PRAME, and S100A1, and combinations thereof. In some embodiments, captured EVs after fixation and/or permeabilization can be read out using a set of detection probes (e.g., as utilized and/or described herein), at least two of which are directed to one or more (e.g., 1, 2, 3, or more) of the following intravesicular proteins: CRABP2, KLK7, MIF, PRAME, and S100A1, and combinations thereof. In some embodiments, a set of detection probes comprises two detection probes each directed to the same intravesicular protein. In some embodiments, a set of detection probes comprises two detection probes each directed to a distinct intravesicular protein.

In some embodiments, captured EVs after fixation and/or permeabilization can be read out using (i) at least one or more (e.g., 1, 2, 3, or more) of the following intravesicular proteins: CRABP2, KLK7, MIF, PRAME, and S100A1, and combinations thereof; and (ii) at least one or more (e.g., 1, 2, 3, or more) of EV surface proteins described in Example 3. In some embodiments, captured EVs after fixation and/or permeabilization can be read out using a set of detection probes (e.g., as utilized and/or described herein), which comprises (i) a first detection probe directed to one or more (e.g., 1, 2, 3, or more) of the following intravesicular proteins: CRABP2, KLK7, MIF, PRAME, and S100A1, and combinations thereof; and (ii) a second detection probe directed to one or more (e.g., 1, 2, 3, or more) of EV surface proteins described in Example 3. In some embodiments, captured EVs after fixation and/or permeabilization can be read out by detecting an EV intravesicular protein and an EV intravesicular mRNA together in a single sample as described in Example 4 above.

### Example 6: Development of an Ovarian Cancer Liquid Biopsy Assay

The present Example describes development of an ovarian cancer liquid biopsy assay, for example, for screening hereditary- and average-risk women. Despite being the fifth largest killer of women among all cancers (Howlader et al., 2019; which is incorporated herein by reference for the purpose described herein), there is currently no recommended ovarian cancer screening tool for average-risk women. This is due, in part, to the poor performance of proposed ovarian cancer screening technologies. Given the incidence of ovarian cancer in average-risk women, inadequate test specificities (<99.5%) result in false positive results that outnumber true positives by more than an order of magnitude. This places a significant burden on the healthcare system and on the women being screened as false positive results lead to additional tests, unnecessary surgeries, and emotional/physical distress (Buys et al., 2011; which is incorporated herein by reference for the purpose described herein). As a result, it may be desirable to develop an ovarian cancer screening test that may exhibit two features to provide clinical utility: (1) ultrahigh specificity (>99.5%) to minimize the number of false positives, and (2) high sensitivity (>40%) for stage I and II ovarian cancer when prognosis is most favorable. The development of such a test has the potential to save tens of thousands of lives each year.

Several different biomarker classes have been studied for an ovarian cancer liquid biopsy assay including circulating tumor DNA (ctDNA), circulating tumor cells (CTCs), bulk proteins, and extracellular vesicles (EVs). EVs are particularly promising due to their abundance and stability in the bloodstream relative to ctDNA and CTCs, suggesting improved sensitivity for early-stage cancers. Moreover, EVs contain cargo (e.g., proteins, RNA, metabolites) that originated from the same cell, providing superior specificity over bulk protein measurements. While the diagnostic utility EVs has been studied, much of this work has pertained to bulk EV measurements or low-throughput single-EV analyses.

This present Example describes one aspect of an exemplary approach for early stage ovarian cancer detection through the profiling of individual extracellular vesicles (EVs) in human plasma. EVs, including exosomes and microvesicles, contain co-localized proteins, RNAs, metabolites, and other compounds representative of their cell of origin (Kosaka et al., 2019; which is incorporated herein by reference for the purpose described herein). The detection of strategically chosen co-localized markers within a single EV can enable the identification of cell type with ultrahigh specificity, including the ability to distinguish cancer cells from normal tissues. As opposed to other cancer diagnostic approaches that rely on cell death for biomarkers to enter the blood (i.e., cfDNA), EVs are released at a high rate by functioning cells. Single cells have been shown to release as many as 10,000 EVs per day in vitro (Balaj et al., 2011; which is incorporated herein by reference for the purpose described herein). In addition, it is widely accepted that cancer cells release EVs at a higher rate than healthy cells (Bebelman et al. 2018; which is incorporated herein by reference for the purpose described herein).

In one aspect, the present disclosure provides insights and technologies involving identification of genes that are upregulated in ovarian cancer versus healthy tissues using Applicant’s proprietary bioinformatic biomarker discovery process. From a list of upregulated biomarkers, biomarker combinations that are predicted to exhibit high sensitivity and specificity for ovarian cancer are designed. Using an exemplary individual EV assay (see, e.g., illustrated in FIGS. 1 or 2 and/or described herein), co-localization of such biomarkers on an individual vesicle is detected, indicating that the grouping of biomarkers originated from the same cell. This provides superior specificity to bulk biomarker measurements, including bulk EV assays, given that many upregulated cancer biomarkers, such as MUC16, are expressed by one or more healthy tissues. Through the careful design of biomarker combinations, signals from competing tissues can be reduced or eliminated, including those closely related to ovarian cancer. In some embodiments, the present disclosure provides technologies with ultrahigh specificity that is particularly helpful as an ovarian cancer screening test for which the prevalence of disease is low and a high positive-predictive value (>10%) is required (Seltzer et al., 1995; which is incorporated herein by reference for the purpose described herein).

### Biomarker Discovery

In some embodiments, a biomarker discovery process leverages bioinformatic analysis of large databases and an understanding of the biology of ovarian cancer and extracellular vesicles.

### Individual Extracellular Vesicle Analysis

The detection of tumor-derived EVs in the blood requires an assay that has sufficient selectivity and sensitivity to detect relatively few tumor-derived EVs per milliliter of plasma in a background of 10 billion EVs from a diverse range of healthy tissues. The present disclosure, among other things, provides technologies that address this challenge. For example, in some embodiments, an assay for individual extracellular vesicle analysis is illustrated in FIG. 1, which is performed in three key steps as outlined below:
1. EVs are purified from patient plasma using size-exclusion chromatography (SEC), which removes greater than 99% of soluble proteins and other interfering compounds.
2. Tumor-specific EVs are captured using antibody-functionalized magnetic beads specific to a membrane-bound protein.
3. A modified version of proximity-ligation-immuno quantitative polymerase chain reaction (pliq-PCR) is performed to determine the co-localization of additional protein biomarkers contained on or within the captured EVs.

In many embodiments of a modified version of a pliq-PCR assay, two or more different antibody-oligonucleotide conjugates are added to the EVs captured by the antibody-functionalized magnetic bead and the antibodies subsequently bind to their protein targets. The oligonucleotides are composed of dsDNA with single-stranded overhangs that are complementary, and thus, capable of hybridizing when in close proximity (i.e., when the corresponding protein targets are located on the same EV). After washing away unbound antibody-oligonucleotide species, adjacently bound antibody-oligonucleotide species are ligated using a standard DNA ligase reaction. Subsequent qPCR of the ligated template strands enables the detection and relative quantification of co-localized protein species. In some embodiments, two to twenty distinct antibody-oligonucleotide probes can be incorporated into such an assay, e.g., as described in U.S. Application No. 16/805,637, and International Application PCT/US2020/020529, both filed Feb. 28, 2020 and entitled “Systems, Compositions, and Methods for Target Entity Detection”; which are both incorporated herein by reference in their entirety for any purpose.

pliq-PCR has numerous advantages over other technologies to profile EVs. For example, pliq-PCR has a sensitivity three orders of magnitude greater than other standard immunoassays, such as ELISAs (Darmanis et al., 2010; which is incorporated herein by reference for the purpose described herein). The ultra-low LOD of a well-optimized pliq-PCR reaction enables detection of trace levels of tumor-derived EVs, down to a thousand EVs per mL. This compares favorably with other emerging EV analysis technologies, including the Nanoplasmic Exosome (nPLEX) Sensor (Im et al., 2014; which is incorporated herein by reference for the purpose described herein) and the Integrated Magnetic-Electrochemical Exosome (iMEX) Sensor (Jeong et al., 2016; which is incorporated herein by reference for the purpose described herein), which have reported LODs of ~10³ and ~10⁴ EVs, respectively (Shao et al., 2018; which is incorporated herein by reference for the purpose described herein). Moreover, in some embodiments, a modified version of pliq-PCR approach does not require complicated equipment and can uniquely detect the co-localization of multiple biomarkers on individual EVs.

In some embodiments, to further improve the sensitivity and specificity of an individual EV profiling assay, other classes of EV biomarkers include mRNA and intravesicular proteins (in addition to EV surface proteins) can be identified and included in an assay.

### Preliminary Work

Through preliminary studies, a workflow is developed in which biomarker candidates are validated to be present in EVs and capable of being detected by commercially available antibodies or mRNA primer-probe sets. For a given biomarker of interest, one or more cell lines expressing (positive control) and not expressing the biomarker of interest (negative control) can be cultured to harvest their EVs through concentrating their cell culture media and performing purification to isolate nanoparticles having a size range of interest (e.g., using SEC). Typically, extracellular vesicles may range from 30 nm to several micrometers in diameter. See, e.g., Chuo et al., “Imaging extracellular vesicles: current and emerging methods” Journal of Biomedical Sciences 25: 91 (2018) which is incorporated herein by reference for the purpose described herein, which provides information of sizes for different extracellular vesicle (EV) subtypes: migrasomes (0.5-3 µm), microvesicles (0.1-1 µm), oncosomes (1-10 µm), exomeres (<50 nm), small exosomes (60-80 nm), and large exosomes (90-120 nm). In some embodiments, nanoparticles having a size range of about 30 nm to 1000 nm may be isolated for detection assay. In some embodiments, specific EV subtype(s) may be isolated for detection assay.

Through a proprietary biomarker discovery process, membrane-bound protein biomarkers (MUC16 and SLC34A2) that are upregulated in HGSOC versus healthy tissues were identified and used in proof-of-concept experiments in cell-line EVs and ovarian cancer patient samples. MUC16, a membrane protein, is the most widely studied biomarker in ovarian cancer. As described herein, the concentration of CA-125 (which is a portion of a MUC16 polypeptide) in serum has been conventionally monitored in women at hereditary-risk for ovarian cancer and for the recurrence of ovarian cancer. CA-125 is historically measured in serum, in its cleaved form. However, in some embodiments of the present disclosure, MUC16 is measured as an intact trans-membrane protein. In some embodiments of the present disclosure, MUC16 is measured in its cleaved form. While not being bound by theory, SLC34A2 is a multi-pass membrane transporter than has been studied as a therapeutic target for ovarian and non-small cell lung cancer (Lin et al., 2015; which is incorporated herein by reference for the purpose described herein).

To detect assay signal from EVs that contain co-localized MUC16 and SLC34A2 markers, in some embodiments, an assay configuration involving SLC34A2 immunoaffinity capture and two distinct antibody-oligonucleotide probes specific to MUC16 was developed.

Purified cell-line EVs were captured using anti-SLC34A2-functionalized magnetic beads. FIG. 3 (Panel A) provides representative qPCR traces for two positive control cell lines and one negative control cell line, for example. The data demonstrate the influence of gene expression on assay signal, in which the higher expressing cell line exhibited a 36-fold increase (2^{5.2}) in signal relative to the lower expressing cell line. These results demonstrate that in some embodiments, a single EV profiling assay (e.g., ones described herein) is capable of detecting co-localized membrane-bound protein markers on single EVs with very high sensitivity.

Following the validation of MUC16 and SLC34A2 in ovarian cancer cell-line EVs, a pilot study (eventually expanded to 320 patient samples) was performed on ovarian cancer patient plasma samples using an optimized and operator-blinded assay protocol. All plasma samples were purchased from the same source, were processed according to the same blood collection protocol, and patient samples were collected prior to the initiation of any treatment (i.e., treatment naive). The patient cohorts included in the study of this Example are described in FIG. 4 (Panel A).

The results of this clinical pilot study are provided in FIG. 7 for all ovarian cancers and for high-grade serous ovarian cancer (HGSOC), respectively. In some embodiments, specificity was determined by assuming a log-normal distribution around all healthy controls (n=172) and setting a cutoff at 2.879 standard deviations above the mean (Cutoff 1) for 99.8% specificity and 2.055 standard deviations above the mean (Cutoff 2) for 98% specificity. In some embodiments, specificity was determined by assuming a log-normal distribution for all healthy controls (n=172) and setting a cutoff at 2.576 standard deviations from the mean (Cutoff 1) for 99.5% specificity (95% confidence interval (CI): 98.4% to 100%). Cutoff 2 was drawn 2.055 standard deviations from the mean for 98% specificity (95% CI: 95.9% to 100%). The sensitivities of stage I/II detection of HGSOC for Cutoffs 1 and 2 were 39.1% (95% CI: 19.2% to 59.0%) and 60.9% (95% CI: 41.0% to 80.8%), respectively. In some embodiments, Cutoff 1, with 99.5% specificity, can be used to impute the PPV for screening post-menopausal symptomatic women, where the prevalence of ovarian cancer is assumed to be 1 per 500 women (Goff et al., 2007; which is incorporated herein by reference for the purpose described herein). In some embodiments, the 98% specificity cutoff can be used to impute the PPV for screening women at hereditary risk where the prevalence is assumed to be 1 per 100 women. These separate cutoffs were established to account for the difference in false-positive tolerance among different patient populations. The resulting PPVs for HGSOC are well above 10% (17.6% and 27% for post-menopausal symptomatic and hereditary-risk women, respectively), demonstrating that in some embodiments, a single EV profiling assay (e.g., as described herein).has great potential for being used as an ovarian cancer screening test. Through the evaluation of additional, complementary biomarker combinations, the sensitivity of such an assay may be increased to obtain a stage I/II PPV of at least 10% in average-risk women. In some embodiments, the sensitivity of such an assay may be increased to obtain an even higher PPV at stage I/II in women subjects at hereditary risk for ovarian cancer, or in post-menopausal women subject who may be experiencing one or more symptoms associated with ovarian cancer, e.g., abdominal pain and/or pelvic pain.

To further improve the performance of an exemplary single EV profile assay (e.g., ones described herein) for detection of ovarian cancer, in some embodiments, additional biomarker candidates including membrane-bound proteins and intravesicular mRNAs/proteins can be identified.

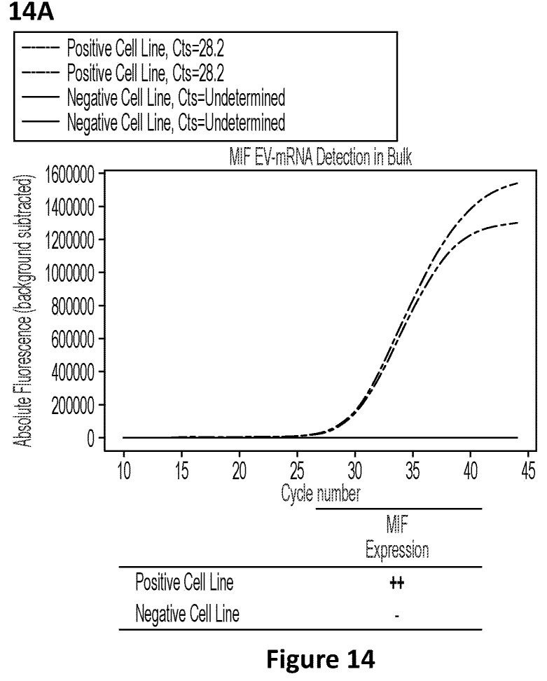

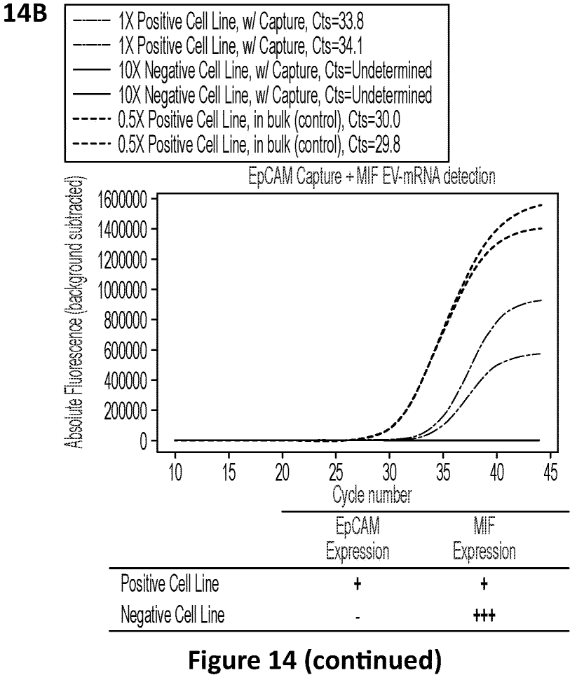

In some embodiments, it was demonstrated the feasibility of EV-mRNA detection using purified cell-line EVs in bulk, as shown in FIG. 14 (Panel A) for the detection of macrophage inhibitory factor (MIF) transcripts. Through immunoaffinity capture of a membrane bound protein marker, this approach enables the detection of two co-localized biomarkers. Moreover, EV-mRNA detection requires a simpler protocol because RT-qPCR can be performed directly after immunoaffinity capture. mRNA detection using EVs was demonstrated in FIG. 14(Panel B), where EVs were first captured using anti-epithelial cell adhesion molecule (EpCAM) modified magnetic beads and MIF mRNA was detected. Both positive and negative cell lines express MIF, however, only the positive cell line expresses EpCAM. Selective detection of the positive cell line, was demonstrated, even at an order of magnitude higher concentration of negative cell line EVs with such a detection system.

### Example 7: Development of Exemplary Ovarian Cancer Liquid Biopsy Assays

The present Example describes development of exemplary ovarian cancer liquid biopsy assays, for example, for screening women with or without symptoms who have average risk, hereditary risk, life-history risk, and/or post-menopausal risk. Steps leading to biomarker discovery were performed as described in Example 6 of the present disclosure.

As discussed in Example 6 and FIG. 7, sensitivity of an exemplary ovarian cancer liquid biopsy assay (e.g., as described herein) may be improved when using a 99.5% specificity cutoff for the detection of stage I and II ovarian cancer patients with a single biomarker combination of SLC34A2 immunoaffinity capture and MUC16 + MUC16 pliq-PCR readout. This may partly be attributed to the large range in assay signal across healthy women, likely due to healthy tissues that co-express SLC34A2 and MUC16.

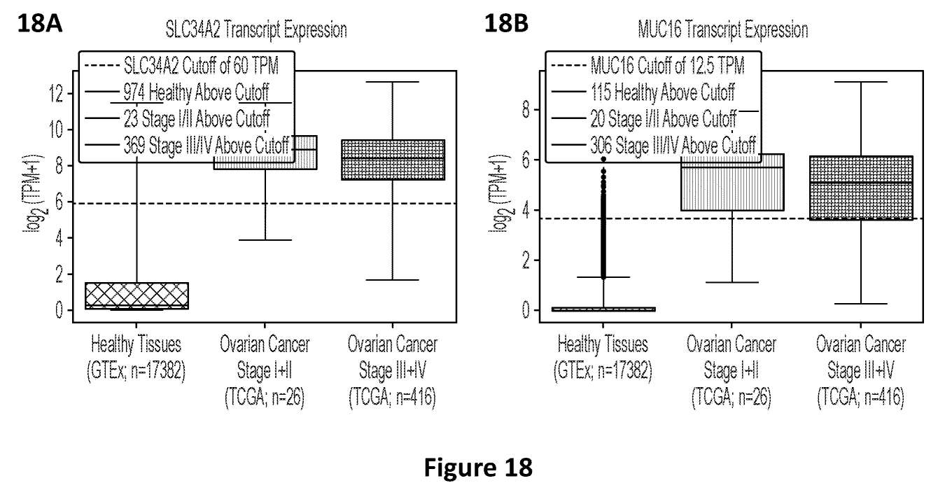

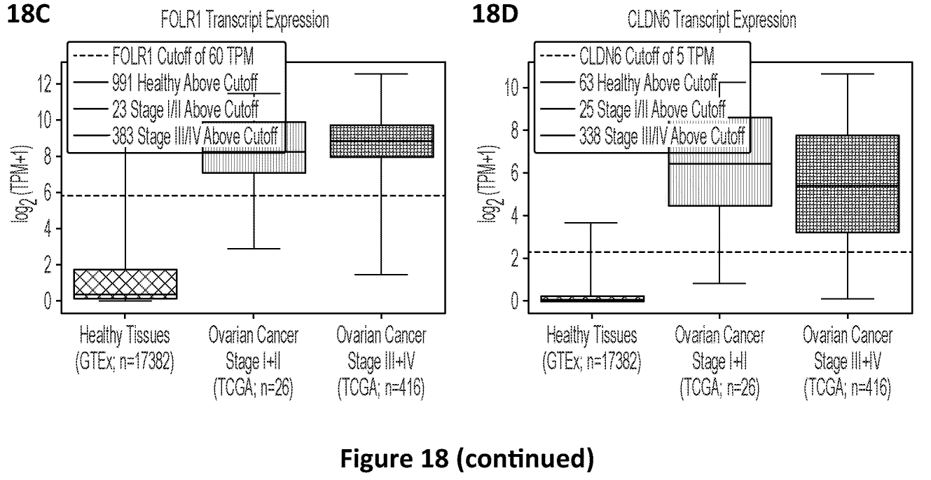

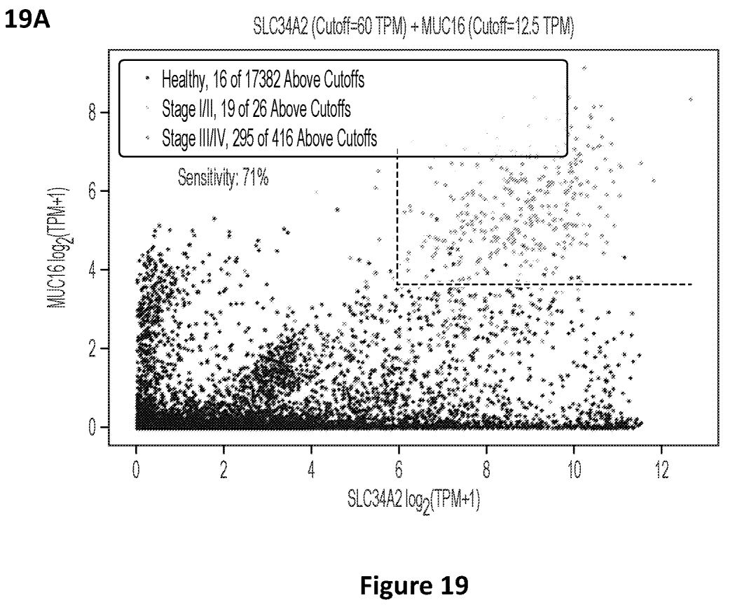

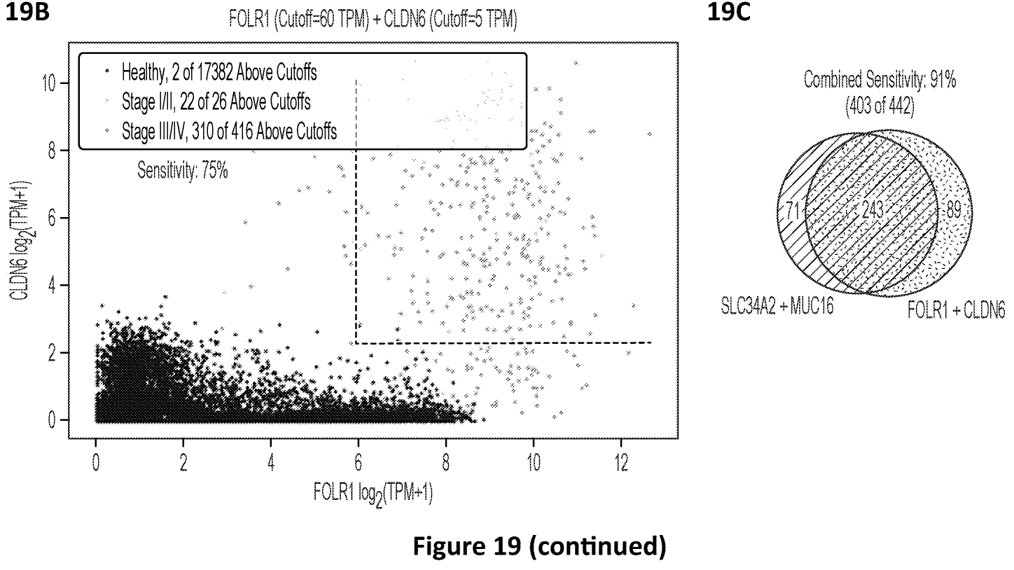

In order to improve the sensitivity and specificity, biomarker panels were expanded to include additional targets bioinformatically-predicted to be overexpressed in ovarian cancer (see FIG. 18 Panel A-D for a subset of generated data). In some embodiments, a list comprising polypeptide biomarkers FOLR1, CLDN3, CLDN6, AQP5, and LRRTM1 was devised. AQP5 (Aquaporin 5) is a water channel protein, a member of the Aquaporin family of integral membrane proteins involved in generation of saliva, tears, and pulmonary secretions. CLDN3 and CLDN6 (Claudin 3 and 6 respectively) are integral membrane proteins and components of tight junction strands, both proteins are members of the Claudin family of proteins which act to form continuous seals through cell-cell adhesions in epithelial and/or endothelial cells, preventing solute and water diffusion through the paracellular space. FOLR1 (Folate receptor 1) a member of the folate receptor family and is a transmembrane protein involved in the binding and subsequent intracellular delivery of extracellular folate and reduced folic acid derivatives. LRRTM1 (Leucine Rich Repeat Transmembrane Neuronal 1) is a transmembrane protein generally associated with neuronal function e.g., possibly schizophrenia and handedness, LRRTM1 is thought to be involved in protein—protein interactions at synapses, and transmission of certain chemicals across synapses.

Each of these biomarkers was rigorously screened using commercially-available monoclonal antibodies targeting cell-line EVs to identify antibody clones that exhibit high avidity and selectivity in exemplary assays (data not shown). At least one suitable antibody clone was identified for each biomarker.

To facilitate identification of novel biomarker combinations with high diagnostic utility, a procedure for screening combinations in pooled patient plasma samples was developed. Plasma from 90 healthy women between the ages of 55 and 79 was pooled to estimate the average healthy background protein abundance level for each biomarker combination. Concurrently, an ovarian cancer plasma pool was created from 10 stage III and 10 stage IV ovarian cancer patients to estimate the signal found in advanced ovarian cancer cases. Combinations of three biomarkers (one capture probe and two pliq-PCR readout probes) were generated from a list of seven potential biomarkers comprising AQP5, MUC16, CLDN3, CLDN6, FOLR1, LRRTM1, and SLC34A2. Combinations were screened in the pooled patient and/or control plasma samples. During each screening experiment, 1 mL of pooled patient plasma was purified, for example, using size-exclusion chromatography, and divided into ten aliquots (equivalent to 100 µL of plasma for each biomarker combination). A subset of preliminary biomarker screen data is provided in FIG. 20A, where each data point represents a different biomarker combination. Biomarker combinations in FIG. 20A that did not show a large difference in assay signal across plasma pools exhibited very low signal strength overall, suggesting few EVs contained those colocalized biomarkers. FIG. 20B provides the difference in assay signal between pooled healthy plasma and pooled ovarian cancer plasma for seven different exemplary biomarker combinations. The results demonstrate that biomarker combinations as described herein can detect a 100- to 1000-fold difference in signal between 100 µL of pooled healthy plasma when compared to pooled ovarian cancer plasma.

Improvements in biomarker combination screening may be achieved by testing pooled plasma from additional patient cohorts including pre-menopausal healthy women, women with benign gynecological tumors, women with other inflammatory conditions, and/or early stage HGSOC cases.

The data in FIGS. 20A and 20B indicate that several exemplary biomarker combinations as described herein (e.g., MUC16 capture probe with MUC16 + MUC16 pliq-PCR probes, SLC34A2 capture probe with MUC16 + FOLR1 pliq-PCR probes, SLC34A2 capture probe with FOLR1 + FOLR1 pliq-PCR probes, SLC34A2 capture probe with MUC16 + MUC16 pliq-PCR probes, MUC16 capture probe with MUC16 + CLDN6 pliq-PCR probes, FOLR1 capture probe with FOLR1 + CLDN6 pliq-PCR probes, etc.) can distinguish between healthy patients and those with ovarian cancer. To demonstrate that using multiple orthogonal and complimentary biomarker combinations would improve the performance of the exemplary assays described herein (e.g., by distinguishing different cancer patient subpopulations from the healthy cohort) several biomarker combinations were tested against a subset of patient samples previously investigated and/or described in Example 6 and FIG. 7. The healthy and HGSOC cohorts were specifically chosen to cover the entire range of signals observed in FIG. 7.

Provided in FIG. 21 are results showing performance of an exemplary assay described herein comprising at least two biomarker combinations, which in some embodiments may comprise 1) SLC34A2 capture, MUC16 + MUC16 pliq-PCR readout (the same combination used in the immediate FIG. 7 study, e.g., example 6); and 2) SLC34A2 capture, FOLR1 + FOLR1 pliq-PCR readout. The data demonstrate that exemplary assays as described herein (e.g., involving at least two biomarker combinations, e.g., at least two orthogonal biomarker combinations (e.g., at least two biomarker combinations that are statistically independent) was able to distinguish HGSOC patients from the healthy and benign patient cohorts. This orthogonality across biomarker combinations improved sensitivity, as compared to that observed in an assay involving a single biomarker combination. For example, in some embodiments where two or more biomarker combinations as described herein (e.g., orthogonal biomarker combinations as described herein) were utilized in an assay, there was an increase in overall assay sensitivity to 59.1% for the stage I and II ovarian cancer cases (n=22) and 63.9% for all ovarian cancer cases (n=36) at a specificity cutoff of 99.5%. These results demonstrate that incorporating multiple (e.g., at least two or more) biomarker combinations into exemplary assays described herein can materially improve performance (e.g., sensitivity and/or specificity) of the assays. For example, in some embodiments, the sensitivity of an exemplary assay involving at least two biomarker combinations (e.g., (1) SLC34A2 capture, MUC16 + MUC16 pliq-PCR readout; and (2) SLC34A2 capture, FOLR1 + FOLR1 pliq-PCR readout) at a 98% specificity cutoff was 66.7%. While the overall increase in sensitivity using both biomarker combinations in this Example (wherein each combination has a common biomarker, SLC34A2) is around 8.3%, certain embodiments where biomarker combinations that do not share a common biomarker can provide an even larger boost in sensitivity.

To further improve the performance of an exemplary single EV profile assay (e.g., ones described herein) for detection of ovarian cancer, in some embodiments, additional target biomarker including, e.g., membrane-bound proteins and intravesicular mRNAs/proteins as described herein can be incorporated in an exemplary single EV profile assay.

In some embodiments, biomarker panels were expanded to include additional targets bioinformatically-predicted to be overexpressed in ovarian cancer. Polypeptide biomarkers including CLDN6, AQP5, CLDN3, CLDN16, LEMD1, LRRTM1, FOLR1, MUC16, CHODL, CDH6, HTR3A, SLC34A2, EpCAM, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, and TNFRSF12A were considered. Certain biomarker combinations screened through an assay as described in Example 1 are depicted in Table 1. Following these primary screens, certain combinations were chosen for further analysis; exemplary results are described below.

Secondary screening results shown in Table 3 for biomarker combinations comparing healthy pooled samples (pool consists of 90 individual samples) or benign pooled (pool consists of 10 individual samples, from patients with serous cystadenoma, mucinous cystadenoma, endometrioid cyst, serous papillary cystadenoma, serous cystadenofibroma, or mature teratoma) samples to late stage ovarian cancer samples (pool consists of 20 individual samples, from patients with stage III or IV ovarian adenocarcinoma) or early stage ovarian cancer samples (pool consists of 16 individual samples, from patients with stage I or II ovarian adenocarcinoma) documented that certain biomarker combinations successfully differentiated ovarian cancer samples from reference samples (when measured by at least a 4 fold difference in amplification performed as described in example 1 with the biomarker combinations depicted).

As shown below, various biomarker combinations distinguished between the late-stage ovarian cancer pool and the benign gynecological tumor pool. Among other things, this finding demonstrates that these biomarker combinations, used in accordance with the present disclosure, specifically distinguish ovarian malignancies over benign tumors, thereby documenting one particularly advantageous feature of disclosed technologies. In some situations, this feature is especially important, as benign tumors can generate similar symptoms to malignant tumors and are often difficult to classify by conventional screening methods.

As shown below, various biomarker combinations distinguished the early-stage ovarian cancer pool from the healthy pool and/or the benign gynecological tumor pool. Among other things, these findings demonstrate that these biomarker combinations, used in accordance with the present disclosure, specifically distinguish ovarian malignancies over healthy tissue and/or benign tumors in early stage ovarian cancer (e.g., stage I/II), thereby documenting one particularly advantageous feature of disclosed technologies. In some situations, this feature is especially important, as correctly differentiating ovarian cancer samples from benign gynecological tumors while still detecting and diagnosing ovarian cancer at an early stage can be of critical importance in successfully combating the disease while maintaining patient wellbeing.

Among other things, results presented in the present Example demonstrate that provided technologies detect ovarian cystadenocarcinoma, and specifically demonstrate that provided technologies (e.g., using biomarker combinations as described herein to detect biomarkers in and/or on EVs) distinguish ovarian cancer-derived EVs from those originating from healthy tissues and/or benign gynecological tumors.

The present Example demonstrates effectiveness of provided technologies (e.g., proximity ligation technologies as described herein) that detect co-localized biomarker signatures (e.g., that detect sets of biomarkers in or on individual EVs), to detect advanced ovarian cancer, to detect early stage ovarian cancer, including with a signal at least four times greater than a pool of plasma from 90 healthy women, and/or to distinguish ovarian cancer plasma from benign tumor plasma. The present Example particularly documents effectiveness of such provided technologies to detect co-localized surface biomarker signatures (e.g., on surfaces of individual EVs).

Table 4 provides results from a secondary screen for certain biomarker combinations, while Table 5 provides tertiary screening results for certain biomarker signatures comprising at least three EV surface polypeptide biomarkers, the combinations of which were devised from the findings presented in Table 4. The results shown compare healthy pooled samples (pool consists of 90 individual samples), non-ovarian cancer pooled samples (pool consists of 26 individual samples, from patients with cancers such as breast cancer, colon cancer, lung cancer, pancreatic cancer, uterine cancer, or diffuse large B-cell lymphoma), and/or benign gynecological pooled samples (pool consists of 14 individual samples, from patients with serous cystadenoma, mucinous cystadenoma, serous cystadenofibroma, endometrioid cysts, endometrioid cystadenoma, mature teratoma, endometriosis, or leiomyoma) to late stage ovarian cancer samples (pool consists of 12 individual samples), early stage ovarian cancer samples delineated by serum CA-125 levels, e.g., high CA-125 (>35 U/mL, pool consists of 12 individual samples), or low CA-125 (<35 U/mL, pool consists of 11 individual samples). Suitable positive and negative controls were conducted for each biomarker combination to validate the assay worked appropriately (e.g., samples comprising no extracellular vesicles or comprising extracellular vesicles from ovarian cancer cell lines). The results documented that certain biomarker combinations successfully differentiate ovarian cancer samples from noted reference samples (when measured by at least a 2 fold difference in amplification performed as described in example 1 with the biomarker combinations depicted).

Among other things, as shown below, the findings demonstrate that certain of these biomarker combinations, used in accordance with the present disclosure, specifically distinguish ovarian malignancies from healthy tissues, benign gynecological tumors, and/or other cancer types, thereby documenting a particularly advantages feature of disclosed technologies. Furthermore, the results as shown below, demonstrate various biomarker combinations distinguished early stage high CA-125 ovarian cancer sample pools and/or early stage low CA-125 ovarian cancer sample pools from healthy sample pools, non-ovarian cancer sample pools, and/or benign gynecological tumor sample pools. In certain scenarios, this feature is especially important, as current standard of care non-invasive detection methods rely on measurement of serum CA-125 levels (as described above in the detailed description). Thus, the present Example describes biomarker signatures, used in accordance with the present disclosure, that may provide a more efficacious diagnostic system for early stage low CA-125 ovarian cancer diagnosis than the current standard of care. In addition, the present Examples findings describe assays that differentiate between ovarian cancer samples and benign gynecological tumors and/or other cancer types. In some situations, this feature is especially important, as benign tumors and/or other cancer types can generate similar symptoms and/or molecular signatures to malignant ovarian cancers, and these conditions are often difficult to distinguish when using conventional screening methods.

Among other things, results presented in the present Example demonstrate that provided technologies detect ovarian cystadenocarcinoma, and specifically demonstrate that provided technologies (e.g., using biomarker combinations as described herein to detect biomarkers in and/or on EVs) distinguish ovarian cancer-derived EVs from those originating from healthy tissues, benign gynecological tumors, and/or derived from certain other cancer types.

The present Example demonstrates effectiveness of provided technologies (e.g., proximity ligation technologies as described herein) that detect co-localized biomarker signatures (e.g., that detect sets of biomarkers in or on individual EVs) to detect advanced ovarian cancer, including with a signal at least two times greater than a pool of plasma from 90 healthy women, and/or to distinguish ovarian cancer plasma from benign gynecological tumor plasma and/or to distinguish ovarian cancer plasma from certain other cancer types. The present Example particularly documents effectiveness of such provided technologies to detect co-localized surface biomarker signatures (e.g., on surfaces of individual EVs).

The present disclosure further defines particular biomarkers, and sets thereof, that are particularly useful and/or effective in assays as described herein. In some embodiments, particular exemplified biomarkers and/or sets, are useful as exemplified. In some embodiments, multiple particular exemplified biomarkers and/or sets may be applied to the same samples (e.g., to different aliquots of the same sample) and/or to analogous samples from the same individual(s) and/or sources. In some embodiments, a plurality of separate biomarkers, or sets thereof (e.g., biomarker signatures) are assessed, and outcome score(s) (e.g., reflective of a particular level of binding and/or of a particular level of sensitivity and/or specificity) are determined for each. In some embodiments, diagnostic certainty is improved when multiple such assessments achieve scores indicative of the same status (e.g., cancerous or not, stage of cancer, etc). Alternatively or additionally, in some embodiments, diagnostic certainty is improved when determination and/or consideration of such scores is combined with one or more other diagnostic assessments or strategies (e.g., TVUS and/or serum CA-125 level, etc.).

Table 3 depicting certain biomarker combinations that differentiated ovarian cancer pooled samples when compared to healthy or benign samples. Detection/differentiation is denoted as * and corresponds to a 4 fold amplification difference between cancerous samples when compared to the noted reference samples. Abbreviations: late stage ovarian cancer (LS), early stage ovarian cancer (ES), target of capture probe (Capture), target of detection probe (Det), combination (Combo).

| | | | | Compared to Healthy Pool | | Compared to Benign Pool | |
|---|---|---|---|---|---|---|---|
| Combo | Capture | Det 1 | Det 2 | LS | ES | LS | ES |
| 1 | SLC34A2 | SLC34A2 | FOLR1 | * | | * | |
| 2 | SLC34A2 | MUC16 | MUC16 | * | | * | |
| 3 | SLC34A2 | MUC16 | FOLR1 | * | * | * | |
| 4 | SLC34A2 | FOLR1 | FOLR1 | * | * | * | |
| 5 | MUC16 | SLC34A2 | MUC16 | * | * | * | |
| 6 | MUC16 | SLC34A2 | FOLR1 | * | * | | |
| 7 | MUC16 | MUC16 | MUC16 | * | * | * | |
| 8 | MUC16 | MUC16 | FOLR1 | * | * | * | * |
| 9 | MUC16 | MUC16 | CLDN3 | * | * | * | * |
| 10 | MUC16 | MUC16 | CLDN6 | * | | * | |
| 11 | MUC16 | MUC16 | AQP5 | * | | * | |
| 12 | MUC16 | FOLR1 | FOLR1 | * | | * | |
| 13 | MUC16 | FOLR1 | CLDN3 | * | * | * | * |
| 14 | MUC16 | FOLR1 | AQP5 | * | | * | |
| 15 | FOLR1 | MUC16 | MUC16 | * | | * | |
| 16 | FOLR1 | MUC16 | FOLR1 | * | | * | |
| 17 | FOLR1 | MUC16 | CLDN3 | * | | * | |
| 18 | FOLR1 | FOLR1 | CLDN6 | | | * | |
| 19 | FOLR1 | SLC34A2 | MUC16 | * | | * | |
| 20 | FOLR1 | FOLR1 | FOLR1 | * | | * | |
| 21 | FOLR1 | FOLR1 | CLDN3 | * | | * | |
| 22 | FOLR1 | FOLR1 | AQP5 | * | | * | |
| 23 | CLDN3 | FOLR1 | FOLR1 | * | * | * | |
| 24 | CLDN3 | SLC34A2 | MUC16 | * | | * | |
| 25 | CLDN3 | MUC16 | MUC16 | * | | * | |
| 26 | CLDN3 | MUC16 | FOLR1 | * | | * | |
| 27 | CLDN3 | MUC16 | CLDN3 | * | | * | |
| 28 | CLDN3 | MUC16 | CLDN6 | * | | * | |
| 29 | LRRTM1 | MUC16 | MUC16 | * | * | | * |

Table 4 depicting certain exemplary two biomarker combinations that were shown in a primary screen to differentiate late stage ovarian cancer pooled samples from healthy pooled samples. Noted combinations were then utilized to compare early stage ovarian cancer samples (differentiated by CA-125 levels) to healthy sample pools, non-ovarian cancer sample pools, or benign gynecological tumor sample pools. Detection/differentiation is denoted as * and corresponds to a 2 fold amplification difference between cancerous samples when compared to the noted reference samples; note that in Table 4, no distinction is made between biomarkers used for immunoaffinity capture and biomarkers used for assay readout. Abbreviations: late stage ovarian cancer (LS), early stage high CA-125 ovarian cancer (ES High), early stage low CA-125 ovarian cancer (ES Low), target of capture and/or detection probe (Target), combination (Combo)

| | | | Compared to Healthy pool | | | Compared to Benign Tumor Pool | | Compared to Non-Ovarian Cancer pool | |
|---|---|---|---|---|---|---|---|---|---|
| Combo | Target 1 | Target 2 | LS | ES High | ES Low | ES High | ES Low | ES High | ES Low |
| 1 | ALPL | FOLR1 | * | * | | * | | * | |
| 2 | BST2 | MUC16 | * | * | | * | | * | |
| 3 | FOLR1 | MSLN | * | * | | * | | * | |
| 4 | FOLR1 | MUC16 | * | * | | | | * | * |
| 5 | MSLN | MUC16 | * | * | | * | | | |
| 6 | MSLN | MUC1 | * | * | | * | | * | * |
| 7 | MSLN | SLC2A1 | * | | | | | | |
| 8 | MUC1 | sTn | * | * | * | * | * | * | |
| 9 | MUC1 | MUC16 | * | * | | * | * | * | |
| 10 | MUC1 | FOLR1 | * | * | | * | | * | |
| 11 | MUC16 | sTn | * | * | | * | | * | |
| 12 | PTGS 1 | MUC16 | * | * | | | | | |
| 13 | SLC34A2 | FOLR1 | * | * | | | | * | |
| 14 | SLC34A2 | MUC16 | * | | | | | * | |
| 15 | sTn | FOLR1 | * | * | | * | | * | * |
| 16 | sTn | MSLN | * | * | | * | * | * | * |
| 17 | TACSTD2 | MUC16 | * | * | * | * | * | * | * |
| 18 | TACSTD2 | sTn | * | * | | * | | * | |

Table 5 depicting certain exemplary three target biomarker combinations. Comparisons are of late stage ovarian cancer and/or early stage ovarian cancer samples (differentiated by CA-125 levels) to healthy sample pools, non-ovarian cancer sample pools, or benign gynecological tumor sample pools. Detection/differentiation is denoted as * and corresponds to a 2 fold amplification difference between cancerous samples when compared to the noted reference samples. Abbreviations: late stage ovarian cancer (LS), early stage high CA-125 ovarian cancer (ES High), early stage low CA-125 ovarian cancer (ES Low), target of capture probe (Capture), target of detection probe (Det), combination (Combo).

| | | | | Compared to Healthy Pool | | | Compared to Benign Tumor pool | | | Compared to Non-Ovarian Cancer pool | | |
|---|---|---|---|---|---|---|---|---|---|---|---|---|
| Combo | Capture | Det 1 | Det 2 | LS | ES High | ES Low | LS | ES High | ES Low | LS | ES High | ES Low |
| 1 | TACSTD2 | MUC16 | sTn | * | * | * | | * | | * | * | * |
| 2 | TACSTD2 | MUC16 | MUC1 | * | * | * | | | * | | | * |
| 3 | TACSTD2 | MUC16 | MSLN | | | | | | | | | * |
| 4 | TACSTD2 | MUC16 | FOLR1 | * | | * | | | | * | | * |
| 5 | TACSTD2 | sTn | MUC1 | * | * | * | * | | | * | * | * |
| 6 | TACSTD2 | sTn | MSLN | | | * | | | | | | * |
| 7 | TACSTD2 | sTn | FOLR1 | | | | * | * | | * | * | |
| 8 | TACSTD2 | MUC1 | MSLN | | | | | | | | | * |
| 9 | TACSTD2 | MUC1 | FOLR1 | | | | | | | | | |
| 10 | TACSTD2 | MSLN | FOLR1 | | | | | | | | | |
| 11 | MUC1 | MUC16 | sTn | * | * | | * | * | * | * | * | |
| 12 | MUC1 | MUC16 | MSLN | * | * | | * | * | | * | * | * |
| 13 | MUC1 | MUC16 | FOLR1 | * | * | | * | * | | * | * | * |
| 14 | MUC1 | sTn | MSLN | * | * | | * | * | | * | * | * |
| 15 | MUC1 | sTn | FOLR1 | * | * | | * | * | * | * | * | |
| 16 | MUC1 | MSLN | FOLR1 | * | * | | * | * | | * | * | |
| 17 | MUC16 | sTn | MUC1 | * | * | | * | * | * | * | * | |
| 18 | MUC16 | sTn | MSLN | * | * | | * | * | * | * | * | |
| 19 | MUC16 | sTn | FOLR1 | * | * | | * | * | | * | * | |
| 20 | MUC16 | MUC1 | MSLN | | | | | | | * | * | * |
| 21 | MUC16 | MUC1 | FOLR1 | | | | | | | | | |
| 22 | MUC16 | MSLN | FOLR1 | | | | | | | * | * | * |
| 23 | sTn | MUC16 | MUC1 | * | * | | * | * | | * | * | |
| 24 | sTn | MUC16 | MSLN | * | * | | * | * | | * | * | * |
| 25 | sTn | MUC16 | FOLR1 | * | * | | * | * | | * | * | |
| 26 | sTn | MUC1 | MSLN | * | * | | * | * | * | * | * | * |
| 27 | sTn | MUC1 | FOLR1 | * | * | | * | * | * | * | * | * |
| 28 | sTn | MSLN | FOLR1 | * | * | | * | | | * | * | * |
| 29 | MSLN | MUC16 | sTn | * | * | | * | * | | * | * | |
| 30 | MSLN | MUC16 | MUC1 | * | * | | * | * | | * | * | * |
| 31 | MSLN | MUC16 | FOLR1 | * | * | | * | * | | * | * | |
| 32 | MSLN | sTn | MUC1 | * | * | * | * | * | * | * | * | |
| 33 | MSLN | sTn | FOLR1 | * | * | | * | * | | * | * | |
| 34 | MSLN | MUC1 | FOLR1 | * | * | * | * | * | * | * | * | * |
| 35 | SLC34A2 | MUC16 | FOLR1 | * | * | | | | | * | * | |
| 36 | SLC34A2 | MUC16 | MSLN | * | | | * | | | * | | |
| 37 | SLC34A2 | MUC16 | sTn | * | | | * | * | | * | * | |
| 38 | SLC34A2 | FOLR1 | sTn | * | | | * | * | | * | * | |

### Example 8: Further Characterization of Exemplary Ovarian Cancer Liquid Biopsy Assays

This example is directed towards further characterization of various biomarker combinations in exemplary ovarian cancer liquid biopsy assays using different cell populations and patient populations. Useful biomarker combinations for detection of ovarian cancer can be determined by screening various combinations of biomarkers disclosed herein (e.g., surface protein target biomarkers such as, e.g., SLC34A2, MUC16, FOLR1, CLDN3, CLDN6, AQP5, LRRTM1, CLDN16, LEMD1, MUC16, CHODL, CDH6, HTR3A, SLC34A2, EpCAM, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof) in pooled control and patient plasma samples. Pooled samples can provide an estimation for the average assay signal among various patient cohorts, facilitating biomarker combination triage.

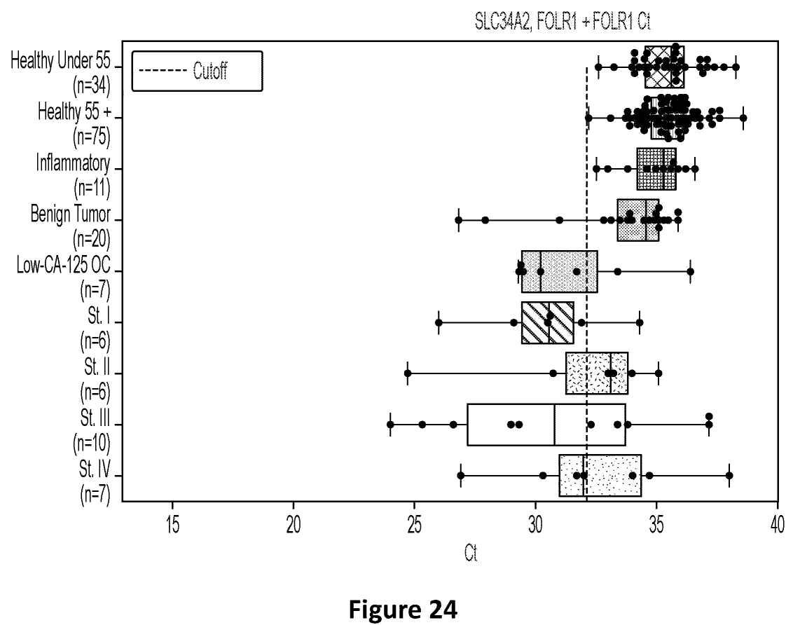

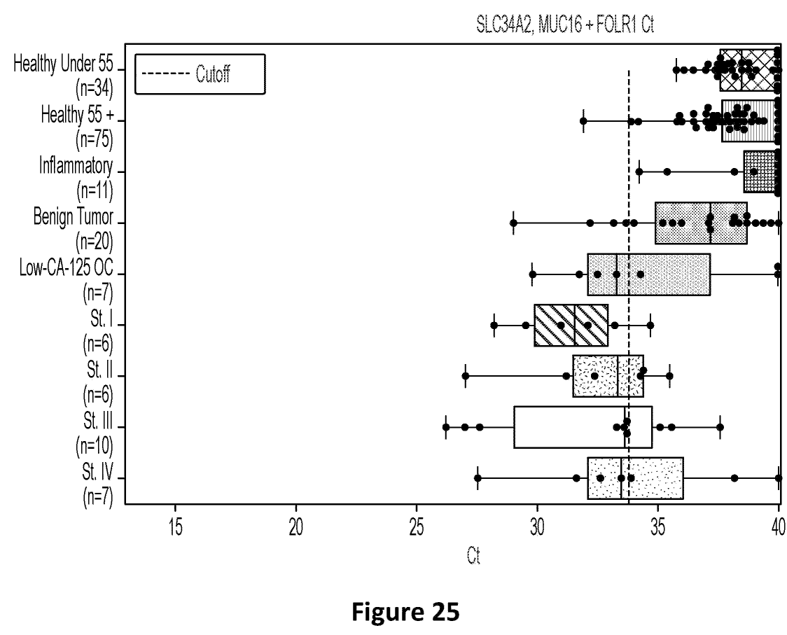

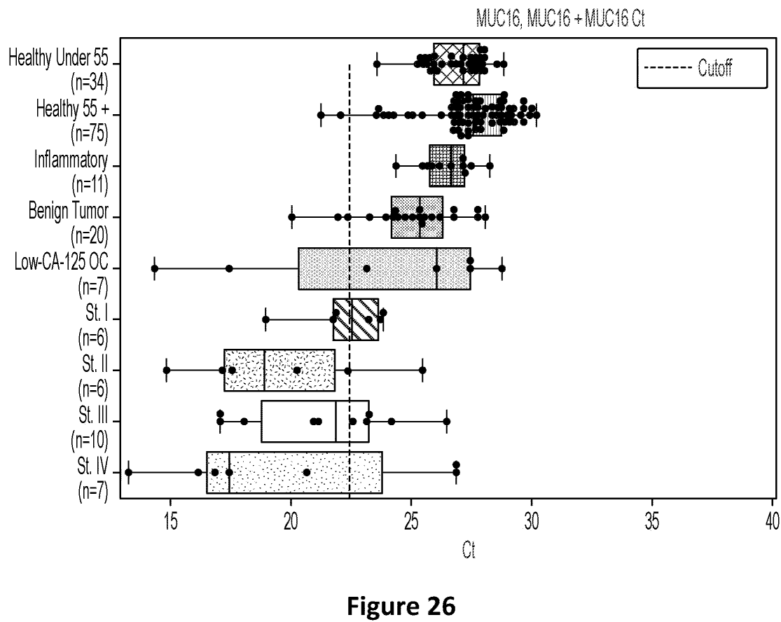

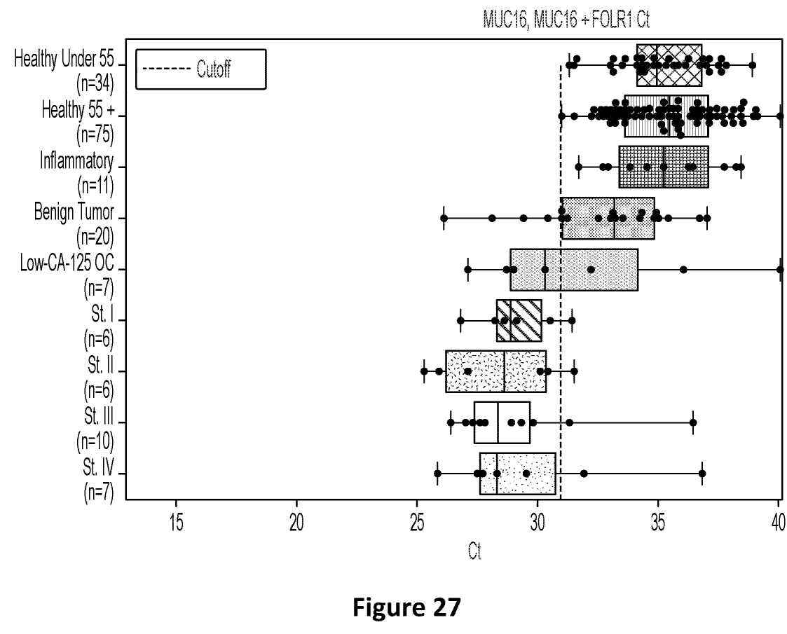

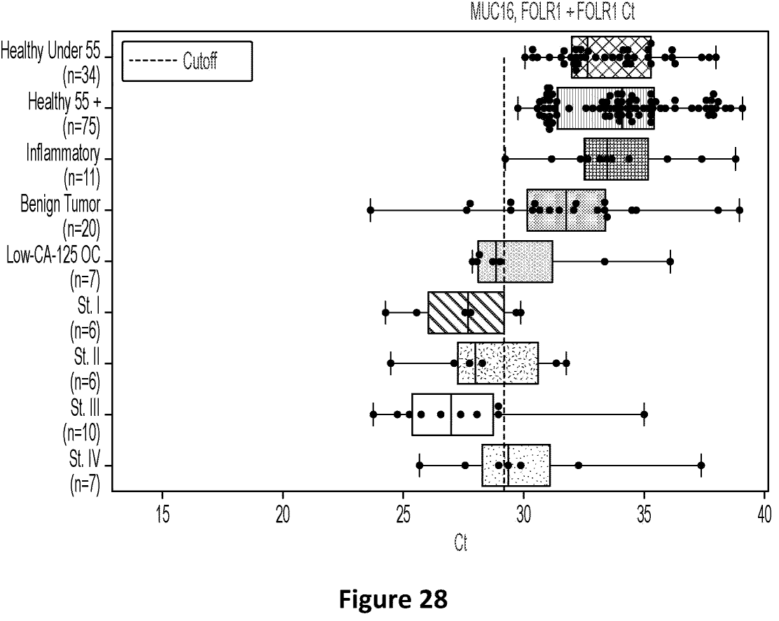

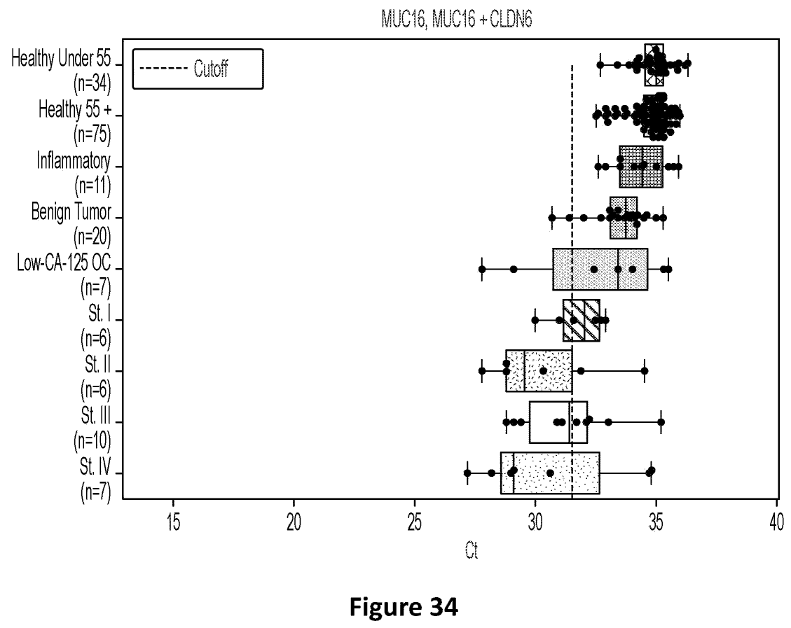

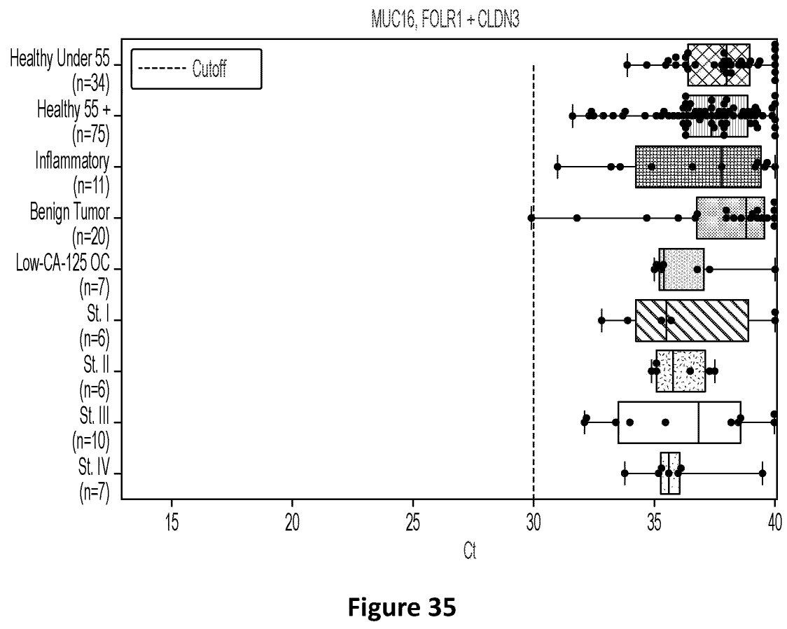

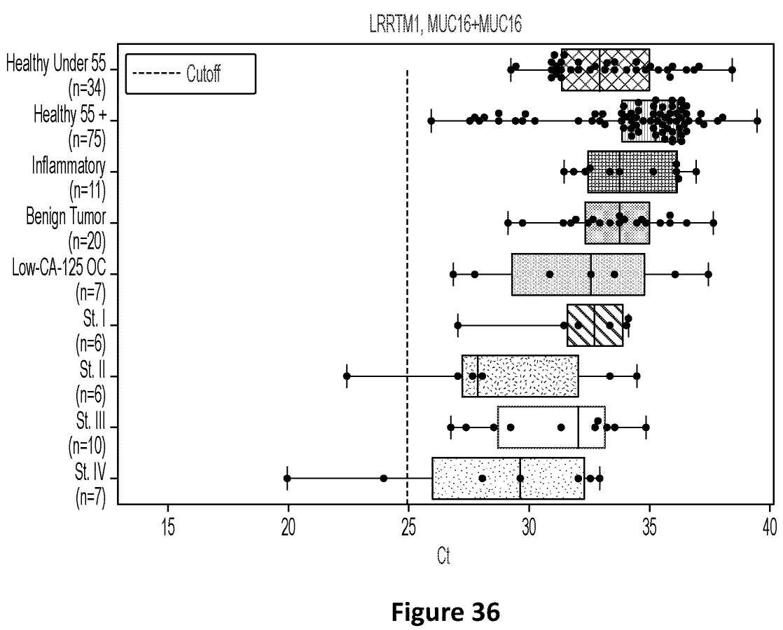

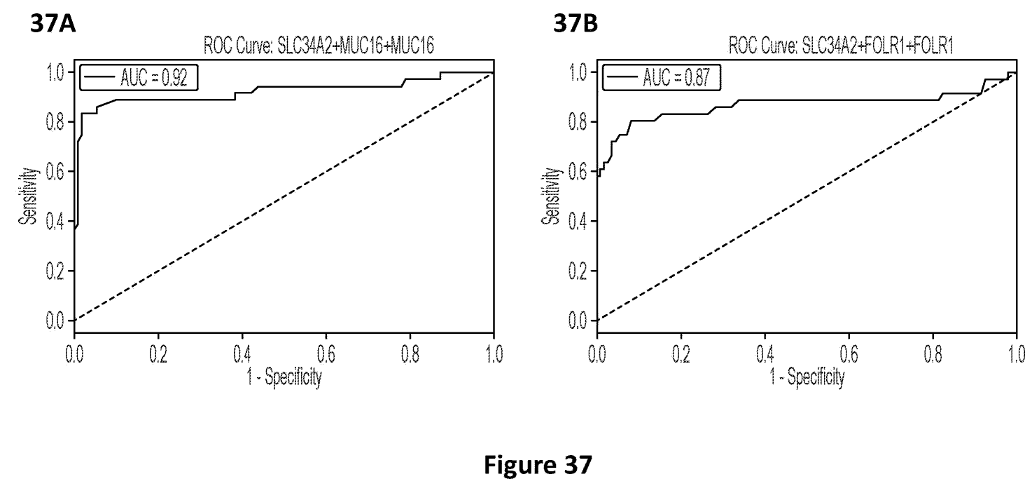

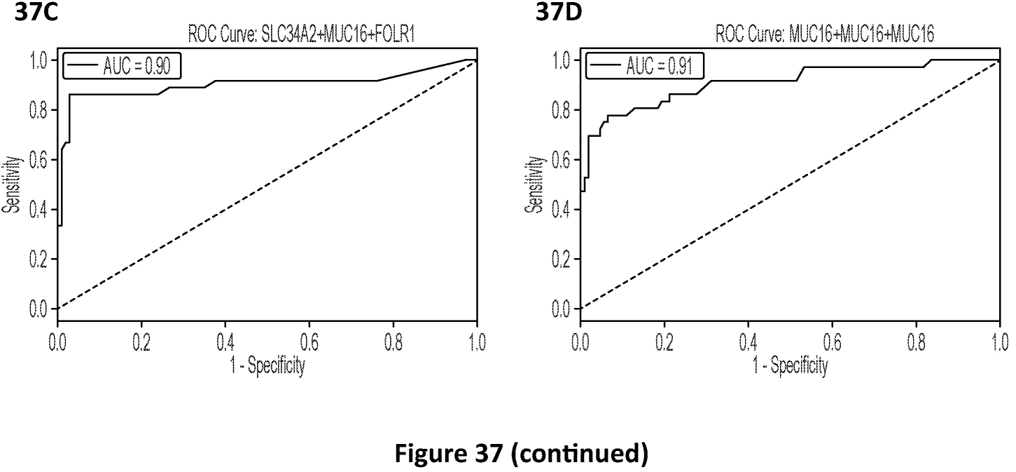

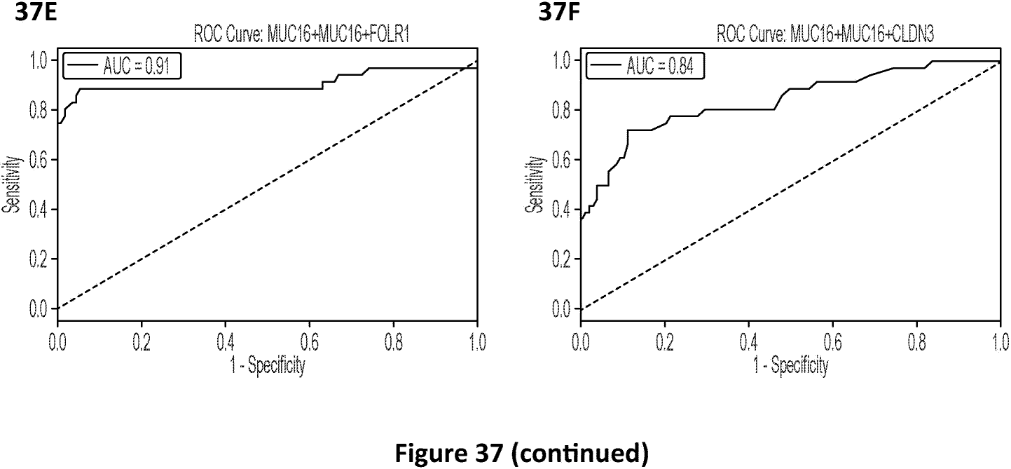

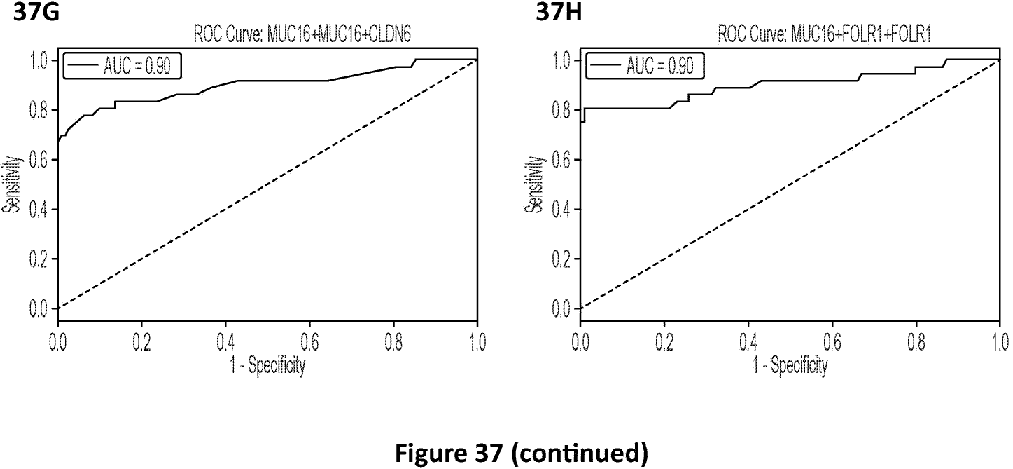

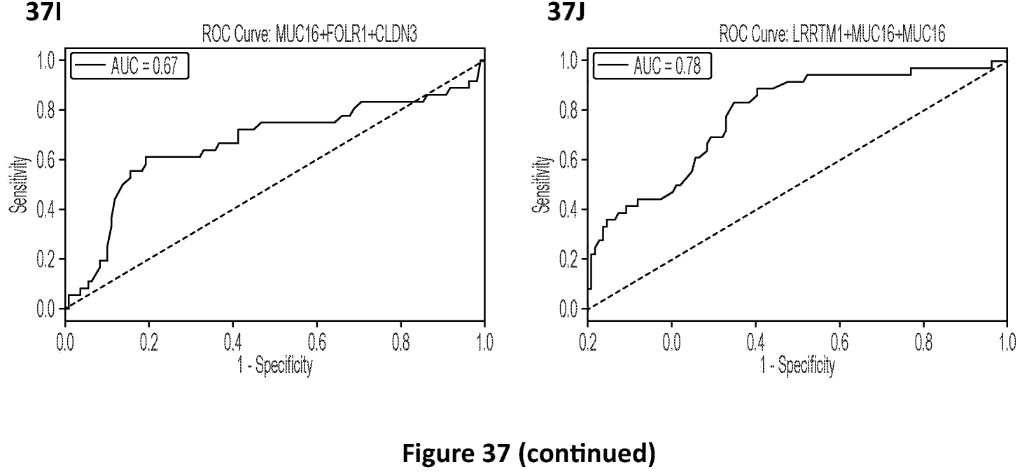

In some embodiments, a biomarker combination for detection of ovarian cancer can comprise one, two, three, four, five, six, seven or more biomarkers (e.g., ones described herein), wherein such a combination comprises at least one biomarker for capturing extracellular vesicles (e.g., by immunoaffinity capture) and at least one biomarker (including, e.g., one, two, three, four, five, six, seven, or more biomarkers) for detection by pliq-PCR assay. In some embodiments, a target biomarker for capturing extracellular vesicles (e.g., immunoaffinity capture) can be same as at least one target biomarker for pliq-PCR based analysis. In some embodiments, target biomarkers for capturing extracellular vesicles and pliq-PCR based analysis can each be distinct. Examples of various exemplary biomarker combinations are shown in FIG. 20B, FIGS. 22-28, and FIGS. 33-36. Examples of a diagnostic capacity of various exemplary biomarker combinations are shown in FIGS. 37A-J.

Various biomarker combinations can be validated in pooled patient cohorts. Patient cohorts can include appropriate patient and/or control populations. In certain embodiments, a patient cohort can comprise post-menopausal healthy women (e.g., healthy women aged between 55 and 79 years of age), pre-menopausal healthy women (e.g., women aged between 20 and 54 years of age), women with advanced HGSOC (e.g., stage III and stage IV HGSOC cases), women with early stage HGSOC (e.g., stage I and stage II HGSOC cases), women with benign gynecological tumors (e.g., women with benign gynecological growths), and/or women with inflammatory diseases, disorders, or conditions (e.g., women with inflammatory conditions including endometriosis, pelvic inflammatory disease, inflammatory bowel disease (e.g., Crohn’s disease and/or ulcerative colitis, etc.), and/or other inflammatory diseases).

In some embodiments, a two-step screening procedure can characterize performance of various biomarker combinations. For example, various biomarker combinations (e.g., various combinations of biomarkers for a capture probe (e.g., for immunoaffinity capture) and detection probes) can initially be screened in healthy background pools (from various age groups) and an advanced stage HGSOC pool. In some embodiments, biomarker combinations that exhibit poor separation between the healthy and ovarian cancer pools (e.g., a ΔCt less than 4, corresponding to less than a 16-fold difference) can be eliminated from further study as a biomarker combination to use in isolation, however, such biomarker combinations may be useful when combined with additional biomarker combinations as described herein. In some embodiments, biomarker combinations that exhibit poor separation between the healthy and ovarian cancer pools (e.g., a ΔCt less than 2, corresponding to less than a 4-fold difference) can be eliminated from further study as a biomarker combination to use in isolation, however, such biomarker combinations may be useful when combined with additional biomarker combinations as described herein. In some embodiments, biomarker combinations that exhibit poor separation between the healthy and ovarian cancer pools (e.g., a ΔCt less than 1, corresponding to less than a 2-fold difference) can be eliminated from further study as a biomarker combination to use in isolation, however, such biomarker combinations may be useful when combined with additional biomarker combinations as described herein.

In some embodiments, a two-step screening procedure can characterize performance of various biomarker combinations. For example, various biomarker combinations (e.g., various combinations of biomarkers for a capture probe (e.g., for immunoaffinity capture) and detection probes) can initially be screened in healthy background pools (from various age groups) and an advanced stage HGSOC pool. In some embodiments, biomarker combinations that exhibit good diagnostic performance (e.g., a ΔCt greater than 1, corresponding to greater than a 2-fold difference) can undergo a second round of screening with pooled early stage HGSOC, benign gynecological tumors, and/or inflammatory conditions. In some embodiments, biomarker combinations that exhibit good diagnostic performance (e.g., a ΔCt greater than 2, corresponding to greater than a 4-fold difference) can undergo a second round of screening with pooled early stage HGSOC, benign gynecological tumors, and/or inflammatory conditions. In some embodiments, biomarker combinations that exhibit good diagnostic performance (e.g., a ΔCt greater than 4, corresponding to greater than a 16-fold difference) can undergo a second round of screening with pooled early stage HGSOC, benign gynecological tumors, and/or inflammatory conditions. In some embodiments, top biomarker combinations (e.g., the best performing approximately 20 biomarker combinations) that can distinguish early and late stage HGSOC from the control pools can be further evaluated.

Incorporation of one or more additional biomarker combinations in an exemplary assay (e.g., ones described herein) can improve its sensitivity. As noted in Example 7, by utilizing at least two biomarker combinations, improved performance of an assay (e.g., at least approximately 80% sensitivity at 98% specificity; or at least approximately 70% sensitivity at 99.5% specificity) can be achieved. Pooled patient samples can approximate the assay signals across individual patient populations, a trait which can provide a realistic matrix to assess a large number of biomarker combinations in an efficient manner. The top biomarker combinations (e.g., up to 20 biomarker combinations) identified can be further tested in individual patient plasma based pilot studies. In some embodiments, an individual patient plasma based pilot study can comprise control patients who are either pre- or post- menopausal, control patients who are asymptomatic, control patients who are symptomatic, control patients with benign gynecological tumors, and/or control patients with non-ovarian cancer inflammatory health conditions. In some embodiments, an individual patient plasma based pilot study can comprise test patients who are either pre- or post- menopausal, test patients who are symptomatic, test patients who are asymptomatic, test patients with stage I or stage II HGSOC, and/or test patients with stage III or stage IV HGSOC. Non-HGSOC health conditions can be aged-matched to the ovarian cancer cohort. It can be expected that the control samples (e.g., healthy controls, benign gynecological tumors, and/or other off-target conditions) can provide an estimate of a log-normal distribution to set signal cutoffs pertaining to a 98% specificity (with approximately 95% CI: 95.3% to 100%) for hereditary risk screening assays and 99.5% specificity (with approximately 95% CI: 98.1% to 100%) for symptomatic triaging assays. Biomarker combination performance characteristics can be evaluated using bivariate associations between combinations to assess independence, and top biomarker combinations can be further tested using a three-variable logistic regression model for predicting ovarian cancer.

The identification of novel biomarker combinations that can distinguish HGSOC cases from controls and identify orthogonality between combinations can be achieved. This orthogonality can aid in distinguishing HGSOC cases from the control cohorts, increasing the sensitivity of exemplary assays as described herein. To assess racial diversity in the patient cohort, samples can be obtained from vendors which source from diverse populations. In certain embodiments, exemplary biomarker combinations may be specific to particular racial and/or ethnic groups. In certain embodiments, testing exemplary biomarker combinations in diverse racial and/or ethnic groups can identify biomarker combinations of appropriate diagnostic value for specific racial and/or ethnic groups.

Validation of additional ovarian cancer biomarker combinations in primary patient samples can improve the diagnostic performance of exemplary assays. In a cohort of 149 women, exemplary assays as described herein achieved a 66.7% sensitivity at 98% specificity and a 63.9% sensitivity at 99.5% specificity using two biomarker combinations (as described in Example 7 and illustrated in FIG. 21). Through the incorporation of additional, orthogonalbiomarker combinations, assay performance can improve beyond 80% sensitivity at 98% specificity and 70% sensitivity at 99.5% specificity.

### Example 9: Additional Assessment of Other Biomarker Combinations for Detection of Ovarian Cancer

In this Example, 10 different biomarker combinations were assessed using various patient samples including, e.g., post-menopausal healthy women (e.g., healthy women aged between 55 and 79 years of age), pre-menopausal healthy women (e.g., women aged between 20 and 54 years of age), women with advanced HGSOC (e.g., stage III and stage IV HGSOC cases), women with early stage HGSOC (e.g., stage I and stage II HGSOC cases), women with benign gynecological tumors (e.g., women with benign gynecological growths), and/or women with inflammatory diseases, disorders, or conditions (e.g., women with endometriosis, Crohn’s diseases, ulcerative colitis, etc.). FIG. 22 depicts performance of an exemplary assay (e.g., as described in Example 1) involving individual exemplary biomarker combinations to distinguish control subjects (e.g., healthy woman subjects and/or subjects with benign gynecological tumors and/or inflammatory conditions) from ovarian cancer patients. Exemplary individual biomarker combinations are presented in Table 6:

TABLE 6

certain exemplary combinations of capture and detection probes

| | | |
|---|---|---|
| Target of Capture Probe | Target of Detection Probe 1 | Target of Detection Probe 2 |
| SLC34A2 | MUC16 | MUC16 |
| SLC34A2 | FOLR1 | FOLR1 |
| SLC34A2 | MUC16 | FOLR1 |
| MUC16 | MUC16 | MUC16 |
| MUC16 | MUC16 | FOLR1 |
| MUC16 | MUC16 | CLDN3 |
| MUC16 | FOLR1 | CLDN3 |
| MUC16 | MUC16 | CLDN6 |
| MUC16 | FOLR1 | FOLR1 |
| LRRTM1 | MUC16 | MUC16 |

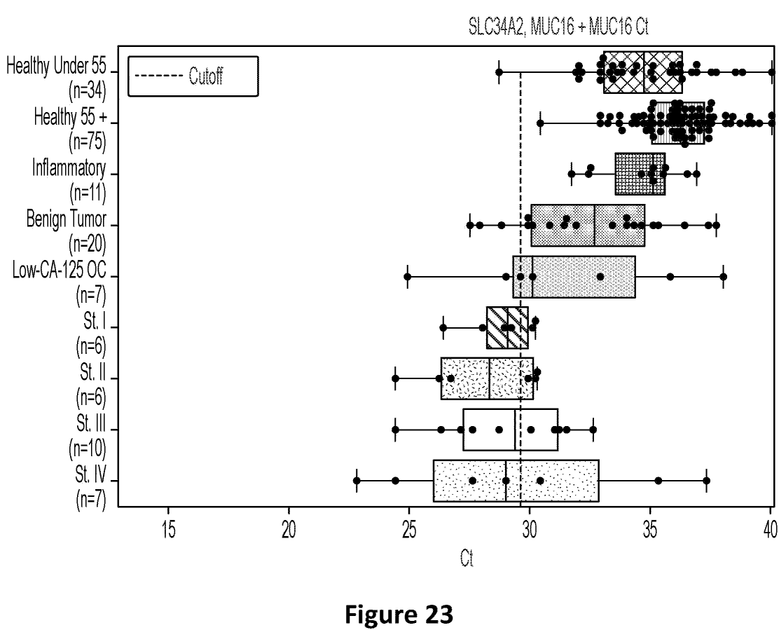

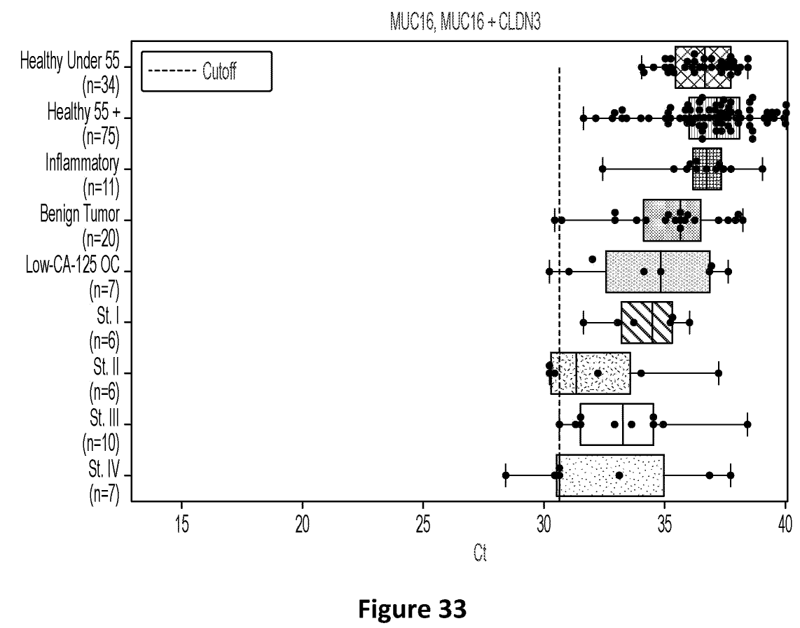

FIGS. 23-28 depict performance of exemplary assays (e.g., as described in Example 1) involving certain biomarker combinations shown in the table above. In each assay, the cut-off value was determined by selecting the less restrictive of either (i) 2.93 standard deviations away from mean of healthy control subjects and subjects with inflammatory conditions (e.g., to exclude 99.83% of healthy subjects in the distribution) or (ii) a maximum assay signal from healthy control subjects. In some embodiments, benign ovarian tumor samples may be less of a concern for off-target signals than healthy control subjects and/or subjects with inflammatory conditions (e.g., Crohn’s disease, ulcerative colitis, endometriosis, etc.). Accordingly, in some such embodiments, benign ovarian tumor samples may not be included to determine a cutoff value. FIGS. 33-36 depict performance of additional exemplary assays (e.g., performed as described in Example 1) involving certain biomarker combinations shown in the table above.

Among the biomarker combinations tested, FIG. 29 depicts performance (including sensitivity) of three selected biomarker combinations, which are: (Panel A) A capture agent directed to SLC34A2 and a set of at least a first detection probe directed to MUC16 and a second detection probe directed to MUC16; (Panel B) A capture agent directed to SLC34A2 and a set of at least a first detection probe directed to FOLR1 and a second detection probe directed to FOLR1; and (Panel C) a capture agent directed to MUC16 and a set of at least a first detection probe directed to MUC16 and a second detection probe directed to FOLR1. As shown in the figure, each individual assay achieved a sensitivity of at least 50% or above.

To further increase the sensitivity of an ovarian cancer detection assay, combinations of at least two biomarker combinations described herein can be used. For example, when using all three biomarker combinations as shown in FIG. 29 (i.e., a positive indication of ovarian cancer only when a sample meets or exceeds a classification cutoff including cutoffs of all three individual biomarker combinations), sensitivity of such an assay is improved - ovarian cancer stage I patients: 83.3%; ovarian cancer stage II patients: 83.3%; ovarian cancer stage III patients: 90.0%; ovarian cancer stage IV patients: 71.4%; ovarian cancer patients with low serum CA-125 level: 71.4%; women with benign ovarian tumors: 30.0%; and healthy subjects: 0.8%. This shows that use of two or more biomarker combinations (e.g., as described herein) can achieve an assay sensitivity of at least 80-81% and an assay specificity of at least 99.2% or higher (up to 100% specificity).

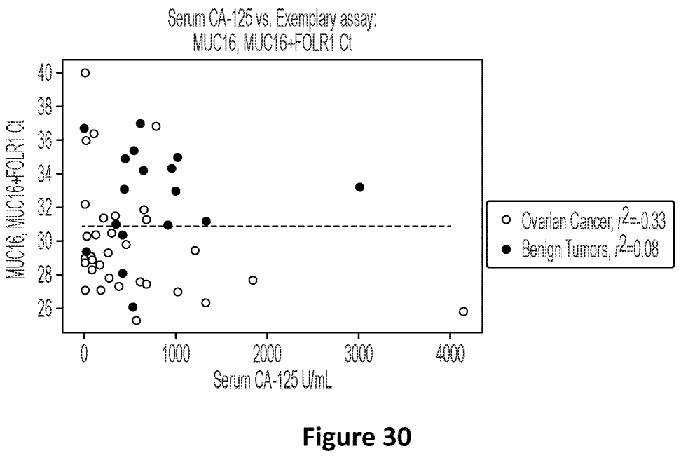

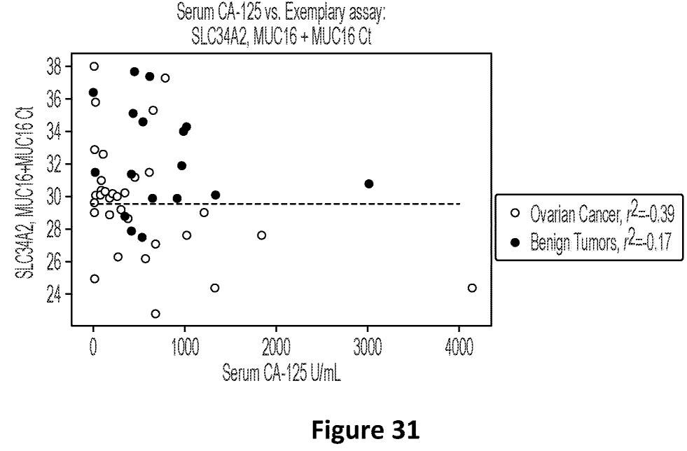

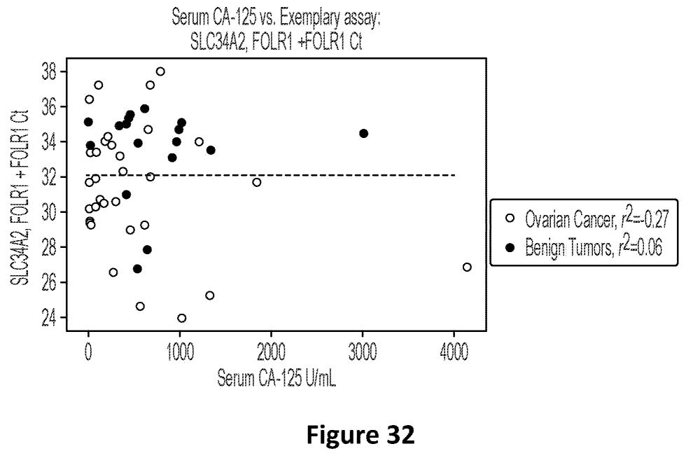

FIGS. 30-32 demonstrate ability of exemplary assays (e.g., as described in Example 1) using certain biomarker combinations can detect ovarian cancer patients with normal serum CA-125 (e.g., under 35 U/mL), who would have been otherwise missed by conventional serum CA-125 assays. Further, the results show that biomarker combinations (e.g., described herein) can distinguish from ovarian cancer patients many non-ovarian cancer patients with elevated serum CA-125 (e.g., ones having benign gynecological tumors), who would have been otherwise falsely diagnosed with ovarian cancer. These results show that serum CA-125 may not necessarily correlate with women’s risk for ovarian cancer, while technologies provided herein can successfully identify ovarian cancer subjects independent of their serum CA-125 levels.

### Example 10: Validation of Exemplary Ovarian Cancer Liquid Biopsy Assays

This example relates to the validation of exemplary ovarian cancer liquid biopsy assays in additional populations/cohorts. Independent validation studies to assess exemplary ovarian cancer diagnostic assays as described herein using additional cohorts of patient samples can be performed. Samples can be obtained from any appropriate source. Technical experts are blinded to sample designations prior to any result analysis, and/or assay results are analyzed by an outside independent technical expert. To ensure sampling from a population representative of the United States, at least approximately 20% of samples can be sourced from non-white ethnicities (United States Census Bureau, 2018), depending on sample availability.

In certain embodiments, patient cohorts analyzed can include: patients at hereditary risk for ovarian cancer prior to undertaking any risk-reducing operations (e.g., patients with familial cases of ovarian cancer and/or BRCA1 or BRCA2 pathological variant carriers who have not undergone bilateral salpingo-oophorectomy or similar procedures, post-menopausal women with chronic conditions associated with abdominal pain (e.g., women >54 years of age with chronic inflammatory conditions, patients with benign gynecological tumors, and/or patients with HGSOC (e.g., individuals with stage I/II ovarian cancer, and individuals with stage III/IV ovarian cancer). In certain embodiments, a biomarker combination (e.g., ones described herein) can provide at least an approximately 80% sensitivity (95% CI: 68.7% to 91.3%) at approximately 98% specificity (95% CI: 95.5% to 100%) in women with hereditary risk. In certain embodiments, a biomarker combination (e.g., ones described herein) can provide at least approximately 70% sensitivity (95% CI: 57% to 83%) at approximately 99.5% specificity (95% CI: 98.2% to 100%) for post-menopausal symptomatic women. Moreover, in certain embodiments, exemplary assays can differentiate between women with benign tumors and those with HGSOC, resulting in few false positives.

In certain embodiments, additional validation studies of exemplary assays as described herein can be conducted utilizing longitudinally-collected samples from independent sources. In some embodiments, samples can be obtained or derived from longitudinally collected blood draws (e.g., blood draws collected at temporally distinct time points) from ovarian cancer cases. In addition, blood draws from age-matched controls can be analyzed. Using exemplary logistic regression models for a 98% specificity cutoff, assessments can be made of how many years prior to diagnosis (e.g., however many years prior to ovarian cancer diagnosis blood draws are available from) exemplary assay are capable of detecting ovarian cancer while maintaining an annual specificity of 98%. Assay sensitivity can be calculated as a function of year prior to diagnosis while ensuring specificity is maintained in the control samples at 98%.

In some embodiments, at least an approximately 80% sensitivity (95% CI: 63.3% to 96.7%) for HGSOC at approximately 98% specificity (95% CI: 93.8% to 100%) for samples collected at the time of diagnosis can be expected. Given that ovarian cancer tumors have been shown to exist as stage I/II disease for four or more years (Brown and Palmer, 2009), exemplary assays can be expected to achieve at least approximately 60% sensitivity (95% CI: 39.5% to 80.5%) for HGSOC two years prior to clinical diagnosis. This would pertain to a PPV of 23.3% in this high-risk patient cohort two years before clinical diagnosis, a PPV far better than the current standard of care for women at hereditary risk.

### References Cited

Balaj, L., Lessard, R., Dai, L., Cho, Y.J., Pomeroy, S.L., Breakefield, X.O. and Skog, J., 2011. Tumour microvesicles contain retrotransposon elements and amplified oncogene sequences. Nature communications, 2, p.180.

Bebelman, M.P., Smit, M.J., Pegtel, D.M., and Baglio, S.R., 2018. Biogenesis and function of extracellular vesicles in cancer. Pharmacology & Therapeutics, 188, pp.1-11.

Brett M. Reid, Jennifer B. Permuth, Thomas A. Sellers., 2017. Epidemiology of ovarian cancer: a review, Cancer Biol Med 2017. doi: 10.20892/j.issn.2095-3941.2016.0084

Buys, S.S., Partridge, E., Black, A., Johnson, C.C., Lamerato, L., Isaacs, C., Reding, D.J., Greenlee, R.T., Yokochi, L.A., Kessel, B. and Crawford, E.D., 2011. Effect of screening on ovarian cancer mortality: the Prostate, Lung, Colorectal and Ovarian (PLCO) cancer screening randomized controlled trial. Jama, 305(22), pp.2295-2303.

Darmanis, S., Nong, R.Y., Hammond, M., Gu, J., Alderborn, A., Vänelid, J., Siegbahn, A., Gustafsdottir, S., Ericsson, O., Landegren, U. and Kamali-Moghaddam, M., 2010. Sensitive plasma protein analysis by microparticle-based proximity ligation assays. Molecular & cellular proteomics, 9(2), pp.327-335.

Howlader N, Noone AM, Krapcho M, Miller D, Brest A, Yu M, Ruhl J, Tatalovich Z, Mariotto A, Lewis DR, Chen HS, Feuer EJ, Cronin KA (eds). SEER Cancer Statistics Review, 1975-2016, National Cancer Institute. Bethesda, MD, https://seer.cancer.gov/csr/1975_2016/, based on November 2018 SEER data submission, posted to the SEER web site, April 2019.

Im, H., Shao, H., Park, Y.I., Peterson, V.M., Castro, C.M., Weissleder, R. and Lee, H., 2014. Label-free detection and molecular profiling of exosomes with a nano-plasmonic sensor. Nature biotechnology, 32(5), p.490.

Jeong, S., Park, J., Pathania, D., Castro, C.M., Weissleder, R. and Lee, H., 2016. Integrated magneto-electrochemical sensor for exosome analysis. ACS nano, 10(2), pp.1802-1809.

Shao, H., Im, H., Castro, C.M., Breakefield, X., Weissleder, R. and Lee, H., 2018. New technologies for analysis of extracellular vesicles. Chemical reviews, 118(4), pp.1917-1950.

Sun, L., Brentnall, A., Patel, S., Buist, D.S., Bowles, E.J., Evans, D.G.R., Eccles, D., Hopper, J., Li, S., Southey, M. and Duffy, S., 2019. A cost-effectiveness analysis of multigene testing for all patients with breast cancer. JAMA oncology.

Torre, L.A., Trabert, B., DeSantis, C.E., Miller, K.D., Samimi, G., Runowicz, C.D., Gaudet, M.M., Jemal, A. and Siegel, R.L., 2018. Ovarian cancer statistics, 2018. CA: a cancer journal for clinicians, 68(4), pp.284-296.

Ursula A. Matulonis, Anil K. Sood, Lesley Fallowfield, Brooke E. Howitt, Jalid Sehouli and Beth Y. Karlan. Ovarian Cancer. Nature Reviews Disease Primers 2016 Aug 25;2:16061. Doi: 10.1038/nrdp.2016.61

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

What is claimed is:

1. A method comprising steps of:
(a) providing or obtaining a blood-derived sample from a subject;
(b) detecting, in the blood-derived sample, extracellular vesicles expressing a first target biomarker signature (“first target biomarker signature-expressing extracellular vesicles”), the first target biomarker signature comprising:
at least one extracellular vesicle-associated membrane-bound polypeptide biomarker and
at least one target biomarker selected from the group consisting of: surface protein biomarkers, intravesicular protein biomarkers, and intravesicular RNA biomarkers, wherein:
the surface protein biomarkers are selected from CLDN6, AQP5, CLDN16, CLDN3, FOLR1, EpCAM, LEMD1, LRRTM1, MUC16, CHODL, CDH6, HTR3A, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof;
the intravesicular protein biomarkers are selected from CRABP2, KLK7, MIF, PRAME, and S100A1, and combinations thereof;
the intravesicular RNA biomarkers are selected from CRABP2, MIF, CLDN6, PRAME, S100A1, KLK7, and combinations thereof;
(c) comparing sample information indicative of level of the first target biomarker signature-expressing extracellular vesicles in the blood-derived sample to reference information including a first reference threshold level;
(d) classifying the subject as having or being susceptible to ovarian cancer when the blood-derived sample shows an elevated level of first target biomarker signature-expressing extracellular vesicles relative to a classification cutoff referencing the first reference threshold level.

2. The method of claim 1, wherein when the at least one target biomarker is selected from one or more of the surface protein biomarkers, the selected surface protein biomarker(s) and the at least one extracellular vesicle-associated membrane-bound polypeptide biomarker are different.

3. The method of claim 1 or 2, wherein the steps of (b) and (c) are repeated for at least a second target biomarker signature, and wherein the classification cutoff references the first reference threshold level and at least a second reference threshold level corresponding to the at least a second target biomarker signature.

4. The method of any one of claims 1-3, wherein the extracellular vesicle-associated membrane-bound polypeptide biomarker is or comprises CLDN3, CLDN6, AQP5, CLDN16, EpCAM, FOLR1, LEMD1, LRRTM1, MUC16, CHODL, CDH6, HTR3A, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof.

5. The method of any one of claims 1-4, wherein the extracellular vesicle-associated membrane-bound polypeptide biomarker is or comprises a SLC34A2 polypeptide, a FOLR1 polypeptide, a MUC16 polypeptide, a CLDN3 polypeptide, a CLDN6 polypeptide, an ALPL polypeptide, a BST2 polypeptide, a MSLN polypeptide, a MUC1 polypeptide, a PTGS1 polypeptide, a sTn glycosylated polypeptide, a TACSTD2 polypeptide, an EpCAM polypeptide, and/or a LRRTM1 polypeptide.

6. The method of any one of claims 1-5, wherein the first and/or second target biomarker signature comprises (i) at least one extracellular vesicle-associated membrane-bound polypeptide biomarker, which is or comprises a SCL34A2 polypeptide and/or a CLDN6 polypeptide; and (ii) at least one target biomarker MUC16.

7. The method of any one of claims 1-5, wherein the first and/or second target biomarker signature comprises (i) at least one extracellular vesicle-associated membrane-bound polypeptide biomarker, which is or comprises a SCL34A2 polypeptide, and (ii) at least one target biomarker FOLR1.

8. The method of any one of claims 1-5, wherein the first and/or second target biomarker signature comprises (i) at least one extracellular vesicle-associated membrane-bound polypeptide biomarker, which is or comprises a SCL34A2 polypeptide, and (ii) at least two target biomarkers, which are or comprise MUC16 and FOLR1.

9. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least one target biomarker MUC16.

10. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least two target biomarkers, which are or comprise MUC16 and CLDN6.

11. The method of any one of claims 1-5, wherein the first and/or second target biomarker signature comprises (i) at least one extracellular vesicle-associated membrane-bound polypeptide biomarker, which is or comprises a FOLR1 polypeptide, and (ii) at least two target biomarkers, which are FOLR1 and CLDN6.

12. The method of any one of claims 1-5, wherein the first and/or second target biomarker signature comprises (i) at least one extracellular vesicle-associated membrane-bound polypeptide biomarker, which is or comprises a FOLR1 polypeptide, and (ii) at least two target biomarkers, which are SLC34A2 and CLDN3.

13. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least two target biomarkers, which are SLC34A2 and MUC16.

14. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least two target biomarkers, which are SLC34A2 and FOLR1.

15. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least two target biomarkers, which are FOLR1 and MUC16.

16. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least two target biomarkers, which are CLDN3 and MUC16.

17. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least two target biomarkers, which are CLDN3 and FOLR1.

18. The method of any one of claims 1-5, wherein the first and/or second target biomarker signature comprises (i) at least one extracellular vesicle-associated membrane-bound polypeptide biomarker, which is or comprises a CLDN3 polypeptide, and (ii) at least one target biomarker FOLR1.

19. The method of any one of claims 1-5, wherein the first and/or second target biomarker signature comprises (i) at least one extracellular vesicle-associated membrane-bound polypeptide biomarker, which is or comprises a LRRTM1 polypeptide, and (ii) at least one target biomarker MUC16.

20. The method of any one of claims 1-5, wherein the first and/or second target biomarker signature comprises (i) at least one extracellular vesicle-associated membrane-bound polypeptide biomarker, which is or comprises an ALPL polypeptide, and (ii) at least one target biomarker FOLR1.

21. The method of any one of claims 1-5, wherein the first and/or second target biomarker signature comprises (i) at least one extracellular vesicle-associated membrane-bound polypeptide biomarker, which is or comprises a BST2 polypeptide, and (ii) at least one target biomarker MUC16.

22. The method of any one of claims 1-5, wherein the first and/or second target biomarker signature comprises (i) at least one extracellular vesicle-associated membrane-bound polypeptide biomarker, which is or comprises a FOLR1 polypeptide, and (ii) at least one target biomarker MSLN.

23. The method of any one of claims 1-5, wherein the first and/or second target biomarker signature comprises (i) at least one extracellular vesicle-associated membrane-bound polypeptide biomarker, which is or comprises a FOLR1 polypeptide, and (ii) at least one target biomarker MUC16.

24. The method of any one of claims 1-5, wherein the first and/or second target biomarker signature comprises (i) at least one extracellular vesicle-associated membrane-bound polypeptide biomarker, which is or comprises a MSLN polypeptide, and (ii) at least one target biomarker MUC16.

25. The method of any one of claims 1-5, wherein the first and/or second target biomarker signature comprises (i) at least one extracellular vesicle-associated membrane-bound polypeptide biomarker, which is or comprises a MSLN polypeptide, and (ii) at least one target biomarker MUC1.

26. The method of any one of claims 1-5, wherein the first and/or second target biomarker signature comprises (i) at least one extracellular vesicle-associated membrane-bound polypeptide biomarker, which is or comprises a MUC1 polypeptide, and (ii) at least one target biomarker sTn glycosylated polypeptide.

27. The method of any one of claims 1-5, wherein the first and/or second target biomarker signature comprises (i) at least one extracellular vesicle-associated membrane-bound polypeptide biomarker, which is or comprises a MUC1 polypeptide, and (ii) at least one target biomarker MUC16.

28. The method of any one of claims 1-5, wherein the first and/or second target biomarker signature comprises (i) at least one extracellular vesicle-associated membrane-bound polypeptide biomarker, which is or comprises a MUC1 polypeptide, and (ii) at least one target biomarker FOLR1.

29. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least one target biomarker sTn glycosylated polypeptide.

30. The method of any one of claims 1-5, wherein the first and/or second target biomarker signature comprises (i) at least one extracellular vesicle-associated membrane-bound polypeptide biomarker, which is or comprises a PTGS1 polypeptide, and (ii) at least one target biomarker MUC16.

31. The method of any one of claims 1-5, wherein the first and/or second target biomarker signature comprises (i) at least one extracellular vesicle-associated membrane-bound polypeptide biomarker, which is or comprises a SLC34A2 polypeptide, and (ii) at least one target biomarker MUC16.

32. The method of any one of claims 1-5, wherein the first and/or second target biomarker signature comprises (i) at least one extracellular vesicle-associated membrane-bound polypeptide biomarker, which is or comprises a sTn glycosylated polypeptide, and (ii) at least one target biomarker FOLR1.

33. The method of any one of claims 1-5, wherein the first and/or second target biomarker signature comprises (i) at least one extracellular vesicle-associated membrane-bound polypeptide biomarker, which is or comprises a sTn glycosylated polypeptide, and (ii) at least one target biomarker MSLN.

34. The method of any one of claims 1-5, wherein the first and/or second target biomarker signature comprises (i) at least one extracellular vesicle-associated membrane-bound polypeptide biomarker, which is or comprises a TACSTD2 polypeptide, and (ii) at least one target biomarker MUC16.

35. The method of any one of claims 1-5, wherein the first and/or second target biomarker signature comprises (i) at least one extracellular vesicle-associated membrane-bound polypeptide biomarker, which is or comprises a TACSTD2 polypeptide, and (ii) at least one target biomarker sTn glycosylated polypeptide.

36. The method of any one of claims 1-5, wherein the first and/or second target biomarker signature comprises (i) at least one extracellular vesicle-associated membrane-bound polypeptide biomarker, which is or comprises a TACSTD2 polypeptide, and (ii) at least two target biomarkers, which are MUC16 and a sTn glycosylated polypeptide.

37. The method of any one of claims 1-5, wherein the first and/or second target biomarker signature comprises (i) at least one extracellular vesicle-associated membrane-bound polypeptide biomarker, which is or comprises a TACSTD2 polypeptide, and (ii) at least two target biomarkers, which are MUC16 and MUC1.

38. The method of any one of claims 1-5, wherein the first and/or second target biomarker signature comprises (i) at least one extracellular vesicle-associated membrane-bound polypeptide biomarker, which is or comprises a TACSTD2 polypeptide, and (ii) at least two target biomarkers, which are MUC16 and MSLN.

39. The method of any one of claims 1-5, wherein the first and/or second target biomarker signature comprises (i) at least one extracellular vesicle-associated membrane-bound polypeptide biomarker, which is or comprises a TACSTD2 polypeptide, and (ii) at least two target biomarkers, which are MUC16 and FOLR1.

40. The method of any one of claims 1-5, wherein the first and/or second target biomarker signature comprises (i) at least one extracellular vesicle-associated membrane-bound polypeptide biomarker, which is or comprises a TACSTD2 polypeptide, and (ii) at least two target biomarkers, which are a sTn glycosylated polypeptide and MUC1.

41. The method of any one of claims 1-5, wherein the first and/or second target biomarker signature comprises (i) at least one extracellular vesicle-associated membrane-bound polypeptide biomarker, which is or comprises a TACSTD2 polypeptide, and (ii) at least two target biomarkers, which are a sTn glycosylated polypeptide and MSLN.

42. The method of any one of claims 1-5, wherein the first and/or second target biomarker signature comprises (i) at least one extracellular vesicle-associated membrane-bound polypeptide biomarker, which is or comprises a TACSTD2 polypeptide, and (ii) at least two target biomarkers, which are a sTn glycosylated polypeptide and FOLR1.

43. The method of any one of claims 1-5, wherein the first and/or second target biomarker signature comprises (i) at least one extracellular vesicle-associated membrane-bound polypeptide biomarker, which is or comprises a TACSTD2 polypeptide, and (ii) at least two target biomarkers, which are MUC1 and MSLN.

44. The method of any one of claims 1-5, wherein the first and/or second target biomarker signature comprises (i) at least one extracellular vesicle-associated membrane-bound polypeptide biomarker, which is or comprises a MUC1 polypeptide, and (ii) at least two target biomarkers, which are MUC16 and a sTn glycosylated polypeptide.

45. The method of any one of claims 1-5, wherein the first and/or second target biomarker signature comprises (i) at least one extracellular vesicle-associated membrane-bound polypeptide biomarker, which is or comprises a MUC1 polypeptide, and (ii) at least two target biomarkers, which are MUC16 and MSLN.

46. The method of any one of claims 1-5, wherein the first and/or second target biomarker signature comprises (i) at least one extracellular vesicle-associated membrane-bound polypeptide biomarker, which is or comprises a MUC1 polypeptide, and (ii) at least two target biomarkers, which are MUC16 and FOLR1.

47. The method of any one of claims 1-5, wherein the first and/or second target biomarker signature comprises (i) at least one extracellular vesicle-associated membrane-bound polypeptide biomarker, which is or comprises a MUC1 polypeptide, and (ii) at least two target biomarkers, which are a sTn glycosylated polypeptide and MSLN.

48. The method of any one of claims 1-5, wherein the first and/or second target biomarker signature comprises (i) at least one extracellular vesicle-associated membrane-bound polypeptide biomarker, which is or comprises a MUC1 polypeptide, and (ii) at least two target biomarkers, which are a sTn glycosylated polypeptide and FOLR1.

49. The method of any one of claims 1-5, wherein the first and/or second target biomarker signature comprises (i) at least one extracellular vesicle-associated membrane-bound polypeptide biomarker, which is or comprises a MUC1 polypeptide, and (ii) at least two target biomarkers, which are MSLN and FOLR1.

50. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least two target biomarkers, which are a sTn glycosylated polypeptide and MUC1.

51. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least two target biomarkers, which are a sTn glycosylated polypeptide and MSLN.

52. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least two target biomarkers, which are a sTn glycosylated polypeptide and FOLR1.

53. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least two target biomarkers, which are MUC1 and MSLN.

54. The method of any one of claims 1-5, wherein the first and/or second target biomarker signature comprises (i) at least one extracellular vesicle-associated membrane-bound polypeptide biomarker, which is or comprises a MUC16 polypeptide, and (ii) at least two target biomarkers, which are MSLN and FOLR1.

55. The method of any one of claims 1-5, wherein the first and/or second target biomarker signature comprises (i) at least one extracellular vesicle-associated membrane-bound polypeptide biomarker, which is or comprises a sTn glycosylated polypeptide, and (ii) at least two target biomarkers, which are MUC16 and MUC1.

56. The method of any one of claims 1-5, wherein the first and/or second target biomarker signature comprises (i) at least one extracellular vesicle-associated membrane-bound polypeptide biomarker, which is or comprises a sTn glycosylated polypeptide, and (ii) at least two target biomarkers, which are MUC16 and MSLN.

57. The method of any one of claims 1-5, wherein the first and/or second target biomarker signature comprises (i) at least one extracellular vesicle-associated membrane-bound polypeptide biomarker, which is or comprises a sTn glycosylated polypeptide, and (ii) at least two target biomarkers, which are MUC16 and FOLR1.

58. The method of any one of claims 1-5, wherein the first and/or second target biomarker signature comprises (i) at least one extracellular vesicle-associated membrane-bound polypeptide biomarker, which is or comprises a sTn glycosylated polypeptide, and (ii) at least two target biomarkers, which are MUC1 and MSLN.

59. The method of any one of claims 1-5, wherein the first and/or second target biomarker signature comprises (i) at least one extracellular vesicle-associated membrane-bound polypeptide biomarker, which is or comprises a sTn glycosylated polypeptide, and (ii) at least two target biomarkers, which are MUC1 and FOLR1.

60. The method of any one of claims 1-5, wherein the first and/or second target biomarker signature comprises (i) at least one extracellular vesicle-associated membrane-bound polypeptide biomarker, which is or comprises a sTn glycosylated polypeptide, and (ii) at least two target biomarkers, which are MSLN and FOLR1.

61. The method of any one of claims 1-5, wherein the first and/or second target biomarker signature comprises (i) at least one extracellular vesicle-associated membrane-bound polypeptide biomarker, which is or comprises a MSLN polypeptide, and (ii) at least two target biomarkers, which are MUC16 and a sTn glycosylated polypeptide.

62. The method of any one of claims 1-5, wherein the first and/or second target biomarker signature comprises (i) at least one extracellular vesicle-associated membrane-bound polypeptide biomarker, which is or comprises a MSLN polypeptide, and (ii) at least two target biomarkers, which are MUC16 and MUC1.

63. The method of any one of claims 1-5, wherein the first and/or second target biomarker signature comprises (i) at least one extracellular vesicle-associated membrane-bound polypeptide biomarker, which is or comprises a MSLN polypeptide, and (ii) at least two target biomarkers, which are MUC16 and FOLR1.

64. The method of any one of claims 1-5, wherein the first and/or second target biomarker signature comprises (i) at least one extracellular vesicle-associated membrane-bound polypeptide biomarker, which is or comprises a MSLN polypeptide, and (ii) at least two target biomarkers, which are a sTn glycosylated polypeptide and MUC1.

65. The method of any one of claims 1-5, wherein the first and/or second target biomarker signature comprises (i) at least one extracellular vesicle-associated membrane-bound polypeptide biomarker, which is or comprises a MSLN polypeptide, and (ii) at least two target biomarkers, which are a sTn glycosylated polypeptide and FOLR1.

66. The method of any one of claims 1-5, wherein the first and/or second target biomarker signature comprises (i) at least one extracellular vesicle-associated membrane-bound polypeptide biomarker, which is or comprises a MSLN polypeptide, and (ii) at least two target biomarkers, which are MUC1 and FOLR1.

67. The method of any one of claims 1-5, wherein the first and/or second target biomarker signature comprises (i) at least one extracellular vesicle-associated membrane-bound polypeptide biomarker, which is or comprises a SLC34A2 polypeptide, and (ii) at least two target biomarkers, which are MUC16 and MSLN.

68. The method of any one of claims 1-5, wherein the first and/or second target biomarker signature comprises (i) at least one extracellular vesicle-associated membrane-bound polypeptide biomarker, which is or comprises a SCL34A2 polypeptide, and (ii) at least two target biomarkers, which are a sTn glycosylated polypeptide and MUC16.

69. The method of any one of claims 1-5, wherein the first and/or second target biomarker signature comprises (i) at least one extracellular vesicle-associated membrane-bound polypeptide biomarker, which is or comprises a SCL34A2 polypeptide, and (ii) at least two target biomarkers, which are a sTn glycosylated polypeptide and FOLR1.

70. The method of any one of claims 1-69, wherein the first and/or second reference threshold level is determined by levels of target biomarker signature-expressing extracellular vesicles observed in comparable samples from a population of non-cancer subjects.

71. The method of claim 70, wherein the population of non-cancer subjects comprises one or more of the following subject populations: healthy women subjects, women subjects diagnosed with benign tumors, and women subjects with non-ovarian-related diseases, disorders, and/or conditions.

72. The method of any one of claims 1-71, wherein the blood-derived sample has been subjected to size exclusion chromatography to isolate (e.g., directly from the blood-derived sample) nanoparticles having a size range of interest that includes extracellular vesicles.

73. The method of any one of claims 1-72, wherein the step of detecting comprises a capture assay.

74. The method of claim 73, wherein the capture assay involves contacting the blood-derived sample with a capture agent comprising a target-capture moiety that binds to the at least one extracellular vesicle-associated membrane-bound polypeptide biomarker.

75. The method of claim 74, wherein the capture agent is or comprises a solid substrate comprising the target-capture moiety conjugated thereto.

76. The method of claim 75, wherein the solid substrate comprises a magnetic bead.

77. The method of any one of claims 74–76, wherein the target-capture moiety is or comprises an antibody agent.

78. The method of any one of claims 1-77, wherein the step of detecting comprises a detection assay.

79. The method of any one of claims 1-78, wherein the step of detecting comprises a capture assay and a detection assay, the capture assay being performed prior to the detection assay.

80. The method of any one of claims 78-79, wherein when the first and/or second target biomarker signature comprises at least one intravesicular RNA biomarkers, the detection assay involves reverse transcription qPCR.

81. The method of any one of claims 78-80, wherein when the first and/or second target biomarker signature comprises at least one intravesicular protein biomarker, the target biomarker signature-expressing extracellular vesicles are processed involving fixation and/or permeabilization prior to the detection assay.

82. The method of any one of claims 78-81, wherein when the first and/or second target biomarker signature comprises at least one surface protein biomarker and/or intravesicular protein biomarker, the detection assay involves an immunoassay (including, e.g., immuno-PCR, and/or proximity ligation assay).

83. The method of claim 82, wherein the detection assay involves a proximity ligation assay.

84. The method of claim 83, wherein the proximity ligation assay comprises the steps of:
(a) contacting the target biomarker signature-expressing extracellular vesicles that express the at least one extracellular vesicle-associated membrane-bound polypeptide biomarker (“extracellular vesicle-associated membrane-bound polypeptide biomarker-expressing extracellular vesicles”) with a set of detection probes, each directed to a target biomarker of the target biomarker signature, which set comprises at least two detection probes, so that a combination comprising the extracellular vesicles and the set of detection probes is generated,
wherein the detection probes each comprise:
(i) a target binding moiety directed to the target biomarker of the target biomarker signature; and
(ii) an oligonucleotide domain coupled to the target binding moiety, the oligonucleotide domain comprising a double-stranded portion and a single-stranded overhang portion extended from one end of the oligonucleotide domain,
wherein the single-stranded overhang portions of the detection probes are characterized in that they can hybridize to each other when the detection probes are bound to the same extracellular vesicle,
(b) maintaining the combination under conditions that permit binding of the set of detection probes to their respective targets on the extracellular vesicles such that the at least two detection probes can bind to the same extracellular vesicle that express the target biomarker signature to form a double-stranded complex;
(c) contacting the double-stranded complex with a nucleic acid ligase to generate a ligated template;
(d) detecting the ligated template, wherein presence of the ligated template is indicative of presence in the blood-derived sample of the target biomarker signature-expressing extracellular vesicles; and
(e) optionally repeating steps a through d for at least an additional target biomarker signature.

85. The method of claim 84, wherein the target binding moiety of the at least two detection probes is each directed to MUC16.

86. The method of claim 84, wherein the target binding moiety of the at least two detection probes is each directed to FOLR1.

87. The method of claim 84, wherein the target binding moiety of the at least two detection probes is each directed to MSLN.

88. The method of claim 84, wherein the target binding moiety of the at least two detection probes is each directed to ALPL.

89. The method of claim 84, wherein the target binding moiety of the at least two detection probes is each directed to MUC1.

90. The method of claim 84, wherein the target binding moiety of the at least two detection probes is each directed to a sTn glycosylated polypeptide.

91. The method of claim 84, wherein the target binding moiety of the at least two detection probes is each directed to BST2.

92. The method of claim 84, wherein the target binding moiety of the at least two detection probes is each directed to PTGS1.

93. The method of claim 84, wherein the target binding moiety of the at least two detection probes is each directed to SLC34A2.

94. The method of claim 84, wherein the target binding moiety of the at least two detection probes is each directed to TACSTD2.

95. The method of claim 84, wherein the target binding moieties of the at least two detection probes are directed to MUC16 and FOLR1, respectively.

96. The method of claim 84, wherein the target binding moieties of the at least two detection probes are directed to MUC16 and CLDN6 respectively.

97. The method of claim 84, wherein the target binding moieties of the at least two detection probes are directed to FOLR1 and CLDN6 respectively.

98. The method of claim 84, wherein the target binding moieties of the at least two detection probes are directed to SLC34A2 and CLDN3 respectively.

99. The method of claim 84, wherein the target binding moieties of the at least two detection probes are directed to SLC34A2 and MUC16 respectively.

100. The method of claim 84, wherein the target binding moieties of the at least two detection probes are directed to CLDN3 and MUC16 respectively.

101. The method of claim 84, wherein the target binding moieties of the at least two detection probes are directed to CLDN3 and FOLR1 respectively.

102. The method of claim 84, wherein the target binding moieties of the at least two detection probes are directed to ALPL and FOLR1 respectively.

103. The method of claim 84, wherein the target binding moieties of the at least two detection probes are directed to BST2 and MUC16 respectively.

104. The method of claim 84, wherein the target binding moieties of the at least two detection probes are directed to FOLR1 and MSLN respectively.

105. The method of claim 84, wherein the target binding moieties of the at least two detection probes are directed to FOLR1 and MUC16 respectively.

106. The method of claim 84, wherein the target binding moieties of the at least two detection probes are directed to MSLN and MUC16 respectively.

107. The method of claim 84, wherein the target binding moieties of the at least two detection probes are directed to MSLN and MUC1 respectively.

108. The method of claim 84, wherein the target binding moieties of the at least two detection probes are directed to a sTn glycosylated polypeptide and MUC1 respectively.

109. The method of claim 84, wherein the target binding moieties of the at least two detection probes are directed to MUC1 and MUC16 respectively.

110. The method of claim 84, wherein the target binding moieties of the at least two detection probes are directed to MUC1 and FOLR1 respectively.

111. The method of claim 84, wherein the target binding moieties of the at least two detection probes are directed to a sTn glycosylated polypeptide and MUC16 respectively.

112. The method of claim 84, wherein the target binding moieties of the at least two detection probes are directed to PTGS1 and MUC16 respectively.

113. The method of claim 84, wherein the target binding moieties of the at least two detection probes are directed to SLC34A2 and FOLR1 respectively.

114. The method of claim 84, wherein the target binding moieties of the at least two detection probes are directed to a sTn glycosylated polypeptide and FOLR1 respectively.

115. The method of claim 84, wherein the target binding moieties of the at least two detection probes are directed to a sTn glycosylated polypeptide and MSLN respectively.

116. The method of claim 84, wherein the target binding moieties of the at least two detection probes are directed to TACSTD2 and MUC16 respectively.

117. The method of claim 84, wherein the target binding moieties of the at least two detection probes are directed to a sTn glycosylated polypeptide and TACSTD2 respectively.

118. The method of any one of claims 85-117, wherein the oligonucleotide domain of the at least two detection probes are different.

119. The method of any one of claims 77-118, wherein the target-capture moiety of the capture assay is or comprises at least one antibody agent directed to a SLC34A2 polypeptide.

120. The method of any one of claims 77-118, wherein the target-capture moiety of the capture assay is or comprises at least one antibody agent directed to a MUC16 polypeptide.

121. The method of any one of claims 77-118, wherein the target-capture moiety of the capture assay is or comprises at least one antibody agent directed to a FOLR1 polypeptide.

122. The method of any one of claims 77-118, wherein the target-capture moiety of the capture assay is or comprises at least one antibody agent directed to a MSLN polypeptide.

123. The method of any one of claims 77-118, wherein the target-capture moiety of the capture assay is or comprises at least one antibody agent directed to a sTn glycosylated polypeptide.

124. The method of any one of claims 77-118, wherein the target-capture moiety of the capture assay is or comprises at least one antibody agent directed to a MUC1 polypeptide.

125. The method of any one of claims 77-118, wherein the target-capture moiety of the capture assay is or comprises at least one antibody agent directed to a TACSTD2 polypeptide.

126. The method of any one of claims 77-118, wherein the target-capture moiety of the capture assay is or comprises at least one antibody agent directed to a PTGS1 polypeptide.

127. The method of any one of claims 77-118, wherein the target-capture moiety of the capture assay is or comprises at least one antibody agent directed to a BST2 polypeptide.

128. The method of any one of claims 77-118, wherein the target-capture moiety of the capture assay is or comprises at least one antibody agent directed to an ALPL polypeptide.

129. The method of any one of claims 77-118, wherein the target-capture moiety of the capture assay is or comprises at least one antibody agent directed to a LRRTM1 polypeptide.

130. The method of any one of claims 77-118, wherein the target-capture moiety of the capture assay is or comprises at least one antibody agent directed to a CLDN3 polypeptide.

131. The method of any one of claims 1-130, wherein the method is performed to screen for early-stage ovarian cancer, late-stage ovarian cancer, or recurrent ovarian cancer in the subject.

132. The method of any one of claims 1-131, wherein the subject is determined to have a normal serum CA-125 level.

133. The method of any one of claims 1-132, wherein the subject is determined to have a serum CA-125 level of equal to or higher than a normal serum CA-125 level.

134. The method of any one of claims 1-133, wherein the subject has at least one or more of the following characteristics:
(i) an asymptomatic female (e.g., woman) who is susceptible to ovarian cancer (e.g., at an average population risk (i.e., without hereditary risk) or with hereditary risk for ovarian cancer);
(ii) a post-menopausal woman;
(iii) a female (e.g., woman) with a family history of breast and/or ovarian cancer (e.g., a female (e.g., woman) having one or more first-degree relatives with a history of breast cancer and/or ovarian cancer);
(iv) a female (e.g., woman) determined to have one or more germline mutations in ATM, BRCA1, BRCA2, CDKN2A, MSH2, MLH1, MSH2, EPCAM, PALB2, STK11, TP53, BARD, CHEK2, MRE11A, RAD50, RAD51C, RAD51D and combinations thereof;
(v) a female (e.g., woman) with breast cancer determined to have germline mutations in BRCA1, BRCA2 and/or PALB2;
(vi) an elderly woman e.g., age 65 or above;
(vii) a female (e.g., woman) with one or more non-specific symptoms of ovarian cancer, optionally wherein at least one of the non-specific symptoms is similar to one or more symptoms for irritable bowel syndrome; and
(viii) a female (e.g., woman) recommended for CA-125/transvaginal ultrasound (TVUS) periodic screening;
(ix) a female (e.g., woman) diagnosed with an imaging-confirmed adnexal mass;
(x) a female (e.g., woman) at hereditary risk before undergoing a risk-reducing bilateral salpingo-oophorectomy;
(xi) a female (e.g., woman) with a benign gynecological tumor;
(xii) a female (e.g., woman) who has been previously treated for ovarian cancer; and
(xiii) a female (e.g., woman) with life-history associated risk for ovarian cancer.

135. The method of any one of claims 1-134, wherein the method is used in combination with one or more of the following diagnostic assays:
(i) the subject’s annual physical examination (e.g., including a HPV, and/or Pap smear screening for cervical cancer and a mammogram screening for breast cancer).
(ii) serum CA-125 and/or TVUS screening test;
(iii) a genetic assay to screen blood plasma for genetic mutations in circulating tumor DNA and/or protein biomarkers linked to cancer;
(iv) an assay involving immunofluorescent staining to identify cell phenotype and marker expression, followed by amplification and analysis by next-generation sequencing; and
(v) BRCA1 and/or BRCA2 germline and somatic mutation assays, or assays involving cell-free tumor DNA, liquid biopsy, serum protein and cell-free DNA, OVA1 and OVERA tests, and/or circulating tumor cells.

136. The method of any one of claims 1-135, wherein the ovarian cancer is high-grade serous ovarian cancer, endometrioid ovarian cancer, clear-cell ovarian cancer, low-grade serous ovarian cancer, or mucinous ovarian cancer.

137. The method of any one of claims 1-136, wherein the ovarian cancer is high-grade serous ovarian cancer.

138. The method of claim 137, the high-grade serous ovarian cancer is at an early stage.

139. The method of any one of claims 1-138, wherein the method is performed to monitor an ovarian cancer patient for response to treatment of an anti-ovarian cancer therapy (e.g., olaparib, cisplatin, rucaparib, niraparib, talazoparib) and/or for cancer recurrence/metastasis.

140. A kit for detection of ovarian cancer comprising:
(a) a capture agent comprising a target-capture moiety directed to an extracellular vesicle-associated membrane-bound polypeptide biomarker; and
(b) a set of detection probes, which set comprises at least two detection probes each directed to a target biomarker of a target biomarker signature for ovarian cancer, wherein the detection probes each comprise:
(i) a target binding moiety directed at the target biomarker of the target biomarker signature for ovarian cancer; and
(ii) an oligonucleotide domain coupled to the target binding moiety, the oligonucleotide domain comprising a double-stranded portion and a single-stranded overhang portion extended from one end of the oligonucleotide domain,
wherein the single-stranded overhang portions of the at least two detection probes are characterized in that they can hybridize to each other when the at least two detection probes are bound to the same extracellular vesicle;
wherein the target biomarker signature for ovarian cancer comprises:
at least one extracellular vesicle-associated membrane-bound polypeptide biomarker and
at least one target biomarker selected from the group consisting of: surface protein biomarkers, intravesicular protein biomarkers, and intravesicular RNA biomarkers, wherein:
the surface protein biomarkers are selected from CLDN6, CLDN3, AQP5, CLDN16, EpCAM, FOLR1, LEMD1, LRRTM1, MUC16, CHODL, CDH6, HTR3A, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof;
the intravesicular protein biomarkers are selected from CRABP2, KLK7, MIF, PRAME, and S100A1, and combinations thereof;
the intravesicular RNA biomarkers are selected from CRABP2, MIF, CLDN6, PRAME, S100A1, KLK7, and combinations thereof;.

141. The kit of claim 140, wherein when the at least one target biomarker is selected from one or more of the surface protein biomarkers, the selected surface protein biomarker(s) and the at least one extracellular vesicle-associated membrane-bound polypeptide biomarker are different.

142. The kit of claim 140 or 141, wherein the extracellular vesicle-associated membrane-bound polypeptide biomarker is or comprises CLDN3, CLDN6, AQP5, CLDN16, EpCAM, FOLR1, LEMD1, LRRTM1, MUC16, CHODL, CDH6, HTR3A, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof.

143. The kit of any one of claims 47-49, wherein the extracellular vesicle-associated membrane-bound polypeptide biomarker is or comprises a SLC34A2 polypeptide, a MUC16 polypeptide, an EpCAM polypeptide, a FOLR1 polypeptide, a CLDN3 polypeptide, a CLDN6 polypeptide, an ALPL polypeptide, a BST2 polypeptide, a MSLN polypeptide, a MUC1 polypeptide, a PTGS1 polypeptide, a sTn glycosylated polypeptide, a TACSTD2 polypeptide, and/or a LRRTM1 polypeptide.

144. The kit of any one of claims 140-143, wherein the target binding moiety of the at least two detection probes is each directed to the same target biomarker of the target biomarker signature.

145. The kit of claim 144, wherein the same target biomarker is or comprises MUC16.

146. The kit of claim 144, wherein the same target biomarker is or comprises FOLR1.

148. The kit of claim 144, wherein the same target biomarker is or comprises MSLN.

149. The kit of claim 144, wherein the same target biomarker is or comprises ALPL.

150. The kit of claim 144, wherein the same target biomarker is or comprises MUC1.

151. The kit of claim 144, wherein the same target biomarker is or comprises a sTn glycosylated polypeptide.

152. The kit of claim 144, wherein the same target biomarker is or comprises BST2.

153. The kit of claim 144, wherein the same target biomarker is or comprises PTGS1.

154. The kit of claim 144, wherein the same target biomarker is or comprises SLC34A2.

155. The kit of claim 144, wherein the same target biomarker is or comprises TACSTD2.

156. The kit of any one of claims 140-155, wherein the oligonucleotide domain of the at least two detection probes are different.

157. The kit of any one of claims 140-143, wherein the target binding moiety of the at least two detection probes is each directed to a distinct target biomarker of the target biomarker signature.

158. The kit of any one of claims 140-143, wherein the target binding moieties of the at least two detection probes are directed to MUC16 and FOLR1 respectively.

159. The kit of any one of claims 140-143, wherein the target binding moieties of the at least two detection probes are directed to MUC16 and CLDN6 respectively.

160. The kit of any one of claims 140-143, wherein the target binding moieties of the at least two detection probes are directed to FOLR1 and CLDN6 respectively.

161. The kit of any one of claims 140-143, wherein the target binding moieties of the at least two detection probes are directed to SLC34A2 and CLDN3 respectively.

162. The kit of any one of claims 140-143, wherein the target binding moieties of the at least two detection probes are directed to SLC34A2 and MUC16 respectively.

163. The kit of any one of claims 140-143, wherein the target binding moieties of the at least two detection probes are directed to CLDN3 and MUC16 respectively.

164. The kit of any one of claims 140-143, wherein the target binding moieties of the at least two detection probes are directed to CLDN3 and FOLR1 respectively.

165. The kit of any one of claims 140-143, wherein the target binding moieties of the at least two detection probes are directed to ALPL and FOLR1 respectively.

166. The kit of any one of claims 140-143, wherein the target binding moieties of the at least two detection probes are directed to BST2 and MUC16 respectively.

167. The kit of any one of claims 140-143, wherein the target binding moieties of the at least two detection probes are directed to FOLR1 and MSLN respectively.

168. The kit of any one of claims 140-143, wherein the target binding moieties of the at least two detection probes are directed to FOLR1 and MUC16 respectively.

169. The kit of any one of claims 140-143, wherein the target binding moieties of the at least two detection probes are directed to MSLN and MUC16 respectively.

170. The kit of any one of claims 140-143, wherein the target binding moieties of the at least two detection probes are directed to MSLN and MUC1 respectively.

171. The kit of any one of claims 140-143, wherein the target binding moieties of the at least two detection probes are directed to a sTn glycosylated polypeptide and MUC1 respectively.

172. The kit of any one of claims 140-143, wherein the target binding moieties of the at least two detection probes are directed to MUC1 and MUC16 respectively.

173. The kit of any one of claims 140-143, wherein the target binding moieties of the at least two detection probes are directed to MUC1 and FOLR1 respectively.

174. The kit of any one of claims 140-143, wherein the target binding moieties of the at least two detection probes are directed to a sTn glycosylated polypeptide and MUC16 respectively.

175. The kit of any one of claims 140-143, wherein the target binding moieties of the at least two detection probes are directed to PTGS1 and MUC16 respectively.

176. The kit of any one of claims 140-143, wherein the target binding moieties of the at least two detection probes are directed to SLC34A2 and FOLR1 respectively.

177. The kit of any one of claims 140-143, wherein the target binding moieties of the at least two detection probes are directed to a sTn glycosylated polypeptide and FOLR1 respectively.

178. The kit of any one of claims 140-143, wherein the target binding moieties of the at least two detection probes are directed to a sTn glycosylated polypeptide and MSLN respectively.

179. The kit of any one of claims 140-143, wherein the target binding moieties of the at least two detection probes are directed to TACSTD2 and MUC16 respectively.

180. The kit of any one of claims 140-143, wherein the target binding moieties of the at least two detection probes are directed to a sTn glycosylated polypeptide and TACSTD2 respectively.

181. The method of any one of claims 140-180, wherein the capture agent comprising a target-capture moiety directed to an extracellular vesicle-associated membrane-bound polypeptide biomarker comprises at least one antibody agent directed to a SLC34A2 polypeptide.

182. The method of any one of claims 140-180, wherein the capture agent comprising a target-capture moiety directed to an extracellular vesicle-associated membrane-bound polypeptide biomarker comprises at least one antibody agent directed to a MUC16 polypeptide.

183. The method of any one of claims 140-180, wherein the capture agent comprising a target-capture moiety directed to an extracellular vesicle-associated membrane-bound polypeptide biomarker comprises at least one antibody agent directed to a FOLR1 polypeptide.

184. The method of any one of claims 140-180, wherein the capture agent comprising a target-capture moiety directed to an extracellular vesicle-associated membrane-bound polypeptide biomarker comprises at least one antibody agent directed to a MSLN polypeptide.

185. The method of any one of claims 140-180, wherein the capture agent comprising a target-capture moiety directed to an extracellular vesicle-associated membrane-bound polypeptide biomarker comprises at least one antibody agent directed to a sTn glycosylated polypeptide.

186. The method of any one of claims 140-180, wherein the capture agent comprising a target-capture moiety directed to an extracellular vesicle-associated membrane-bound polypeptide biomarker comprises at least one antibody agent directed to a MUC1 polypeptide.

187. The method of any one of claims 140-180, wherein the capture agent comprising a target-capture moiety directed to an extracellular vesicle-associated membrane-bound polypeptide biomarker comprises at least one antibody agent directed to a TACSTD2 polypeptide.

188. The method of any one of claims 140-180, wherein the capture agent comprising a target-capture moiety directed to an extracellular vesicle-associated membrane-bound polypeptide biomarker comprises at least one antibody agent directed to a PTGS1 polypeptide.

189. The method of any one of claims 140-180, wherein the capture agent comprising a target-capture moiety directed to an extracellular vesicle-associated membrane-bound polypeptide biomarker comprises at least one antibody agent directed to a BST2 polypeptide.

190. The method of any one of claims 140-180, wherein the capture agent comprising a target-capture moiety directed to an extracellular vesicle-associated membrane-bound polypeptide biomarker comprises at least one antibody agent directed to an ALPL polypeptide.

191. The method of any one of claims 140-180, wherein the capture agent comprising a target-capture moiety directed to an extracellular vesicle-associated membrane-bound polypeptide biomarker comprises at least one antibody agent directed to a LRRTM1 polypeptide.

192. The method of any one of claims 140-180, wherein the capture agent comprising a target-capture moiety directed to an extracellular vesicle-associated membrane-bound polypeptide biomarker comprises at least one antibody agent directed to a CLDN3 polypeptide.

193. The kit of any one of claims 140-192, further comprising at least one additional reagent (e.g., a ligase, a fixation agent, and/or a permeabilization agent).

194. The kit of any one of claims 140-146, or 157-161, comprising:
(a) a first capture agent comprising a target-capture moiety directed to a SLC34A2 polypeptide;
(b) a second capture agent comprising a target-capture moiety directed to a MUC16 polypeptide;
(c) at least three sets of detection probes, wherein the detection probes each comprise:
(i) a target binding moiety directed at a target surface protein biomarker; and
(ii) an oligonucleotide domain coupled to the target binding moiety, the oligonucleotide domain comprising a double-stranded portion and a single-stranded overhang portion extended from one end of the oligonucleotide domain,
wherein the single-stranded overhang portions of the at least two detection probes are characterized in that they can hybridize to each other when the at least two detection probes are bound to the same extracellular vesicle;
wherein: at least two detection probes in a first set are each directed at a MUC16 target surface protein;
at least two detection probes in a second set are each directed at a FOLR1 target surface protein; and
at least two detection probes in a third set are directed at a MUC16 polypeptide and a FOLR1 polypeptide, respectively.

195. The kit of any one of claims 140-193, comprising:
(a) a first capture agent comprising a target-capture moiety;
(b) a second capture agent comprising a target-capture moiety;
(c) at least two sets of detection probes, wherein the detection probes each comprise:
(i) a target binding moiety directed at a target surface protein biomarker; and
(ii) an oligonucleotide domain coupled to the target binding moiety, the oligonucleotide domain comprising a double-stranded portion and a single-stranded overhang portion extended from one end of the oligonucleotide domain,
wherein the single-stranded overhang portions of the at least two detection probes are characterized in that they can hybridize to each other when the at least two detection probes are bound to the same extracellular vesicle.

196. The kit of any one of claims 140-193, comprising:
(a) a first capture agent comprising a target-capture moiety;
(b) a second capture agent comprising a target-capture moiety;
(c) a third capture agent comprising a target-capture moiety;
(d) at least three sets of detection probes, wherein the detection probes each comprise:
(i) a target binding moiety directed at a target surface protein biomarker; and
(ii) an oligonucleotide domain coupled to the target binding moiety, the oligonucleotide domain comprising a double-stranded portion and a single-stranded overhang portion extended from one end of the oligonucleotide domain,
wherein the single-stranded overhang portions of the at least two detection probes are characterized in that they can hybridize to each other when the at least two detection probes are bound to the same extracellular vesicle.

197. A complex comprising:
(a) an extracellular vesicle expressing a target biomarker signature for ovarian cancer, wherein the which comprises:
at least one extracellular vesicle-associated membrane-bound polypeptide biomarker and
at least one target biomarker selected from the group consisting of: surface protein biomarkers, intravesicular protein biomarkers, and intravesicular RNA biomarkers, wherein:
the surface protein biomarkers are selected from CLDN3, CLDN6, AQP5, CLDN16, EpCAM, FOLR1, LEMD1, LRRTM1, MUC16, CHODL, CDH6, HTR3A, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof;
the intravesicular protein biomarkers are selected from CRABP2, KLK7, MIF, PRAME, and S100A1, and combinations thereof;
the intravesicular RNA biomarkers are selected from CRABP2, MIF, CLDN6, PRAME, S100A1, KLK7, and combinations thereof;
wherein the extracellular vesicle is immobilized onto a solid substrate comprising a target-capture moiety directed to the extracellular vesicle-associated membrane-bound polypeptide;
(b) a first detection probe and a second detection probe each bound to the extracellular vesicle, wherein each detection probe comprises:
(i) a target binding moiety directed to one of the target biomarker of the tumor target biomarker signature; and
(ii) an oligonucleotide domain coupled to the target binding moiety, the oligonucleotide domain comprising a double-stranded portion and a single-stranded overhang portion extended from one end of the oligonucleotide domain,
wherein the single-stranded overhang portions of the first and second detection probes are hybridized to each other.

198. The complex of claim 197, wherein when the at least one target biomarker is selected from one or more of the surface protein biomarkers, the selected surface protein biomarker(s) and the at least one extracellular vesicle-associated membrane-bound protein biomarker are different.

199. The complex of claim 196 or 197, wherein the extracellular vesicle-associated membrane-bound polypeptide biomarker is or comprises CLDN3, CLDN6, AQP5, CLDN16, EpCAM, FOLR1, LEMD1, LRRTM1, MUC16, CHODL, CDH6, HTR3A, SLC34A2, ALPL, BST2, CD24, MSLN, MUC1, PTGS1, ST14, sTn, TACSTD2, BCAM, CD74, LY6E, SLC2A1, CXCR4, DDR1, EFNB1, NOTCH3, PLXNB1, SPINT2, TNFRSF12A, and combinations thereof.

200. The complex of any one of claims 197-199, wherein the extracellular vesicle-associated membrane-bound polypeptide biomarker is or comprises a SLC34A2 polypeptide, a FOLR1 polypeptide, a MUC16 polypeptide, an EpCAM polypeptide, a CLDN3 polypeptide, a CLDN6 polypeptide, an ALPL polypeptide, a BST2 polypeptide, a MSLN polypeptide, a MUC1 polypeptide, a PTGS1 polypeptide, a sTn glycosylated polypeptide, a TACSTD2 polypeptide, and/or an LRRTM1 polypeptide.

201. The complex of any one of claims 197-200, wherein the target binding moiety of the at least two detection probes is each directed to the same target biomarker of the target biomarker signature.

202. The complex of claim 201, wherein the same target biomarker is or comprises MUC16.

203. The complex of claim 201, wherein the same target biomarker is or comprises FOLR1.

204. The complex of claim 201, wherein the same target biomarker is or comprises MSLN.

205. The complex of claim 201, wherein the same target biomarker is or comprises ALPL.

206. The complex of claim 201, wherein the same target biomarker is or comprises MUC1.

207. The complex of claim 201, wherein the same target biomarker is or comprises a sTn glycosylated polypeptide.

208. The complex of claim 201, wherein the same target biomarker is or comprises BST2.

209. The complex of claim 201, wherein the same target biomarker is or comprises PTGS1.

210. The complex of claim 201, wherein the same target biomarker is or comprises SLC34A2.

211. The complex of claim 201, wherein the same target biomarker is or comprises TACSTD2.

212. The complex of any one of claims 201-211, wherein the oligonucleotide domain of the at least two detection probes are different.

213. The complex of any one of claims 197-200, wherein the target binding moiety of the at least two detection probes is each directed to a distinct target biomarker of the target biomarker signature.

214. The complex of claim 213, wherein the target binding moiety of the at least two detection probes are directed to MUC16 and FOLR1 respectively.

215. The complex of claim 213, wherein the target binding moiety of the at least two detection probes are directed to MUC16 and CLDN6 respectively.

216. The complex of claim 213, wherein the target binding moiety of the at least two detection probes are directed to FOLR1 and CLDN6 respectively.

217. The complex of claim 213, wherein the target binding moiety of the at least two detection probes are directed to SLC34A2 and CLDN3 respectively.

218. The complex of claim 213, wherein the target binding moiety of the at least two detection probes are directed to SLC34A2 and MUC16 respectively.

219. The complex of claim 213, wherein the target binding moiety of the at least two detection probes are directed to CLDN3 and MUC16 respectively.

220. The complex of claim 213, wherein the target binding moiety of the at least two detection probes are directed to CLDN3 and FOLR1 respectively.

221. The complex of claim 213, wherein the target binding moiety of the at least two detection probes are directed to ALPL and FOLR1 respectively.

222. The complex of claim 213, wherein the target binding moiety of the at least two detection probes are directed to BST2 and MUC16 respectively.

223. The complex of claim 213, wherein the target binding moiety of the at least two detection probes are directed to FOLR1 and MSLN respectively.

224. The complex of claim 213, wherein the target binding moiety of the at least two detection probes are directed to FOLR1 and MUC16 respectively.

225. The complex of claim 213, wherein the target binding moiety of the at least two detection probes are directed to MSLN and MUC16 respectively.

226. The complex of claim 213, wherein the target binding moiety of the at least two detection probes are directed to MSLN and MUC1 respectively.

227. The complex of claim 213, wherein the target binding moiety of the at least two detection probes are directed to a sTn glycosylated polypeptide and MUC1 respectively.

228. The complex of claim 213, wherein the target binding moiety of the at least two detection probes are directed to MUC1 and MUC16 respectively.

229. The complex of claim 213, wherein the target binding moiety of the at least two detection probes are directed to MUC1 and FOLR1 respectively.

230. The complex of claim 213, wherein the target binding moiety of the at least two detection probes are directed to a sTn glycosylated polypeptide and MUC16 respectively.

231. The complex of claim 213, wherein the target binding moiety of the at least two detection probes are directed to PTGS1 and MUC16 respectively.

232. The complex of claim 213, wherein the target binding moiety of the at least two detection probes are directed to SLC34A2 and FOLR1 respectively.

233. The complex of claim 213, wherein the target binding moiety of the at least two detection probes are directed to a sTn glycosylated polypeptide and FOLR1 respectively.

234. The complex of claim 213, wherein the target binding moiety of the at least two detection probes are directed to a sTn glycosylated polypeptide and MSLN respectively.

235. The complex of claim 213, wherein the target binding moiety of the at least two detection probes are directed to TACSTD2 and MUC16 respectively.

236. The complex of claim 213, wherein the target binding moiety of the at least two detection probes are directed to a sTn glycosylated polypeptide and TACSTD2 respectively.

237. The complex of any one of claims 197-236, wherein the solid substrate comprises a magnetic bead.

238. The complex of any one of claims 197-236, wherein the target-capture moiety is or comprises an antibody agent.
